(19) United States
(12) Patent Application Publication
(10) Pub. No.: US 2025/0136961 A1
(43) Pub. Date: May 1, 2025

(54) ISOLATED NUCLEASE AND USE THEREOF

(71) Applicant: Beijing AstraGenomics Technology Co., Ltd., Beijing (CN)

(72) Inventors: Daqi YU, Beijing (CN); Ting WEI, Beijing (CN); Yansha LI, Beijing (CN); Chen ZHAO, Beijing (CN); Chengxi SHI, Beijing (CN)

(21) Appl. No.: 18/871,257

(22) PCT Filed: Mar. 22, 2024
(86) PCT No.: PCT/CN2024/083343

§ 371 Date: Dec. 3, 2024

(30) Foreign Application Priority Data
Mar. 27, 2023 (CN) 202310304837.4
Nov. 29, 2023 (WO) PCT/CN2023/135175

(51) Int. Cl. C12N 9/22; C12N 15/11; C12N 15/85

(52) U.S. Cl. CPC C12N 9/22; C12N 15/111; C12N 15/85; C12N 2310/20; C12N 2830/50

(57) ABSTRACT

It relates to the field of molecular biology, and specifically to an isolated nuclease and the use thereof. It further specifically relates to: a nucleic acid and a nucleic acid construct encoding the nuclease, a guide RNA and a nucleic acid construct thereof, and a composition, a recombinant vector, a recombinant host cell and a kit comprising the nuclease. It further specifically relates to: a method for introducing a double-strand break into a targeting gene of a host cell, a method for deleting, replacing or inserting a targeting gene of a host cell, and a method for obtaining a host cell in which a targeting gene is deleted, replaced or inserted.

### CROSS-REFERENCE TO RELATED APPLICATIONS

This application claims priority to Chinese Patent Application No. 202310304837.4 filed on Mar. 27, 2023, and PCT Application No. PCT/CN2023/135175 filed on Nov. 29, 2023, the entire contents of which are hereby incorporated by reference in their entirety for all purpose.

### TECHNICAL FIELD

The present application relates to the field of molecular biology, and specifically to an isolated nuclease and the use thereof. The present application further specifically relates to: a nucleic acid and a nucleic acid construct encoding the nuclease, a guide RNA and a nucleic acid construct thereof, and a composition, a recombinant vector, a recombinant host cell and a kit comprising the nuclease. The present application further specifically relates to: a method for introducing a double-strand break into a targeting gene of a host cell, a method for deleting, replacing or inserting a targeting gene of a host cell, and a method for obtaining a host cell in which a targeting gene is deleted, replaced or inserted. The present application further specifically relates to the use of the nuclease, the nucleic acid and the nucleic acid construct encoding the nuclease, the guide RNA and the nucleic acid construct thereof, the composition, the recombinant vector, or the recombinant host cell for introducing a double-strand break into a targeting gene of a host cell, deleting, replacing or inserting a targeting gene of a host cell, and preparing a drug or a preparation for gene therapy, cell therapy, genome research, and stem cell induction and post-induction differentiation.

### BACKGROUND

With the rapid development of modern biotechnology and the advent of post-genome era, people are entering the stage of rewriting or even redesigning genetic information from the stage of reading biological genetic DNA information. The discovery of CRISPR/Cas9 technology has made a revolutionary breakthrough in gene editing technology. CRISPR/Cas9 is an RNA-mediated targeted gene editing tool, which can specifically recognize and cleave different endogenous DNA sequences through reprogramming of sgRNA. Cas9 has two nuclease domains, RuvC and HNH, which are responsible for the cleavage of either strand of DNA respectively. Mutating either of these sites can convert Cas9 into a single-strand Cas9 nickase. Important new technologies concerning Cas9, such as base editing and prime editing, are all designed based on Cas9 nickase.

However, some shortcomings of CRISPR/Cas9 limit its application: First, the CDS sequence of spCas9 has a length exceeding 4.1 Kb, which exceeds the maximum effective packaging capacity of adenovirus (AAV), and therefore it is difficult for the adenovirus-mediated gene delivery; although lentivirus has a stronger packaging capacity than AAV (with an upper loading limit of about 9 kb), the proportion of proteins in spCas9 is still too high, limiting the potential for subsequent engineering. These shortcomings seriously restrict the application of spCas9 in clinical medicine. Subsequently, CRISPR/Cas12 or 12f system with a smaller molecular weight appears, but the editing efficiency of proteins such as Cas12 is not superior to that of spCas9. Therefore, spCas9 is still widely accepted and used at present. Second, the PAM sequence of spCas9, which is the NGG sequence, is relatively simple and has a higher occurrence rate in the genome. Its advantage lies in the flexibility in reprograming sgRNA to complete the recognition and cleavage of different DNA sequences. However, this flexibility also leads to the off-target effects of suboptimal genome editing outcomes.

Therefore, gene editing technologies realized using RNA-mediated endonuclease, i.e., insertion sequences IscB and TnpB from IS200/IS605 family, appear subsequently. They are widely distributed in microorganisms and have a more compact protein structure, with a size of about 400 aa that is less than ⅓ of spCas9, so they have greater potential for engineering in terms of the application of enzymes. TnpB cleaves DNA next to the 5′ TTGAT transposon-associated motif (TAM) through reRNA (right element RNA, derived from RE element in ISDra2 transposon) mediation, thereby breaking and mutating the DNA sequence in the genome. The DNA cleavage function of TnpB needs to meet two conditions at the same time: (1) TAM sequence; (2) a sequence located at the 3′ end of reRNA that matches with a targeting gene. Different nucleases can recognize different TAM, and therefore the excavation of more highly active nuclease tools and the verification and detection of their functions can provide more, better and flexible choices for the development of gene editing strategies.

It should be noted that methods described in this section are not necessarily methods that have been previously conceived or employed. It should not be assumed that any of the methods described in this section is considered to be the prior art just because they are included in this section, unless otherwise indicated expressly. Similarly, the problem mentioned in this section should not be considered to be universally recognized in any prior art, unless otherwise indicated expressly.

### SUMMARY

In order to solve the above problems, the present application is intended to find RNA-mediated endonucleases having a suitable protein molecular weight and good gene editing effects, and provide more diverse and specific tools for gene editing.

The present application provides an isolated nuclease, wherein the nuclease comprises an amino acid sequence as shown in the following formula:

(X₁)(X₂)ₐ(X₃)(X₄)(X₅)_{b}(X₆)(X₇)_{c}(X₈)(X₉)_{d}(X₁₀)(X₁₁)ₑ(X₁₂)(X₁₃)_{f}(X₁₄)(X₁₅)_{g}(X₁₆)
wherein a, b, c, d, e, f, and g are the numbers of amino acids; (X₁), (X₃), (X₄), (X₆), (X₈), (X₁₀), (X₁₂), (X₁₄), and (X₁₆) are independently polar amino acids or aliphatic amino acids; (X₂) is any amino acid, and a is 15 or 16; (X₅) is any amino acid, and b is 2; (X₇) is any amino acid, and c is 2, 3 or 4; (X₉) is any amino acid, and d is 14, 15, 16, 17 or 18; (X₁₁) is any amino acid, and e is 1 or 2; (X₁₃) is any amino acid, and f is 6; and (X₁₅) is any amino acid, and g is 5.

According to an embodiment of the present application, an isolated nuclease can be provided, wherein the nuclease has a nuclease sequence selected from the following (i) or a variant sequence of the aforementioned nuclease having a nuclease activity in (ii)-(iv): (i) at least one amino acid sequence as shown in any one of SEQ ID NOs: 1-197; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-197; (iii) at least one of amino acid sequences having at least 70%, 80%, 90%, 95% or 99% identity to the amino acid sequence as shown in any one of SEQ ID NOs: 1-197; and (iv) at least one of sequences obtained by further fusing the amino acid sequence as shown in any one of SEQ ID NOs: 1-197 with other sequences.

According to an embodiment of the present application, a guide RNA can be provided, wherein the guide RNA comprises a reRNA, the reRNA comprises a nucleotide sequence as shown in any one of SEQ ID NOs: 198-394 or a variant thereof, and the guide RNA can bind to a specific nuclease.

According to an embodiment of the present application, a nucleic acid can be provided, wherein, the nucleic acid encodes the nuclease described in the present application and/or the guide RNA described in the present application.

According to an embodiment of the present application, a nucleic acid construct can be provided, comprising the nucleic acid described in the present application, and further comprising a promoter.

According to an embodiment of the present application, a composition may be provided, wherein, the composition includes: an IS200/IS605 family nuclease or a functional fragment thereof, or comprises a nucleic acid encoding the IS200/IS605 family nuclease or the functional fragment thereof, and the nuclease or the functional fragment thereof has endonuclease activity; and a guide RNA, or comprises a nucleic acid encoding the guide RNA, and the guide RNA can bind to a specific nuclease.

According to an embodiment of the present application, a recombinant vector can be provided, wherein, the recombinant vector comprises the nucleic acid encoding the nuclease described in the present application, the guide RNA described in the present application, the nucleic acid described in the present application, the nucleic acid construct described in the present application, or the composition described in the present application.

According to an embodiment of the present application, a recombinant host cell can be provided, wherein, the recombinant host cell comprises the nuclease described in the present application, the guide RNA described in the present application, the nucleic acid described in the present application, the nucleic acid construct described in the present application, the composition described in the present application, or the recombinant vector described in the present application.

According to an embodiment of the present application, a method for introducing a double-strand break into a targeting gene of a host cell can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

According to an embodiment of the present application, a method for deleting, replacing or inserting a targeting gene of a host cell can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

According to an embodiment of the present application, a method for obtaining a host cell in which a targeting gene is deleted, replaced or inserted can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for introducing a double-strand break into a targeting gene of a host cell can be provided.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for deleting, replacing or inserting a targeting gene of a host cell can be provided.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for preparing a drug or a preparation for gene therapy, cell therapy, genome research, and stem cell induction and post-induction differentiation can be provided.

According to an embodiment of the present application, a kit can be provided, wherein, the kit comprises the nuclease described in the present application, the guide RNA described in the present application, the nucleic acid described in the present application, the nucleic acid construct described in the present application, the composition described in the present application, the recombinant vector described in the present application, or the recombinant host cell described in the present application.

The protein molecular weight of the nuclease described in the present application is far less than that of spCas9, about less than one third of the latter, which provides more possibilities for a variety of in vivo delivery in subsequent gene therapy, and can solve the problem of having difficulty in the delivery of spCas9 caused by the protein size; and compared with asCas 12 which also has a low protein molecular weight, the nuclease has higher gene editing efficiency, which provides the possibility of same becoming a new gene editing application tool; additionally, since different nucleases can recognize different transposon-associated motifs, the novel nuclease discovered in the present application brings more choices for subsequent application scenarios of different scales.

It should be understood that the content described in this section is not intended to identify critical or important features of the examples of the present application and is not used to limit the scope of the present application. Other features of the present application will be easily understood through the following description.

### BRIEF DESCRIPTION OF THE DRAWINGS

The accompanying drawings exemplarily show embodiments and form a part of the specification, and are used to explain exemplary implementations of the embodiments together with a written description of the specification. The embodiments shown are merely for illustrative purposes and do not limit the scope of the claims. Throughout the accompanying drawings, the same reference numerals denote similar but not necessarily same elements.

FIG. 1 shows a schematic diagram of an RGS dual fluorescence surrogate reporter system in example 1.

FIG. 2 shows flow cytometry plots with percentages of mRFP+eGFP+ cells presented at the top-right Q2 gate.

FIG. 3 shows GFP expression for all the active nuclease candidates of TP_A_1, TP_A_2, TP_A_8, TP_A_12, TP_A_18, TP_B_18, TP_B_41, TP_B_46, TP_B_70, TP_B_71, TP_B_72, TP_B_73, TP_C_23, TP_C_67, TP_C_70, TP_C_74, TP_D_1, TP_D_3, TP_D_4, TP_D_8, TP_D_17, TP_D_18, TP_D_23, TP_D_24, TP_D_25, TP_D_27, TP_D_30, TP_D_32, TP_D_40, TP_D_43, TP_D_51, TP_D_59, TP_D_61, TP_D_66, TP_D_67, TP_D_71, TP_D_72, TP_D_73, TP_E_2, TP_E_15, TP_E_17, TP_E_48, TP_F_56, TP_F_71, TP_F_77, TP_F_80, TP_F_83, TP_F_85, TP_G_14, TP_G_19, TP_G_20, TP_G_24, TP_G_43, TP_G_52, TP_G_53, TP_G_61, TP_G_66, TP_G_72, TP_G_75, TP_G_83, TP_G_84, TP_H_1, TP_H_3, TP_H_4, TP_H_5, TP_H_6, TP_H_9, TP_H_11, TP_H_12, TP_H_13, TP_H_15, TP_H_18, TP_H_19, TP_H_20, TP_H_21, TP_H_23, TP_H_24, TP_H_30, TP_H_31, TP_H_32, TP_H_34, TP_H_38, TP_H_39, TP_H_40, TP_H_43, TP_I_1, TP_I_2, TP_I_3, TP_I_4, TP_I_5, TP_I_6, TP_I_7, TP_I_8, TP_I_9, TP_I_10, TP_I_11, TP_I_12, TP_I_13, TP_I_15, TP_I_16, TP_I_17, TP_I_18, TP_I_19, TP_I_20, TP_I_21, TP_I_22, TP_I_24, TP_I_25, TP_I_26, TP_I_29, TP_I_31, TP_I_35, TP_I_37, TP_I_38, TP_I_40, TP_I_41, TP_I_44, TP_I_45, TP_I_46, TP_I_47, TP_I_48, TP_I_49, TP_I_50, TP_I_51, TP_I_52, TP_I_53, TP_I_55, TP_I_56, TP_I_58, TP_I_59, TP_I_61, TP_I_62, TP_I_64, TP_I_65, TP_I_66, TP_I_67, TP_I_70, TP_I_71, TP_I_76, TP_I_77, TP_I_79, TP_I_80, TP_I_82, TP_I_84, TP_I_85, TP_I_86, TP_I_87, TP_L_1, TP_L_4, TP_L_5, TP_L_8, TP_L_9, TP_L_10, TP_L_11, TP_L_12, TP_L_15, TP_L_16, TP_L_17, TP_L_21, TP_L_22, TP_L_24, TP_L_25, TP_L_26, TP_L_27, TP_L_28, TP_L_31, TP_L_32, TP_L_34, TP_L_36, TP_L_37, TP_L_39, TP_M_1, TP_M_3, TP_M_7, TP_M_11, TP_M_14, TP_M_17, TP_M_19, TP_M_20, TP_M_24, TP_M_31, TP_M_32, TP_M_33, TP_M_34, TP_M_35, TP_M_37, TP_M_40, TP_M_41, TP_M_43, TP_M_46, TP_M_49, TP_M_58, TP_M_65, TP_M_66, TP_M_67, TP_M_70 and TP_M_78. All the results were quantified by flow cytometry assay as previous shown in example 2.

FIG. 4, FIG. 5, FIG. 6, FIG. 7 and FIG. 8 are the partial enlarged pictures of FIG. 3.

FIG. 9 shows endogenous editing efficiency (quantified by the proportion of reads with insertions or deletions at target site) of TP_C_23, TP_D_51, TP_D_67, TP_E_15, TP_F_85, TP_G_24, TP_H_5, TP_H_6, TP_H_9, TP_H_11, TP_H_24, TP_H_30, TP_H_32, TP_H_34, TP_H_38, TP_I_1, TP_I_5, TP_I_6, TP_I_12, TP_I_15, TP_I_18, TP_I_20, TP_I_38, TP_I_49, TP_I_64 and TP_I_79 in example 3.

FIG. 10 shows an evolutionary branching diagram of TP_A_1, TP_A_2, TP_A_8, TP_A_12, TP_A_18, TP_B_18, TP_B_41, TP_B_46, TP_B_70, TP_B_71, TP_B_72, TP_B_73, TP_C_23, TP_C_67, TP_C_70, TP_C_74, TP_D_1, TP_D_3, TP_D_4, TP_D_8, TP_D_17, TP_D_18, TP_D_23, TP_D_24, TP_D_25, TP_D_27, TP_D_30, TP_D_32, TP_D_40, TP_D_43, TP_D_51, TP_D_59, TP_D_61, TP_D_66, TP_D_67, TP_D_71, TP_D_72, TP_D_73, TP_E_2, TP_E_15, TP_E_17, TP_E_48, TP_F_56, TP_F_71, TP_F_77, TP_F_80, TP_F_83, TP_F_85, TP_G_14, TP_G_19, TP_G_20, TP_G_24, TP_G_43, TP_G_52, TP_G_53, TP_G_61, TP_G_66, TP_G_72, TP_G_75, TP_G_83, TP_G_84, TP_H_1, TP_H_3, TP_H_4, TP_H_5, TP_H_6, TP_H_9, TP_H_11, TP_H_12, TP_H_13, TP_H_15, TP_H_18, TP_H_19, TP_H_20, TP_H_21, TP_H_23, TP_H_24, TP_H_30, TP_H_31, TP_H_32, TP_H_34, TP_H_38, TP_H_39, TP_H_40, TP_H_43, TP_I_1, TP_I_2, TP_I_3, TP_I_4, TP_I_5, TP_I_6, TP_I_7, TP_I_8, TP_I_9, TP_I_10, TP_I_11, TP_I_12, TP_I_13, TP_I_15, TP_I_16, TP_I_17, TP_I_18, TP_I_19, TP_I_20, TP_I_21, TP_I_22, TP_I_24, TP_I_25, TP_I_26, TP_I_29, TP_I_31, TP_I_35, TP_I_37, TP_I_38, TP_I_40, TP_I_41, TP_I_44, TP_I_45, TP_I_46, TP_I_47, TP_I_48, TP_I_49, TP_I_50, TP_I_51, TP_I_52, TP_I_53, TP_I_55, TP_I_56, TP_I_58, TP_I_59, TP_I_61, TP_I_62, TP_I_64, TP_I_65, TP_I_66, TP_I_67, TP_I_70, TP_I_71, TP_I_76, TP_I_77, TP_I_79, TP_I_80, TP_I_82, TP_I_84, TP_I_85, TP_I_86, TP_I_87, TP_L_1, TP_L_4, TP_L_5, TP_L_8, TP_L_9, TP_L_10, TP_L_11, TP_L_12, TP_L_15, TP_L_16, TP_L_17, TP_L_21, TP_L_22, TP_L_24, TP_L_25, TP_L_26, TP_L_27, TP_L_28, TP_L_31, TP_L_32, TP_L_34, TP_L_36, TP_L_37, TP_L_39, TP_M_1, TP_M_3, TP_M_7, TP_M_11, TP_M_14, TP_M_17, TP_M_19, TP_M_20, TP_M_24, TP_M_31, TP_M_32, TP_M_33, TP_M_34, TP_M_35, TP_M_37, TP_M_40, TP_M_41, TP_M_43, TP_M_46, TP_M_49, TP_M_58, TP_M_65, TP_M_66, TP_M_67, TP_M_70, TP_M_78 and ISDra2 based on protein sequences in example 2.

FIG. 11 and FIG. 12 are the partial enlarged pictures of FIG. 10.

FIG. 13 shows a schematic diagram illustrating the order of elements in the report vectors in example 4. FIG. 14 shows a schematic diagram demonstrating how the reporter vectors work in example 4.

FIGS. 15, 16, 17, 18, 19, 20, 21, 22, 23, 24, 25, 26, 27, 28, 29, 30, and 31 show YFP expression in example 4 for all the active nuclease candidates of TP_A_1, TP_A_2, TP_A_8, TP_A_12, TP_A_18, TP_B_18, TP_B_41, TP_B_46, TP_B_70, TP_B_71, TP_B_72, TP_B_73, TP_C_23, TP_C_67, TP_C_70, TP_C_74, TP_D_1, TP_D_3, TP_D_4, TP_D_8, TP_D_17, TP_D_18, TP_D_23, TP_D_24, TP_D_25, TP_D_27, TP_D_30, TP_D_32, TP_D_40, TP_D_43, TP_D_51, TP_D_59, TP_D_61, TP_D_66, TP_D_67, TP_D_71, TP_D_72, TP_D_73, TP_E_2, TP_E_15, TP_E_17, TP_E_48, TP_F_56, TP_F_71, TP_F_77, TP_F_80, TP_F_83, TP_F_85, TP_G_14, TP_G_19, TP_G_20, TP_G_24, TP_G_43, TP_G_52, TP_G_53, TP_G_61, TP_G_66, TP_G_72, TP_G_75, TP_G_83, TP_G_84, TP_H_1, TP_H_3, TP_H_4, TP_H_5, TP_H_6, TP_H_9, TP_H_11, TP_H_12, TP_H_13, TP_H_15, TP_H_18, TP_H_19, TP_H_20, TP_H_21, TP_H_23, TP_H_24, TP_H_30, TP_H_31, TP_H_32, TP_H_34, TP_H_38, TP_H_39, TP_H_40, TP_H_43, TP_I_1, TP_I_2, TP_I_3, TP_I_4, TP_I_5, TP_I_6, TP_I_7, TP_I_8, TP_I_9, TP_I_10, TP_I_11, TP_I_12, TP_I_13, TP_I_15, TP_I_16, TP_I_17, TP_I_18, TP_I_19, TP_I_20, TP_I_21, TP_I_22, TP_I_24, TP_I_25, TP_I_26, TP_I_29, TP_I_31, TP_I_35, TP_I_37, TP_I_38, TP_I_40, TP_I_41, TP_I_44, TP_I_45, TP_I_46, TP_I_47, TP_I_48, TP_I_49, TP_I_50, TP_I_51, TP_I_52, TP_I_53, TP_I_55, TP_I_56, TP_I_58, TP_I_59, TP_I_61, TP_I_62, TP_I_64, TP_I_65, TP_I_66, TP_I_67, TP_I_70, TP_I_71, TP_I_76, TP_I_77, TP_I_79, TP_I_80, TP_I_82, TP_I_84, TP_I_85, TP_I_86, TP_I_87, TP_L_1, TP_L_4, TP_L_5, TP_L_8, TP_L_9, TP_L_10, TP_L_11, TP_L_12, TP_L_15, TP_L_16, TP_L_17, TP_L_21, TP_L_22, TP_L_24, TP_L_25, TP_L_26, TP_L_27, TP_L_28, TP_L_31, TP_L_32, TP_L_34, TP_L_36, TP_L_37, TP_L_39, TP_M_1, TP_M_3, TP_M_7, TP_M_11, TP_M_14, TP_M_17, TP_M_19, TP_M_20, TP_M_24, TP_M_31, TP_M_32, TP_M_33, TP_M_34, TP_M_35, TP_M_37, TP_M_40, TP_M_41, TP_M_43, TP_M_46, TP_M_49, TP_M_58, TP_M_65, TP_M_66, TP_M_67, TP_M_70 and TP_M_78.

### DETAILED DESCRIPTION OF EMBODIMENTS

Unless otherwise indicated or contradicts the context, the terms or expressions used herein should be read in conjunction with the entire content of the present disclosure and as understood by those of ordinary skill in the art. All technical and scientific terms used herein have the same meanings as commonly understood by those of ordinary skill in the art, unless otherwise defined.

In the present application, the terms “nucleic acid” and “polynucleotide” are used interchangeably, and refer to polymerization forms of nucleotides of any length, including deoxyribonucleotides, ribonucleotides, combinations thereof, and analogs thereof.

In the present application, the terms “polypeptide” and “peptide” are used interchangeably, and refer to polymers of amino acids of any length. Therefore, polypeptides, oligopeptides, proteins, antibodies and enzymes are all included in the definition of polypeptide.

As described in the present application, the “fragment” of a sequence refers to a portion of a sequence. For example, the fragment of a nucleic acid sequence refers to a portion of the nucleic acid sequence, and the fragment of an amino acid sequence refers to a portion of the amino acid sequence.

As described in the present application, a “variant” of a sequence is a polynucleotide or polypeptide that differs from a reference polynucleotide or polypeptide, respectively, but retains essential properties. A typical variant of a polynucleotide differs in nucleic acid sequence from another reference polynucleotide, and the differences in nucleic acid sequence may or may not alter the amino acid sequence of the polypeptide encoded by the reference polynucleotide. A typical variant of a polypeptide differs in amino acid sequence from another reference polypeptide. Generally, the differences are limited so that the sequences of the reference polypeptide and the variant are generally very similar, and are identical in many regions. A variant polypeptide and a reference polypeptide may differ in amino acid sequence by one or more substitutions, additions, deletions in any combination. The substituted or inserted amino acid residue may or may not be a residue encoded by the genetic code. Variants of polynucleotides or polypeptides may be naturally occurring, such as allelic variations, or they may be unknown naturally occurring variants. Non-naturally occurring polynucleotide and polypeptide variants can be produced by mutagenesis techniques, direct synthesis, and other recombinant methods known to the skilled artisan.

Amino acids are usually classified by the properties of their side chains. For example, side chains may render amino acids weakacids (e.g., amino acids D and E) or weak bases (e.g., amino acids K, R and H); and if the side chains are polar, the amino acids become hydrophilic (e.g., amino acids L and I), or if the side chains are nonpolar, the amino acids become hydrophobic (e.g., amino acids S and C).

As described in the present application, the “aliphatic amino acid” has a side chain that is an aliphatic group. Aliphatic groups cause amino acids to be nonpolar and hydrophobic. The aliphatic group is preferably an unsubstituted branched or linear alkyl group. Non-limiting examples of the aliphatic amino acids are A (alanine), V (valine), L (leucine), I (isoleucine), M (methionine), D (aspartic acid), E (glutamic acid), K (lysine), R (arginine), G (glycine), S (serine), T (threonine), C (cysteine), N (asparagine), and Q (glutamine).

As described in the present application, the “nonpolar amino acid” has a nonpolar side chain that makes the amino acid hydrophobic. Non-limiting examples of the nonpolar amino acid are A (alanine), V (valine), L (leucine), I (isoleucine), F (phenylalanine), W (tryptophan), M (methionine), P (proline), and G (glycine).

As described in the present application, the “polar amino acid” has a polar side chain that makes the amino acid hydrophilic. Non-limiting examples of the polar amino acid are T (threonine), S (serine), C (cysteine), N (asparagine), Q (glutamine), Y (tyrosine), K (lysine), R (arginine), H (histidine), D (aspartic acid), and E (glutamic acid). Polar amino acids can be divided into polar uncharged amino acids or polar charged amino acids.

As described in the present application, the “polar uncharged amino acid” has a polar side chain of uncharged residues. Non-limiting examples of the polar uncharged amino acid are T (threonine), S (serine), C (cysteine), N (asparagine), Q (glutamine), and Y (tyrosine).

As described in the present application, the “polar charged amino acid” has a polar side chain of at least one charged residue. Non-limiting examples of the polar charged amino acid are K (lysine), R (arginine), H (histidine), D (aspartic acid), and E (glutamic acid). Polar charged amino acids can be divided into positively charged amino acids or negatively charged amino acids.

As described in the present application, the “positively charged amino acid” has a polar side chain of at least one positively charged residue. Non-limiting examples of the positively charged amino acid are K (lysine), R (arginine), and H (histidine).

As described in the present application, the “negatively charged amino acid” has a polar side chain of at least one negatively charged residue. Non-limiting examples of the negatively charged amino acid are D (aspartic acid), and E (glutamic acid).

The term “family” as used in the present application refers to a group of nucleic acids or proteins having high structural similarity produced by the same ancestor by means of replication and variation, which usually have related or even the same functions.

The term “nuclease” described in the present application refers to an enzyme capable of cleaving phosphodiester bonds. Nucleases hydrolyze the phosphodiester bonds in the backbone of nucleic acids. The term “endonuclease” described in the present application refers to an enzyme capable of cleaving phosphodiester bonds between nucleotides.

The term “guide RNA” described in the present application refers to any RNA molecule that can form a complex with the nuclease described in the present application. For example, the guide RNA can be a molecule that recognizes a targeting gene. In some embodiments of the present application, the guide RNA comprises a reRNA and a targeted sequence, wherein the reRNA can bind to a particular nuclease, and the targeted sequence can be designed to be complementary to a target strand of a targeting gene.

The term “transposon-associated motif” (TAM) described in the present application refers to a short nucleotide sequence adjacent to a targeting gene, which sequence can be recognized by a complex formed by nuclease and guide RNA described in the present application. If a targeting gene is not adjacent to a transposon-associated motif, the nuclease cannot successfully recognize the targeting gene. Sequences and lengths of the transposon-associated motif in the present application can vary depending on the nuclease.

The terms “targeting gene” “targeting sequence” “targeting nucleic acid” “gene of interest”, “sequence of interest” and “nucleic acid of interest” described in the present application are used interchangeably, and refer to nucleotide sequences on chromosomal DNA, chloroplast DNA, mitochondrial DNA, plasmid DNA, or any other DNA molecule in the genome of cells, which sequences can be recognized, bound to, and selectively cleaved by a complex formed by the nuclease and guide RNA described in the present application.

The term “nucleic acid construct” as used in the present application is defined as a single-stranded or double-stranded nucleic acid molecule herein, and preferably refers to an artificially constructed nucleic acid molecule. Optionally, the nucleic acid construct further includes one or more operably linked regulatory sequences, which can direct the expression of a coding sequence in a suitable host cell under compatible conditions. The term “expression” is understood to include any step involved in the production of a protein or polypeptide, including, but not limited to, transcription, post-transcriptional modification, translation, post-translational modification and secretion. The term “regulatory sequence” includes all components necessary or advantageous for expression of the polypeptide/protein of the present application. Each regulatory sequence may be naturally present or exogenous to the nucleic acid sequence encoding the protein or polypeptide. These regulatory sequences include, but are not limited to, leader sequences, polyadenylation sequences, propeptide sequences, promoters, signal sequences, and transcription terminators. At a minimum, the regulatory sequences should include promoters and termination signals for transcription and translation. Regulatory sequences with linkers can be provided for the purpose of introduction into specific restriction sites for linking the regulatory sequences to the coding region of a nucleic acid sequence encoding a protein or polypeptide.

The term “promoter” as used in the present application refers to a polynucleotide sequence that can control the transcription of a coding sequence. Promoter sequences include specific sequences sufficient to enable RNA polymerase to recognize, bind, and initiate transcription. In addition, promoter sequences may include sequences that optionally modulate the recognition, binding and transcription initiation activities of RNA polymerase in the nucleic acid construct provided in the present application. A promoter can affect the transcription of a gene located on the same nucleic acid molecule as the promoter or a gene located on a different nucleic acid molecule from the promoter.

The term “host cell” as used in the present application include, but are not limited to, an animal cell, a plant cell, an algal cell, a fungal cell, a yeast cell, or a bacterial cell. This term includes a progeny of an original cell into which an exogenous nucleic acid fragment has been introduced. Exemplary host cell includes human embryonic kidney cell HEK293T. It is understood that, due to natural, accidental or intentional mutations, the progeny of a single parent cell may not necessarily be identical to the original parent morphologically or in terms of genome or total DNA complement.

The term “vector” as used in the present application refers to a nucleic acid molecule capable of transporting another nucleic acid molecule connected to it. Examples of vectors include, but are not limited to, plasmids, viruses, bacteria, phages, and insertable DNAfragments. The term “plasmid” refers to a circular double-stranded DNA capable of accepting an exogenous nucleic acid fragment and replicating in prokaryotic or eukaryotic cells.

### Nuclease

The present application provides an isolated nuclease, wherein the nuclease comprises an amino acid sequence as shown in the following formula:

(X₁)(X₂)ₐ(X₃)(X₄)(X₅)_{b}(X₆)(X₇)_{c}(X₈)(X₉)_{d}(X₁₀)(X₁₁)ₑ(X₁₂)(X₁₃)_{f}(X₁₄)(X₁₅)(X₁₆)_{g}
wherein a, b, c, d, e, f, and g are the numbers of amino acids; (X₁), (X₃), (X₄), (X₆), (X₈), (X₁₀), (X₁₂), (X₁₄), and (X₁₆) are independently polar amino acids or aliphatic amino acids; (X₂) is any amino acid, and a is 15 or 16; (X₅) is any amino acid, and b is 2; (X₇) is any amino acid, and c is 2, 3 or 4; (X₉) is any amino acid, and d is 14, 15, 16, 17 or 18; (X₁₁) is any amino acid, and e is 1 or 2; (X₁₃) is any amino acid, and f is 6; and (X₁₅) is any amino acid, and g is 5.

In some embodiments, the (X₁) is a positively charged amino acid; (X₃) is a polar uncharged amino acid; (X₄) is a polar uncharged amino acid; (X₆) is a polar uncharged amino acid; (X₈) is a polar uncharged amino acid; (X₁₀) is a polar uncharged amino acid; (X₁₂) is a polar uncharged amino acid; (X₁₄) is a negatively charged amino acid; and (X₁₆) is a polar uncharged amino acid. In some embodiments, the (X₁) is K. In some embodiments, the (X₃) is S or T. In some embodiments, the (X₄) is S or T. In some embodiments, the (X₆) is C. In some embodiments, the (X₈) is C. In some embodiments, the (X₁₀) is C. In some embodiments, the (X₁₂) is C. In some embodiments, the (X₁₄) is D. In some embodiments, the (X₁₆) is N.

According to an embodiment of the present application, an isolated nuclease can be provided, wherein the nuclease has a nuclease sequence selected from the following (i) or a variant sequence of the aforementioned nuclease having a nuclease activity in (ii)-(iv): (i) at least one amino acid sequence as shown in any one of SEQ ID NOs: 1-197; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-197; (iii) at least one of amino acid sequences having at least 70%, 80%, 90%, 95% or 99% identity to the amino acid sequence as shown in any one of SEQ ID NOs: 1-197; and (iv) at least one of sequences obtained by further fusing the amino acid sequence as shown in any one of SEQ ID NOs: 1-197 with other sequences.

In some embodiments, the nuclease has a nuclease sequence selected from at least one of the following groups (1)-(9): (1) at least one amino acid sequence as shown in any one of SEQ ID NOs: 52 and 113-147; (2) at least one amino acid sequence as shown in any one of SEQ ID NOs: 27-28, 36-38, 62-85 and 148-171; (3) at least one amino acid sequence as shown in any one of SEQ ID NOs: 13, 86-100 and 105-110; (4) at least one amino acid sequence as shown in any one of SEQ ID NOs: 10-11, 17-19, 29-30 and 174-180; (5) at least one amino acid sequence as shown in any one of SEQ ID NOs: 34, 35, 50, 61 and 181-189; (6) at least one amino acid sequence as shown in any one of SEQ ID NOs: 53 and 190-197; (7) at least one amino acid sequence as shown in any one of SEQ ID NOs: 101, 103, 104 and 112; (8) at least one amino acid sequence as shown in any one of SEQ ID NOs: 7 and 23-25; and (9) at least one amino acid sequence as shown in any one of SEQ ID NOs: 3, 21 and 22.

In some embodiments, the nuclease has a nuclease sequence selected from at least one of the following groups (1)-(12): (1) at least one amino acid sequence as shown in any one of SEQ ID NOs: 1, 3-4, 6-7, 21-23, 50, 52, 60-61 and 113-147; (2) at least one amino acid sequence as shown in any one of SEQ ID NOs: 14, 27-28, 36-38, 45-48, 59, 62-85 and 148-171; (3) at least one amino acid sequence as shown in any one of SEQ ID NOs: 13, 43 and 86-112; (4) at least one amino acid sequence as shown in any one of SEQ ID NOs: 15-16, 24-25, 32-35 and 181-189; (5) at least one amino acid sequence as shown in any one of SEQ ID NOs: 9, 11, 17-19, 29 and 174-180; (6) at least one amino acid sequence as shown in any one of SEQ ID NOs: 10, 12, 26, 30, 42 and 58; (7) at least one amino acid sequence as shown in any one of SEQ ID NOs: 2, 20 and 31; (8) at least one amino acid sequence as shown in any one of SEQ ID NOs: 8 and 51; (9) at least one amino acid sequence as shown in any one of SEQ ID NOs: 39 and 49; (10) at least one amino acid sequence as shown in any one of SEQ ID NOs: 54 and 55; (11) at least one amino acid sequence as shown in any one of SEQ ID NOs: 53 and 190-197; and (12) at least one amino acid sequence as shown in any one of SEQ ID NOs: 5, 172 and 173.

In some embodiments, the nuclease belongs to the IS200/IS605 family. In some embodiments, the nuclease belongs to the IS605, or IS1341 subfamily. In some embodiments, the species sources of the nuclease include Bacteria or Archaea. In some embodiments, the species sources of the nuclease include Actinobacteria, Aquificae, Bacteroidetes, Candidatus Poribacteria, Chloroflexi, Cyanobacteria, Deinococcusthermus, Firmicutes, Planctomycetes, Proteobacteria, Spirochaetes, Tenericutes, Thermotogae, Verrucomicrobia, Candidatus Micrarchaeota, Crenarchaeota, or Euryarchaeota.

### Guide RNA

According to an embodiment of the present application, a guide RNA can be provided, wherein the guide RNA comprises a reRNA, the reRNA comprises a nucleotide sequence as shown in any one of SEQ ID NOs: 198-394 or a variant thereof, and the guide RNA can bind to a specific nuclease. In some embodiments, the reRNA comprises at least one of nucleotide sequences having at least 70%, 80%, 90%, 95% or 99% identity to the nucleotide sequence as shown in any one of SEQ ID NOs: 198-394. In some embodiments, the reRNA comprises at least one of the nucleotide sequences as shown in any one of SEQ ID NOs: 198-394. In some embodiments, the reRNA is at least one of the nucleotide sequences as shown in any one of SEQ ID NOs: 198-394.

In some embodiments, the guide RNA further comprises a targeted sequence that can recognize a targeting gene adjacent to a transposon-associated motif. In some embodiments, the targeted sequence is of at least one of 10-50, 10-40, 10-30, or 15-25 nucleotides in length.

Sequences and lengths of the transposon-associated motif in the present application can vary depending on the nuclease, and the transposon-associated motif can be recognized by a complex formed by the nuclease and guide RNA described in the present application. In some embodiments, the transposon-associated motif comprises a nucleotide sequence as shown in the following formula:

(X₁₇)ₕ(X₁₈)(X₁₉)A(X₂₀)
wherein h is the number of nucleotides; A is an adenine deoxyribonucleotide; (X₁₇) is any deoxyribonucleotide, and h is 0 or 1; (X₁₈) is a cytosine deoxyribonucleotide or thymine deoxyribonucleotide; (X₁₉) is a cytosine deoxyribonucleotide, thymine deoxyribonucleotide, or guanine deoxyribonucleotide; and (X₂₀) is any deoxyribonucleotide.

### Nucleicacid, Nucleicacid Construct

According to an embodiment of the present application, a nucleic acid can be provided, wherein, the nucleic acid encodes the nuclease described in the present application and/or the guide RNA described in the present application.

According to an embodiment of the present application, a nucleic acid construct can be provided, comprising the nucleic acid described in the present application. In some embodiments, the nucleic acid construct further comprising a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL.

In some embodiments, the nucleic acid construct is modified by 5′-end capping and/or 3′-end polyadenylating, and the nucleic acid construct retains the activity of nuclease and/or guide RNA. In some embodiments, the nucleic acid construct is modified by thiophosphate bond modification, 2′-MOE (2-O-(2-methoxyethyl)), PNA (peptide nucleic acid), GNA (glycerol nucleic acid), LNA (locked nucleic acid), GalNAc (N-acetylgalactosamine) LNP (lipid nano particle) PNP (peptide nanoparticles). The modification methods of nucleic acid are known in the art, the entire contents of which are hereby incorporated by reference.

In some embodiments, the nucleic acid construct further comprises a poly A sequence. Poly Atailing signal sequences well known in the art, as well as various truncated forms of polyA tailing signals, can be used in the present application.

In some embodiments, the nucleic acid construct further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid construct may further include a suitable leader sequence, that is, an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid construct may further include a propeptide coding region, which encodes an amino acid sequence located at the amino terminus of the polypeptide. The resulting polypeptide is called a zymogen or a propolypeptide. The propolypeptide is usually inactive and can be converted into a mature active polypeptide by catalytic or autocatalytic cleavage of the propeptide from the propolypeptide.

Optionally, the nucleic acid construct may further include a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Composition

According to an embodiment of the present application, a composition may be provided, wherein, the composition includes: an IS200/IS605 family nuclease or a functional fragment thereof, or comprises a nucleic acid encoding the IS200/IS605 family nuclease or the functional fragment thereof, and the nuclease or the functional fragment thereof has endonuclease activity; and a guide RNA, or comprises a nucleic acid encoding the guide RNA, and the guide RNA can bind to a specific nuclease.

In some embodiments, the composition is selected from at least one of the following groups (1)-(198), and any one of the following groups (1)-(198) comprises: a nuclease-related sequence and a guide RNA-related sequence,

(1) the nuclease-related sequence is an amino acid sequence comprising the sequence as shown in SEQ ID NO: 1 or a nucleic acid encoding the amino acid sequence; and the guide RNA-related sequence is a nucleotide sequence comprising the sequence as shown in SEQ ID NO: 198;

(2) the nuclease-related sequence is an amino acid sequence comprising the sequence as shown in SEQ ID NO: 2 or a nucleic acid encoding the amino acid sequence; and the guide RNA-related sequence is a nucleotide sequence comprising the sequence as shown in SEQ ID NO: 199;

(3) the nuclease-related sequence is an amino acid sequence comprising the sequence as shown in SEQ ID NO: 3 or a nucleic acid encoding the amino acid sequence; and the guide RNA-related sequence is a nucleotide sequence comprising the sequence as shown in SEQ ID NO: 200;

(4) the nuclease-related sequence is an amino acid sequence comprising the sequence as shown in SEQ ID NO: 4 or a nucleic acid encoding the amino acid sequence; and the guide RNA-related sequence is a nucleotide sequence comprising the sequence as shown in SEQ ID NO: 201;

(5) the nuclease-related sequence is an amino acid sequence comprising the sequence as shown in SEQ ID NO: 5 or a nucleic acid encoding the amino acid sequence; and the guide RNA-related sequence is a nucleotide sequence comprising the sequence as shown in SEQ ID NO: 202;

(6) the nuclease-related sequence is an amino acid sequence comprising the sequence as shown in SEQ ID NO: 6 or a nucleic acid encoding the amino acid sequence; and the guide RNA-related sequence is a nucleotide sequence comprising the sequence as shown in SEQ ID NO: 203;

(7) the nuclease-related sequence is an amino acid sequence comprising the sequence as shown in SEQ ID NO: 7 or a nucleic acid encoding the amino acid sequence; and the guide RNA-related sequence is a nucleotide sequence comprising the sequence as shown in SEQ ID NO: 204;

(8) the nuclease-related sequence is an amino acid sequence comprising the sequence as shown in SEQ ID NO: 8 or a nucleic acid encoding the amino acid sequence; and the guide RNA-related sequence is a nucleotide sequence comprising the sequence as shown in SEQ ID NO: 205;

(9) the nuclease-related sequence is an amino acid sequence comprising the sequence as shown in SEQ ID NO: 9 or a nucleic acid encoding the amino acid sequence; and the guide RNA-related sequence is a nucleotide sequence comprising the sequence as shown in SEQ ID NO: 206;

(10) the nuclease-related sequence is an amino acid sequence comprising the sequence as shown in SEQ ID NO: 10 or a nucleic acid encoding the amino acid sequence; and the guide RNA-related sequence is a nucleotide sequence comprising the sequence as shown in SEQ ID NO: 207;

(11) the nuclease-related sequence is an amino acid sequence comprising the sequence as shown in SEQ ID NO: 11 or a nucleic acid encoding the amino acid sequence; and the guide RNA-related sequence is a nucleotide sequence comprising the sequence as shown in SEQ ID NO: 208;

(12) the nuclease-related sequence is an amino acid sequence comprising the sequence as shown in SEQ ID NO: 12 or a nucleic acid encoding the amino acid sequence; and the guide RNA-related sequence is a nucleotide sequence comprising the sequence as shown in SEQ ID NO: 209;

(13) the nuclease-related sequence is an amino acid sequence comprising the sequence as shown in SEQ ID NO: 13 or a nucleic acid encoding the amino acid sequence; and the guide RNA-related sequence is a nucleotide sequence comprising the sequence as shown in SEQ ID NO: 210;

(14) the nuclease-related sequence is an amino acid sequence comprising the sequence as shown in SEQ ID NO: 14 or a nucleic acid encoding the amino acid sequence; and the guide RNA-related sequence is a nucleotide sequence comprising the sequence as shown in SEQ ID NO: 211;

(15) the nuclease-related sequence is an amino acid sequence comprising the sequence as shown in SEQ ID NO: 15 or a nucleic acid encoding the amino acid sequence; and the guide RNA-related sequence is a nucleotide sequence comprising the sequence as shown in SEQ ID NO: 212;

(16) the nuclease-related sequence is an amino acid sequence comprising the sequence as shown in SEQ ID NO: 16 or a nucleic acid encoding the amino acid sequence; and the guide RNA-related sequence is a nucleotide sequence comprising the sequence as shown in SEQ ID NO: 213;

(17) the nuclease-related sequence is an amino acid sequence comprising the sequence as shown in SEQ ID NO: 17 or a nucleic acid encoding the amino acid sequence; and the guide RNA-related sequence is a nucleotide sequence comprising the sequence as shown in SEQ ID NO: 214;

(18) the nuclease-related sequence is an amino acid sequence comprising the sequence as shown in SEQ ID NO: 18 or a nucleic acid encoding the amino acid sequence; and the guide RNA-related sequence is a nucleotide sequence comprising the sequence as shown in SEQ ID NO: 215;

(19) the nuclease-related sequence is an amino acid sequence comprising the sequence as shown in SEQ ID NO: 19 or a nucleic acid encoding the amino acid sequence; and the guide RNA-related sequence is a nucleotide sequence comprising the sequence as shown in SEQ ID NO: 216;

(20) the nuclease-related sequence is an amino acid sequence comprising the sequence as shown in SEQ ID NO: 20 or a nucleic acid encoding the amino acid sequence; and the guide RNA-related sequence is a nucleotide sequence comprising the sequence as shown in SEQ ID NO: 217;

(21) the nuclease-related sequence is an amino acid sequence comprising the sequence as shown in SEQ ID NO: 21 or a nucleic acid encoding the amino acid sequence; and the guide RNA-related sequence is a nucleotide sequence comprising the sequence as shown in SEQ ID NO: 218;

(22) the nuclease-related sequence is an amino acid sequence comprising the sequence as shown in SEQ ID NO: 22 or a nucleic acid encoding the amino acid sequence; and the guide RNA-related sequence is a nucleotide sequence comprising the sequence as shown in SEQ ID NO: 219;

(23) the nuclease-related sequence is an amino acid sequence comprising the sequence as shown in SEQ ID NO: 23 or a nucleic acid encoding the amino acid sequence; and the guide RNA-related sequence is a nucleotide sequence comprising the sequence as shown in SEQ ID NO: 220;

(24) the nuclease-related sequence is an amino acid sequence comprising the sequence as shown in SEQ ID NO: 24 or a nucleic acid encoding the amino acid sequence; and the guide RNA-related sequence is a nucleotide sequence comprising the sequence as shown in SEQ ID NO: 221;

(25) the nuclease-related sequence is an amino acid sequence comprising the sequence as shown in SEQ ID NO: 25 or a nucleic acid encoding the amino acid sequence; and the guide RNA-related sequence is a nucleotide sequence comprising the sequence as shown in SEQ ID NO: 222;

(26) the nuclease-related sequence is an amino acid sequence comprising the sequence as shown in SEQ ID NO: 26 or a nucleic acid encoding the amino acid sequence; and the guide RNA-related sequence is a nucleotide sequence comprising the sequence as shown in SEQ ID NO: 223;

(27) the nuclease-related sequence is an amino acid sequence comprising the sequence as shown in SEQ ID NO: 27 or a nucleic acid encoding the amino acid sequence; and the guide RNA-related sequence is a nucleotide sequence comprising the sequence as shown in SEQ ID NO: 224;

(28) the nuclease-related sequence is an amino acid sequence comprising the sequence as shown in SEQ ID NO: 28 or a nucleic acid encoding the amino acid sequence; and the guide RNA-related sequence is a nucleotide sequence comprising the sequence as shown in SEQ ID NO: 225;

(29) the nuclease-related sequence is an amino acid sequence comprising the sequence as shown in SEQ ID NO: 29 or a nucleic acid encoding the amino acid sequence; and the guide RNA-related sequence is a nucleotide sequence comprising the sequence as shown in SEQ ID NO: 226;

(30) the nuclease-related sequence is an amino acid sequence comprising the sequence as shown in SEQ ID NO: 30 or a nucleic acid encoding the amino acid sequence; and the guide RNA-related sequence is a nucleotide sequence comprising the sequence as shown in SEQ ID NO: 227;

(31) the nuclease-related sequence is an amino acid sequence comprising the sequence as shown in SEQ ID NO: 31 or a nucleic acid encoding the amino acid sequence; and the guide RNA-related sequence is a nucleotide sequence comprising the sequence as shown in SEQ ID NO: 228;

(32) the nuclease-related sequence is an amino acid sequence comprising the sequence as shown in SEQ ID NO: 32 or a nucleic acid encoding the amino acid sequence; and the guide RNA-related sequence is a nucleotide sequence comprising the sequence as shown in SEQ ID NO: 229;

(33) the nuclease-related sequence is an amino acid sequence comprising the sequence as shown in SEQ ID NO: 33 or a nucleic acid encoding the amino acid sequence; and the guide RNA-related sequence is a nucleotide sequence comprising the sequence as shown in SEQ ID NO: 230;

(34) the nuclease-related sequence is an amino acid sequence comprising the sequence as shown in SEQ ID NO: 34 or a nucleic acid encoding the amino acid sequence; and the guide RNA-related sequence is a nucleotide sequence comprising the sequence as shown in SEQ ID NO: 231;

(35) the nuclease-related sequence is an amino acid sequence comprising the sequence as shown in SEQ ID NO: 35 or a nucleic acid encoding the amino acid sequence; and the guide RNA-related sequence is a nucleotide sequence comprising the sequence as shown in SEQ ID NO: 232;

(36) the nuclease-related sequence is an amino acid sequence comprising the sequence as shown in SEQ ID NO: 36 or a nucleic acid encoding the amino acid sequence; and the guide RNA-related sequence is a nucleotide sequence comprising the sequence as shown in SEQ ID NO: 233;

(37) the nuclease-related sequence is an amino acid sequence comprising the sequence as shown in SEQ ID NO: 37 or a nucleic acid encoding the amino acid sequence; and the guide RNA-related sequence is a nucleotide sequence comprising the sequence as shown in SEQ ID NO: 234;

(38) the nuclease-related sequence is an amino acid sequence comprising the sequence as shown in SEQ ID NO: 38 or a nucleic acid encoding the amino acid sequence; and the guide RNA-related sequence is a nucleotide sequence comprising the sequence as shown in SEQ ID NO: 235;

(39) the nuclease-related sequence is an amino acid sequence comprising the sequence as shown in SEQ ID NO: 39 or a nucleic acid encoding the amino acid sequence; and the guide RNA-related sequence is a nucleotide sequence comprising the sequence as shown in SEQ ID NO: 236;

(40) the nuclease-related sequence is an amino acid sequence comprising the sequence as shown in SEQ ID NO: 40 or a nucleic acid encoding the amino acid sequence; and the guide RNA-related sequence is a nucleotide sequence comprising the sequence as shown in SEQ ID NO: 237;

(41) the nuclease-related sequence is an amino acid sequence comprising the sequence as shown in SEQ ID NO: 41 or a nucleic acid encoding the amino acid sequence; and the guide RNA-related sequence is a nucleotide sequence comprising the sequence as shown in SEQ ID NO: 238;

(42) the nuclease-related sequence is an amino acid sequence comprising the sequence as shown in SEQ ID NO: 42 or a nucleic acid encoding the amino acid sequence; and the guide RNA-related sequence is a nucleotide sequence comprising the sequence as shown in SEQ ID NO: 239;

(43) the nuclease-related sequence is an amino acid sequence comprising the sequence as shown in SEQ ID NO: 43 or a nucleic acid encoding the amino acid sequence; and the guide RNA-related sequence is a nucleotide sequence comprising the sequence as shown in SEQ ID NO: 240;

(44) the nuclease-related sequence is an amino acid sequence comprising the sequence as shown in SEQ ID NO: 44 or a nucleic acid encoding the amino acid sequence; and the guide RNA-related sequence is a nucleotide sequence comprising the sequence as shown in SEQ ID NO: 241;

(45) the nuclease-related sequence is an amino acid sequence comprising the sequence as shown in SEQ ID NO: 45 or a nucleic acid encoding the amino acid sequence; and the guide RNA-related sequence is a nucleotide sequence comprising the sequence as shown in SEQ ID NO: 242;

(46) the nuclease-related sequence is an amino acid sequence comprising the sequence as shown in SEQ ID NO: 46 or a nucleic acid encoding the amino acid sequence; and the guide RNA-related sequence is a nucleotide sequence comprising the sequence as shown in SEQ ID NO: 243;

(47) the nuclease-related sequence is an amino acid sequence comprising the sequence as shown in SEQ ID NO: 47 or a nucleic acid encoding the amino acid sequence; and the guide RNA-related sequence is a nucleotide sequence comprising the sequence as shown in SEQ ID NO: 244;

(48) the nuclease-related sequence is an amino acid sequence comprising the sequence as shown in SEQ ID NO: 48 or a nucleic acid encoding the amino acid sequence; and the guide RNA-related sequence is a nucleotide sequence comprising the sequence as shown in SEQ ID NO: 245;

(49) the nuclease-related sequence is an amino acid sequence comprising the sequence as shown in SEQ ID NO: 49 or a nucleic acid encoding the amino acid sequence; and the guide RNA-related sequence is a nucleotide sequence comprising the sequence as shown in SEQ ID NO: 246;

(50) the nuclease-related sequence is an amino acid sequence comprising the sequence as shown in SEQ ID NO: 50 or a nucleic acid encoding the amino acid sequence; and the guide RNA-related sequence is a nucleotide sequence comprising the sequence as shown in SEQ ID NO: 247;

(51) the nuclease-related sequence is an amino acid sequence comprising the sequence as shown in SEQ ID NO: 51 or a nucleic acid encoding the amino acid sequence; and the guide RNA-related sequence is a nucleotide sequence comprising the sequence as shown in SEQ ID NO: 248;

(52) the nuclease-related sequence is an amino acid sequence comprising the sequence as shown in SEQ ID NO: 52 or a nucleic acid encoding the amino acid sequence; and the guide RNA-related sequence is a nucleotide sequence comprising the sequence as shown in SEQ ID NO: 249;

(53) the nuclease-related sequence is an amino acid sequence comprising the sequence as shown in SEQ ID NO: 53 or a nucleic acid encoding the amino acid sequence; and the guide RNA-related sequence is a nucleotide sequence comprising the sequence as shown in SEQ ID NO: 250;

(54) the nuclease-related sequence is an amino acid sequence comprising the sequence as shown in SEQ ID NO: 54 or a nucleic acid encoding the amino acid sequence; and the guide RNA-related sequence is a nucleotide sequence comprising the sequence as shown in SEQ ID NO: 251;

(55) the nuclease-related sequence is an amino acid sequence comprising the sequence as shown in SEQ ID NO: 55 or a nucleic acid encoding the amino acid sequence; and the guide RNA-related sequence is a nucleotide sequence comprising the sequence as shown in SEQ ID NO: 252;

(56) the nuclease-related sequence is an amino acid sequence comprising the sequence as shown in SEQ ID NO: 56 or a nucleic acid encoding the amino acid sequence; and the guide RNA-related sequence is a nucleotide sequence comprising the sequence as shown in SEQ ID NO: 253;

(57) the nuclease-related sequence is an amino acid sequence comprising the sequence as shown in SEQ ID NO: 57 or a nucleic acid encoding the amino acid sequence; and the guide RNA-related sequence is a nucleotide sequence comprising the sequence as shown in SEQ ID NO: 254;

(58) the nuclease-related sequence is an amino acid sequence comprising the sequence as shown in SEQ ID NO: 58 or a nucleic acid encoding the amino acid sequence; and the guide RNA-related sequence is a nucleotide sequence comprising the sequence as shown in SEQ ID NO: 255;

(59) the nuclease-related sequence is an amino acid sequence comprising the sequence as shown in SEQ ID NO: 59 or a nucleic acid encoding the amino acid sequence; and the guide RNA-related sequence is a nucleotide sequence comprising the sequence as shown in SEQ ID NO: 256;

(60) the nuclease-related sequence is an amino acid sequence comprising the sequence as shown in SEQ ID NO: 60 or a nucleic acid encoding the amino acid sequence; and the guide RNA-related sequence is a nucleotide sequence comprising the sequence as shown in SEQ ID NO: 257;

(61) the nuclease-related sequence is an amino acid sequence comprising the sequence as shown in SEQ ID NO: 61 or a nucleic acid encoding the amino acid sequence; and the guide RNA-related sequence is a nucleotide sequence comprising the sequence as shown in SEQ ID NO: 258;

(62) the nuclease-related sequence is an amino acid sequence comprising the sequence as shown in SEQ ID NO: 62 or a nucleic acid encoding the amino acid sequence; and the guide RNA-related sequence is a nucleotide sequence comprising the sequence as shown in SEQ ID NO: 259;

(63) the nuclease-related sequence is an amino acid sequence comprising the sequence as shown in SEQ ID NO: 63 or a nucleic acid encoding the amino acid sequence; and the guide RNA-related sequence is a nucleotide sequence comprising the sequence as shown in SEQ ID NO: 260;

(64) the nuclease-related sequence is an amino acid sequence comprising the sequence as shown in SEQ ID NO: 64 or a nucleic acid encoding the amino acid sequence; and the guide RNA-related sequence is a nucleotide sequence comprising the sequence as shown in SEQ ID NO: 261;

(65) the nuclease-related sequence is an amino acid sequence comprising the sequence as shown in SEQ ID NO: 65 or a nucleic acid encoding the amino acid sequence; and the guide RNA-related sequence is a nucleotide sequence comprising the sequence as shown in SEQ ID NO: 262;

(66) the nuclease-related sequence is an amino acid sequence comprising the sequence as shown in SEQ ID NO: 66 or a nucleic acid encoding the amino acid sequence; and the guide RNA-related sequence is a nucleotide sequence comprising the sequence as shown in SEQ ID NO: 263;

(67) the nuclease-related sequence is an amino acid sequence comprising the sequence as shown in SEQ ID NO: 67 or a nucleic acid encoding the amino acid sequence; and the guide RNA-related sequence is a nucleotide sequence comprising the sequence as shown in SEQ ID NO: 264;

(68) the nuclease-related sequence is an amino acid sequence comprising the sequence as shown in SEQ ID NO: 68 or a nucleic acid encoding the amino acid sequence; and the guide RNA-related sequence is a nucleotide sequence comprising the sequence as shown in SEQ ID NO: 265;

(69) the nuclease-related sequence is an amino acid sequence comprising the sequence as shown in SEQ ID NO: 69 or a nucleic acid encoding the amino acid sequence; and the guide RNA-related sequence is a nucleotide sequence comprising the sequence as shown in SEQ ID NO: 266;

(70) the nuclease-related sequence is an amino acid sequence comprising the sequence as shown in SEQ ID NO: 70 or a nucleic acid encoding the amino acid sequence; and the guide RNA-related sequence is a nucleotide sequence comprising the sequence as shown in SEQ ID NO: 267;

(71) the nuclease-related sequence is an amino acid sequence comprising the sequence as shown in SEQ ID NO: 71 or a nucleic acid encoding the amino acid sequence; and the guide RNA-related sequence is a nucleotide sequence comprising the sequence as shown in SEQ ID NO: 268;

(72) the nuclease-related sequence is an amino acid sequence comprising the sequence as shown in SEQ ID NO: 72 or a nucleic acid encoding the amino acid sequence; and the guide RNA-related sequence is a nucleotide sequence comprising the sequence as shown in SEQ ID NO: 269;

(73) the nuclease-related sequence is an amino acid sequence comprising the sequence as shown in SEQ ID NO: 73 or a nucleic acid encoding the amino acid sequence; and the guide RNA-related sequence is a nucleotide sequence comprising the sequence as shown in SEQ ID NO: 270;

(74) the nuclease-related sequence is an amino acid sequence comprising the sequence as shown in SEQ ID NO: 74 or a nucleic acid encoding the amino acid sequence; and the guide RNA-related sequence is a nucleotide sequence comprising the sequence as shown in SEQ ID NO: 271;

(75) the nuclease-related sequence is an amino acid sequence comprising the sequence as shown in SEQ ID NO: 75 or a nucleic acid encoding the amino acid sequence; and the guide RNA-related sequence is a nucleotide sequence comprising the sequence as shown in SEQ ID NO: 272;

(76) the nuclease-related sequence is an amino acid sequence comprising the sequence as shown in SEQ ID NO: 76 or a nucleic acid encoding the amino acid sequence; and the guide RNA-related sequence is a nucleotide sequence comprising the sequence as shown in SEQ ID NO: 273;

(77) the nuclease-related sequence is an amino acid sequence comprising the sequence as shown in SEQ ID NO: 77 or a nucleic acid encoding the amino acid sequence; and the guide RNA-related sequence is a nucleotide sequence comprising the sequence as shown in SEQ ID NO: 274;

(78) the nuclease-related sequence is an amino acid sequence comprising the sequence as shown in SEQ ID NO: 78 or a nucleic acid encoding the amino acid sequence; and the guide RNA-related sequence is a nucleotide sequence comprising the sequence as shown in SEQ ID NO: 275;

(79) the nuclease-related sequence is an amino acid sequence comprising the sequence as shown in SEQ ID NO: 79 or a nucleic acid encoding the amino acid sequence; and the guide RNA-related sequence is a nucleotide sequence comprising the sequence as shown in SEQ ID NO: 276;

(80) the nuclease-related sequence is an amino acid sequence comprising the sequence as shown in SEQ ID NO: 80 or a nucleic acid encoding the amino acid sequence; and the guide RNA-related sequence is a nucleotide sequence comprising the sequence as shown in SEQ ID NO: 277;

(81) the nuclease-related sequence is an amino acid sequence comprising the sequence as shown in SEQ ID NO: 81 or a nucleic acid encoding the amino acid sequence; and the guide RNA-related sequence is a nucleotide sequence comprising the sequence as shown in SEQ ID NO: 278;

(82) the nuclease-related sequence is an amino acid sequence comprising the sequence as shown in SEQ ID NO: 82 or a nucleic acid encoding the amino acid sequence; and the guide RNA-related sequence is a nucleotide sequence comprising the sequence as shown in SEQ ID NO: 279;

(83) the nuclease-related sequence is an amino acid sequence comprising the sequence as shown in SEQ ID NO: 83 or a nucleic acid encoding the amino acid sequence; and the guide RNA-related sequence is a nucleotide sequence comprising the sequence as shown in SEQ ID NO: 280;

(84) the nuclease-related sequence is an amino acid sequence comprising the sequence as shown in SEQ ID NO: 84 or a nucleic acid encoding the amino acid sequence; and the guide RNA-related sequence is a nucleotide sequence comprising the sequence as shown in SEQ ID NO: 281;

(85) the nuclease-related sequence is an amino acid sequence comprising the sequence as shown in SEQ ID NO: 85 or a nucleic acid encoding the amino acid sequence; and the guide RNA-related sequence is a nucleotide sequence comprising the sequence as shown in SEQ ID NO: 282;

(86) the nuclease-related sequence is an amino acid sequence comprising the sequence as shown in SEQ ID NO: 86 or a nucleic acid encoding the amino acid sequence; and the guide RNA-related sequence is a nucleotide sequence comprising the sequence as shown in SEQ ID NO: 283;

(87) the nuclease-related sequence is an amino acid sequence comprising the sequence as shown in SEQ ID NO: 87 or a nucleic acid encoding the amino acid sequence; and the guide RNA-related sequence is a nucleotide sequence comprising the sequence as shown in SEQ ID NO: 284;

(88) the nuclease-related sequence is an amino acid sequence comprising the sequence as shown in SEQ ID NO: 88 or a nucleic acid encoding the amino acid sequence; and the guide RNA-related sequence is a nucleotide sequence comprising the sequence as shown in SEQ ID NO: 285;

(89) the nuclease-related sequence is an amino acid sequence comprising the sequence as shown in SEQ ID NO: 89 or a nucleic acid encoding the amino acid sequence; and the guide RNA-related sequence is a nucleotide sequence comprising the sequence as shown in SEQ ID NO: 286;

(90) the nuclease-related sequence is an amino acid sequence comprising the sequence as shown in SEQ ID NO: 90 or a nucleic acid encoding the amino acid sequence; and the guide RNA-related sequence is a nucleotide sequence comprising the sequence as shown in SEQ ID NO: 287;

(91) the nuclease-related sequence is an amino acid sequence comprising the sequence as shown in SEQ ID NO: 91 or a nucleic acid encoding the amino acid sequence; and the guide RNA-related sequence is a nucleotide sequence comprising the sequence as shown in SEQ ID NO: 288;

(92) the nuclease-related sequence is an amino acid sequence comprising the sequence as shown in SEQ ID NO: 92 or a nucleic acid encoding the amino acid sequence; and the guide RNA-related sequence is a nucleotide sequence comprising the sequence as shown in SEQ ID NO: 289;

(93) the nuclease-related sequence is an amino acid sequence comprising the sequence as shown in SEQ ID NO: 93 or a nucleic acid encoding the amino acid sequence; and the guide RNA-related sequence is a nucleotide sequence comprising the sequence as shown in SEQ ID NO: 290;

(94) the nuclease-related sequence is an amino acid sequence comprising the sequence as shown in SEQ ID NO: 94 or a nucleic acid encoding the amino acid sequence; and the guide RNA-related sequence is a nucleotide sequence comprising the sequence as shown in SEQ ID NO: 291;

(95) the nuclease-related sequence is an amino acid sequence comprising the sequence as shown in SEQ ID NO: 95 or a nucleic acid encoding the amino acid sequence; and the guide RNA-related sequence is a nucleotide sequence comprising the sequence as shown in SEQ ID NO: 292;

(96) the nuclease-related sequence is an amino acid sequence comprising the sequence as shown in SEQ ID NO: 96 or a nucleic acid encoding the amino acid sequence; and the guide RNA-related sequence is a nucleotide sequence comprising the sequence as shown in SEQ ID NO: 293;

(97) the nuclease-related sequence is an amino acid sequence comprising the sequence as shown in SEQ ID NO: 97 or a nucleic acid encoding the amino acid sequence; and the guide RNA-related sequence is a nucleotide sequence comprising the sequence as shown in SEQ ID NO: 294;

(98) the nuclease-related sequence is an amino acid sequence comprising the sequence as shown in SEQ ID NO: 98 or a nucleic acid encoding the amino acid sequence; and the guide RNA-related sequence is a nucleotide sequence comprising the sequence as shown in SEQ ID NO: 295;

(99) the nuclease-related sequence is an amino acid sequence comprising the sequence as shown in SEQ ID NO: 99 or a nucleic acid encoding the amino acid sequence; and the guide RNA-related sequence is a nucleotide sequence comprising the sequence as shown in SEQ ID NO: 296;

(100) the nuclease-related sequence is an amino acid sequence comprising the sequence as shown in SEQ ID NO: 100 or a nucleic acid encoding the amino acid sequence; and the guide RNA-related sequence is a nucleotide sequence comprising the sequence as shown in SEQ ID NO: 297;

(101) the nuclease-related sequence is an amino acid sequence comprising the sequence as shown in SEQ ID NO: 101 or a nucleic acid encoding the amino acid sequence; and the guide RNA-related sequence is a nucleotide sequence comprising the sequence as shown in SEQ ID NO: 298;

(102) the nuclease-related sequence is an amino acid sequence comprising the sequence as shown in SEQ ID NO: 102 or a nucleic acid encoding the amino acid sequence; and the guide RNA-related sequence is a nucleotide sequence comprising the sequence as shown in SEQ ID NO: 299;

(103) the nuclease-related sequence is an amino acid sequence comprising the sequence as shown in SEQ ID NO: 103 or a nucleic acid encoding the amino acid sequence; and the guide RNA-related sequence is a nucleotide sequence comprising the sequence as shown in SEQ ID NO: 300;

(104) the nuclease-related sequence is an amino acid sequence comprising the sequence as shown in SEQ ID NO: 104 or a nucleic acid encoding the amino acid sequence; and the guide RNA-related sequence is a nucleotide sequence comprising the sequence as shown in SEQ ID NO: 301;

(105) the nuclease-related sequence is an amino acid sequence comprising the sequence as shown in SEQ ID NO: 105 or a nucleic acid encoding the amino acid sequence; and the guide RNA-related sequence is a nucleotide sequence comprising the sequence as shown in SEQ ID NO: 302;

(106) the nuclease-related sequence is an amino acid sequence comprising the sequence as shown in SEQ ID NO: 106 or a nucleic acid encoding the amino acid sequence; and the guide RNA-related sequence is a nucleotide sequence comprising the sequence as shown in SEQ ID NO: 303;

(107) the nuclease-related sequence is an amino acid sequence comprising the sequence as shown in SEQ ID NO: 107 or a nucleic acid encoding the amino acid sequence; and the guide RNA-related sequence is a nucleotide sequence comprising the sequence as shown in SEQ ID NO: 304;

(108) the nuclease-related sequence is an amino acid sequence comprising the sequence as shown in SEQ ID NO: 108 or a nucleic acid encoding the amino acid sequence; and the guide RNA-related sequence is a nucleotide sequence comprising the sequence as shown in SEQ ID NO: 305;

(109) the nuclease-related sequence is an amino acid sequence comprising the sequence as shown in SEQ ID NO: 109 or a nucleic acid encoding the amino acid sequence; and the guide RNA-related sequence is a nucleotide sequence comprising the sequence as shown in SEQ ID NO: 306;

(110) the nuclease-related sequence is an amino acid sequence comprising the sequence as shown in SEQ ID NO: 110 or a nucleic acid encoding the amino acid sequence; and the guide RNA-related sequence is a nucleotide sequence comprising the sequence as shown in SEQ ID NO: 307;

(111) the nuclease-related sequence is an amino acid sequence comprising the sequence as shown in SEQ ID NO: 111 or a nucleic acid encoding the amino acid sequence; and the guide RNA-related sequence is a nucleotide sequence comprising the sequence as shown in SEQ ID NO: 308;

(112) the nuclease-related sequence is an amino acid sequence comprising the sequence as shown in SEQ ID NO: 112 or a nucleic acid encoding the amino acid sequence; and the guide RNA-related sequence is a nucleotide sequence comprising the sequence as shown in SEQ ID NO: 309;

(113) the nuclease-related sequence is an amino acid sequence comprising the sequence as shown in SEQ ID NO: 113 or a nucleic acid encoding the amino acid sequence; and the guide RNA-related sequence is a nucleotide sequence comprising the sequence as shown in SEQ ID NO: 310;

(114) the nuclease-related sequence is an amino acid sequence comprising the sequence as shown in SEQ ID NO: 114 or a nucleic acid encoding the amino acid sequence; and the guide RNA-related sequence is a nucleotide sequence comprising the sequence as shown in SEQ ID NO: 311;

(115) the nuclease-related sequence is an amino acid sequence comprising the sequence as shown in SEQ ID NO: 115 or a nucleic acid encoding the amino acid sequence; and the guide RNA-related sequence is a nucleotide sequence comprising the sequence as shown in SEQ ID NO: 312;

(116) the nuclease-related sequence is an amino acid sequence comprising the sequence as shown in SEQ ID NO: 116 or a nucleic acid encoding the amino acid sequence; and the guide RNA-related sequence is a nucleotide sequence comprising the sequence as shown in SEQ ID NO: 313;

(117) the nuclease-related sequence is an amino acid sequence comprising the sequence as shown in SEQ ID NO: 117 or a nucleic acid encoding the amino acid sequence; and the guide RNA-related sequence is a nucleotide sequence comprising the sequence as shown in SEQ ID NO: 314;

(118) the nuclease-related sequence is an amino acid sequence comprising the sequence as shown in SEQ ID NO: 118 or a nucleic acid encoding the amino acid sequence; and the guide RNA-related sequence is a nucleotide sequence comprising the sequence as shown in SEQ ID NO: 315;

(119) the nuclease-related sequence is an amino acid sequence comprising the sequence as shown in SEQ ID NO: 119 or a nucleic acid encoding the amino acid sequence; and the guide RNA-related sequence is a nucleotide sequence comprising the sequence as shown in SEQ ID NO: 316;

(120) the nuclease-related sequence is an amino acid sequence comprising the sequence as shown in SEQ ID NO: 120 or a nucleic acid encoding the amino acid sequence; and the guide RNA-related sequence is a nucleotide sequence comprising the sequence as shown in SEQ ID NO: 317;

(121) the nuclease-related sequence is an amino acid sequence comprising the sequence as shown in SEQ ID NO: 121 or a nucleic acid encoding the amino acid sequence; and the guide RNA-related sequence is a nucleotide sequence comprising the sequence as shown in SEQ ID NO: 318;

(122) the nuclease-related sequence is an amino acid sequence comprising the sequence as shown in SEQ ID NO: 122 or a nucleic acid encoding the amino acid sequence; and the guide RNA-related sequence is a nucleotide sequence comprising the sequence as shown in SEQ ID NO: 319;

(123) the nuclease-related sequence is an amino acid sequence comprising the sequence as shown in SEQ ID NO: 123 or a nucleic acid encoding the amino acid sequence; and the guide RNA-related sequence is a nucleotide sequence comprising the sequence as shown in SEQ ID NO: 320;

(124) the nuclease-related sequence is an amino acid sequence comprising the sequence as shown in SEQ ID NO: 124 or a nucleic acid encoding the amino acid sequence; and the guide RNA-related sequence is a nucleotide sequence comprising the sequence as shown in SEQ ID NO: 321;

(125) the nuclease-related sequence is an amino acid sequence comprising the sequence as shown in SEQ ID NO: 125 or a nucleic acid encoding the amino acid sequence; and the guide RNA-related sequence is a nucleotide sequence comprising the sequence as shown in SEQ ID NO: 322;

(126) the nuclease-related sequence is an amino acid sequence comprising the sequence as shown in SEQ ID NO: 126 or a nucleic acid encoding the amino acid sequence; and the guide RNA-related sequence is a nucleotide sequence comprising the sequence as shown in SEQ ID NO: 323;

(127) the nuclease-related sequence is an amino acid sequence comprising the sequence as shown in SEQ ID NO: 127 or a nucleic acid encoding the amino acid sequence; and the guide RNA-related sequence is a nucleotide sequence comprising the sequence as shown in SEQ ID NO: 324;

(128) the nuclease-related sequence is an amino acid sequence comprising the sequence as shown in SEQ ID NO: 128 or a nucleic acid encoding the amino acid sequence; and the guide RNA-related sequence is a nucleotide sequence comprising the sequence as shown in SEQ ID NO: 325;

(129) the nuclease-related sequence is an amino acid sequence comprising the sequence as shown in SEQ ID NO: 129 or a nucleic acid encoding the amino acid sequence; and the guide RNA-related sequence is a nucleotide sequence comprising the sequence as shown in SEQ ID NO: 326;

(130) the nuclease-related sequence is an amino acid sequence comprising the sequence as shown in SEQ ID NO: 130 or a nucleic acid encoding the amino acid sequence; and the guide RNA-related sequence is a nucleotide sequence comprising the sequence as shown in SEQ ID NO: 327;

(131) the nuclease-related sequence is an amino acid sequence comprising the sequence as shown in SEQ ID NO: 131 or a nucleic acid encoding the amino acid sequence; and the guide RNA-related sequence is a nucleotide sequence comprising the sequence as shown in SEQ ID NO: 328;

(132) the nuclease-related sequence is an amino acid sequence comprising the sequence as shown in SEQ ID NO: 132 or a nucleic acid encoding the amino acid sequence; and the guide RNA-related sequence is a nucleotide sequence comprising the sequence as shown in SEQ ID NO: 329;

(133) the nuclease-related sequence is an amino acid sequence comprising the sequence as shown in SEQ ID NO: 133 or a nucleic acid encoding the amino acid sequence; and the guide RNA-related sequence is a nucleotide sequence comprising the sequence as shown in SEQ ID NO: 330;

(134) the nuclease-related sequence is an amino acid sequence comprising the sequence as shown in SEQ ID NO: 134 or a nucleic acid encoding the amino acid sequence; and the guide RNA-related sequence is a nucleotide sequence comprising the sequence as shown in SEQ ID NO: 331;

(135) the nuclease-related sequence is an amino acid sequence comprising the sequence as shown in SEQ ID NO: 135 or a nucleic acid encoding the amino acid sequence; and the guide RNA-related sequence is a nucleotide sequence comprising the sequence as shown in SEQ ID NO: 332;

(136) the nuclease-related sequence is an amino acid sequence comprising the sequence as shown in SEQ ID NO: 136 or a nucleic acid encoding the amino acid sequence; and the guide RNA-related sequence is a nucleotide sequence comprising the sequence as shown in SEQ ID NO: 333;

(137) the nuclease-related sequence is an amino acid sequence comprising the sequence as shown in SEQ ID NO: 137 or a nucleic acid encoding the amino acid sequence; and the guide RNA-related sequence is a nucleotide sequence comprising the sequence as shown in SEQ ID NO: 334;

(138) the nuclease-related sequence is an amino acid sequence comprising the sequence as shown in SEQ ID NO: 138 or a nucleic acid encoding the amino acid sequence; and the guide RNA-related sequence is a nucleotide sequence comprising the sequence as shown in SEQ ID NO: 335;

(139) the nuclease-related sequence is an amino acid sequence comprising the sequence as shown in SEQ ID NO: 139 or a nucleic acid encoding the amino acid sequence; and the guide RNA-related sequence is a nucleotide sequence comprising the sequence as shown in SEQ ID NO: 336;

(140) the nuclease-related sequence is an amino acid sequence comprising the sequence as shown in SEQ ID NO: 140 or a nucleic acid encoding the amino acid sequence; and the guide RNA-related sequence is a nucleotide sequence comprising the sequence as shown in SEQ ID NO: 337;

(141) the nuclease-related sequence is an amino acid sequence comprising the sequence as shown in SEQ ID NO: 141 or a nucleic acid encoding the amino acid sequence; and the guide RNA-related sequence is a nucleotide sequence comprising the sequence as shown in SEQ ID NO: 338;

(142) the nuclease-related sequence is an amino acid sequence comprising the sequence as shown in SEQ ID NO: 142 or a nucleic acid encoding the amino acid sequence; and the guide RNA-related sequence is a nucleotide sequence comprising the sequence as shown in SEQ ID NO: 339;

(143) the nuclease-related sequence is an amino acid sequence comprising the sequence as shown in SEQ ID NO: 143 or a nucleic acid encoding the amino acid sequence; and the guide RNA-related sequence is a nucleotide sequence comprising the sequence as shown in SEQ ID NO: 340;

(144) the nuclease-related sequence is an amino acid sequence comprising the sequence as shown in SEQ ID NO: 144 or a nucleic acid encoding the amino acid sequence; and the guide RNA-related sequence is a nucleotide sequence comprising the sequence as shown in SEQ ID NO: 341;

(145) the nuclease-related sequence is an amino acid sequence comprising the sequence as shown in SEQ ID NO: 145 or a nucleic acid encoding the amino acid sequence; and the guide RNA-related sequence is a nucleotide sequence comprising the sequence as shown in SEQ ID NO: 342;

(146) the nuclease-related sequence is an amino acid sequence comprising the sequence as shown in SEQ ID NO: 146 or a nucleic acid encoding the amino acid sequence; and the guide RNA-related sequence is a nucleotide sequence comprising the sequence as shown in SEQ ID NO: 343;

(147) the nuclease-related sequence is an amino acid sequence comprising the sequence as shown in SEQ ID NO: 147 or a nucleic acid encoding the amino acid sequence; and the guide RNA-related sequence is a nucleotide sequence comprising the sequence as shown in SEQ ID NO: 344;

(148) the nuclease-related sequence is an amino acid sequence comprising the sequence as shown in SEQ ID NO: 148 or a nucleic acid encoding the amino acid sequence; and the guide RNA-related sequence is a nucleotide sequence comprising the sequence as shown in SEQ ID NO: 345;

(149) the nuclease-related sequence is an amino acid sequence comprising the sequence as shown in SEQ ID NO: 149 or a nucleic acid encoding the amino acid sequence; and the guide RNA-related sequence is a nucleotide sequence comprising the sequence as shown in SEQ ID NO: 346;

(150) the nuclease-related sequence is an amino acid sequence comprising the sequence as shown in SEQ ID NO: 150 or a nucleic acid encoding the amino acid sequence; and the guide RNA-related sequence is a nucleotide sequence comprising the sequence as shown in SEQ ID NO: 347;

(151) the nuclease-related sequence is an amino acid sequence comprising the sequence as shown in SEQ ID NO: 151 or a nucleic acid encoding the amino acid sequence; and the guide RNA-related sequence is a nucleotide sequence comprising the sequence as shown in SEQ ID NO: 348;

(152) the nuclease-related sequence is an amino acid sequence comprising the sequence as shown in SEQ ID NO: 152 or a nucleic acid encoding the amino acid sequence; and the guide RNA-related sequence is a nucleotide sequence comprising the sequence as shown in SEQ ID NO: 349;

(153) the nuclease-related sequence is an amino acid sequence comprising the sequence as shown in SEQ ID NO: 153 or a nucleic acid encoding the amino acid sequence; and the guide RNA-related sequence is a nucleotide sequence comprising the sequence as shown in SEQ ID NO: 350;

(154) the nuclease-related sequence is an amino acid sequence comprising the sequence as shown in SEQ ID NO: 154 or a nucleic acid encoding the amino acid sequence; and the guide RNA-related sequence is a nucleotide sequence comprising the sequence as shown in SEQ ID NO: 351;

(155) the nuclease-related sequence is an amino acid sequence comprising the sequence as shown in SEQ ID NO: 155 or a nucleic acid encoding the amino acid sequence; and the guide RNA-related sequence is a nucleotide sequence comprising the sequence as shown in SEQ ID NO: 352;

(156) the nuclease-related sequence is an amino acid sequence comprising the sequence as shown in SEQ ID NO: 156 or a nucleic acid encoding the amino acid sequence; and the guide RNA-related sequence is a nucleotide sequence comprising the sequence as shown in SEQ ID NO: 353;

(157) the nuclease-related sequence is an amino acid sequence comprising the sequence as shown in SEQ ID NO: 157 or a nucleic acid encoding the amino acid sequence; and the guide RNA-related sequence is a nucleotide sequence comprising the sequence as shown in SEQ ID NO: 354;

(158) the nuclease-related sequence is an amino acid sequence comprising the sequence as shown in SEQ ID NO: 158 or a nucleic acid encoding the amino acid sequence; and the guide RNA-related sequence is a nucleotide sequence comprising the sequence as shown in SEQ ID NO: 355;

(159) the nuclease-related sequence is an amino acid sequence comprising the sequence as shown in SEQ ID NO: 159 or a nucleic acid encoding the amino acid sequence; and the guide RNA-related sequence is a nucleotide sequence comprising the sequence as shown in SEQ ID NO: 356;

(160) the nuclease-related sequence is an amino acid sequence comprising the sequence as shown in SEQ ID NO: 160 or a nucleic acid encoding the amino acid sequence; and the guide RNA-related sequence is a nucleotide sequence comprising the sequence as shown in SEQ ID NO: 357;

(161) the nuclease-related sequence is an amino acid sequence comprising the sequence as shown in SEQ ID NO: 161 or a nucleic acid encoding the amino acid sequence; and the guide RNA-related sequence is a nucleotide sequence comprising the sequence as shown in SEQ ID NO: 358;

(162) the nuclease-related sequence is an amino acid sequence comprising the sequence as shown in SEQ ID NO: 162 or a nucleic acid encoding the amino acid sequence; and the guide RNA-related sequence is a nucleotide sequence comprising the sequence as shown in SEQ ID NO: 359;

(163) the nuclease-related sequence is an amino acid sequence comprising the sequence as shown in SEQ ID NO: 163 or a nucleic acid encoding the amino acid sequence; and the guide RNA-related sequence is a nucleotide sequence comprising the sequence as shown in SEQ ID NO: 360;

(164) the nuclease-related sequence is an amino acid sequence comprising the sequence as shown in SEQ ID NO: 164 or a nucleic acid encoding the amino acid sequence; and the guide RNA-related sequence is a nucleotide sequence comprising the sequence as shown in SEQ ID NO: 361;

(165) the nuclease-related sequence is an amino acid sequence comprising the sequence as shown in SEQ ID NO: 165 or a nucleic acid encoding the amino acid sequence; and the guide RNA-related sequence is a nucleotide sequence comprising the sequence as shown in SEQ ID NO: 362;

(166) the nuclease-related sequence is an amino acid sequence comprising the sequence as shown in SEQ ID NO: 166 or a nucleic acid encoding the amino acid sequence; and the guide RNA-related sequence is a nucleotide sequence comprising the sequence as shown in SEQ ID NO: 363;

(167) the nuclease-related sequence is an amino acid sequence comprising the sequence as shown in SEQ ID NO: 167 or a nucleic acid encoding the amino acid sequence; and the guide RNA-related sequence is a nucleotide sequence comprising the sequence as shown in SEQ ID NO: 364;

(168) the nuclease-related sequence is an amino acid sequence comprising the sequence as shown in SEQ ID NO: 168 or a nucleic acid encoding the amino acid sequence; and the guide RNA-related sequence is a nucleotide sequence comprising the sequence as shown in SEQ ID NO: 365;

(169) the nuclease-related sequence is an amino acid sequence comprising the sequence as shown in SEQ ID NO: 169 or a nucleic acid encoding the amino acid sequence; and the guide RNA-related sequence is a nucleotide sequence comprising the sequence as shown in SEQ ID NO: 366;

(170) the nuclease-related sequence is an amino acid sequence comprising the sequence as shown in SEQ ID NO: 170 or a nucleic acid encoding the amino acid sequence; and the guide RNA-related sequence is a nucleotide sequence comprising the sequence as shown in SEQ ID NO: 367;

(171) the nuclease-related sequence is an amino acid sequence comprising the sequence as shown in SEQ ID NO: 171 or a nucleic acid encoding the amino acid sequence; and the guide RNA-related sequence is a nucleotide sequence comprising the sequence as shown in SEQ ID NO: 368;

(172) the nuclease-related sequence is an amino acid sequence comprising the sequence as shown in SEQ ID NO: 172 or a nucleic acid encoding the amino acid sequence; and the guide RNA-related sequence is a nucleotide sequence comprising the sequence as shown in SEQ ID NO: 369;

(173) the nuclease-related sequence is an amino acid sequence comprising the sequence as shown in SEQ ID NO: 173 or a nucleic acid encoding the amino acid sequence; and the guide RNA-related sequence is a nucleotide sequence comprising the sequence as shown in SEQ ID NO: 370;

(174) the nuclease-related sequence is an amino acid sequence comprising the sequence as shown in SEQ ID NO: 174 or a nucleic acid encoding the amino acid sequence; and the guide RNA-related sequence is a nucleotide sequence comprising the sequence as shown in SEQ ID NO: 371;

(175) the nuclease-related sequence is an amino acid sequence comprising the sequence as shown in SEQ ID NO: 175 or a nucleic acid encoding the amino acid sequence; and the guide RNA-related sequence is a nucleotide sequence comprising the sequence as shown in SEQ ID NO: 372;

(176) the nuclease-related sequence is an amino acid sequence comprising the sequence as shown in SEQ ID NO: 176 or a nucleic acid encoding the amino acid sequence; and the guide RNA-related sequence is a nucleotide sequence comprising the sequence as shown in SEQ ID NO: 373;

(177) the nuclease-related sequence is an amino acid sequence comprising the sequence as shown in SEQ ID NO: 177 or a nucleic acid encoding the amino acid sequence; and the guide RNA-related sequence is a nucleotide sequence comprising the sequence as shown in SEQ ID NO: 374;

(178) the nuclease-related sequence is an amino acid sequence comprising the sequence as shown in SEQ ID NO: 178 or a nucleic acid encoding the amino acid sequence; and the guide RNA-related sequence is a nucleotide sequence comprising the sequence as shown in SEQ ID NO: 375;

(179) the nuclease-related sequence is an amino acid sequence comprising the sequence as shown in SEQ ID NO: 179 or a nucleic acid encoding the amino acid sequence; and the guide RNA-related sequence is a nucleotide sequence comprising the sequence as shown in SEQ ID NO: 376;

(180) the nuclease-related sequence is an amino acid sequence comprising the sequence as shown in SEQ ID NO: 180 or a nucleic acid encoding the amino acid sequence; and the guide RNA-related sequence is a nucleotide sequence comprising the sequence as shown in SEQ ID NO: 377;

(181) the nuclease-related sequence is an amino acid sequence comprising the sequence as shown in SEQ ID NO: 181 or a nucleic acid encoding the amino acid sequence; and the guide RNA-related sequence is a nucleotide sequence comprising the sequence as shown in SEQ ID NO: 378;

(182) the nuclease-related sequence is an amino acid sequence comprising the sequence as shown in SEQ ID NO: 182 or a nucleic acid encoding the amino acid sequence; and the guide RNA-related sequence is a nucleotide sequence comprising the sequence as shown in SEQ ID NO: 379;

(183) the nuclease-related sequence is an amino acid sequence comprising the sequence as shown in SEQ ID NO: 183 or a nucleic acid encoding the amino acid sequence; and the guide RNA-related sequence is a nucleotide sequence comprising the sequence as shown in SEQ ID NO: 380;

(184) the nuclease-related sequence is an amino acid sequence comprising the sequence as shown in SEQ ID NO: 184 or a nucleic acid encoding the amino acid sequence; and the guide RNA-related sequence is a nucleotide sequence comprising the sequence as shown in SEQ ID NO: 381;

(185) the nuclease-related sequence is an amino acid sequence comprising the sequence as shown in SEQ ID NO: 185 or a nucleic acid encoding the amino acid sequence; and the guide RNA-related sequence is a nucleotide sequence comprising the sequence as shown in SEQ ID NO: 382;

(186) the nuclease-related sequence is an amino acid sequence comprising the sequence as shown in SEQ ID NO: 186 or a nucleic acid encoding the amino acid sequence; and the guide RNA-related sequence is a nucleotide sequence comprising the sequence as shown in SEQ ID NO: 383;

(187) the nuclease-related sequence is an amino acid sequence comprising the sequence as shown in SEQ ID NO: 187 or a nucleic acid encoding the amino acid sequence; and the guide RNA-related sequence is a nucleotide sequence comprising the sequence as shown in SEQ ID NO: 384;

(188) the nuclease-related sequence is an amino acid sequence comprising the sequence as shown in SEQ ID NO: 188 or a nucleic acid encoding the amino acid sequence; and the guide RNA-related sequence is a nucleotide sequence comprising the sequence as shown in SEQ ID NO: 385;

(189) the nuclease-related sequence is an amino acid sequence comprising the sequence as shown in SEQ ID NO: 189 or a nucleic acid encoding the amino acid sequence; and the guide RNA-related sequence is a nucleotide sequence comprising the sequence as shown in SEQ ID NO: 386;

(190) the nuclease-related sequence is an amino acid sequence comprising the sequence as shown in SEQ ID NO: 190 or a nucleic acid encoding the amino acid sequence; and the guide RNA-related sequence is a nucleotide sequence comprising the sequence as shown in SEQ ID NO: 387;

(191) the nuclease-related sequence is an amino acid sequence comprising the sequence as shown in SEQ ID NO: 191 or a nucleic acid encoding the amino acid sequence; and the guide RNA-related sequence is a nucleotide sequence comprising the sequence as shown in SEQ ID NO: 388;

(192) the nuclease-related sequence is an amino acid sequence comprising the sequence as shown in SEQ ID NO: 192 or a nucleic acid encoding the amino acid sequence; and the guide RNA-related sequence is a nucleotide sequence comprising the sequence as shown in SEQ ID NO: 389;

(193) the nuclease-related sequence is an amino acid sequence comprising the sequence as shown in SEQ ID NO: 193 or a nucleic acid encoding the amino acid sequence; and the guide RNA-related sequence is a nucleotide sequence comprising the sequence as shown in SEQ ID NO: 390;

(194) the nuclease-related sequence is an amino acid sequence comprising the sequence as shown in SEQ ID NO: 194 or a nucleic acid encoding the amino acid sequence; and the guide RNA-related sequence is a nucleotide sequence comprising the sequence as shown in SEQ ID NO: 391;

(195) the nuclease-related sequence is an amino acid sequence comprising the sequence as shown in SEQ ID NO: 195 or a nucleic acid encoding the amino acid sequence; and the guide RNA-related sequence is a nucleotide sequence comprising the sequence as shown in SEQ ID NO: 392;

(196) the nuclease-related sequence is an amino acid sequence comprising the sequence as shown in SEQ ID NO: 196 or a nucleic acid encoding the amino acid sequence; and the guide RNA-related sequence is a nucleotide sequence comprising the sequence as shown in SEQ ID NO: 393;

(197) the nuclease-related sequence is an amino acid sequence comprising the sequence as shown in SEQ ID NO: 197 or a nucleic acid encoding the amino acid sequence; and the guide RNA-related sequence is a nucleotide sequence comprising the sequence as shown in SEQ ID NO: 394;

(198) a variant of any one of the aforementioned groups (1)-(197),
wherein the nuclease-related sequence is the amino acid sequence of the variant of the nuclease in each group or a nucleic acid sequence encoding the variant, and the variant has a variant sequence of the aforementioned nuclease having a nuclease activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the nuclease in each group;
(ii) at least one of amino acid sequences having at least 70%, 80%, 90%, 95% or 99% identity to the amino acid sequence as shown in any one of SEQ ID NOs: 1-197; and
(iii) at least one sequence obtained by further fusing the amino acid sequence as shown in any one of SEQ ID NO: 1-197 with other sequences.

In some embodiments, the guide RNA-related sequence further comprises a targeted sequence that can recognize a targeting gene adjacent to a transposon-associated motif. In some embodiments, the targeted sequence is of at least one of 10-50, 10-40, 10-30, or 15-25 nucleotides in length.

Sequences and lengths of the transposon-associated motif in the present application can vary depending on the nuclease, and the transposon-associated motif can be recognized by a complex formed by the nuclease and guide RNA described in the present application. In some embodiments, the transposon-associated motif comprises a nucleotide sequence as shown in the following formula:

(X₁₇)ₕ(X₁₈)(X₁₉)A(X₂₀)
wherein h is the number of nucleotides; A is an adenine deoxyribonucleotide; (X₁₇) is any deoxyribonucleotide, and h is 0 or 1; (X₁₈) is a cytosine deoxyribonucleotid e or thymine deoxyribonucleotide; (X₁₉) is a cytosine deoxyribonucleotide, thymine deoxyribonucleotide, or guanine deoxyribonucleotide; and (X₂₀) is any deoxyribonucleotide.

The targeting gene in the present application includes any gene of interest, e.g., a gene of a natural functional protein, an artificial chimeric gene, or a gene of a non-coding RNA. In some embodiments, the gene of a natural functional protein includes a fluorescein reporter gene, a luciferase gene, and a resistance gene. In some embodiments, the artificial chimeric gene includes a gene of a chimeric antigen receptor. In some embodiments, the fluorescein reporter gene includes a gene encoding a green fluorescent protein, a red fluorescent protein, a blue fluorescent protein, or a yellow fluorescent protein. In some embodiments, the luciferase gene includes a gene encoding firefly luciferase or sea kidney luciferase. In some embodiments, the resistance gene includes a gene encoding puromycin resistance, G418 resistance, kanamycin resistance, tetracycline resistance, or bleomycin resistance.

In some embodiments, the nucleic acid encoding the amino acid sequence and/or the nucleic acid encoding the guide RNA further comprises a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL. In some embodiments, the nucleic acid encoding the amino acid sequence and/or the nucleic acid encoding the guide RNA further comprises a polyA sequence. PolyA tailing signal sequences well known in the art, as well as various truncated forms of polyA tailing signals, can be used in the present application.

In some embodiments, the nucleic acid encoding the amino acid sequence and/or the nucleic acid encoding the guide RNA further comprises any transcription termination sequence that controls the expression of the exogenous nucleic acid fragment, i.e., a sequence that is recognized by a host cell to terminate transcription. Any terminator that is functional in the host cell of choice can be used in the present invention.

In some embodiments, the nucleic acid encoding the amino acid sequence and/or the nucleic acid encoding the guide RNA further comprises any transcription termination sequence, i.e., a sequence that is recognized by a host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid encoding the amino acid sequence and/or the nucleic acid encoding the guide RNA may further comprise a suitable leader sequence, i.e., an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid encoding the amino acid sequence and/or the nucleic acid encoding the guide RNA may further comprise a propeptide coding region, which encodes an amino acid sequence located at the amino terminus of the polypeptide. The resulting polypeptide is called a zymogen or a propolypeptide. The propolypeptide is usually inactive and can be converted into a mature active polypeptide by catalytic or autocatalytic cleavage of the propeptide from the propolypeptide.

Optionally, the nucleic acid encoding the amino acid sequence and/or the nucleic acid encoding the guide RNA may further comprise a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Recombinant Vector, Recombinant Host Cell and Kit

According to an embodiment of the present application, a recombinant vector can be provided, wherein, the recombinant vector comprises the nucleic acid encoding the nuclease described in the present application, the guide RNA described in the present application, the nucleic acid described in the present application, the nucleic acid construct described in the present application, or the composition described in the present application. The recombinant vector can be any suitable vector. In some embodiments, the recombinant vector includes, but is not limited to, a recombinant cloning vector, a recombinant eukaryotic expression plasmid, or a recombinant viral vector. In some embodiments, the recombinant eukaryotic expression plasmid includes pcDNA3.1, pCMV, pUC18, pUC19, pUC57, pBAD, pET, pENTR, pGenlenti, or pAAV. In some embodiments, the recombinant virus vector includes a recombinant adenovirus vector, a recombinant adeno-associated virus vector, a recombinant retrovirus vector, a recombinant herpes simplex virus vector, or a recombinant vaccinia virus vector. The recombinant vector of the present invention can be constructed using methods well known in the art. For example, depending on the restriction sites contained in the backbone vector used, appropriate restriction sites can be added to both ends of the nucleic acid construct of the present invention, and then loaded into the backbone vector.

According to an embodiment of the present application, a recombinant host cell can be provided, wherein, the recombinant host cell comprises the nuclease described in the present application, the guide RNA described in the present application, the nucleic acid described in the present application, the nucleic acid construct described in the present application, the composition described in the present application, or the recombinant vector described in the present application. The recombinant host cell can be any host cell in which nucleases can be used. In some embodiments, the recombinant host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof. In some embodiments, the plant cell includes a monocot cell or a dicot cell. In some embodiments, the monocot cell or the dicot cell includes rice cell, maize cell, or soybean cell.

According to an embodiment of the present application, a kit can be provided, wherein, the kit comprises the nuclease described in the present application, the guide RNA described in the present application, the nucleic acid described in the present application, the nucleic acid construct described in the present application, the composition described in the present application, the recombinant vector described in the present application, or the recombinant host cell described in the present application.

### Method and Use

The nuclease-based gene editing tools and methods provided in the present application can be applied to many fields such as gene therapy, molecular breeding in animals and plants, industrial microorganism engineering, model animal engineering, and scientific research. Particularly in the field of gene therapy, it can be applied for gene knockout based on DNA double-strand breaks in human genome.

According to an embodiment of the present application, a method for introducing a double-strand break into a targeting gene of a host cell can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

According to an embodiment of the present application, a method for deleting, replacing or inserting a targeting gene of a host cell can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

According to an embodiment of the present application, a method for obtaining a host cell in which a targeting gene is deleted, replaced or inserted can be provided, wherein the method comprises: delivering the nuclease described in the present application, the guide RNA described in the present application, the nucleic acid described in the present application, the nucleic acid construct described in the present application, the composition described in the present application, or the recombinant vector described in the present application into a host cell.

The method of delivery into the host cell can be any suitable method. In some embodiments, the delivery method includes but is not limited to cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun. The methods of cell transfection and culture are routine methods in the art, and appropriate transfection and culture methods can be selected according to different cell types.

The host cell can be any host cell in which nucleases can be used. In some embodiments, the host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof. In some embodiments, the plant cell includes a monocot cell or a dicot cell. In some embodiments, the monocot cell or the dicot cell includes rice cell, maize cell, or soybean cell.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for introducing a double-strand break into a targeting gene of a host cell can be provided.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for deleting, replacing or inserting a targeting gene of a host cell can be provided.

The host cell can be any host cell in which nucleases can be used. In some embodiments, the host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW. 4, R1, and D3) and differentiated cells thereof, or an induced pluripotent stem cell line and differentiated cells thereof. In some embodiments, the plant cell includes a monocot cell or a dicot cell. In some embodiments, the monocot cell or the dicot cell includes rice cell, maize cell, or soybean cell.

According to an embodiment of the present application, the use of the nuclease described in the present application, the guide RNA described in the present application, the nucleic acid described in the present application, the nucleic acid construct described in the present application, the composition described in the present application, the recombinant vector described in the present application, or the recombinant host cell described in the present application for preparing a drug or a preparation for gene therapy, cell therapy, genome research, and stem cell induction and post-induction differentiation can be provided.

The above various embodiments and preferences for the present application can be combined with each other (as long as they are not inherently contradictory to each other) and are suitable for the use of the present application, and the various embodiments formed by such combinations are considered as a part of the present application.

### EXAMPLES

Exemplary embodiments of the present application are described below in conjunction with the accompanying drawings, where various details of the examples of the present application are included to facilitate understanding. It should be understood that they are considered to be exemplary only and not intended to limit the protection scope of the present application. The protection scope of the present application is only defined by the claims. Therefore, those of ordinary skill in the art should be aware that various changes and modifications can be made to the examples described herein, without departing from the scope of the present application. Likewise, for clarity and conciseness, the description of well-known functions and structures is omitted in the following description.

Unless otherwise stated, the reagents and instruments used in the following examples are conventional products that are commercially available. Unless otherwise stated, experiments are performed under conventional conditions or conditions recommended by the manufacturer.

### Example 1: Construction of Nuclease Activity Detection System

A set of an RGS dual fluorescence surrogate reporter system was established to verify the activity of candidate nucleases.

Plasmid 1 consists of a complete set of elements capable of transcribing and expressing candidate nuclease proteins, comprising a constitutive promoter CMV (sequence as shown in SEQ ID NO: 405) that can initiate transcription in an eukaryotic cell, a candidate nuclease sequence (as shown in Table 1), a 5′-nuclear localization signal peptide sequence (sequence as shown in SEQ ID NO: 406), a 3′-nuclear localization signal peptide sequence (sequence as shown in SEQ ID NO: 407), a polyA sequence (sequence as shown in SEQ ID NO:408) that terminates transcription, and an ampicillin resistance gene sequence (sequence as shown in SEQ ID NO: 409).

Method for constructing plasmid 1: The amino acid sequence (or nucleotide sequence) of the candidate nuclease protein was synthesized through conventional gene synthesis by BGI Tech Solutions (Beijing Liuhe) Co., Ltd., with an ECORI cleavage site inserted into the upstream 5′ end of the sequence, and a BamH1 cleavage site inserted into the downstream 3′ end. Plasmid construction was also performed by the company responsible for the gene synthesis, and the specific construction method was as follows: 1. Preparation of vector. The plasmid backbone of a pcDNA3.1 plasmid vector was subjected to a double enzymatic cleavage digestion reaction using the single restriction endonuclease cleavage sites ECORI and BamHI on the plasmid vector, a linearized plasmid vector fragment was obtained by agarose gel electrophoresis, and the enzymatic cleavage band was excised from the gel for recovery to obtain the purified linearized plasmid vector fragment. 2. Ligation. The nucleotide sequence of the candidate nuclease protein obtained through conventional gene synthesis was ligated with the linearized pcDNA3.1 vector fragment using a T4 DNA ligase. 3. Transformation and verification. Monoclonal transformants were obtained through a LB agar plate for screening ampicillin resistance, and the correct clone identified by sequencing was used as a candidate plasmid for later use.

Plasmid 2 comprises a reRNA sequence (as shown in Table 1), with a 20 nt targeted sequence GCTCGGAGATCATCATTGCG inserted at the 3′ end of the reRNA sequence, a U6 promoter (sequence as shown in SEQ ID NO: 410), a PBR322 replication origin (sequence as shown in SEQ ID NO: 411), and an ampicillin resistance gene sequence (sequence as shown in SEQ ID NO: 409).

Method for constructing plasmid 2: Guide reRNA was synthesized through conventional gene synthesis by Beijing Tsingke Biotech Co., Ltd. or General Biosystems (Anhui) Co., Ltd. Plasmid construction was also performed by the company responsible for the gene synthesis, and the specific construction method was as follows: 1. Preparation of vector. A pUC19-U6 vector was subjected to enzymatic cleavage using BbsI, a linearized plasmid vector fragment was obtained by agarose gel electrophoresis, and the enzymatic cleavage band was excised from the gel for recovery to obtain the purified linearized plasmid vector fragment. 2. Ligation. The nucleotide sequence of the guide reRNA obtained through gene synthesis was ligated with the linearized pUC19-U6 vector fragment using a ligation method of seamless cloning. 3. Transformation and verification. Monoclonal transformants were obtained through a LB agar plate for screening ampicillin resistance, and the correct clone identified by sequencing was used as a candidate plasmid for later use.

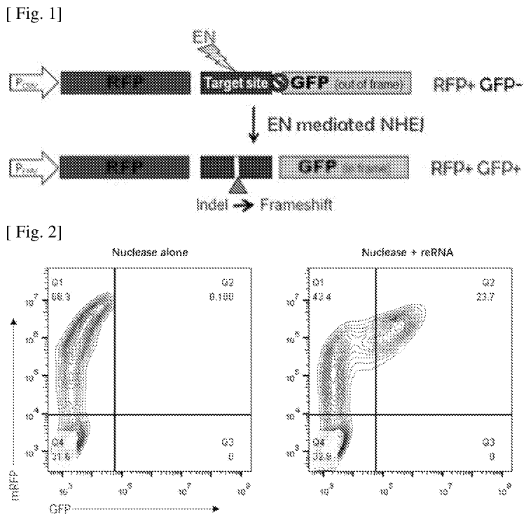

Plasmid 3 comprises a TAM sequence (as shown in Table 1), with a 20 nt targeted sequence GCTCGGAGATCATCATTGCG inserted at the 3′ end of the TAM sequence, a CMV promoter (sequence as shown in SEQ ID NO: 412), an ampicillin resistance gene sequence (sequence as shown in SEQ ID NO: 409), and a surrogate reporter gene. The surrogate reporter gene can encode two fluorescent proteins (RFP sequence as shown in SEQ ID NO: 413, and GFP sequence as shown in SEQ ID NO: 414). By means of the insertion of an endonuclease downstream of RFP and the insertion of an endonuclease upstream of GFP, TAM and a 20 nt targeted sequence at the 3′ end of TAM can be recognized. When there is no endonuclease activity according to the detection system, the reporter gene only expresses RFP to indicate the reference gene expression level of the reporter system, while GFP is designed outside the open reading frame (ORF) and therefore is not expressed. When the candidate has endonuclease activity, it can induce a double-strand break at the targeting site before GFP, which leads to the frameshift mutation of the reading frame when DNA is repaired through non-homology end joining (NHEJ), resulting in GFP shifting from an out of frame state to an in frame state and beginning to express. The stronger the cleavage activity of a nuclease, the higher the proportion of GFP expressed after frameshift. Therefore, the expression intensity of GFP is positively correlated with the cleavage activity of the nuclease. The working mode of the detection system is as shown in FIG. 1.

Method for constructing plasmid 3: Through an oligo synthesis method, TAM, and a 20 nt targeted sequence with an ECORI enzymatic cleavage site inserted at the 5′ end of the upstream sequence and a BamH1 enzymatic cleavage site inserted at the 3′ end of the downstream sequence were subjected to whole synthesis. The specific construction was as follows: 1. Preparation of vector. The plasmid backbone of an RGS-pcDNA3.1 plasmid vector was subjected to a double enzymatic cleavage digestion reaction using the single restriction endonuclease cleavage sites ECORI and BamHI on the plasmid vector, a linearized plasmid vector fragment was obtained by agarose gel electrophoresis, and the enzymatic cleavage band was excised from the gel for recovery to obtain the purified linearized plasmid vector fragment. 2. Ligation. The nucleotide sequence of the guide reRNA obtained through gene synthesis was ligated with the linearized pUC19-U6 vector fragment using a ligation method of seamless cloning. 3. Transformation and verification. Monoclonal transformants were obtained through a LB agar plate for screening ampicillin resistance, and the correct clone identified by sequencing was used as a candidate plasmid for later use.

TABLE 1
Plasmid construction related sequences

| Name | Nuclease sequence | reRNA sequence | TAM sequence |
|---|---|---|---|
| TP_A_1 | SEQ ID NO: 1 | SEQ ID NO: 198 | TTAT |
| TP_A_2 | SEQ ID NO: 2 | SEQ ID NO: 199 | GCTAC |
| TP_A_8 | SEQ ID NO: 3 | SEQ ID NO: 200 | TTAT |
| TP_A_12 | SEQ ID NO: 4 | SEQ ID NO: 201 | TTAT |
| TP_A_18 | SEQ ID NO: 5 | SEQ ID NO: 202 | TTGAT |
| TP_B_18 | SEQ ID NO: 6 | SEQ ID NO: 203 | TTAT |
| TP_B_41 | SEQ ID NO: 7 | SEQ ID NO: 204 | TTAT |
| TP_B_46 | SEQ ID NO: 8 | SEQ ID NO: 205 | TTTAT |
| TP_B_70 | SEQ ID NO: 9 | SEQ ID NO: 206 | TGAC |
| TP_B_71 | SEQ ID NO: 10 | SEQ ID NO: 207 | TGAT |
| TP_B_72 | SEQ ID NO: 11 | SEQ ID NO: 208 | TGAC |
| TP_B_73 | SEQ ID NO: 12 | SEQ ID NO: 209 | TGAT |
| TP_C_23 | SEQ ID NO: 13 | SEQ ID NO: 210 | CCAT |
| TP_C_67 | SEQ ID NO: 14 | SEQ ID NO: 211 | TTTAA |
| TP_C_70 | SEQ ID NO: 15 | SEQ ID NO: 212 | TTAC |
| TP_C_74 | SEQ ID NO: 16 | SEQ ID NO: 213 | TTAC |
| TP_D_1 | SEQ ID NO: 17 | SEQ ID NO: 214 | TGAC |
| TP_D_3 | SEQ ID NO: 18 | SEQ ID NO: 215 | TGAC |
| TP_D_4 | SEQ ID NO: 19 | SEQ ID NO: 216 | TGAC |
| TP_D_8 | SEQ ID NO: 20 | SEQ ID NO: 217 | GCTAC |
| TP_D_17 | SEQ ID NO: 21 | SEQ ID NO: 218 | TTAT |
| TP_D_18 | SEQ ID NO: 22 | SEQ ID NO: 219 | TTAT |
| TP_D_23 | SEQ ID NO: 23 | SEQ ID NO: 220 | TTAT |
| TP_D_24 | SEQ ID NO: 24 | SEQ ID NO: 221 | TTAC |
| TP_D_25 | SEQ ID NO: 25 | SEQ ID NO: 222 | TTAC |
| TP_D_27 | SEQ ID NO: 26 | SEQ ID NO: 223 | TGAT |
| TP_D_30 | SEQ ID NO: 27 | SEQ ID NO: 224 | TTTAA |
| TP_D_32 | SEQ ID NO: 28 | SEQ ID NO: 225 | TTTAA |
| TP_D_40 | SEQ ID NO: 29 | SEQ ID NO: 226 | TGAC |
| TP_D_43 | SEQ ID NO: 30 | SEQ ID NO: 227 | TGAT |
| TP_D_51 | SEQ ID NO: 31 | SEQ ID NO: 228 | GCTAC |
| TP_D_59 | SEQ ID NO: 32 | SEQ ID NO: 229 | TTAC |
| TP_D_61 | SEQ ID NO: 33 | SEQ ID NO: 230 | TTAC |
| TP_D_66 | SEQ ID NO: 34 | SEQ ID NO: 231 | TTAC |
| TP_D_67 | SEQ ID NO: 35 | SEQ ID NO: 232 | TTAC |
| TP_D_71 | SEQ ID NO: 36 | SEQ ID NO: 233 | TTTAA |
| TP_D_72 | SEQ ID NO: 37 | SEQ ID NO: 234 | TTTAA |
| TP_D_73 | SEQ ID NO: 38 | SEQ ID NO: 235 | TTTAA |
| TP_E_2 | SEQ ID NO: 39 | SEQ ID NO: 236 | TTAG |
| TP_E_15 | SEQ ID NO: 40 | SEQ ID NO: 237 | TTCAA |
| TP_E_17 | SEQ ID NO: 41 | SEQ ID NO: 238 | TCAA |
| TP_E_48 | SEQ ID NO: 42 | SEQ ID NO: 239 | TGAT |
| TP_F_56 | SEQ ID NO: 43 | SEQ ID NO: 240 | CCAT |
| TP_F_71 | SEQ ID NO: 44 | SEQ ID NO: 241 | GTGAC |
| TP_F_77 | SEQ ID NO: 45 | SEQ ID NO: 242 | TTTAA |
| TP_F_80 | SEQ ID NO: 46 | SEQ ID NO: 243 | TTTAA |
| TP_F_83 | SEQ ID NO: 47 | SEQ ID NO: 244 | TTTAA |
| TP_F_85 | SEQ ID NO: 48 | SEQ ID NO: 245 | TTTAA |
| TP_G_14 | SEQ ID NO: 49 | SEQ ID NO: 246 | TTAG |
| TP_G_19 | SEQ ID NO: 50 | SEQ ID NO: 247 | TTAT |
| TP_G_20 | SEQ ID NO: 51 | SEQ ID NO: 248 | TTTAT |
| TP_G_24 | SEQ ID NO: 52 | SEQ ID NO: 249 | TTAT |
| TP_G_43 | SEQ ID NO: 53 | SEQ ID NO: 250 | TCAC |
| TP_G_52 | SEQ ID NO: 54 | SEQ ID NO: 251 | TCAT |
| TP_G_53 | SEQ ID NO: 55 | SEQ ID NO: 252 | TCAT |
| TP_G_61 | SEQ ID NO: 56 | SEQ ID NO: 253 | TTCAT |
| TP_G_66 | SEQ ID NO: 57 | SEQ ID NO: 254 | TTGAA |
| TP_G_72 | SEQ ID NO: 58 | SEQ ID NO: 255 | TGAT |
| TP_G_75 | SEQ ID NO: 59 | SEQ ID NO: 256 | TTTAA |
| TP_G_83 | SEQ ID NO: 60 | SEQ ID NO: 257 | TTAT |
| TP_G_84 | SEQ ID NO: 61 | SEQ ID NO: 258 | TTAT |
| TP_H_1 | SEQ ID NO: 62 | SEQ ID NO: 259 | TTTAA |
| TP_H_3 | SEQ ID NO: 63 | SEQ ID NO: 260 | TTTAA |
| TP_H_4 | SEQ ID NO: 64 | SEQ ID NO: 261 | TTTAA |
| TP_H_5 | SEQ ID NO: 65 | SEQ ID NO: 262 | TTTAA |
| TP_H_6 | SEQ ID NO: 66 | SEQ ID NO: 263 | TTTAA |
| TP_H_9 | SEQ ID NO: 67 | SEQ ID NO: 264 | TTTAA |
| TP_H_11 | SEQ ID NO: 68 | SEQ ID NO: 265 | TTTAA |
| TP_H_12 | SEQ ID NO: 69 | SEQ ID NO: 266 | TTTAA |
| TP_H_13 | SEQ ID NO: 70 | SEQ ID NO: 267 | TTTAA |
| TP_H_15 | SEQ ID NO: 71 | SEQ ID NO: 268 | TTTAA |
| TP_H_18 | SEQ ID NO: 72 | SEQ ID NO: 269 | TTTAA |
| TP_H_19 | SEQ ID NO: 73 | SEQ ID NO: 270 | TTTAA |
| TP_H_20 | SEQ ID NO: 74 | SEQ ID NO: 271 | TTTAA |
| TP_H_21 | SEQ ID NO: 75 | SEQ ID NO: 272 | TTTAA |
| TP_H_23 | SEQ ID NO: 76 | SEQ ID NO: 273 | TTTAA |
| TP_H_24 | SEQ ID NO: 77 | SEQ ID NO: 274 | TTTAA |
| TP_H_30 | SEQ ID NO: 78 | SEQ ID NO: 275 | TTTAA |
| TP_H_31 | SEQ ID NO: 79 | SEQ ID NO: 276 | TTTAA |
| TP_H_32 | SEQ ID NO: 80 | SEQ ID NO: 277 | TTTAA |
| TP_H_34 | SEQ ID NO: 81 | SEQ ID NO: 278 | TTTAA |
| TP_H_38 | SEQ ID NO: 82 | SEQ ID NO: 279 | TTTAA |
| TP_H_39 | SEQ ID NO: 83 | SEQ ID NO: 280 | TTTAA |
| TP_H_40 | SEQ ID NO: 84 | SEQ ID NO: 281 | TTTAA |
| TP_H_43 | SEQ ID NO: 85 | SEQ ID NO: 282 | TTTAA |
| TP_I_1 | SEQ ID NO: 86 | SEQ ID NO: 283 | CCAT |
| TP_I_2 | SEQ ID NO: 87 | SEQ ID NO: 284 | CCAT |
| TP_I_3 | SEQ ID NO: 88 | SEQ ID NO: 285 | CCAT |
| TP_1_4 | SEQ ID NO: 89 | SEQ ID NO: 286 | CCAT |
| TP_I_5 | SEQ ID NO: 90 | SEQ ID NO: 287 | CCAT |
| TP_1_6 | SEQ ID NO: 91 | SEQ ID NO: 288 | CCAT |
| TP_I_7 | SEQ ID NO: 92 | SEQ ID NO: 289 | CCAT |
| TP_I_8 | SEQ ID NO: 93 | SEQ ID NO: 290 | CCAT |
| TP_I_9 | SEQ ID NO: 94 | SEQ ID NO: 291 | CCAT |
| TP_I_10 | SEQ ID NO: 95 | SEQ ID NO: 292 | CCAT |
| TP_I_11 | SEQ ID NO: 96 | SEQ ID NO: 293 | CCAT |
| TP_I_12 | SEQ ID NO: 97 | SEQ ID NO: 294 | CCAT |
| TP_I_13 | SEQ ID NO: 98 | SEQ ID NO: 295 | CCAT |
| TP_1_15 | SEQ ID NO: 99 | SEQ ID NO: 296 | CCAT |
| TP_I_16 | SEQ ID NO: 100 | SEQ ID NO: 297 | CCAT |
| TP_I_17 | SEQ ID NO: 101 | SEQ ID NO: 298 | CCAT |
| TP_I_18 | SEQ ID NO: 102 | SEQ ID NO: 299 | CCAT |
| TP_I_19 | SEQ TD NO: 103 | SEQ ID NO: 300 | CCAT |
| TP_I_20 | SEQ ID NO: 104 | SEQ ID NO: 301 | CCAT |
| TP_I_21 | SEQ ID NO: 105 | SEQ ID NO: 302 | CCAT |
| TP_I_22 | SEQ ID NO: 106 | SEQ ID NO: 303 | CCAT |
| TP_I_24 | SEQ ID NO: 107 | SEQ ID NO: 304 | CCAT |
| TP_I_25 | SEQ ID NO: 108 | SEQ ID NO: 305 | CCAT |
| TP_I_26 | SEQ ID NO: 109 | SEQ ID NO: 306 | CCAT |
| TP_I_29 | SEQ ID NO: 110 | SEQ ID NO: 307 | CCAT |
| TP_I_31 | SEQ ID NO: 111 | SEQ ID NO: 308 | CCAT |
| TP_I_35 | SEQ ID NO: 112 | SEQ ID NO: 309 | CCAT |
| TP_I_37 | SEQ ID NO: 113 | SEQ ID NO: 310 | TTAT |
| TP_I_38 | SEQ ID NO: 114 | SEQ ID NO: 311 | TTAT |
| TP_I_40 | SEQ ID NO: 115 | SEQ ID NO: 312 | TTAT |
| TP_I_41 | SEQ ID NO: 116 | SEQ ID NO: 313 | TTAT |
| TP_I_44 | SEQ ID NO: 117 | SEQ ID NO: 314 | TTAT |
| TP_I_45 | SEQ ID NO: 118 | SEQ ID NO: 315 | TTAT |
| TP_I_46 | SEQ ID NO: 119 | SEQ ID NO: 316 | TTAT |
| TP_I_47 | SEQ ID NO: 120 | SEQ ID NO: 317 | TTAT |
| TP_I_48 | SEQ ID NO: 121 | SEQ ID NO: 318 | TTAT |
| TP_I_49 | SEQ ID NO: 122 | SEQ ID NO: 319 | TTAT |
| TP_I_50 | SEQ ID NO: 123 | SEQ ID NO: 320 | TTAT |
| TP_I_51 | SEQ ID NO: 124 | SEQ ID NO: 321 | TTAT |
| TP_I_52 | SEQ ID NO: 125 | SEQ ID NO: 322 | TTAT |
| TP_I_53 | SEQ ID NO: 126 | SEQ ID NO: 323 | TTAT |
| TP_I_55 | SEQ ID NO: 127 | SEQ ID NO: 324 | TTAT |
| TP_I_56 | SEQ ID NO: 128 | SEQ ID NO: 325 | TTAT |
| TP_I_58 | SEQ ID NO: 129 | SEQ ID NO: 326 | TTAT |
| TP_I_59 | SEQ ID NO: 130 | SEQ ID NO: 327 | TTAT |
| TP_I_61 | SEQ ID NO: 131 | SEQ ID NO: 328 | TTAT |
| TP_I_62 | SEQ ID NO: 132 | SEQ ID NO: 329 | TTAT |
| TP_I_64 | SEQ ID NO: 133 | SEQ ID NO: 330 | TTAT |
| TP_I_65 | SEQ ID NO: 134 | SEQ ID NO: 331 | TTAT |
| TP_I_66 | SEQ ID NO: 135 | SEQ ID NO: 332 | TTAT |
| TP_I_67 | SEQ ID NO: 136 | SEQ ID NO: 333 | TTAT |
| TP_I_70 | SEQ ID NO: 137 | SEQ ID NO: 334 | TTAT |
| TP_I_71 | SEQ ID NO: 138 | SEQ ID NO: 335 | TTAT |
| TP_I_76 | SEQ ID NO: 139 | SEQ ID NO: 336 | TTAT |
| TP_I_77 | SEQ TD NO: 140 | SEQ ID NO: 337 | TTAT |
| TP_I_80 | SEQ ID NO: 141 | SEQ ID NO: 338 | TTAT |
| TP_I_82 | SEQ ID NO: 142 | SEQ ID NO: 339 | TTAT |
| TP_I_84 | SEQ ID NO: 143 | SEQ ID NO: 340 | TTAT |
| TP_I_86 | SEQ ID NO: 144 | SEQ ID NO: 341 | TTAT |
| TP_I_87 | SEQ ID NO: 145 | SEQ ID NO: 342 | TTAT |
| TP_I_79 | SEQ ID NO: 146 | SEQ ID NO: 343 | TTAT |
| TP_I_85 | SEQ ID NO: 147 | SEQ ID NO: 344 | TTAT |
| TP_L_1 | SEQ ID NO: 148 | SEQ ID NO: 345 | TTTAA |
| TP_L_4 | SEQ ID NO: 149 | SEQ ID NO: 346 | TTTAA |
| TP_L_5 | SEQ TD NO: 150 | SEQ ID NO: 347 | TTTAA |
| TP_L_8 | SEQ ID NO: 151 | SEQ ID NO: 348 | TTTAA |
| TP_L_9 | SEQ ID NO: 152 | SEQ ID NO: 349 | TTTAA |
| TP_L_10 | SEQ ID NO: 153 | SEQ ID NO: 350 | TTTAA |
| TP_L_11 | SEQ ID NO: 154 | SEQ ID NO: 351 | TTTAA |
| TP_L_12 | SEQ ID NO: 155 | SEQ ID NO: 352 | TTTAA |
| TP_L_15 | SEQ ID NO: 156 | SEQ ID NO: 353 | TTTAA |
| TP_L_16 | SEQ ID NO: 157 | SEQ ID NO: 354 | TTTAA |
| TP_L_17 | SEQ ID NO: 158 | SEQ ID NO: 355 | TTTAA |
| TP_L_21 | SEQ ID NO: 159 | SEQ ID NO: 356 | TTTAA |
| TP_L_22 | SEQ ID NO: 160 | SEQ ID NO: 357 | TTTAA |
| TP_L_24 | SEQ ID NO: 161 | SEQ ID NO: 358 | TTTAA |
| TP_L_25 | SEQ ID NO: 162 | SEQ ID NO: 359 | TTTAA |
| TP_L_26 | SEQ ID NO: 163 | SEQ ID NO: 360 | TTTAA |
| TP_L_27 | SEQ ID NO: 164 | SEQ ID NO: 361 | TTTAA |
| TP_L_28 | SEQ ID NO: 165 | SEQ ID NO: 362 | TTTAA |
| TP_L_31 | SEQ ID NO: 166 | SEQ ID NO: 363 | TTTAA |
| TP_L_32 | SEQ ID NO: 167 | SEQ ID NO: 364 | TTTAA |
| TP_L_34 | SEQ ID NO: 168 | SEQ ID NO: 365 | TTTAA |
| TP_L_36 | SEQ ID NO: 169 | SEQ ID NO: 366 | TTTAA |
| TP_L_37 | SEQ ID NO: 170 | SEQ ID NO: 367 | TTTAA |
| TP_L_39 | SEQ ID NO: 171 | SEQ ID NO: 368 | TTTAA |
| TP_M_1 | SEQ ID NO: 172 | SEQ ID NO: 369 | TTGAT |
| TP_M_3 | SEQ ID NO: 173 | SEQ ID NO: 370 | TTGAT |
| TP_M_7 | SEQ ID NO: 174 | SEQ ID NO: 371 | TGAC |
| TP_M_11 | SEQ ID NO: 175 | SEQ ID NO: 372 | TGAC |
| TP_M_14 | SEQ ID NO: 176 | SEQ ID NO: 373 | TGAC |
| TP_M_17 | SEQ TD NO: 177 | SEQ ID NO: 374 | TGAC |
| TP_M_19 | SEQ ID NO: 178 | SEQ ID NO: 375 | TGAC |
| TP_M_20 | SEQ ID NO: 179 | SEQ ID NO: 376 | TGAC |
| TP_M_24 | SEQ TD NO: 180 | SEQ ID NO: 377 | TGAC |
| TP_M_31 | SEQ ID NO: 181 | SEQ TD NO: 378 | TTAC |
| TP_M_32 | SEQ ID NO: 182 | SEQ ID NO: 379 | TTAC |
| TP_M_33 | SEQ ID NO: 183 | SEQ LD NO: 380 | TTAC |
| TP_M_34 | SEQ ID NO: 184 | SEQ ID NO: 381 | TTAC |
| TP_M_35 | SEQ ID NO: 185 | SEQ ID NO: 382 | TTAC |
| TP_M_37 | SEQ ID NO: 186 | SEQ ID NO: 383 | TTAG |
| TP_M_40 | SEQ ID NO: 187 | SEQ ID NO: 384 | TTAC |
| TP_M_41 | SEQ ID NO: 188 | SEQ ID NO: 385 | TTAC |
| TP_M_43 | SEQ ID NO: 189 | SEQ ID NO: 386 | TTAC |
| TP_M_46 | SEQ ID NO: 190 | SEQ ID NO: 387 | TCAC |
| TP_M_49 | SEQ ID NO: 191 | SEQ ID NO: 388 | TCAC |
| TP_M_58 | SEQ ID NO: 192 | SEQ ID NO: 389 | TCAC |
| TP_M_65 | SEQ ID NO: 193 | SEQ ID NO: 390 | TCAC |
| TP_M_66 | SEQ ID NO: 194 | SEQ ID NO: 391 | TCAC |
| TP_M_67 | SEQ ID NO: 195 | SEQ ID NO: 392 | TCAC |
| TP_M_70 | SEQ ID NO: 196 | SEQ ID NO: 393 | TCAC |
| TP_M_78 | SEQ ID NO: 197 | SEQ ID NO: 394 | TCAC |
| TP_A_24 | SEQ ID NO: 395 | SEQ ID NO: 400 | TTTAA |
| TP_A_54 | SEQ ID NO: 396 | SEQ ID NO: 401 | TTTAG |
| TP_B_23 | SEQ ID NO: 397 | SEQ ID NO: 402 | TTGAT |
| TP_D_44 | SEQ ID NO: 398 | SEQ ID NO: 403 | TTGAT |
| TP_F_76 | SEQ ID NO: 399 | SEQ ID NO: 404 | ITTAA |

### Example 2: Detection of Nuclease Activity

### 2.1 Cell Treatment:

After HEK293T cells (commercially purchased) were cultured to the logarithmic growth phase, they were trypsinized into single cells with 0.25% Trypsin (Thermo), and added to a 96-well cell culture plate pre-coated with PDL (Sigma) at a cell concentration of 3× 10⁴ cells/well, and cultured overnight at 37° C. in 5% CO₂.

### 2.2 Cell Transfection:

The three functional plasmids described in example 1 (the nuclease plasmid, the reRNA-targeted sequence plasmid and the RGS dual fluorescence reporter system plasmid) were co-transfected into HEK293T cells, wherein 60 ng of the nuclease plasmid, 40 ng of the reRNA-targeted sequence plasmid and 100 ng of the RGS dual fluorescence reporter system plasmid were added to a 96-well cell culture plate, respectively, and transfection was performed using Lipofectamine™ 2000 (Invitrogen, Cat. No. 11668019) at a ratio of transfection reagent volume (μL):plasmid mass (μg) of 2:1.

### 2.3 Obtaining Results

After transfection, the cells were cultured for 48 h, then typsinized and collected, and detected by a flow cytometry. The final screening results were analyzed on the basis of the positive expression of GFP.

### 2.4 Detection Results

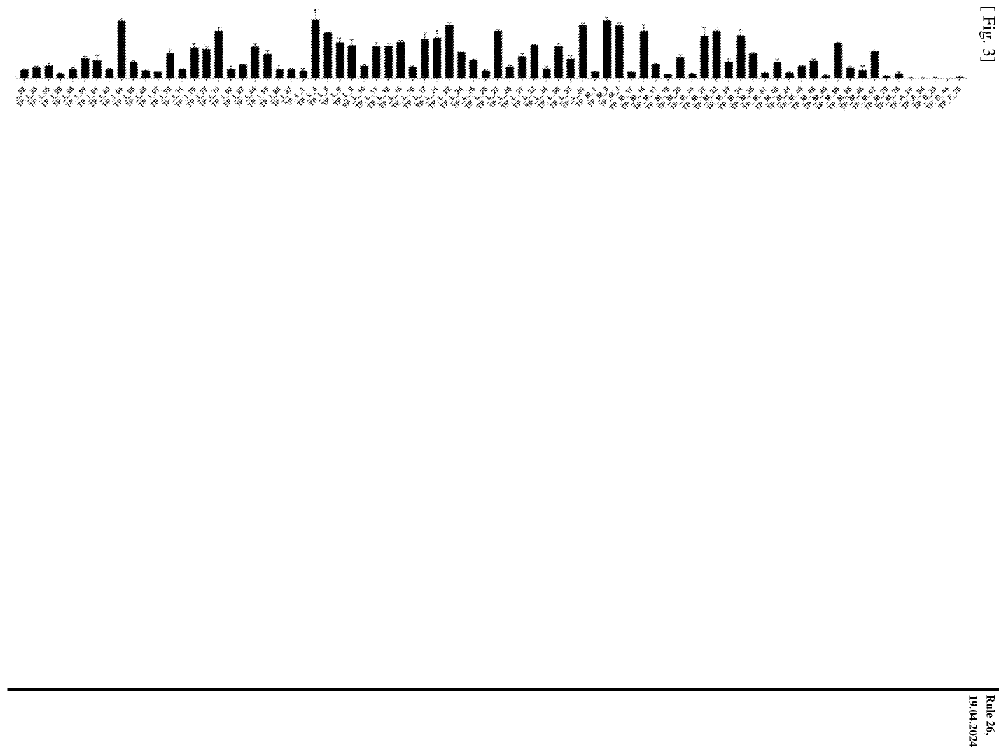

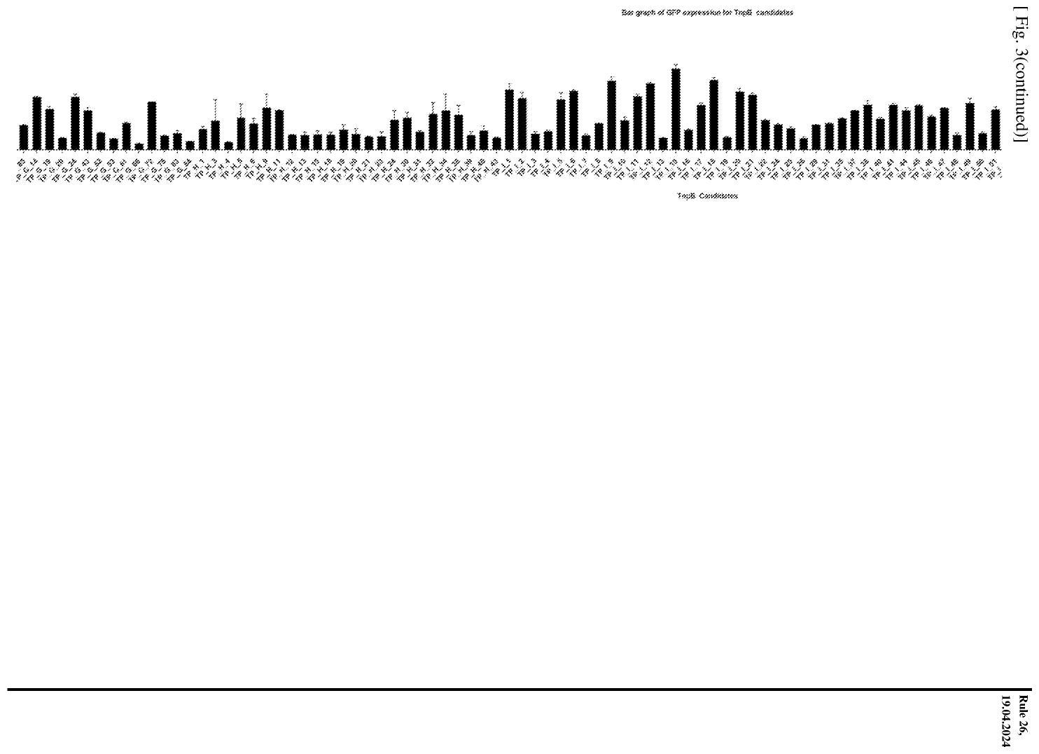

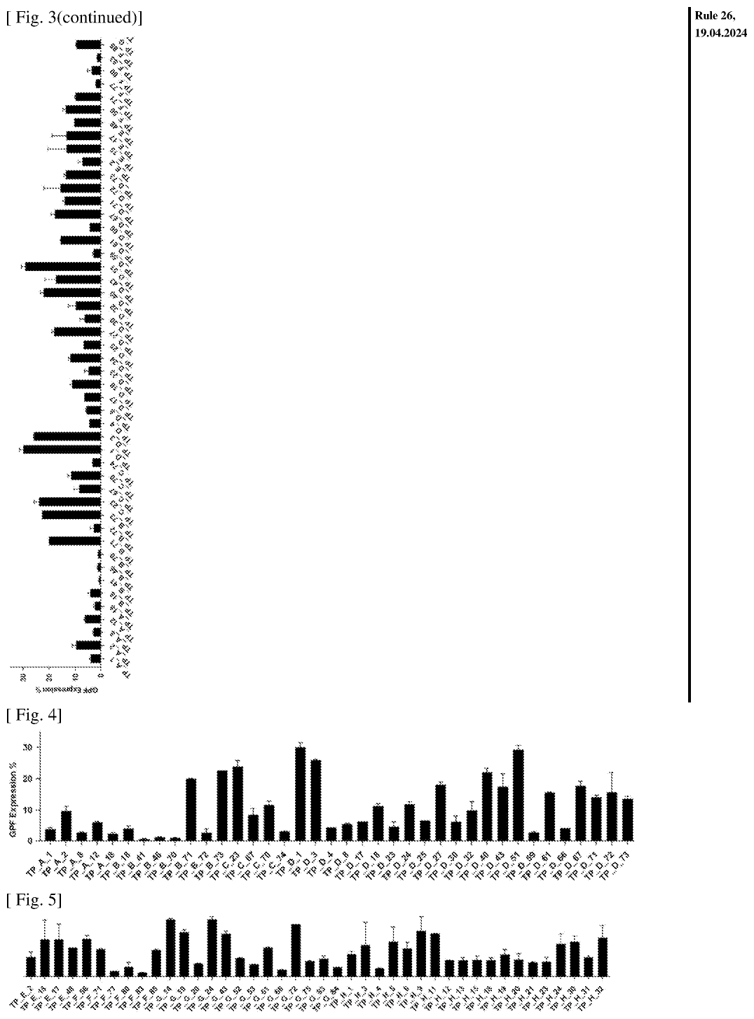

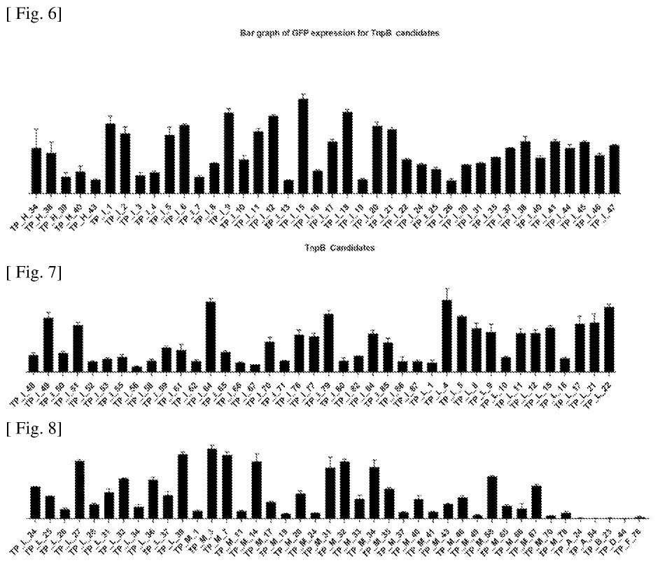

The results of nuclease activity were obtained by flow cytometry, and the data were as shown in FIG. 2, with the ordinate in the figure showing the RFP expression (%) and the abscissa showing the GFP expression (%), which reflected the cleavage activity of the nuclease. Furthermore, the statistical results of the GFP expressions reflecting the activities of all nucleases were as shown in FIG. 3 and Table 2. The results showed that the 197 nucleases (TP_A_1, TP_A_2, TP_A_8, TP_A_12, TP_A_18, TP_B_18, TP_B_41, TP_B_46, TP_B_70, TP_B_71, TP_B_72, TP_B_73, TP_C_23, TP_C_67, TP_C_70, TP_C_74, TP_D_1, TP_D_3, TP_D_4, TP_D_8, TP_D_17, TP_D_18, TP_D_23, TP_D_24, TP_D_25, TP_D_27, TP_D_30, TP_D_32, TP_D_40, TP_D_43, TP_D_51, TP_D_59, TP_D_61, TP_D_66, TP_D_67, TP_D_71, TP_D_72, TP_D_73, TP_E_2, TP_E_15, TP_E_17, TP_E_48, TP_F_56, TP_F_71, TP_F_77, TP_F_80, TP_F_83, TP_F_85, TP_G_14, TP_G_19, TP_G_20, TP_G_24, TP_G_43, TP_G_52, TP_G_53, TP_G_61, TP_G_66, TP_G_72, TP_G_75, TP_G_83, TP_G_84, TP_H_1, TP_H_3, TP_H_4, TP_H_5, TP_H_6, TP_H_9, TP_H_11, TP_H_12, TP_H_13, TP_H_15, TP_H_18, TP_H_19, TP_H_20, TP_H_21, TP_H_23, TP_H_24, TP_H_30, TP_H_31, TP_H_32, TP_H_34, TP_H_38, TP_H_39, TP_H_40, TP_H_43, TP_I_1, TP_I_2, TP_I_3, TP_I_4, TP_I_5, TP_I_6, TP_I_7, TP_I_8, TP_I_9, TP_I_10, TP_I_11, TP_I_12, TP_I_13, TP_I_15, TP_I_16, TP_I_17, TP_I_18, TP_I_19, TP_I_20, TP_I_21, TP_I_22, TP_I_24, TP_I_25, TP_I_26, TP_I_29, TP_I_31, TP_I_35, TP_I_37, TP_I_38, TP_I_40, TP_I_41, TP_I_44, TP_I_45, TP_I_46, TP_I_47, TP_I_48, TP_I_49, TP_I_50, TP_I_51, TP_I_52, TP_I_53, TP_I_55, TP_I_56, TP_I_58, TP_I_59, TP_I_61, TP_I_62, TP_I_64, TP_I_65, TP_I_66, TP_I_67, TP_I_70, TP_I_71, TP_I_76, TP_I_77, TP_I_79, TP_I_80, TP_I_82, TP_I_84, TP_I_85, TP_I_86, TP_I_87, TP_L_1, TP_L_4, TP_L_5, TP_L_8, TP_L_9, TP_L_10, TP_L_11, TP_L_12, TP_L_15, TP_L_16, TP_L_17, TP_L_21, TP_L_22, TP_L_24, TP_L_25, TP_L_26, TP_L_27, TP_L_28, TP_L_31, TP_L_32, TP_L_34, TP_L_36, TP_L_37, TP_L_39, TP_M_1, TP_M_3, TP_M_7, TP_M_11, TP_M_14, TP_M_17, TP_M_19, TP_M_20, TP_M_24, TP_M_31, TP_M_32, TP_M_33, TP_M_34, TP_M_35, TP_M_37, TP_M_40, TP_M_41, TP_M_43, TP_M_46, TP_M_49, TP_M_58, TP_M_65, TP_M_66, TP_M_67, TP_M_70 and TP_M_78) in the present application had good activity.

Meanwhile, a large number of nucleases with inactive or low cleavage activity were also found during the screening process (e.g. TP_A_24, TP_A_54, TP_B_23, TP_D_44, and TP_F_76 in Table 1 of this application). Compared with these nucleases with inactive or low activity, the cleavage activity of the 197 nucleases of the present application were markedly higher.

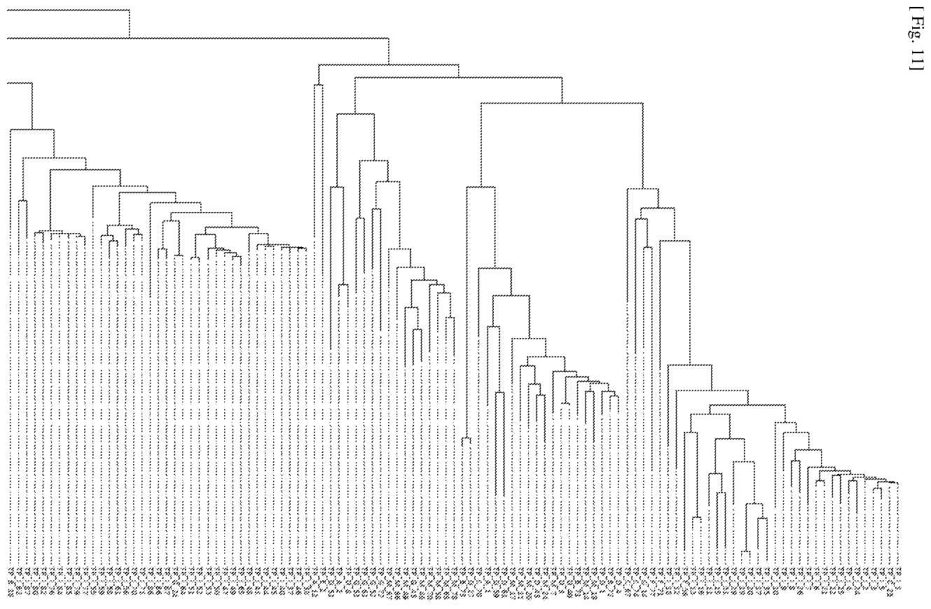

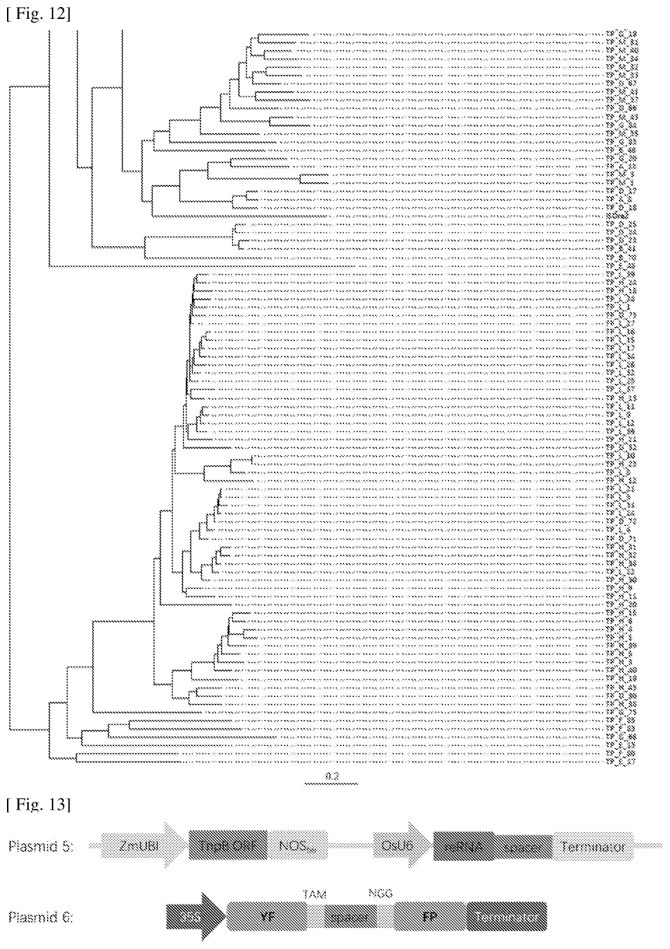

In addition, FIG. 11 showed an evolutionary branching diagram of the nucleases in the present application based on protein sequences. The result showed that these nucleases covered different branches of the superfamily, and ISDra2 was also included.

TABLE 2
The results of nuclease activity in example 2

| | Nuclease activity |
|---|---|
| Name | (mean ± standard deviation) |
| TP_A_1 | 3.92 ± 0.44 |
| TP_A_2 | 9.70 ± 1.46 |
| TP_A_8 | 2.78 ± 0.15 |
| TP_A_12 | 6.10 ± 0.30 |
| TP_A_18 | 2.36 ± 0.40 |
| TP_B_18 | 4.10 ± 0.78 |
| TP_B_41 | 0.66 ± 0.16 |
| TP_B_46 | 1.18 ± 0.13 |
| TP_B_70 | 0.95 ± 0.13 |
| TP_B_71 | 19.94 ± 0.06 |
| TP_B_72 | 2.66 ± 1.31 |
| TP_B_73 | 22.61 ± 0.01 |
| TP_C_23 | 23.83 ± 1.97 |
| TP_C_67 | 8.39 ± 2.07 |
| TP_C_70 | 11.56 ± 1.23 |
| TP_C_74 | 3.16 ± 0.12 |
| TP_D_1 | 29.99 ± 1.48 |
| TP_D_3 | 25.85 ± 0.28 |
| TP_D_4 | 4.44 ± 0 |
| TP_D_8 | 5.53 ± 0.12 |
| TP_D_17 | 6.29 ± 0 |
| TP_D_18 | 11.25 ± 0.71 |
| TP_D_23 | 4.66 ± 1.51 |
| TP_D_24 | 11.89 ± 0.71 |
| TP_D_25 | 6.61 ± 0 |
| TP_D_27 | 18.14 ± 0.78 |
| TP_D_30 | 6.20 ± 1.93 |
| TP_D_32 | 9.84 ± 2.88 |
| TP_D_40 | 22.01 ± 1.27 |
| TP_D_43 | 17.44 ± 4.17 |
| TP_D_51 | 29.25 ± 1.34 |
| TP_D_59 | 2.81 ± 0.21 |
| TP_D_61 | 15.55 ± 0.14 |
| TP_D_66 | 4.27 ± 0 |
| TP_D_67 | 17.73 ± 1.48 |
| TP_D_71 | 14.06 ± 0.64 |
| TP_D_72 | 15.62 ± 6.36 |
| TP_D_73 | 13.55 ± 0.78 |
| TP_E_2 | 7.03 ± 1.84 |
| TP_E_15 | 13.41 ± 6.99 |
| TP_E_17 | 13.41 ± 5.44 |
| TP_E_48 | 10.37 ± 0 |
| TP_F_56 | 13.65 ± 1.09 |
| TP_F_71 | 9.91 ± 0.27 |
| TP_F_77 | 1.91 ± 0.04 |
| TP_F_80 | 3.59 ± 1.51 |
| TP_F_83 | 1.38 ± 0.09 |
| TP_F_85 | 9.67 ± 0.16 |
| TP_G_14 | 20.62 ± 0.34 |
| TP_G_19 | 15.99 ± 0.87 |
| TP_G_20 | 4.64 ± 0.12 |
| TP_G_24 | 20.69 ± 1.04 |
| TP_G_43 | 15.33 ± 1.07 |
| TP_G_52 | 6.75 ± 0.09 |
| TP_G_53 | 4.42 ± 0.11 |
| TP_G_61 | 10.36 ± 0.33 |
| TP_G_66 | 2.41 ± 0.21 |
| TP_G_72 | 18.87 ± 0 |
| TP_G_75 | 5.53 ± 0.17 |
| TP_G_83 | 6.50 ± 1.00 |
| TP_G_84 | 3.35 ± 0.07 |
| TP_H_1 | 8.11 ± 0.94 |
| TP_H_3 | 11.32 ± 8.28 |
| TP_H_4 | 3.01 ± 0.08 |
| TP_H_5 | 12.54 ± 5.42 |
| TP_H_6 | 10.25 ± 2.01 |
| TP_H_9 | 16.47 ± 5.23 |
| TP_H_11 | 15.42 ± 0.07 |
| TP_H_12 | 5.88 ± 0.10 |
| TP_H_13 | 5.74 ± 1.20 |
| TP_H_15 | 6.02 ± 1.40 |
| TP_H_18 | 5.88 ± 0.94 |
| TP_H_19 | 7.93 ± 1.90 |
| TP_H_20 | 6.17 ± 2.12 |
| TP_H_21 | 5.12 ± 0.15 |
| TP_H_23 | 5.29 ± 1.75 |
| TP_H_24 | 11.74 ± 3.59 |
| TP_H_30 | 12.57 ± 1.98 |
| TP_H_31 | 6.94 ± 0.43 |
| TP_H_32 | 14.02 ± 4.45 |
| TP_H_34 | 15.37 ± 6.36 |
| TP_H_38 | 13.62 ± 3.75 |
| TP_H_39 | 5.74 ± 1.44 |
| TP_H_40 | 7.45 ± 1.87 |
| TP_H_43 | 4.79 ± 0.23 |
| TP_I_1 | 23.47 ± 2.46 |
| TP_I_2 | 20.17 ± 2.18 |
| TP_I_3 | 6.26 ± 0.93 |
| TP_I_4 | 7.27 ± 0.38 |
| TP_I_5 | 19.67 ± 2.60 |
| TP_I_6 | 22.97 ± 0.37 |
| TP_I_7 | 5.72 ± 0.45 |
| TP_I_8 | 10.32 ± 0.13 |
| TP_I_9 | 27.07 ± 1.50 |
| TP_I_10 | 11.47 ± 1.36 |
| TP_I_11 | 20.92 ± 0.86 |
| TP_I_12 | 26.02 ± 0.41 |
| TP_I_13 | 4.65 ± 0.09 |
| TP_I_15 | 31.77 ± 1.61 |
| TP_I_16 | 7.86 ± 0.20 |
| TP_I_17 | 17.47 ± 0.79 |
| TP_I_18 | 27.32 ± 0.98 |
| TP_I_19 | 4.95 ± 0.21 |
| TP_I_20 | 22.72 ± 1.29 |
| TP_I_21 | 21.52 ± 0.58 |
| TP_I_22 | 11.62 ± 0.27 |
| TP_I_24 | 9.92 ± 0.27 |
| TP_I_25 | 8.37 ± 0.58 |
| TP_I_26 | 4.58 ± 0.49 |
| TP_I_29 | 9.77 ± 0.06 |
| TP_I_31 | 10.32 ± 0.27 |
| TP_I_35 | 12.32 ± 0.01 |
| TP_I_37 | 15.32 ± 0.16 |
| TP_I_38 | 17.57 ± 1.61 |
| TP_I_40 | 12.12 ± 0.58 |
| TP_I_41 | 17.62 ± 0.44 |
| TP_I_44 | 15.32 ± 1.15 |
| TP_I_45 | 17.37 ± 0.34 |
| TP_I_46 | 12.97 ± 0.51 |
| TP_I_47 | 16.32 ± 0.16 |
| TP_I_48 | 5.77 ± 0.74 |
| TP_I_49 | 18.27 ± 1.78 |
| TP_I_50 | 6.47 ± 0.47 |
| TP_I_51 | 15.67 ± 1.05 |
| TP_I_52 | 3.55 ± 0.18 |
| TP_I_53 | 4.40 ± 0.42 |
| TP_I_55 | 5.21 ± 0.71 |
| TP_I_56 | 1.96 ± 0.16 |
| TP_I_58 | 3.85 ± 0.48 |
| TP_I_59 | 8.22 ± 0.65 |
| TP_I_61 | 7.36 ± 1.98 |
| TP_I_62 | 3.69 ± 0.47 |
| TP_I_64 | 23.57 ± 1.22 |
| TP_I_65 | 6.70 ± 0.48 |
| TP_I_66 | 3.18 ± 0.17 |
| TP_I_67 | 2.60 ± 0.01 |
| TP_I_70 | 10.21 ± 1.45 |
| TP_I_71 | 3.84 ± 0.06 |
| TP_I_76 | 12.62 ± 1.57 |
| TP_I_77 | 11.97 ± 1.19 |
| TP_I_80 | 19.52 ± 1.15 |
| TP_I_82 | 3.80 ± 1.04 |
| TP_I_84 | 5.49 ± 0.06 |
| TP_I_86 | 12.97 ± 1.07 |
| TP_I_87 | 9.93 ± 1.39 |
| TP_I_79 | 3.64 ± 1.52 |
| TP_I_85 | 3.64 ± 0.40 |
| TP_L_1 | 3.24 ± 0.79 |
| TP_L_4 | 24.19 ± 3.80 |
| TP_L_5 | 18.79 ± 0.27 |
| TP_L_8 | 14.69 ± 1.82 |
| TP_L_9 | 13.44 ± 2.63 |
| TP_L_10 | 5.07 ± 0.25 |
| TP_L_11 | 13.14 ± 1.36 |
| TP_L_12 | 13.24 ± 0.91 |
| TP_L_15 | 14.94 ± 0.62 |
| TP_L_16 | 4.67 ± 0.33 |
| TP_L_17 | 16.19 ± 2.56 |
| TP_L_21 | 16.64 ± 2.91 |
| TP_L_22 | 21.84 ± 0.93 |
| TP_L_24 | 10.79 ± 0.01 |
| TP_L_25 | 7.68 ± 0.10 |
| TP_L_26 | 3.24 ± 0.31 |
| TP_L_27 | 19.54 ± 0.48 |
| TP_L_28 | 4.83 ± 0.45 |
| TP_L_31 | 8.91 ± 1.12 |
| TP_L_32 | 13.59 ± 0.16 |
| TP_L_34 | 4.09 ± 0.78 |
| TP_L_36 | 13.14 ± 0.93 |
| TP_L_37 | 8.00 ± 1.20 |
| TP_L_39 | 21.79 ± 0.72 |
| TP_M_1 | 2.59 ± 0.30 |
| TP_M_3 | 23.78 ± 1.16 |
| TP_M_7 | 21.65 ± 0.92 |
| TP_M_11 | 2.52 ± 0.30 |
| TP_M_14 | 19.35 ± 2.62 |
| TP_M_17 | 5.69 ± 0.30 |
| TP_M_19 | 1.66 ± 10.11 |
| TP_M_20 | 8.51 ± 0.96 |
| TP_M_24 | 1.90 ± 0.08 |
| TP_M_31 | 17.35 ± 3.49 |
| TP_M_32 | 19.40 ± 0.73 |
| TP_M_33 | 6.75 ± 1.16 |
| TP_M_34 | 17.50 ± 2.43 |
| TP_M_35 | 10.15 ± 0.37 |
| TP_M_37 | 2.15 ± 0.18 |
| TP_M_40 | 6.67 ± 1.19 |
| TP_M_41 | 2.26 ± 0.21 |
| TP_M_43 | 4.97 ± 0.24 |
| TP_M_46 | 7.25 ± 0.61 |
| TP_M_49 | 1.22 ± 0.21 |
| TP_M_58 | 14.35 ± 0.21 |
| TP_M_65 | 4.35 ± 0.40 |
| TP_M_66 | 3.41 ± 1.68 |
| TP_M_67 | 11.16 ± 0.47 |
| TP_M_70 | 1.03 ± 0.01 |
| TP_M_78 | 1.94 ± 0.50 |
| TP_A_24 | 0.25 ± 0.08 |
| TP_A_54 | 0.11 ± 0.09 |
| TP_B_23 | 0.24 ± 0.08 |
| TP_D_44 | 0.18 ± 0 |
| TP_F_76 | 0.48 ± 0.34 |

### Example 3: Detection of Editing Efficiency at Endogenous Loci

### 3.1 Construction of Plasmids:

The nuclease plasmid (plasmid 1) comprised a complete set of elements capable of transcribing and expressing candidate nuclease proteins, including a constitutive promoter CMV (sequence as shown in SEQ ID NO: 405) that can initiate transcription in an eukaryotic cell, a candidate nuclease sequence (as shown in Table 1), a 5′-nuclear localization signal peptide sequence (sequence as shown in SEQ ID NO: 406), a 3′-nuclear localization signal peptide sequence (sequence as shown in SEQ ID NO: 407), a polyA sequence (sequence as shown in SEQ ID NO: 408) that terminates transcription, and an ampicillin resistance gene sequence (sequence as shown in SEQ ID NO: 409). The method for constructing plasmid 1 is described in example 1.

The reRNA-targeted sequence plasmid (plasmid 4) comprises a reRNA sequence (as shown in Table 1), with a 20 nt targeted sequence of endogenous gene inserted at the 3′ end of the reRNA sequence (as shown in Table 3), a U6 promoter (sequence as shown in SEQ ID NO: 410), a PBR322 replication origin (sequence as shown in SEQ ID NO: 411), and an ampicillin resistance gene sequence (sequence as shown in SEQ ID NO: 409). In addition, different targeted sequences of endogenous genes (as shown in Table 2) can identify different targeting genes adjacent to the TAM sequences.

### Method for Constructing Plasmid 4:

1. Preparation of vector. A reRNA plasmid containing BBSI-BBSI fragment (pUC19-U6-reRNA-BbsI_BbsI) was subjected to enzymatic cleavage using BbsI, a linearized plasmid vector fragment was obtained by agarose gel electrophoresis, and the enzymatic cleavage band was excised from the gel for recovery to obtain the purified linearized plasmid vector fragment. 2. Preparation of 20 nt targeted sequences of endogenous genes. Firstly, the 20 bp DNA sequence adjacent to the 3′ end of the TAM sequence was searched in the endogenous gene sequence, and then oligonucleotides of targeted sequences with BbsI excision end were synthesized through primer synthesis. Finally, a double-stranded oligonucleotide with sticky ends was synthesized by annealing bonding. 3. Ligation. The targeted sequence of endogenous gene was ligated with the linearized pUC19-U6-reRNA-BbsI_BbsI vector fragment using T4 ligase. 4. Transformation and verification. Monoclonal transformants were obtained through a LB agar plate for screening ampicillin resistance, and the correct clone identified by sequencing was used as a candidate plasmid for later use.

### 3.2 Cell Treatment:

After HEK293T cells (commercially purchased) were cultured to the logarithmic growth phase, they were typsinized into single cells with 0.25% Trypsin (Thermo), and added to a 48-well cell culture plate pre-coated with PDL (Sigma) at a cell concentration of 1× 10⁵ cells/well, and cultured overnight at 37° C. in 5% CO₂.

### 3.3 Cell Transfection:

The two functional plasmids described in 3.1 (the nuclease plasmid and the reRNA-targeted sequence plasmid) were co-transfected into HEK293T cells, wherein 300 ng of the nuclease plasmid and 200 ng of the reRNA-targeted sequence plasmid were added to a 48-well cell culture plate, respectively, and transfection was performed using Lipofectamine™ 2000 (Invitrogen, Cat. No. 11668019) at a ratio of transfection reagent volume (μL):plasmid mass (μg) of 2:1.

### 3.4 PCR Amplification and NGS Second Generation Sequencing

After transfection, the cells were cultured for 48 h, then typsinized and collected, and the genome DNA was extracted. PCR primers were designed near the targeted sequence of endogenous gene to amplify a length of about 200 bp PCR product including 20 nt targeted sequence. The PCR products were sequenced by the next generation sequencing.

### 3.5 Detection Results

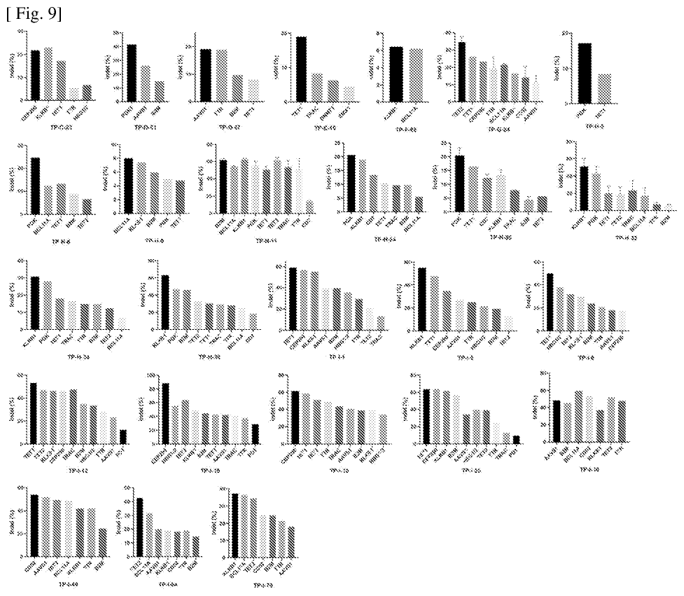

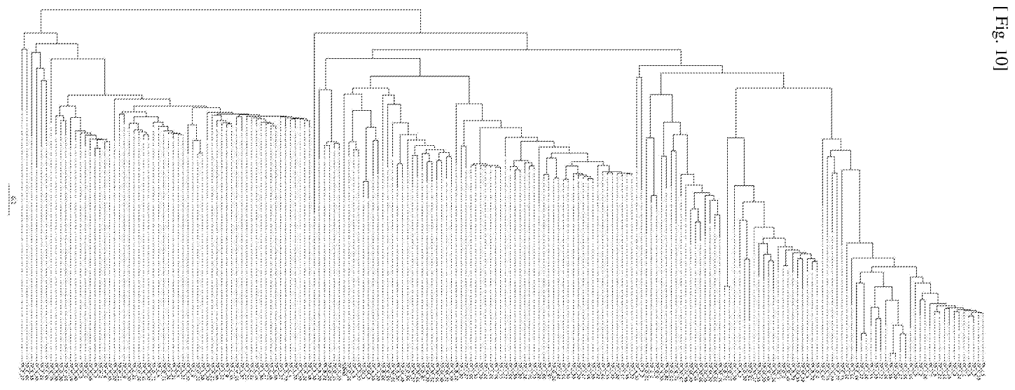

The results of endogenous gene editing efficiency were as shown in FIG. 9 and Table 3.

By analyzing the sequence data generated by the next generation sequencing technology, the endogenous gene editing activity of nuclease was determined by counting the base insertions and deletions (Indel %) generated on the targeted sequence of endogenous gene. The results showed that the nucleases in this application showed good editing efficiency on different endogenous genes.

It should be stated that the above are only the preferred examples of the present application and are not intended to limit the present application. For those of ordinary skill in the art, various modifications and changes can be made to the present application. Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents. It is important that, as the technology evolves, many elements described herein may be replaced with equivalent elements that appear after the present application.

TABLE 3
The results of endogenous gene editing activity in example 3

| Nuclease | Targeted sequence of | Targeting | Endogenous gene |
|---|---|---|---|
| name | endogenous gene | gene | editing activity |
| TP_C_23 | SEQ ID NO: 415 | CEP290 | 21.860 |
| | SEQ ID NO: 416 | HBG | 6.821 |
| | SEQ ID NO: 417 | KLKB1 | 22.947 |
| | SEQ ID NO: 418 | TET1 | 17.241 |
| | SEQ ID NO: 419 | TTR | 5.520 |
| TP_D_51 | SEQ ID NO: 420 | PGK1 | 41.613 |
| | SEQ ID NO: 421 | AAVS1 | 26.304 |
| | SEQ ID NO: 422 | B2M | 14.865 |
| TP_D_67 | SEQ ID NO: 423 | AAVS1 | 19.088 |
| | SEQ ID NO: 424 | B2M | 9.761 |
| | SEQ ID NO: 425 | TET1 | 8.216 |
| | SEQ ID NO: 426 | TTR | 18.903 |
| TP_E_15 | SEQ ID NO: 427 | TET1 | 18.951 |
| | SEQ ID NO: 428 | TRAC | 8.351 |
| | SEQ ID NO: 429 | DNMT1 | 6.336 |
| | SEQ ID NO: 430 | EMX1 | 4.493 |
| TP_F_85 | SEQ ID NO: 431 | BCL [symbol] 1A | 6.215 |
| | SEQ ID NO: 432 | KLKB1 | 6.420 |
| TP_G_24 | SEQ ID NO: 433 | TET2 | 34.649 |
| | SEQ ID NO: 434 | TET1 | 26.251 |
| | SEQ ID NO: 435 | CEP290 | 23.352 |
| | SEQ ID NO: 436 | TTR | 19.559 |
| | SEQ ID NO: 437 | BCL11A | 21.510 |
| | SEQ ID NO: 438 | KLKB1 | 16.525 |
| | SEQ ID NO: 439 | CD52 | 14.159 |
| | SEQ ID NO: 440 | AAVS1 | 11.543 |
| TP_H_5 | SEQ ID NO: 441 | PGK | 17.174 |
| | SEQ ID NO: 442 | TET1 | 8.492 |
| TP_H_6 | SEQ ID NO: 443 | PGK | 24.552 |
| | SEQ ID NO: 444 | BCL11A | 12.442 |
| | SEQ ID NO: 445 | TET1 | 13.245 |
| | SEQ ID NO: 446 | B2M | 8.950 |
| | SEQ ID NO: 447 | TET2 | 6.501 |
| TP_H_9 | SEQ ID NO: 448 | BCL11A | 7.978 |
| | SEQ ID NO: 449 | KLKB1 | 7.351 |
| | SEQ ID NO: 450 | B2M | 5.903 |
| | SEQ ID NO: 451 | PGK | 4.980 |
| | SEQ ID NO: 452 | TET [symbol] | 4.783 |
| TP_H_11 | SEQ ID NO: 453 | B2M | 61.666 |
| | SEQ ID NO: 454 | BCL11A | 54.506 |
| | SEQ ID NO: 455 | KLKB1 | 62.239 |
| | SEQ ID NO: 456 | PGK | 54.994 |
| | SEQ ID NO: 457 | TET1 | 50.273 |
| | SEQ ID NO: 458 | TET2 | 61.705 |
| | SEQ ID NO: 459 | TRAC | 53.345 |
| | SEQ ID NO: 460 | TTR | 50.796 |
| | SEQ ID NO: 461 | CD7 | 13.834 |
| TP_H_24 | SEQ ID NO: 462 | PGK | 20.581 |
| | SEQ ID NO: 463 | KLKB1 | 18.775 |
| | SEQ ID NO: 464 | CD7 | 13.384 |
| | SEQ ID NO: 465 | TET [symbol] | 10.356 |
| | SEQ ID NO: 466 | TRAC | 9.612 |
| | SEQ ID NO: 467 | B2M | 9.692 |
| | SEQ ID NO: 468 | BCL11A | 5.489 |
| TP_H_30 | SEQ ID NO: 469 | PGK | 20.436 |
| | SEQ ID NO: 470 | TET1 | 16.469 |
| | SEQ ID NO: 471 | CD7 | 12.278 |
| | SEQ ID NO: 472 | KLKB1 | 13.315 |
| | SEQ ID NO: 473 | TRAC | 7.763 |
| | SEQ ID NO: 474 | B2M | 4.450 |
| | SEQ ID NO: 475 | TET2 | 5.619 |
| TP_H_32 | SEQ ID NO: 476 | KLKB1 | 51.101 |
| | SEQ ID NO: 477 | PGK | 42.658 |
| | SEQ ID NO: 478 | TET1 | 19.984 |
| | SEQ ID NO: 479 | TET2 | 19.217 |
| | SEQ ID NO: 480 | TRAC | 23.268 |
| | SEQ ID NO: 481 | BCL11A | 17.306 |
| | SEQ ID NO: 482 | TTR | 7.225 |
| | SEQ ID NO: 483 | B2M | 6.167 |
| TP_H_34 | SEQ ID NO: 484 | KLKB1 | 30.825 |
| | SEQ ID NO: 485 | PGK | 27.967 |
| | SEQ ID NO: 486 | TET1 | 17.912 |
| | SEQ ID NO: 487 | TRAC | 16.485 |
| | SEQ ID NO: 488 | TTR | 14.895 |
| | SEQ ID NO: 489 | B2M | 14.660 |
| | SEQ ID NO: 490 | TET2 | 12.292 |
| | SEQ ID NO: 491 | BCL11A | 6.957 |
| TP_H_38 | SEQ ID NO: 492 | KLKB1 | 63.297 |
| | SEQ ID NO: 493 | PGK | 46.872 |
| | SEQ ID NO: 494 | B2M | 45.881 |
| | SEQ ID NO: 495 | TET2 | 32.409 |
| | SEQ ID NO: 496 | TET1 | 30.409 |
| | SEQ ID NO: 497 | TRAC | 29.131 |
| | SEQ ID NO: 498 | TTR | 27.788 |
| | SEQ ID NO: 499 | BCL11A | 25.266 |
| | SEQ ID NO: 500 | CD7 | 18.525 |
| TP_I_1 | SEQ ID NO: 501 | TET1 | 59.225 |
| | SEQ ID NO: 502 | CEP290 | 57.060 |
| | SEQ ID NO: 503 | KLKB1 | 55.270 |
| | SEQ ID NO: 504 | AAVS1 | 38.955 |
| | SEQ ID NO: 505 | B2M | 39.494 |
| | SEQ ID NO: 506 | HBG1_2 | 35.612 |
| | SEQ ID NO: 507 | TTR | 29.442 |
| | SEQ ID NO: 508 | TET2 | 21.489 |
| | SEQ ID NO: 509 | TRAC | 13.207 |
| TP_I_5 | SEQ ID NO: 510 | KLKB1 | 55.169 |
| | SEQ ID NO: 511 | TET1 | 47.776 |
| | SEQ ID NO: 512 | CEP290 | 34.743 |
| | SEQ ID NO: 513 | AAVS1 | 26.766 |
| | SEQ ID NO: 514 | TTR | 25.127 |
| | SEQ ID NO: 515 | HBG1_2 | 21.574 |
| | SEQ ID NO: 516 | B2M | 19.316 |
| | SEQ ID NO: 517 | TET2 | 12.666 |
| TP_I_6 | SEQ ID NO: 518 | TET1 | 50.081 |
| | SEQ ID NO: 519 | HBG1_2 | 37.687 |
| | SEQ ID NO: 520 | TET2 | 31.753 |
| | SEQ ID NO: 521 | KLKB1 | 29.755 |
| | SEQ ID NO: 522 | B2M | 23.804 |
| | SEQ ID NO: 523 | TTR | 20.689 |
| | SEQ ID NO: 524 | AAVS1 | 18.009 |
| | SEQ ID NO: 525 | CEP290 | 17.971 |
| TP_I_12 | SEQ ID NO: 526 | TET1 | 53.183 |
| | SEQ ID NO: 527 | TET2 | 46.974 |
| | SEQ ID NO: 528 | KLKB1 | 46.170 |
| | SEQ ID NO: 529 | CEP290 | 46.364 |
| | SEQ ID NO: 530 | TRAC | 47.434 |
| | SEQ ID NO: 531 | B2M | 34.654 |
| | SEQ ID NO: 532 | HBG1_2 | 33.385 |
| | SEQ ID NO: 533 | TTR | 28.052 |
| | SEQ ID NO: 534 | AAVS1 | 22.908 |
| | SEQ ID NO: 535 | PD_1 | 12.380 |
| TP_I_15 | SEQ ID NO: 536 | CEP290 | 88.371 |
| | SEQ ID NO: 537 | HBG1/2 | 55.103 |
| | SEQ ID NO: 538 | TET2 | 64.135 |
| | SEQ ID NO: 539 | KLKB1 | 48.094 |
| | SEQ ID NO: 540 | B2M | 44.417 |
| | SEQ ID NO: 541 | TET1 | 42.320 |
| | SEQ ID NO: 542 | AAVS1 | 41.814 |
| | SEQ ID NO: 543 | TRAC | 41.858 |
| | SEQ ID NO: 544 | TTR | 37.542 |
| | SEQ ID NO: 545 | PD [symbol] | 28.804 |
| TP_I_18 | SEQ ID NO: 546 | CEP290 | 61.648 |
| | SEQ ID NO: 547 | TET1 | 58.509 |
| | SEQ ID NO: 548 | TET2 | 51.308 |
| | SEQ ID NO: 549 | TTR | 49.338 |
| | SEQ ID NO: 550 | TRAC | 43.432 |
| | SEQ ID NO: 551 | AAVS1 | 40.697 |
| | SEQ ID NO: 552 | B2M | 38.666 |
| | SEQ ID NO: 553 | KLKB1 | 39.519 |
| | SEQ ID NO: 554 | HBG1/2 | 33.877 |
| TP_I_20 | SEQ ID NO: 555 | TET1 | 63.758 |
| | SEQ ID NO: 556 | CEP290 | 63.903 |
| | SEQ ID NO: 557 | KLKB1 | 61.443 |
| | SEQ ID NO: 558 | B2M | 56.965 |
| | SEQ ID NO: 559 | AAVS1 | 34.039 |
| | SEQ ID NO: 560 | HBG1/2 | 39.727 |
| | SEQ ID NO: 561 | TET2 | 39.042 |
| | SEQ ID NO: 562 | TTR | 25.227 |
| | SEQ ID NO: 563 | TRAC | 12.553 |
| | SEQ ID NO: 564 | PD1 | 9.582 |
| TP_I_38 | SEQ ID NO: 565 | AAVS1 | 48.722 |
| | SEQ ID NO: 566 | B2M | 45.242 |
| | SEQ ID NO: 567 | BCL11A | 59.706 |
| | SEQ ID NO: 568 | CD52 | 53.727 |
| | SEQ ID NO: 569 | KLKB1 | 37.065 |
| | SEQ ID NO: 570 | TET2 | 52.323 |
| | SEQ ID NO: 571 | TTR | 47.838 |
| TP_I_49 | SEQ ID NO: 572 | CD52 | 54.098 |
| | SEQ ID NO: 573 | AAVS1 | 51.949 |
| | SEQ ID NO: 574 | TET2 | 49.506 |
| | SEQ ID NO: 575 | BCL11A | 48.896 |
| | SEQ ID NO: 576 | KLKB1 | 42.187 |
| | SEQ ID NO: 577 | TTR | 42.214 |
| | SEQ ID NO: 578 | B2M | 24.369 |
| TP_I_64 | SEQ ID NO: 579 | TET2 | 42.606 |
| | SEQ ID NO: 580 | BCL11A | 31.702 |
| | SEQ ID NO: 581 | AAVS1 | 19.840 |
| | SEQ ID NO: 582 | KLKB1 | 19.125 |
| | SEQ ID NO: 583 | CD52 | 18.134 |
| | SEQ ID NO: 584 | TTR | 18.923 |
| | SEQ ID NO: 585 | B2M | 14.583 |
| TP_I_79 | SEQ ID NO: 586 | KLKB1 | 37.171 |
| | SEQ ID NO: 587 | BCL11A | 36.493 |
| | SEQ ID NO: 588 | TET2 | 34.402 |
| | SEQ ID NO: 589 | CD52 | 24.807 |
| | SEQ ID NO: 590 | B2M | 24.635 |
| | SEQ ID NO: 591 | TTR | 21.356 |
| | SEQ ID NO: 592 | AAVS1 | 17.943 |

[symbol]

indicates data missing or illegible when filed

### Example 4: Detection of the Nuclease Activity in Rice Protoplasts

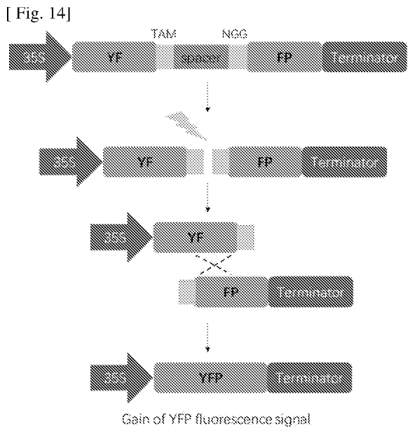

In this example, nuclease activity in rice protoplasts was evaluated using a pair of synthetic YFP gene report vectors (plasmids 5 and 6), which were constructed using the method described in example 1 (as shown in FIG. 13).

Plasmid 5 comprising a promoter ZmUBI (SEQ ID NO: 593), a candidate nuclease sequence (as shown in Table 1), a NOS terminator (SEQ ID NO: 594), a promoter OsU6 (SEQ ID NO: 595), a reRNA sequence corresponding to a specific nuclease (as shown in Table 1), a spacer sequence (SEQ ID NO: 596), and a terminator (SEQ ID NO: 597).

In plasmid 6, the YFP sequence (SEQ ID NO: 598) was segmented by spacer sequence (SEQ ID NO: 596) and the TAM sequence (as shown in Table 1) corresponding to a specific nuclease in plasmid 5. And The YFP sequence in the first half overlapped with the YFP sequence in the second half. Plasmid 6 also comprising a promoter 35S (SEQ ID NO: 599) and a terminator (SEQ ID NO: 600).

After co-transforming plasmid 5 and plasmid 6 into rice protoplasts, once the spacer sequence in plasmid 6 is cut by nuclease, the partially overlapping fragment (derived from the middle segment of YFP) promotes DSB repair through homologous dependent DNA repair pathway, thus restoring normal YFP gene (as shown in FIG. 14). Therefore, the cleavage activity of nuclease can be evaluated by observing the number of YFP-positive cells.

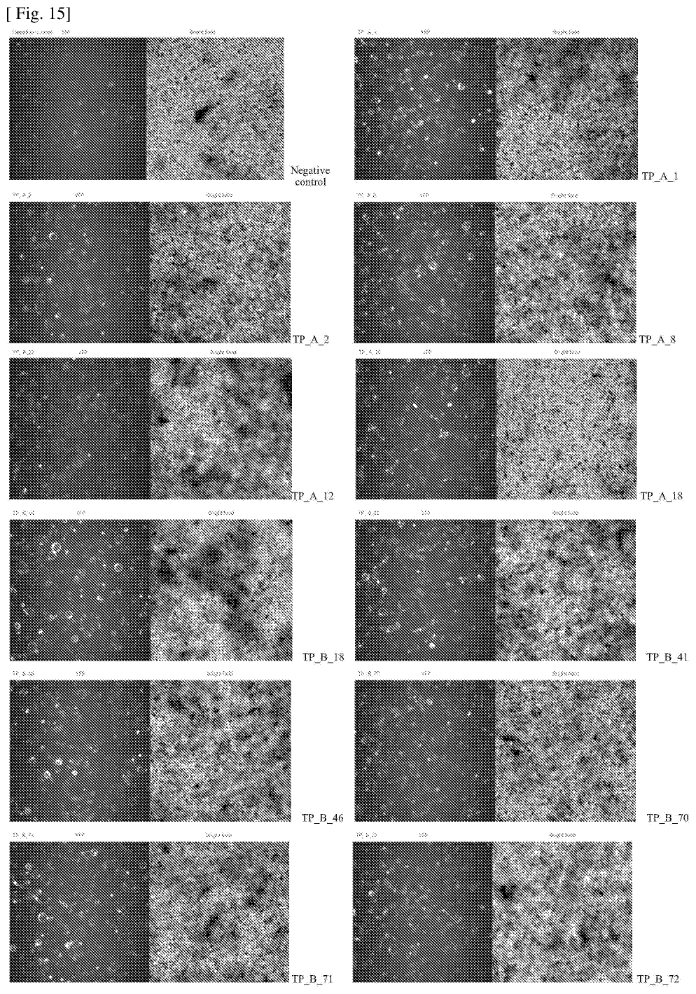

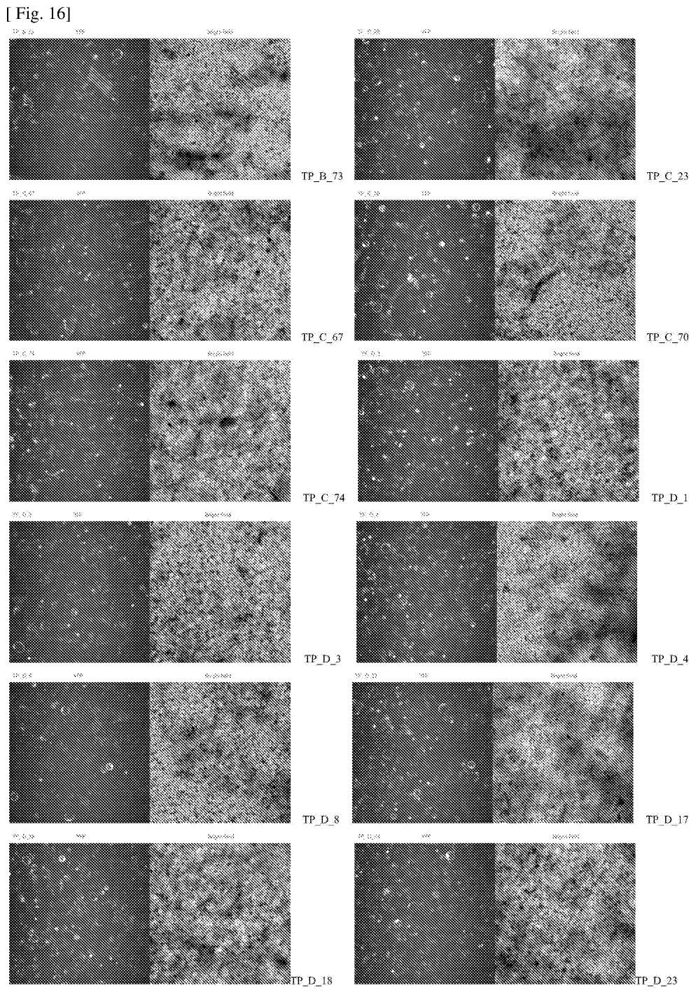

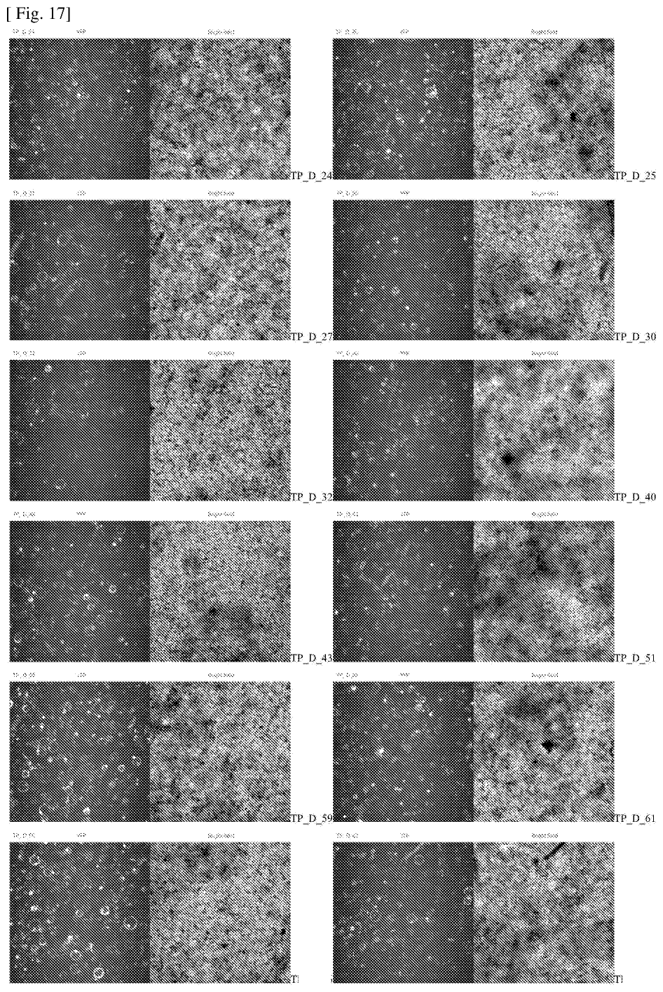

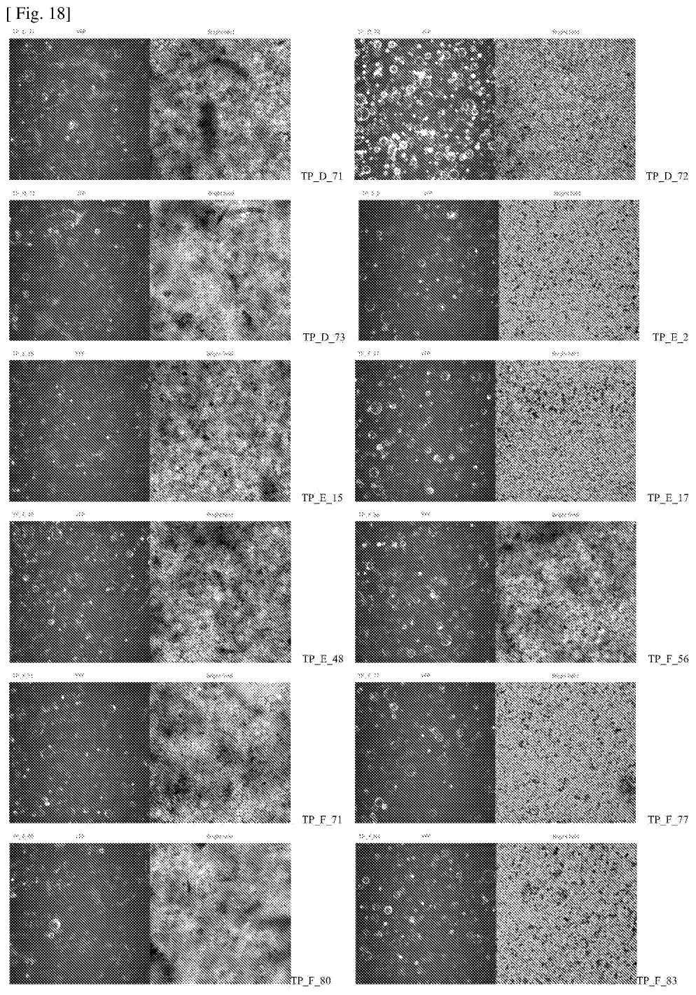

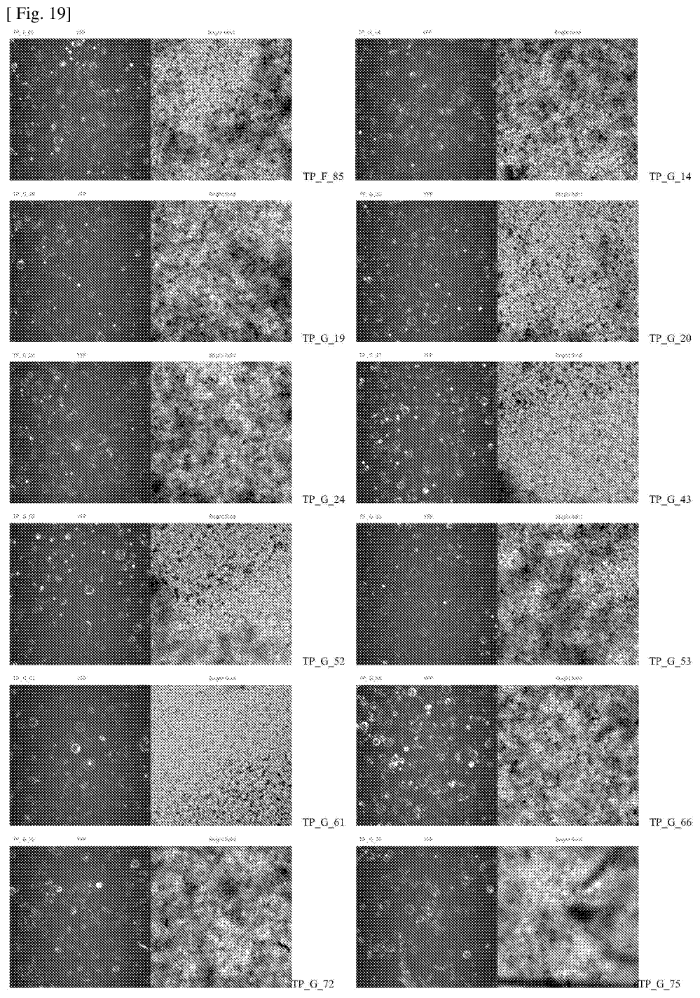

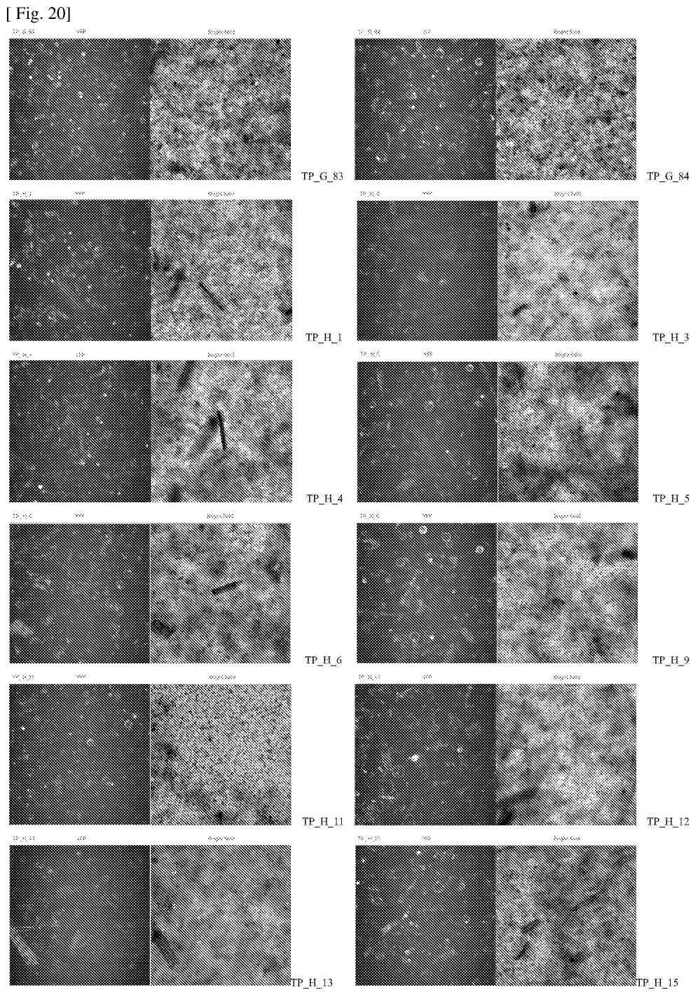

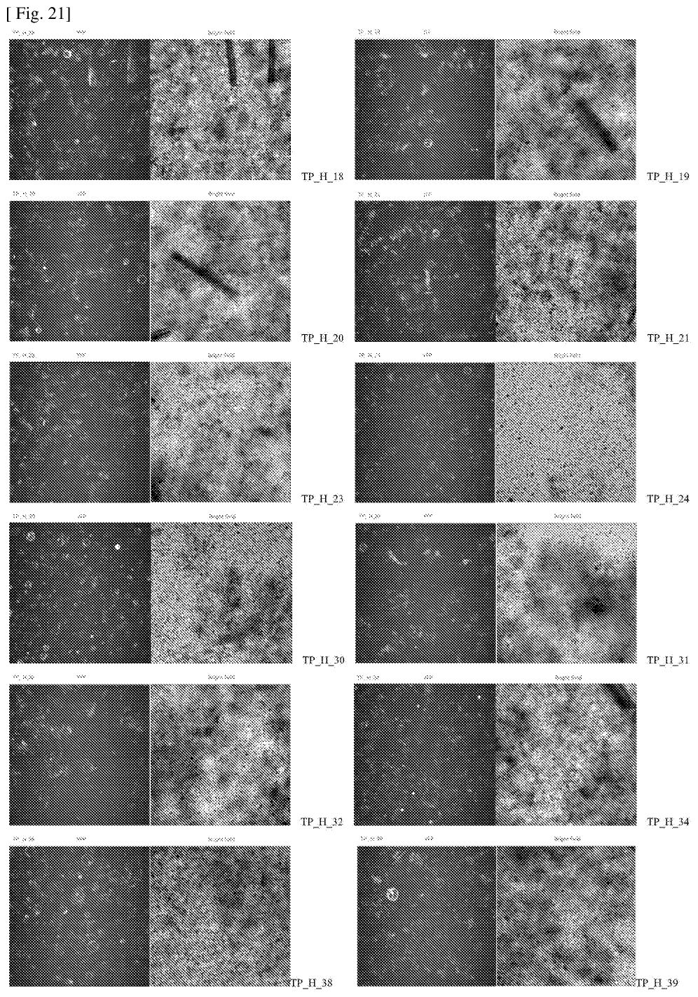

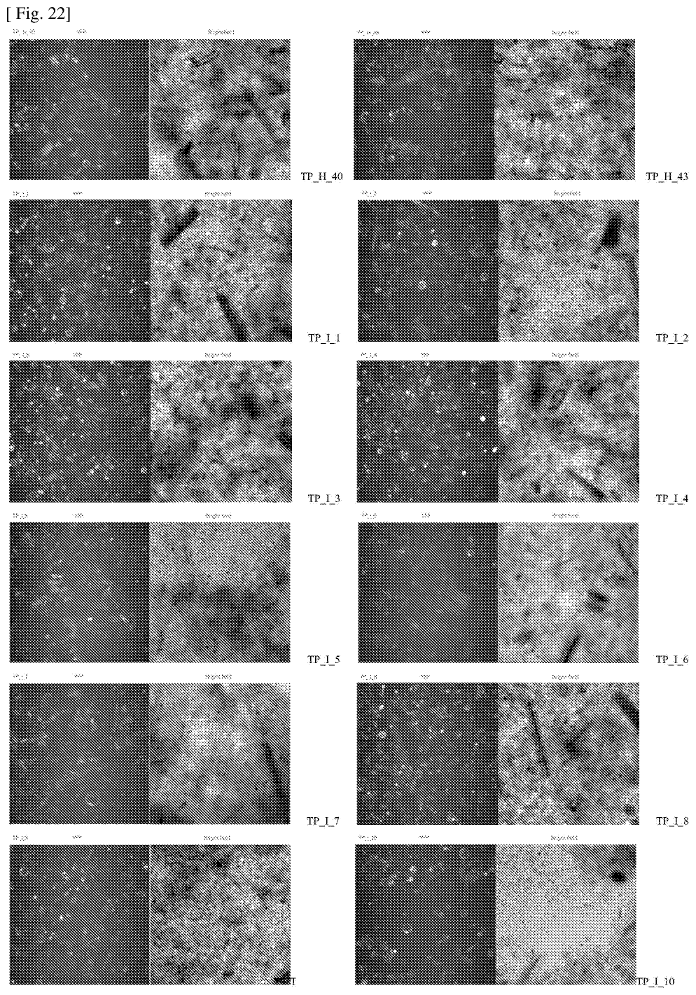

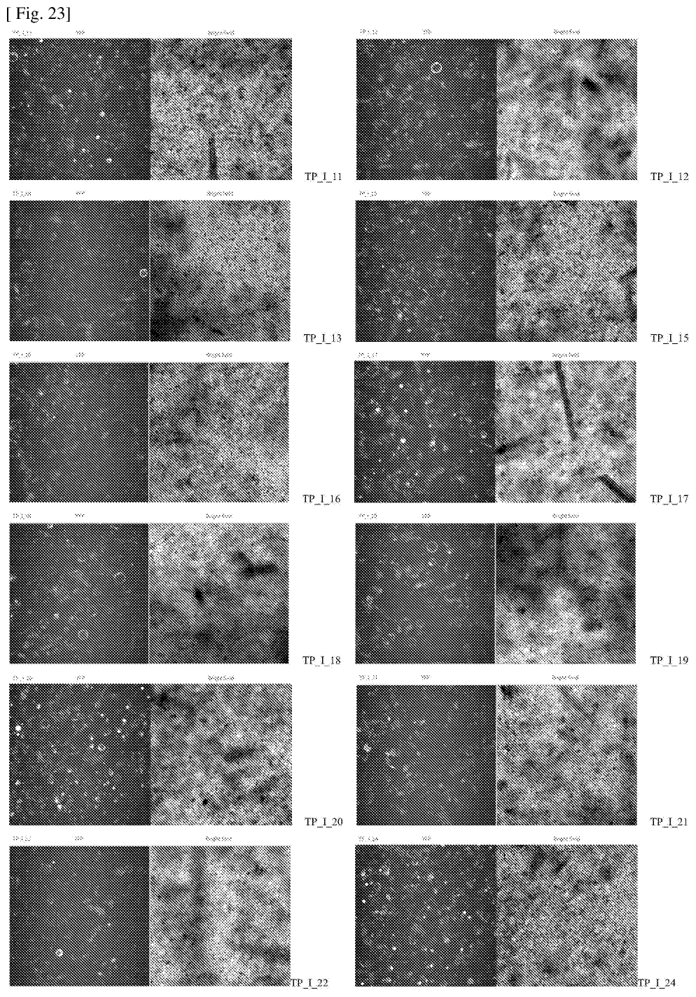

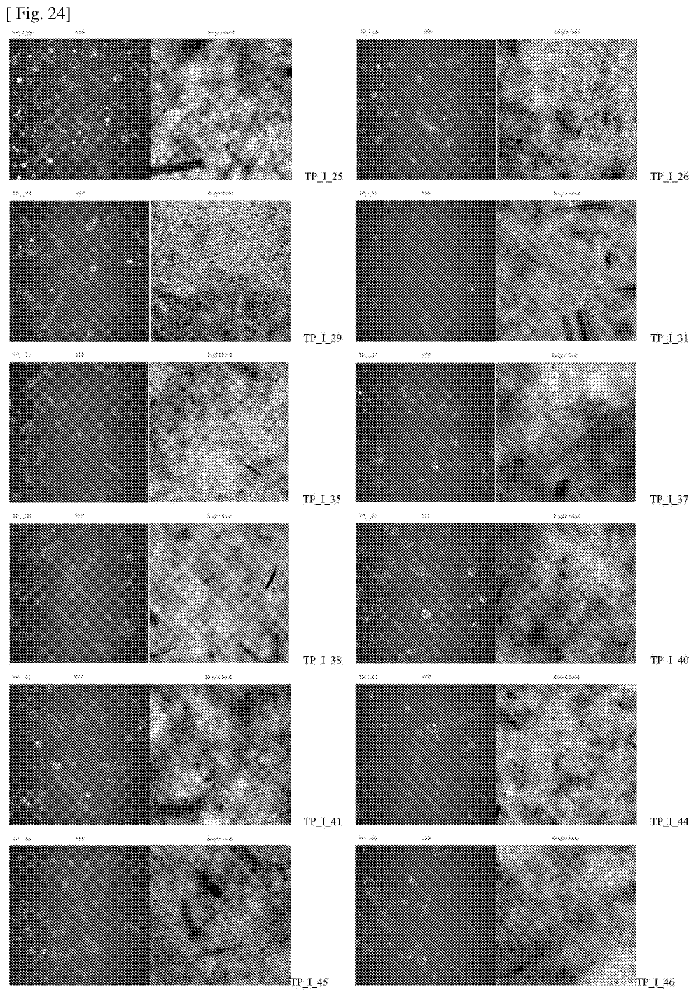

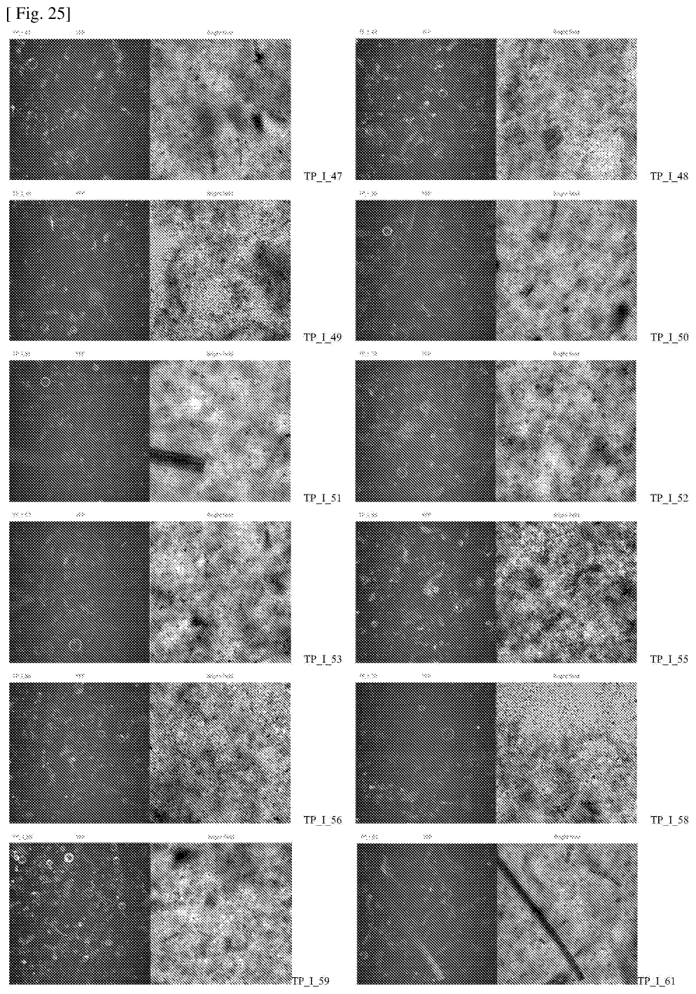

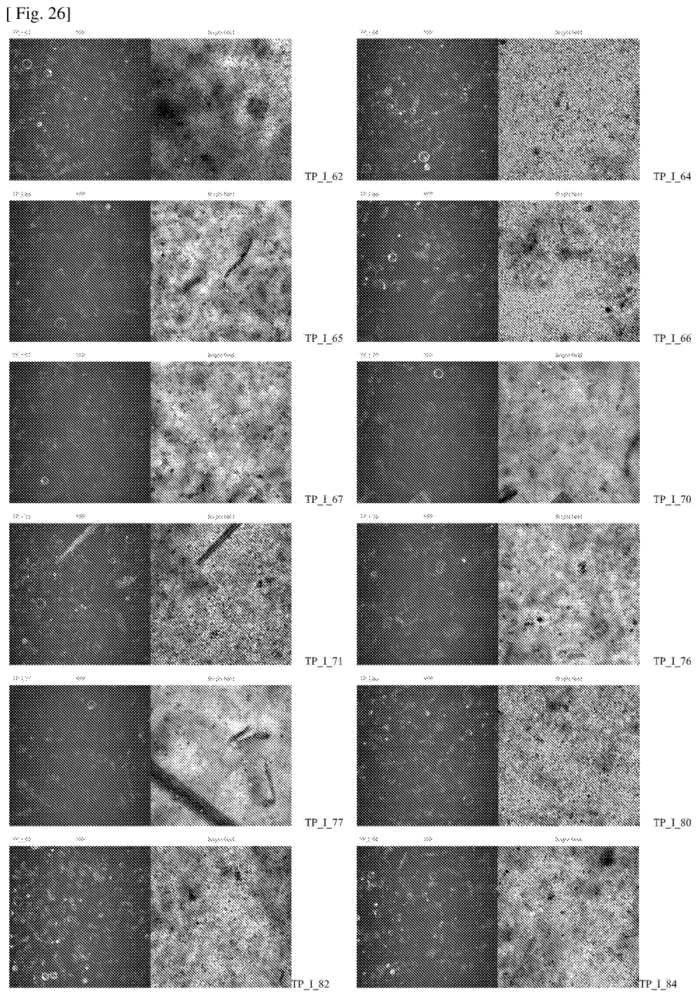

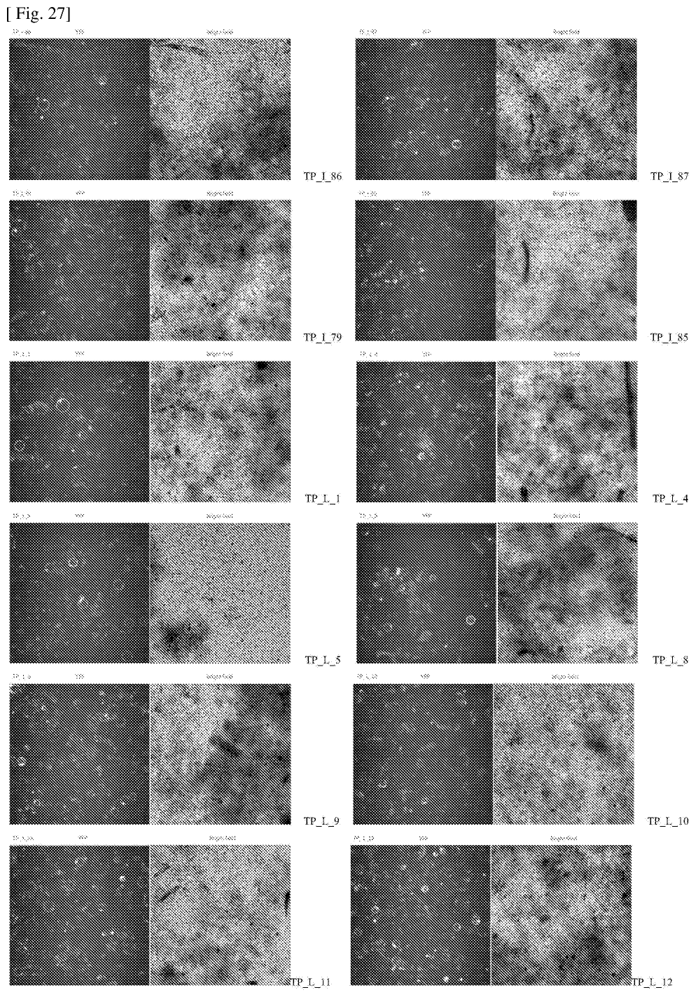

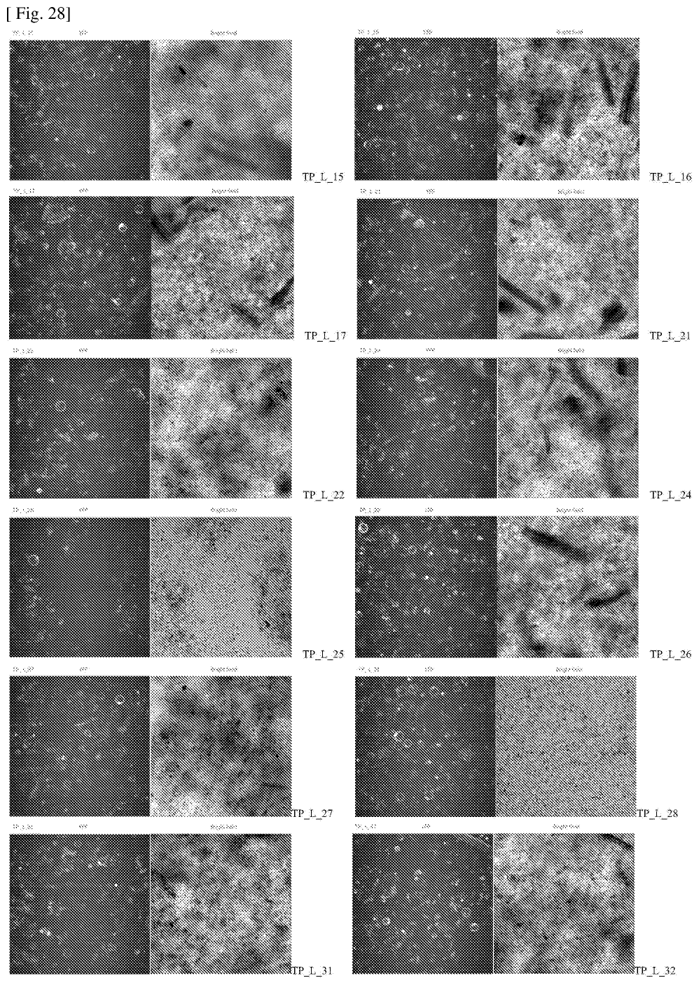

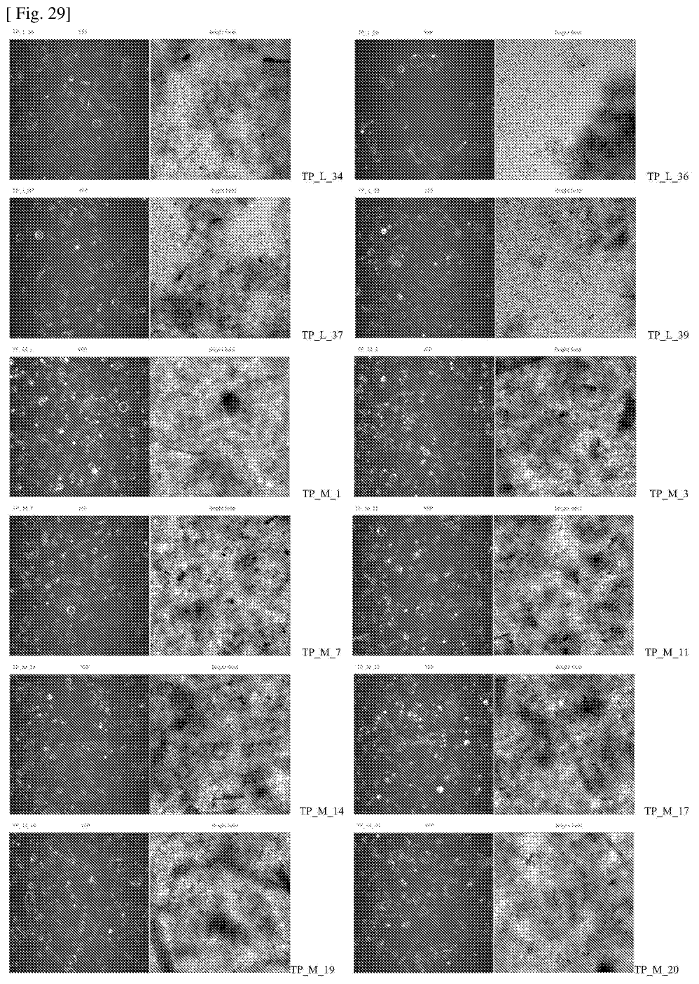

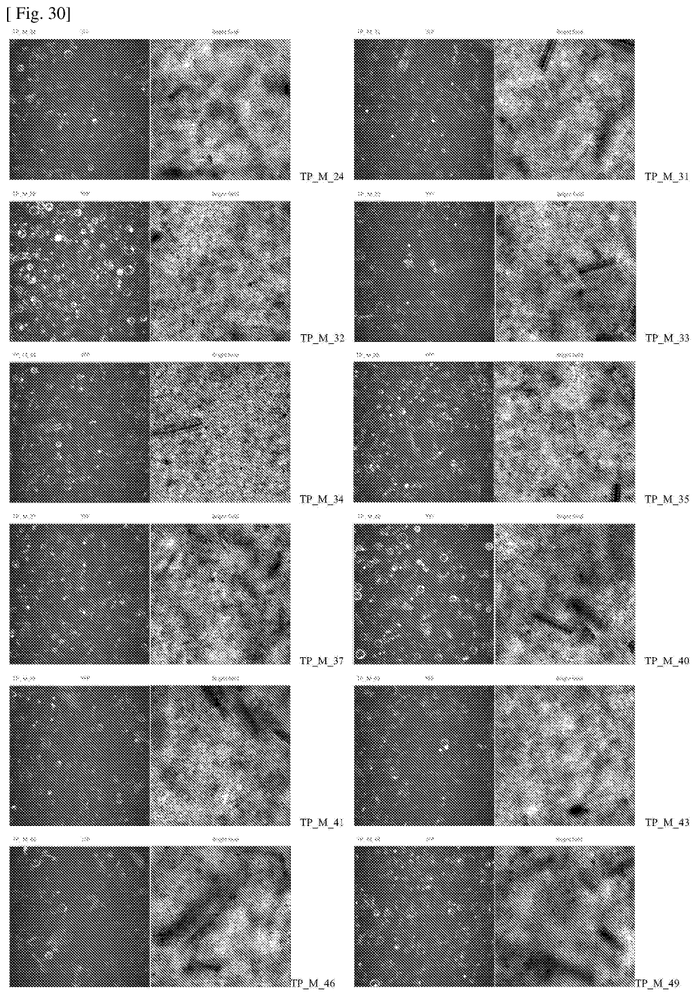

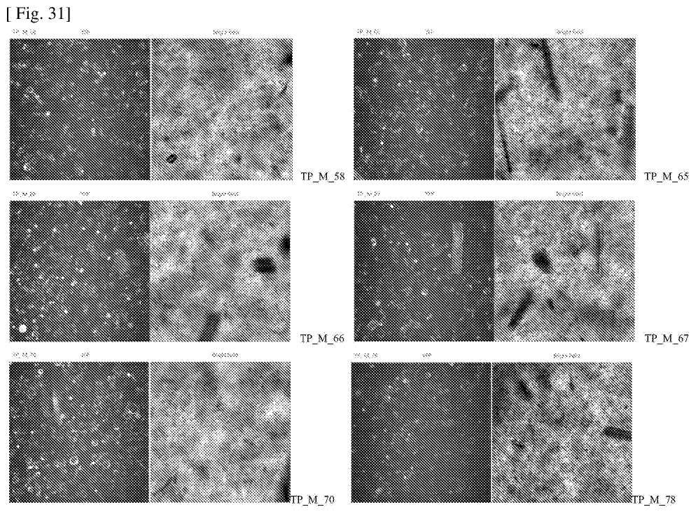

The results of YFP fluorescence were as shown in FIG. 15-31, which showed that the 197 nucleases in the present application had good cleavage activity in rice protoplasts as well (TP_A_1, TP_A_2, TP_A_8, TP_A_12, TP_A_18, TP_B_18, TP_B_41, TP_B_46, TP_B_70, TP_B_71, TP_B_72, TP_B_73, TP_C_23, TP_C_67, TP_C_70, TP_C_74, TP_D_1, TP_D_3, TP_D_4, TP_D_8, TP_D_17, TP_D_18, TP_D_23, TP_D_24, TP_D_25, TP_D_27, TP_D_30, TP_D_32, TP_D_40, TP_D_43, TP_D_51, TP_D_59, TP_D_61, TP_D_66, TP_D_67, TP_D_71, TP_D_72, TP_D_73, TP_E_2, TP_E_15, TP_E_17, TP_E_48, TP_F_56, TP_F_71, TP_F_77, TP_F_80, TP_F_83, TP_F_85, TP_G_14, TP_G_19, TP_G_20, TP_G_24, TP_G_43, TP_G_52, TP_G_53, TP_G_61, TP_G_66, TP_G_72, TP_G_75, TP_G_83, TP_G_84, TP_H_1, TP_H_3, TP_H_4, TP_H_5, TP_H_6, TP_H_9, TP_H_11, TP_H_12, TP_H_13, TP_H_15, TP_H_18, TP_H_19, TP_H_20, TP_H_21, TP_H_23, TP_H_24, TP_H_30, TP_H_31, TP_H_32, TP_H_34, TP_H_38, TP_H_39, TP_H_40, TP_H_43, TP_I_1, TP_I_2, TP_I_3, TP_I_4, TP_I_5, TP_I_6, TP_I_7, TP_I_8, TP_I_9, TP_I_10, TP_I_11, TP_I_12, TP_I_13, TP_I_15, TP_I_16, TP_I_17, TP_I_18, TP_I_19, TP_I_20, TP_I_21, TP_I_22, TP_I_24, TP_I_25, TP_I_26, TP_I_29, TP_I_31, TP_I_35, TP_I_37, TP_I_38, TP_I_40, TP_I_41, TP_I_44, TP_I_45, TP_I_46, TP_I_47, TP_I_48, TP_I_49, TP_I_50, TP_I_51, TP_I_52, TP_I_53, TP_I_55, TP_I_56, TP_I_58, TP_I_59, TP_I_61, TP_I_62, TP_I_64, TP_I_65, TP_I_66, TP_I_67, TP_I_70, TP_I_71, TP_I_76, TP_I_77, TP_I_79, TP_I_80, TP_I_82, TP_I_84, TP_I_85, TP_I_86, TP_I_87, TP_L_1, TP_L_4, TP_L_5, TP_L_8, TP_L_9, TP_L_10, TP_L_11, TP_L_12, TP_L_15, TP_L_16, TP_L_17, TP_L_21, TP_L_22, TP_L_24, TP_L_25, TP_L_26, TP_L_27, TP_L_28, TP_L_31, TP_L_32, TP_L_34, TP_L_36, TP_L_37, TP_L_39, TP_M_1, TP_M_3, TP_M_7, TP_M_11, TP_M_14, TP_M_17, TP_M_19, TP_M_20, TP_M_24, TP_M_31, TP_M_32, TP_M_33, TP_M_34, TP_M_35, TP_M_37, TP_M_40, TP_M_41, TP_M_43, TP_M_46, TP_M_49, TP_M_58, TP_M_65, TP_M_66, TP_M_67, TP_M_70 and TP_M_78).

1. An isolated nuclease, wherein the nuclease comprises an amino acid sequence as shown in the following formula:
(X₁)(X₂)ₐ(X₃)(X₄)(X₅)_{b}(X₆)(X₇)_{c}(X₈)(X₉)_{d}(X₁₀)(X₁₁)ₑ(X₁₂)(X₁₃)_{f}(X₁₄)(X₁₅)_{g}(X₁₆)
wherein,
a, b, c, d, e, f, and g are the numbers of amino acids;
(X₁), (X₃), (X₄), (X₆), (X₈), (X₁₀), (X₁₂), (X₁₄), and (X₁₆) are independently polar amino acids or aliphatic amino acids;
(X₂) is any amino acid, and a is 15 or 16;
(X₅) is any amino acid, and b is 2;
(X₇) is any amino acid, and c is 2, 3 or 4;
(X₉) is any amino acid, and dis 14, 15, 16, 17 or 18;
(X₁₁) is any amino acid, and e is 1 or 2;
(X₁₃) is any amino acid, and f is 6; and
(X₁₅) is any amino acid, and g is 5.

2. The nuclease according to claim 1, wherein the
(X₁) is a positively charged amino acid;
(X₃) is a polar uncharged amino acid;
(X₄) is a polar uncharged amino acid;
(X₆) is a polar uncharged amino acid;
(X₈) is a polar uncharged amino acid;
(X₁₀) is a polar uncharged amino acid;
(X₁₂) is a polar uncharged amino acid;
(X₁₄) is a negatively charged amino acid; and
(X₁₆) is a polar uncharged amino acid.

3. The nuclease according to claim 2, wherein the (X1) is K; (X3) is S or T; (X4) is S or T; (X6) is C; (X8) is C; (X10) is C; (X12) is C; (X14) is D; and (X16) is N.

4.-11. (canceled)

12. An isolated nuclease, wherein the nuclease has a nuclease sequence selected from the following (i) or a variant sequence of the aforementioned nuclease having a nuclease activity in (ii)-(iv):
(i) at least one amino acid sequence as shown in any one of SEQ ID NOs: 1-197;
(ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-197;
(iii) at least one of amino acid sequences having at least 70%, 80%, 90%, 95% or 99% identity to the amino acid sequence as shown in any one of SEQ ID NOs: 1-197; and
(iv) at least one of sequences obtained by further fusing the amino acid sequence as shown in any one of SEQ ID NOs: 1-197 with other sequences.

13. The nuclease according to claim 12, wherein the nuclease has a nuclease sequence selected from at least one of the following groups (1)-(9):
(1) at least one amino acid sequence as shown in any one of SEQ ID NOs: 52 and 113-147;
(2) at least one amino acid sequence as shown in any one of SEQ ID NOs: 27-28, 36-38, 62-85 and 148-171;
(3) at least one amino acid sequence as shown in any one of SEQ ID NOs: 13, 86-100 and 105-110;
(4) at least one amino acid sequence as shown in any one of SEQ ID NOs: 10-11, 17-19, 29-30 and 174-180;
(5) at least one amino acid sequence as shown in any one of SEQ ID NOs: 34, 35, 50, 61 and 181-189;
(6) at least one amino acid sequence as shown in any one of SEQ ID NOs: 53 and 190-197;
(7) at least one amino acid sequence as shown in any one of SEQ ID NOs: 101, 103, 104 and 112;
(8) at least one amino acid sequence as shown in any one of SEQ ID NOs: 7 and 23-25; and
(9) at least one amino acid sequence as shown in any one of SEQ ID NOs: 3, 21 and 22.

14. The nuclease according to claim 12, wherein the nuclease has a nuclease sequence selected from at least one of the following groups (1)-(12):
(1) at least one amino acid sequence as shown in any one of SEQ ID NOs: 1, 3-4, 6-7, 21-23, 50, 52, 60-61 and 113-147;
(2) at least one amino acid sequence as shown in any one of SEQ ID NOs: 14, 27-28, 36-38, 45-48, 59, 62-85 and 148-171;
(3) at least one amino acid sequence as shown in any one of SEQ ID NOs: 13, 43 and 86-112;
(4) at least one amino acid sequence as shown in any one of SEQ ID NOs: 15-16, 24-25, 32-35 and 181-189;
(5) at least one amino acid sequence as shown in any one of SEQ ID NOs: 9, 11, 17-19, 29 and 174-180;
(6) at least one amino acid sequence as shown in any one of SEQ ID NOs: 10, 12, 26, 30, 42 and 58;
(7) at least one amino acid sequence as shown in any one of SEQ ID NOs: 2, 20 and 31;
(8) at least one amino acid sequence as shown in any one of SEQ ID NOs: 8 and 51;
(9) at least one amino acid sequence as shown in any one of SEQ ID NOs: 39 and 49;
(10) at least one amino acid sequence as shown in any one of SEQ ID NOs: 54 and 55;
(11) at least one amino acid sequence as shown in any one of SEQ ID NOs: 53 and 190-197; and
(12) at least one amino acid sequence as shown in any one of SEQ ID NOs: 5, 172 and 173.

15.-18. (canceled)

19. A guide RNA, wherein the guide RNA comprises a reRNA, the reRNA comprises a nucleotide sequence as shown in any one of SEQ ID NOs: 198-394 or a variant thereof, and the guide RNA can bind to a specific nuclease.

20. The guide RNA according to claim 19, wherein the reRNA comprises at least one of nucleotide sequences having at least 70%, 80%, 90%, 95% or 99% identity to the nucleotide sequence as shown in any one of SEQ ID NOs: 198-394.

21.-22. (canceled)

23. The guide RNA according to claim 20, wherein the guide RNA further comprises a targeted sequence that can recognize a targeting gene adjacent to a transposon-associated motif.

24. The guide RNA according to claim 23, wherein the targeted sequence is of at least one of 10-50, 10-40, 10-30, or 15-25 nucleotides in length.

25. The guide RNA according to claim 23, wherein the transposon-associated motif comprises a nucleotide sequence as shown in the following formula:
(X₁₇)ₕ(X₁₈)(X₁₉)A(X₂₀)
wherein,
h is the number of nucleotides;
A is an adenine deoxyribonucleotide;
(X₁₇) is any deoxyribonucleotide, and h is 0 or 1;
(X₁₈) is a cytosine deoxyribonucleotide or thymine deoxyribonucleotide;
(X₁₉) is a cytosine deoxyribonucleotide, thymine deoxyribonucleotide, or guanine deoxyribonucleotide; and
(X₂₀) is any deoxyribonucleotide.

26. A nucleic acid, wherein, the nucleic acid encodes the nuclease according to claim 12.

27. A nucleic acid construct, comprising the nucleic acid according to claim 26.

28. (canceled)

29. The nucleic acid construct according to claim 27, wherein the nucleic acid construct is modified by 5′-end capping and/or 3′-end polyadenylating.

30. The nucleic acid construct according to claim 27, wherein the nucleic acid construct is modified by thiophosphate bond modification, 2′-MOE (2-O-(2-methoxyethyl)), PNA (peptide nucleic acid), GNA (glycerol nucleic acid), LNA (locked nucleic acid), GalNAc (N-acetylgalactosamine), LNP (lipid nano particle) PNP (peptide nanoparticles).

31. A composition, wherein, the composition includes:
an IS200/IS605 family nuclease or a functional fragment thereof, or comprises a nucleic acid encoding the IS200/IS605 family nuclease or the functional fragment thereof, and the nuclease or the functional fragment thereof has endonuclease activity; and
a guide RNA, or comprises a nucleic acid encoding the guide RNA, and the guide RNA can bind to a specific nuclease.

32. The composition according to claim 31, wherein the composition is selected from at least one of the following groups (1)-(198), and any one of the following groups (1)-(198) comprises: a nuclease-related sequence and a guide RNA-related sequence,
(1) the nuclease-related sequence is an amino acid sequence comprising the sequence as shown in SEQ ID NO: 1 or a nucleic acid encoding the amino acid sequence; and the guide RNA-related sequence is a nucleotide sequence comprising the sequence as shown in SEQ ID NO: 198;
(2) the nuclease-related sequence is an amino acid sequence comprising the sequence as shown in SEQ ID NO: 2 or a nucleic acid encoding the amino acid sequence; and the guide RNA-related sequence is a nucleotide sequence comprising the sequence as shown in SEQ ID NO: 199;
(3) the nuclease-related sequence is an amino acid sequence comprising the sequence as shown in SEQ ID NO: 3 or a nucleic acid encoding the amino acid sequence; and the guide RNA-related sequence is a nucleotide sequence comprising the sequence as shown in SEQ ID NO: 200;
(4) the nuclease-related sequence is an amino acid sequence comprising the sequence as shown in SEQ ID NO: 4 or a nucleic acid encoding the amino acid sequence; and the guide RNA-related sequence is a nucleotide sequence comprising the sequence as shown in SEQ ID NO: 201;
(5) the nuclease-related sequence is an amino acid sequence comprising the sequence as shown in SEQ ID NO: 5 or a nucleic acid encoding the amino acid sequence; and the guide RNA-related sequence is a nucleotide sequence comprising the sequence as shown in SEQ ID NO: 202;
(6) the nuclease-related sequence is an amino acid sequence comprising the sequence as shown in SEQ ID NO: 6 or a nucleic acid encoding the amino acid sequence; and the guide RNA-related sequence is a nucleotide sequence comprising the sequence as shown in SEQ ID NO: 203;
(7) the nuclease-related sequence is an amino acid sequence comprising the sequence as shown in SEQ ID NO: 7 or a nucleic acid encoding the amino acid sequence; and the guide RNA-related sequence is a nucleotide sequence comprising the sequence as shown in SEQ ID NO: 204;
(8) the nuclease-related sequence is an amino acid sequence comprising the sequence as shown in SEQ ID NO: 8 or a nucleic acid encoding the amino acid sequence; and the guide RNA-related sequence is a nucleotide sequence comprising the sequence as shown in SEQ ID NO: 205;
(9) the nuclease-related sequence is an amino acid sequence comprising the sequence as shown in SEQ ID NO: 9 or a nucleic acid encoding the amino acid sequence; and the guide RNA-related sequence is a nucleotide sequence comprising the sequence as shown in SEQ ID NO: 206;
(10) the nuclease-related sequence is an amino acid sequence comprising the sequence as shown in SEQ ID NO: 10 or a nucleic acid encoding the amino acid sequence; and the guide RNA-related sequence is a nucleotide sequence comprising the sequence as shown in SEQ ID NO: 207;
(11) the nuclease-related sequence is an amino acid sequence comprising the sequence as shown in SEQ ID NO: 11 or a nucleic acid encoding the amino acid sequence; and the guide RNA-related sequence is a nucleotide sequence comprising the sequence as shown in SEQ ID NO: 208;
(12) the nuclease-related sequence is an amino acid sequence comprising the sequence as shown in SEQ ID NO: 12 or a nucleic acid encoding the amino acid sequence; and the guide RNA-related sequence is a nucleotide sequence comprising the sequence as shown in SEQ ID NO: 209;
(13) the nuclease-related sequence is an amino acid sequence comprising the sequence as shown in SEQ ID NO: 13 or a nucleic acid encoding the amino acid sequence; and the guide RNA-related sequence is a nucleotide sequence comprising the sequence as shown in SEQ ID NO: 210;
(14) the nuclease-related sequence is an amino acid sequence comprising the sequence as shown in SEQ ID NO: 14 or a nucleic acid encoding the amino acid sequence; and the guide RNA-related sequence is a nucleotide sequence comprising the sequence as shown in SEQ ID NO: 211;
(15) the nuclease-related sequence is an amino acid sequence comprising the sequence as shown in SEQ ID NO: 15 or a nucleic acid encoding the amino acid sequence; and the guide RNA-related sequence is a nucleotide sequence comprising the sequence as shown in SEQ ID NO: 212;
(16) the nuclease-related sequence is an amino acid sequence comprising the sequence as shown in SEQ ID NO: 16 or a nucleic acid encoding the amino acid sequence; and the guide RNA-related sequence is a nucleotide sequence comprising the sequence as shown in SEQ ID NO: 213;
(17) the nuclease-related sequence is an amino acid sequence comprising the sequence as shown in SEQ ID NO: 17 or a nucleic acid encoding the amino acid sequence; and the guide RNA-related sequence is a nucleotide sequence comprising the sequence as shown in SEQ ID NO: 214;
(18) the nuclease-related sequence is an amino acid sequence comprising the sequence as shown in SEQ ID NO: 18 or a nucleic acid encoding the amino acid sequence; and the guide RNA-related sequence is a nucleotide sequence comprising the sequence as shown in SEQ ID NO: 215;
(19) the nuclease-related sequence is an amino acid sequence comprising the sequence as shown in SEQ ID NO: 19 or a nucleic acid encoding the amino acid sequence; and the guide RNA-related sequence is a nucleotide sequence comprising the sequence as shown in SEQ ID NO: 216;
(20) the nuclease-related sequence is an amino acid sequence comprising the sequence as shown in SEQ ID NO: 20 or a nucleic acid encoding the amino acid sequence; and the guide RNA-related sequence is a nucleotide sequence comprising the sequence as shown in SEQ ID NO: 217;
(21) the nuclease-related sequence is an amino acid sequence comprising the sequence as shown in SEQ ID NO: 21 or a nucleic acid encoding the amino acid sequence; and the guide RNA-related sequence is a nucleotide sequence comprising the sequence as shown in SEQ ID NO: 218;
(22) the nuclease-related sequence is an amino acid sequence comprising the sequence as shown in SEQ ID NO: 22 or a nucleic acid encoding the amino acid sequence; and the guide RNA-related sequence is a nucleotide sequence comprising the sequence as shown in SEQ ID NO: 219;
(23) the nuclease-related sequence is an amino acid sequence comprising the sequence as shown in SEQ ID NO: 23 or a nucleic acid encoding the amino acid sequence; and the guide RNA-related sequence is a nucleotide sequence comprising the sequence as shown in SEQ ID NO: 220;
(24) the nuclease-related sequence is an amino acid sequence comprising the sequence as shown in SEQ ID NO: 24 or a nucleic acid encoding the amino acid sequence; and the guide RNA-related sequence is a nucleotide sequence comprising the sequence as shown in SEQ ID NO: 221;
(25) the nuclease-related sequence is an amino acid sequence comprising the sequence as shown in SEQ ID NO: 25 or a nucleic acid encoding the amino acid sequence; and the guide RNA-related sequence is a nucleotide sequence comprising the sequence as shown in SEQ ID NO: 222;
(26) the nuclease-related sequence is an amino acid sequence comprising the sequence as shown in SEQ ID NO: 26 or a nucleic acid encoding the amino acid sequence; and the guide RNA-related sequence is a nucleotide sequence comprising the sequence as shown in SEQ ID NO: 223;
(27) the nuclease-related sequence is an amino acid sequence comprising the sequence as shown in SEQ ID NO: 27 or a nucleic acid encoding the amino acid sequence; and the guide RNA-related sequence is a nucleotide sequence comprising the sequence as shown in SEQ ID NO: 224;
(28) the nuclease-related sequence is an amino acid sequence comprising the sequence as shown in SEQ ID NO: 28 or a nucleic acid encoding the amino acid sequence; and the guide RNA-related sequence is a nucleotide sequence comprising the sequence as shown in SEQ ID NO: 225;
(29) the nuclease-related sequence is an amino acid sequence comprising the sequence as shown in SEQ ID NO: 29 or a nucleic acid encoding the amino acid sequence; and the guide RNA-related sequence is a nucleotide sequence comprising the sequence as shown in SEQ ID NO: 226;
(30) the nuclease-related sequence is an amino acid sequence comprising the sequence as shown in SEQ ID NO: 30 or a nucleic acid encoding the amino acid sequence; and the guide RNA-related sequence is a nucleotide sequence comprising the sequence as shown in SEQ ID NO: 227;
(31) the nuclease-related sequence is an amino acid sequence comprising the sequence as shown in SEQ ID NO: 31 or a nucleic acid encoding the amino acid sequence; and the guide RNA-related sequence is a nucleotide sequence comprising the sequence as shown in SEQ ID NO: 228;
(32) the nuclease-related sequence is an amino acid sequence comprising the sequence as shown in SEQ ID NO: 32 or a nucleic acid encoding the amino acid sequence; and the guide RNA-related sequence is a nucleotide sequence comprising the sequence as shown in SEQ ID NO: 229;
(33) the nuclease-related sequence is an amino acid sequence comprising the sequence as shown in SEQ ID NO: 33 or a nucleic acid encoding the amino acid sequence; and the guide RNA-related sequence is a nucleotide sequence comprising the sequence as shown in SEQ ID NO: 230;
(34) the nuclease-related sequence is an amino acid sequence comprising the sequence as shown in SEQ ID NO: 34 or a nucleic acid encoding the amino acid sequence; and the guide RNA-related sequence is a nucleotide sequence comprising the sequence as shown in SEQ ID NO: 231;
(35) the nuclease-related sequence is an amino acid sequence comprising the sequence as shown in SEQ ID NO: 35 or a nucleic acid encoding the amino acid sequence; and the guide RNA-related sequence is a nucleotide sequence comprising the sequence as shown in SEQ ID NO: 232;
(36) the nuclease-related sequence is an amino acid sequence comprising the sequence as shown in SEQ ID NO: 36 or a nucleic acid encoding the amino acid sequence; and the guide RNA-related sequence is a nucleotide sequence comprising the sequence as shown in SEQ ID NO: 233;
(37) the nuclease-related sequence is an amino acid sequence comprising the sequence as shown in SEQ ID NO: 37 or a nucleic acid encoding the amino acid sequence; and the guide RNA-related sequence is a nucleotide sequence comprising the sequence as shown in SEQ ID NO: 234;
(38) the nuclease-related sequence is an amino acid sequence comprising the sequence as shown in SEQ ID NO: 38 or a nucleic acid encoding the amino acid sequence; and the guide RNA-related sequence is a nucleotide sequence comprising the sequence as shown in SEQ ID NO: 235;
(39) the nuclease-related sequence is an amino acid sequence comprising the sequence as shown in SEQ ID NO: 39 or a nucleic acid encoding the amino acid sequence; and the guide RNA-related sequence is a nucleotide sequence comprising the sequence as shown in SEQ ID NO: 236;
(40) the nuclease-related sequence is an amino acid sequence comprising the sequence as shown in SEQ ID NO: 40 or a nucleic acid encoding the amino acid sequence; and the guide RNA-related sequence is a nucleotide sequence comprising the sequence as shown in SEQ ID NO: 237;
(41) the nuclease-related sequence is an amino acid sequence comprising the sequence as shown in SEQ ID NO: 41 or a nucleic acid encoding the amino acid sequence; and the guide RNA-related sequence is a nucleotide sequence comprising the sequence as shown in SEQ ID NO: 238;
(42) the nuclease-related sequence is an amino acid sequence comprising the sequence as shown in SEQ ID NO: 42 or a nucleic acid encoding the amino acid sequence; and the guide RNA-related sequence is a nucleotide sequence comprising the sequence as shown in SEQ ID NO: 239;
(43) the nuclease-related sequence is an amino acid sequence comprising the sequence as shown in SEQ ID NO: 43 or a nucleic acid encoding the amino acid sequence; and the guide RNA-related sequence is a nucleotide sequence comprising the sequence as shown in SEQ ID NO: 240;
(44) the nuclease-related sequence is an amino acid sequence comprising the sequence as shown in SEQ ID NO: 44 or a nucleic acid encoding the amino acid sequence; and the guide RNA-related sequence is a nucleotide sequence comprising the sequence as shown in SEQ ID NO: 241;
(45) the nuclease-related sequence is an amino acid sequence comprising the sequence as shown in SEQ ID NO: 45 or a nucleic acid encoding the amino acid sequence; and the guide RNA-related sequence is a nucleotide sequence comprising the sequence as shown in SEQ ID NO: 242;
(46) the nuclease-related sequence is an amino acid sequence comprising the sequence as shown in SEQ ID NO: 46 or a nucleic acid encoding the amino acid sequence; and the guide RNA-related sequence is a nucleotide sequence comprising the sequence as shown in SEQ ID NO: 243;
(47) the nuclease-related sequence is an amino acid sequence comprising the sequence as shown in SEQ ID NO: 47 or a nucleic acid encoding the amino acid sequence; and the guide RNA-related sequence is a nucleotide sequence comprising the sequence as shown in SEQ ID NO: 244;
(48) the nuclease-related sequence is an amino acid sequence comprising the sequence as shown in SEQ ID NO: 48 or a nucleic acid encoding the amino acid sequence; and the guide RNA-related sequence is a nucleotide sequence comprising the sequence as shown in SEQ ID NO: 245;
(49) the nuclease-related sequence is an amino acid sequence comprising the sequence as shown in SEQ ID NO: 49 or a nucleic acid encoding the amino acid sequence; and the guide RNA-related sequence is a nucleotide sequence comprising the sequence as shown in SEQ ID NO: 246;
(50) the nuclease-related sequence is an amino acid sequence comprising the sequence as shown in SEQ ID NO: 50 or a nucleic acid encoding the amino acid sequence; and the guide RNA-related sequence is a nucleotide sequence comprising the sequence as shown in SEQ ID NO: 247;
(51) the nuclease-related sequence is an amino acid sequence comprising the sequence as shown in SEQ ID NO: 51 or a nucleic acid encoding the amino acid sequence; and the guide RNA-related sequence is a nucleotide sequence comprising the sequence as shown in SEQ ID NO: 248;
(52) the nuclease-related sequence is an amino acid sequence comprising the sequence as shown in SEQ ID NO: 52 or a nucleic acid encoding the amino acid sequence; and the guide RNA-related sequence is a nucleotide sequence comprising the sequence as shown in SEQ ID NO: 249;
(53) the nuclease-related sequence is an amino acid sequence comprising the sequence as shown in SEQ ID NO: 53 or a nucleic acid encoding the amino acid sequence; and the guide RNA-related sequence is a nucleotide sequence comprising the sequence as shown in SEQ ID NO: 250;
(54) the nuclease-related sequence is an amino acid sequence comprising the sequence as shown in SEQ ID NO: 54 or a nucleic acid encoding the amino acid sequence; and the guide RNA-related sequence is a nucleotide sequence comprising the sequence as shown in SEQ ID NO: 251;
(55) the nuclease-related sequence is an amino acid sequence comprising the sequence as shown in SEQ ID NO: 55 or a nucleic acid encoding the amino acid sequence; and the guide RNA-related sequence is a nucleotide sequence comprising the sequence as shown in SEQ ID NO: 252;
(56) the nuclease-related sequence is an amino acid sequence comprising the sequence as shown in SEQ ID NO: 56 or a nucleic acid encoding the amino acid sequence; and the guide RNA-related sequence is a nucleotide sequence comprising the sequence as shown in SEQ ID NO: 253;
(57) the nuclease-related sequence is an amino acid sequence comprising the sequence as shown in SEQ ID NO: 57 or a nucleic acid encoding the amino acid sequence; and the guide RNA-related sequence is a nucleotide sequence comprising the sequence as shown in SEQ ID NO: 254;
(58) the nuclease-related sequence is an amino acid sequence comprising the sequence as shown in SEQ ID NO: 58 or a nucleic acid encoding the amino acid sequence; and the guide RNA-related sequence is a nucleotide sequence comprising the sequence as shown in SEQ ID NO: 255;
(59) the nuclease-related sequence is an amino acid sequence comprising the sequence as shown in SEQ ID NO: 59 or a nucleic acid encoding the amino acid sequence; and the guide RNA-related sequence is a nucleotide sequence comprising the sequence as shown in SEQ ID NO: 256;
(60) the nuclease-related sequence is an amino acid sequence comprising the sequence as shown in SEQ ID NO: 60 or a nucleic acid encoding the amino acid sequence; and the guide RNA-related sequence is a nucleotide sequence comprising the sequence as shown in SEQ ID NO: 257;
(61) the nuclease-related sequence is an amino acid sequence comprising the sequence as shown in SEQ ID NO: 61 or a nucleic acid encoding the amino acid sequence; and the guide RNA-related sequence is a nucleotide sequence comprising the sequence as shown in SEQ ID NO: 258;
(62) the nuclease-related sequence is an amino acid sequence comprising the sequence as shown in SEQ ID NO: 62 or a nucleic acid encoding the amino acid sequence; and the guide RNA-related sequence is a nucleotide sequence comprising the sequence as shown in SEQ ID NO: 259;
(63) the nuclease-related sequence is an amino acid sequence comprising the sequence as shown in SEQ ID NO: 63 or a nucleic acid encoding the amino acid sequence; and the guide RNA-related sequence is a nucleotide sequence comprising the sequence as shown in SEQ ID NO: 260;
(64) the nuclease-related sequence is an amino acid sequence comprising the sequence as shown in SEQ ID NO: 64 or a nucleic acid encoding the amino acid sequence; and the guide RNA-related sequence is a nucleotide sequence comprising the sequence as shown in SEQ ID NO: 261;
(65) the nuclease-related sequence is an amino acid sequence comprising the sequence as shown in SEQ ID NO: 65 or a nucleic acid encoding the amino acid sequence; and the guide RNA-related sequence is a nucleotide sequence comprising the sequence as shown in SEQ ID NO: 262;
(66) the nuclease-related sequence is an amino acid sequence comprising the sequence as shown in SEQ ID NO: 66 or a nucleic acid encoding the amino acid sequence; and the guide RNA-related sequence is a nucleotide sequence comprising the sequence as shown in SEQ ID NO: 263;
(67) the nuclease-related sequence is an amino acid sequence comprising the sequence as shown in SEQ ID NO: 67 or a nucleic acid encoding the amino acid sequence; and the guide RNA-related sequence is a nucleotide sequence comprising the sequence as shown in SEQ ID NO: 264;
(68) the nuclease-related sequence is an amino acid sequence comprising the sequence as shown in SEQ ID NO: 68 or a nucleic acid encoding the amino acid sequence; and the guide RNA-related sequence is a nucleotide sequence comprising the sequence as shown in SEQ ID NO: 265;
(69) the nuclease-related sequence is an amino acid sequence comprising the sequence as shown in SEQ ID NO: 69 or a nucleic acid encoding the amino acid sequence; and the guide RNA-related sequence is a nucleotide sequence comprising the sequence as shown in SEQ ID NO: 266;
(70) the nuclease-related sequence is an amino acid sequence comprising the sequence as shown in SEQ ID NO: 70 or a nucleic acid encoding the amino acid sequence; and the guide RNA-related sequence is a nucleotide sequence comprising the sequence as shown in SEQ ID NO: 267;
(71) the nuclease-related sequence is an amino acid sequence comprising the sequence as shown in SEQ ID NO: 71 or a nucleic acid encoding the amino acid sequence; and the guide RNA-related sequence is a nucleotide sequence comprising the sequence as shown in SEQ ID NO: 268;
(72) the nuclease-related sequence is an amino acid sequence comprising the sequence as shown in SEQ ID NO: 72 or a nucleic acid encoding the amino acid sequence; and the guide RNA-related sequence is a nucleotide sequence comprising the sequence as shown in SEQ ID NO: 269;
(73) the nuclease-related sequence is an amino acid sequence comprising the sequence as shown in SEQ ID NO: 73 or a nucleic acid encoding the amino acid sequence; and the guide RNA-related sequence is a nucleotide sequence comprising the sequence as shown in SEQ ID NO: 270;
(74) the nuclease-related sequence is an amino acid sequence comprising the sequence as shown in SEQ ID NO: 74 or a nucleic acid encoding the amino acid sequence; and the guide RNA-related sequence is a nucleotide sequence comprising the sequence as shown in SEQ ID NO: 271;
(75) the nuclease-related sequence is an amino acid sequence comprising the sequence as shown in SEQ ID NO: 75 or a nucleic acid encoding the amino acid sequence; and the guide RNA-related sequence is a nucleotide sequence comprising the sequence as shown in SEQ ID NO: 272;
(76) the nuclease-related sequence is an amino acid sequence comprising the sequence as shown in SEQ ID NO: 76 or a nucleic acid encoding the amino acid sequence; and the guide RNA-related sequence is a nucleotide sequence comprising the sequence as shown in SEQ ID NO: 273;
(77) the nuclease-related sequence is an amino acid sequence comprising the sequence as shown in SEQ ID NO: 77 or a nucleic acid encoding the amino acid sequence; and the guide RNA-related sequence is a nucleotide sequence comprising the sequence as shown in SEQ ID NO: 274;
(78) the nuclease-related sequence is an amino acid sequence comprising the sequence as shown in SEQ ID NO: 78 or a nucleic acid encoding the amino acid sequence; and the guide RNA-related sequence is a nucleotide sequence comprising the sequence as shown in SEQ ID NO: 275;
(79) the nuclease-related sequence is an amino acid sequence comprising the sequence as shown in SEQ ID NO: 79 or a nucleic acid encoding the amino acid sequence; and the guide RNA-related sequence is a nucleotide sequence comprising the sequence as shown in SEQ ID NO: 276;
(80) the nuclease-related sequence is an amino acid sequence comprising the sequence as shown in SEQ ID NO: 80 or a nucleic acid encoding the amino acid sequence; and the guide RNA-related sequence is a nucleotide sequence comprising the sequence as shown in SEQ ID NO: 277;
(81) the nuclease-related sequence is an amino acid sequence comprising the sequence as shown in SEQ ID NO: 81 or a nucleic acid encoding the amino acid sequence; and the guide RNA-related sequence is a nucleotide sequence comprising the sequence as shown in SEQ ID NO: 278;
(82) the nuclease-related sequence is an amino acid sequence comprising the sequence as shown in SEQ ID NO: 82 or a nucleic acid encoding the amino acid sequence; and the guide RNA-related sequence is a nucleotide sequence comprising the sequence as shown in SEQ ID NO: 279;
(83) the nuclease-related sequence is an amino acid sequence comprising the sequence as shown in SEQ ID NO: 83 or a nucleic acid encoding the amino acid sequence; and the guide RNA-related sequence is a nucleotide sequence comprising the sequence as shown in SEQ ID NO: 280;
(84) the nuclease-related sequence is an amino acid sequence comprising the sequence as shown in SEQ ID NO: 84 or a nucleic acid encoding the amino acid sequence; and the guide RNA-related sequence is a nucleotide sequence comprising the sequence as shown in SEQ ID NO: 281;
(85) the nuclease-related sequence is an amino acid sequence comprising the sequence as shown in SEQ ID NO: 85 or a nucleic acid encoding the amino acid sequence; and the guide RNA-related sequence is a nucleotide sequence comprising the sequence as shown in SEQ ID NO: 282;
(86) the nuclease-related sequence is an amino acid sequence comprising the sequence as shown in SEQ ID NO: 86 or a nucleic acid encoding the amino acid sequence; and the guide RNA-related sequence is a nucleotide sequence comprising the sequence as shown in SEQ ID NO: 283;
(87) the nuclease-related sequence is an amino acid sequence comprising the sequence as shown in SEQ ID NO: 87 or a nucleic acid encoding the amino acid sequence; and the guide RNA-related sequence is a nucleotide sequence comprising the sequence as shown in SEQ ID NO: 284;
(88) the nuclease-related sequence is an amino acid sequence comprising the sequence as shown in SEQ ID NO: 88 or a nucleic acid encoding the amino acid sequence; and the guide RNA-related sequence is a nucleotide sequence comprising the sequence as shown in SEQ ID NO: 285;
(89) the nuclease-related sequence is an amino acid sequence comprising the sequence as shown in SEQ ID NO: 89 or a nucleic acid encoding the amino acid sequence; and the guide RNA-related sequence is a nucleotide sequence comprising the sequence as shown in SEQ ID NO: 286;
(90) the nuclease-related sequence is an amino acid sequence comprising the sequence as shown in SEQ ID NO: 90 or a nucleic acid encoding the amino acid sequence; and the guide RNA-related sequence is a nucleotide sequence comprising the sequence as shown in SEQ ID NO: 287;
(91) the nuclease-related sequence is an amino acid sequence comprising the sequence as shown in SEQ ID NO: 91 or a nucleic acid encoding the amino acid sequence; and the guide RNA-related sequence is a nucleotide sequence comprising the sequence as shown in SEQ ID NO: 288;
(92) the nuclease-related sequence is an amino acid sequence comprising the sequence as shown in SEQ ID NO: 92 or a nucleic acid encoding the amino acid sequence; and the guide RNA-related sequence is a nucleotide sequence comprising the sequence as shown in SEQ ID NO: 289;
(93) the nuclease-related sequence is an amino acid sequence comprising the sequence as shown in SEQ ID NO: 93 or a nucleic acid encoding the amino acid sequence; and the guide RNA-related sequence is a nucleotide sequence comprising the sequence as shown in SEQ ID NO: 290;
(94) the nuclease-related sequence is an amino acid sequence comprising the sequence as shown in SEQ ID NO: 94 or a nucleic acid encoding the amino acid sequence; and the guide RNA-related sequence is a nucleotide sequence comprising the sequence as shown in SEQ ID NO: 291;
(95) the nuclease-related sequence is an amino acid sequence comprising the sequence as shown in SEQ ID NO: 95 or a nucleic acid encoding the amino acid sequence; and the guide RNA-related sequence is a nucleotide sequence comprising the sequence as shown in SEQ ID NO: 292;
(96) the nuclease-related sequence is an amino acid sequence comprising the sequence as shown in SEQ ID NO: 96 or a nucleic acid encoding the amino acid sequence; and the guide RNA-related sequence is a nucleotide sequence comprising the sequence as shown in SEQ ID NO: 293;
(97) the nuclease-related sequence is an amino acid sequence comprising the sequence as shown in SEQ ID NO: 97 or a nucleic acid encoding the amino acid sequence; and the guide RNA-related sequence is a nucleotide sequence comprising the sequence as shown in SEQ ID NO: 294;
(98) the nuclease-related sequence is an amino acid sequence comprising the sequence as shown in SEQ ID NO: 98 or a nucleic acid encoding the amino acid sequence; and the guide RNA-related sequence is a nucleotide sequence comprising the sequence as shown in SEQ ID NO: 295;
(99) the nuclease-related sequence is an amino acid sequence comprising the sequence as shown in SEQ ID NO: 99 or a nucleic acid encoding the amino acid sequence; and the guide RNA-related sequence is a nucleotide sequence comprising the sequence as shown in SEQ ID NO: 296;
(100) the nuclease-related sequence is an amino acid sequence comprising the sequence as shown in SEQ ID NO: 100 or a nucleic acid encoding the amino acid sequence; and the guide RNA-related sequence is a nucleotide sequence comprising the sequence as shown in SEQ ID NO: 297;
(101) the nuclease-related sequence is an amino acid sequence comprising the sequence as shown in SEQ ID NO: 101 or a nucleic acid encoding the amino acid sequence; and the guide RNA-related sequence is a nucleotide sequence comprising the sequence as shown in SEQ ID NO: 298;
(102) the nuclease-related sequence is an amino acid sequence comprising the sequence as shown in SEQ ID NO: 102 or a nucleic acid encoding the amino acid sequence; and the guide RNA-related sequence is a nucleotide sequence comprising the sequence as shown in SEQ ID NO: 299;
(103) the nuclease-related sequence is an amino acid sequence comprising the sequence as shown in SEQ ID NO: 103 or a nucleic acid encoding the amino acid sequence; and the guide RNA-related sequence is a nucleotide sequence comprising the sequence as shown in SEQ ID NO: 300;
(104) the nuclease-related sequence is an amino acid sequence comprising the sequence as shown in SEQ ID NO: 104 or a nucleic acid encoding the amino acid sequence; and the guide RNA-related sequence is a nucleotide sequence comprising the sequence as shown in SEQ ID NO: 301;
(105) the nuclease-related sequence is an amino acid sequence comprising the sequence as shown in SEQ ID NO: 105 or a nucleic acid encoding the amino acid sequence; and the guide RNA-related sequence is a nucleotide sequence comprising the sequence as shown in SEQ ID NO: 302;
(106) the nuclease-related sequence is an amino acid sequence comprising the sequence as shown in SEQ ID NO: 106 or a nucleic acid encoding the amino acid sequence; and the guide RNA-related sequence is a nucleotide sequence comprising the sequence as shown in SEQ ID NO: 303;
(107) the nuclease-related sequence is an amino acid sequence comprising the sequence as shown in SEQ ID NO: 107 or a nucleic acid encoding the amino acid sequence; and the guide RNA-related sequence is a nucleotide sequence comprising the sequence as shown in SEQ ID NO: 304;
(108) the nuclease-related sequence is an amino acid sequence comprising the sequence as shown in SEQ ID NO: 108 or a nucleic acid encoding the amino acid sequence; and the guide RNA-related sequence is a nucleotide sequence comprising the sequence as shown in SEQ ID NO: 305;
(109) the nuclease-related sequence is an amino acid sequence comprising the sequence as shown in SEQ ID NO: 109 or a nucleic acid encoding the amino acid sequence; and the guide RNA-related sequence is a nucleotide sequence comprising the sequence as shown in SEQ ID NO: 306;
(110) the nuclease-related sequence is an amino acid sequence comprising the sequence as shown in SEQ ID NO: 110 or a nucleic acid encoding the amino acid sequence; and the guide RNA-related sequence is a nucleotide sequence comprising the sequence as shown in SEQ ID NO: 307;
(111) the nuclease-related sequence is an amino acid sequence comprising the sequence as shown in SEQ ID NO: 111 or a nucleic acid encoding the amino acid sequence; and the guide RNA-related sequence is a nucleotide sequence comprising the sequence as shown in SEQ ID NO: 308;
(112) the nuclease-related sequence is an amino acid sequence comprising the sequence as shown in SEQ ID NO: 112 or a nucleic acid encoding the amino acid sequence; and the guide RNA-related sequence is a nucleotide sequence comprising the sequence as shown in SEQ ID NO: 309;
(113) the nuclease-related sequence is an amino acid sequence comprising the sequence as shown in SEQ ID NO: 113 or a nucleic acid encoding the amino acid sequence; and the guide RNA-related sequence is a nucleotide sequence comprising the sequence as shown in SEQ ID NO: 310;
(114) the nuclease-related sequence is an amino acid sequence comprising the sequence as shown in SEQ ID NO: 114 or a nucleic acid encoding the amino acid sequence; and the guide RNA-related sequence is a nucleotide sequence comprising the sequence as shown in SEQ ID NO: 311;
(115) the nuclease-related sequence is an amino acid sequence comprising the sequence as shown in SEQ ID NO: 115 or a nucleic acid encoding the amino acid sequence; and the guide RNA-related sequence is a nucleotide sequence comprising the sequence as shown in SEQ ID NO: 312;
(116) the nuclease-related sequence is an amino acid sequence comprising the sequence as shown in SEQ ID NO: 116 or a nucleic acid encoding the amino acid sequence; and the guide RNA-related sequence is a nucleotide sequence comprising the sequence as shown in SEQ ID NO: 313;
(117) the nuclease-related sequence is an amino acid sequence comprising the sequence as shown in SEQ ID NO: 117 or a nucleic acid encoding the amino acid sequence; and the guide RNA-related sequence is a nucleotide sequence comprising the sequence as shown in SEQ ID NO: 314;
(118) the nuclease-related sequence is an amino acid sequence comprising the sequence as shown in SEQ ID NO: 118 or a nucleic acid encoding the amino acid sequence; and the guide RNA-related sequence is a nucleotide sequence comprising the sequence as shown in SEQ ID NO: 315;
(119) the nuclease-related sequence is an amino acid sequence comprising the sequence as shown in SEQ ID NO: 119 or a nucleic acid encoding the amino acid sequence; and the guide RNA-related sequence is a nucleotide sequence comprising the sequence as shown in SEQ ID NO: 316;
(120) the nuclease-related sequence is an amino acid sequence comprising the sequence as shown in SEQ ID NO: 120 or a nucleic acid encoding the amino acid sequence; and the guide RNA-related sequence is a nucleotide sequence comprising the sequence as shown in SEQ ID NO: 317;
(121) the nuclease-related sequence is an amino acid sequence comprising the sequence as shown in SEQ ID NO: 121 or a nucleic acid encoding the amino acid sequence; and the guide RNA-related sequence is a nucleotide sequence comprising the sequence as shown in SEQ ID NO: 318;
(122) the nuclease-related sequence is an amino acid sequence comprising the sequence as shown in SEQ ID NO: 122 or a nucleic acid encoding the amino acid sequence; and the guide RNA-related sequence is a nucleotide sequence comprising the sequence as shown in SEQ ID NO: 319;
(123) the nuclease-related sequence is an amino acid sequence comprising the sequence as shown in SEQ ID NO: 123 or a nucleic acid encoding the amino acid sequence; and the guide RNA-related sequence is a nucleotide sequence comprising the sequence as shown in SEQ ID NO: 320;
(124) the nuclease-related sequence is an amino acid sequence comprising the sequence as shown in SEQ ID NO: 124 or a nucleic acid encoding the amino acid sequence; and the guide RNA-related sequence is a nucleotide sequence comprising the sequence as shown in SEQ ID NO: 321;
(125) the nuclease-related sequence is an amino acid sequence comprising the sequence as shown in SEQ ID NO: 125 or a nucleic acid encoding the amino acid sequence; and the guide RNA-related sequence is a nucleotide sequence comprising the sequence as shown in SEQ ID NO: 322;
(126) the nuclease-related sequence is an amino acid sequence comprising the sequence as shown in SEQ ID NO: 126 or a nucleic acid encoding the amino acid sequence; and the guide RNA-related sequence is a nucleotide sequence comprising the sequence as shown in SEQ ID NO: 323;
(127) the nuclease-related sequence is an amino acid sequence comprising the sequence as shown in SEQ ID NO: 127 or a nucleic acid encoding the amino acid sequence; and the guide RNA-related sequence is a nucleotide sequence comprising the sequence as shown in SEQ ID NO: 324;
(128) the nuclease-related sequence is an amino acid sequence comprising the sequence as shown in SEQ ID NO: 128 or a nucleic acid encoding the amino acid sequence; and the guide RNA-related sequence is a nucleotide sequence comprising the sequence as shown in SEQ ID NO: 325;
(129) the nuclease-related sequence is an amino acid sequence comprising the sequence as shown in SEQ ID NO: 129 or a nucleic acid encoding the amino acid sequence; and the guide RNA-related sequence is a nucleotide sequence comprising the sequence as shown in SEQ ID NO: 326;
(130) the nuclease-related sequence is an amino acid sequence comprising the sequence as shown in SEQ ID NO: 130 or a nucleic acid encoding the amino acid sequence; and the guide RNA-related sequence is a nucleotide sequence comprising the sequence as shown in SEQ ID NO: 327;
(131) the nuclease-related sequence is an amino acid sequence comprising the sequence as shown in SEQ ID NO: 131 or a nucleic acid encoding the amino acid sequence; and the guide RNA-related sequence is a nucleotide sequence comprising the sequence as shown in SEQ ID NO: 328;
(132) the nuclease-related sequence is an amino acid sequence comprising the sequence as shown in SEQ ID NO: 132 or a nucleic acid encoding the amino acid sequence; and the guide RNA-related sequence is a nucleotide sequence comprising the sequence as shown in SEQ ID NO: 329;
(133) the nuclease-related sequence is an amino acid sequence comprising the sequence as shown in SEQ ID NO: 133 or a nucleic acid encoding the amino acid sequence; and the guide RNA-related sequence is a nucleotide sequence comprising the sequence as shown in SEQ ID NO: 330;
(134) the nuclease-related sequence is an amino acid sequence comprising the sequence as shown in SEQ ID NO: 134 or a nucleic acid encoding the amino acid sequence; and the guide RNA-related sequence is a nucleotide sequence comprising the sequence as shown in SEQ ID NO: 331;
(135) the nuclease-related sequence is an amino acid sequence comprising the sequence as shown in SEQ ID NO: 135 or a nucleic acid encoding the amino acid sequence; and the guide RNA-related sequence is a nucleotide sequence comprising the sequence as shown in SEQ ID NO: 332;
(136) the nuclease-related sequence is an amino acid sequence comprising the sequence as shown in SEQ ID NO: 136 or a nucleic acid encoding the amino acid sequence; and the guide RNA-related sequence is a nucleotide sequence comprising the sequence as shown in SEQ ID NO: 333;
(137) the nuclease-related sequence is an amino acid sequence comprising the sequence as shown in SEQ ID NO: 137 or a nucleic acid encoding the amino acid sequence; and the guide RNA-related sequence is a nucleotide sequence comprising the sequence as shown in SEQ ID NO: 334;
(138) the nuclease-related sequence is an amino acid sequence comprising the sequence as shown in SEQ ID NO: 138 or a nucleic acid encoding the amino acid sequence; and the guide RNA-related sequence is a nucleotide sequence comprising the sequence as shown in SEQ ID NO: 335;
(139) the nuclease-related sequence is an amino acid sequence comprising the sequence as shown in SEQ ID NO: 139 or a nucleic acid encoding the amino acid sequence; and the guide RNA-related sequence is a nucleotide sequence comprising the sequence as shown in SEQ ID NO: 336;
(140) the nuclease-related sequence is an amino acid sequence comprising the sequence as shown in SEQ ID NO: 140 or a nucleic acid encoding the amino acid sequence; and the guide RNA-related sequence is a nucleotide sequence comprising the sequence as shown in SEQ ID NO: 337;
(141) the nuclease-related sequence is an amino acid sequence comprising the sequence as shown in SEQ ID NO: 141 or a nucleic acid encoding the amino acid sequence; and the guide RNA-related sequence is a nucleotide sequence comprising the sequence as shown in SEQ ID NO: 338;
(142) the nuclease-related sequence is an amino acid sequence comprising the sequence as shown in SEQ ID NO: 142 or a nucleic acid encoding the amino acid sequence; and the guide RNA-related sequence is a nucleotide sequence comprising the sequence as shown in SEQ ID NO: 339;
(143) the nuclease-related sequence is an amino acid sequence comprising the sequence as shown in SEQ ID NO: 143 or a nucleic acid encoding the amino acid sequence; and the guide RNA-related sequence is a nucleotide sequence comprising the sequence as shown in SEQ ID NO: 340;
(144) the nuclease-related sequence is an amino acid sequence comprising the sequence as shown in SEQ ID NO: 144 or a nucleic acid encoding the amino acid sequence; and the guide RNA-related sequence is a nucleotide sequence comprising the sequence as shown in SEQ ID NO: 341;
(145) the nuclease-related sequence is an amino acid sequence comprising the sequence as shown in SEQ ID NO: 145 or a nucleic acid encoding the amino acid sequence; and the guide RNA-related sequence is a nucleotide sequence comprising the sequence as shown in SEQ ID NO: 342;
(146) the nuclease-related sequence is an amino acid sequence comprising the sequence as shown in SEQ ID NO: 146 or a nucleic acid encoding the amino acid sequence; and the guide RNA-related sequence is a nucleotide sequence comprising the sequence as shown in SEQ ID NO: 343;
(147) the nuclease-related sequence is an amino acid sequence comprising the sequence as shown in SEQ ID NO: 147 or a nucleic acid encoding the amino acid sequence; and the guide RNA-related sequence is a nucleotide sequence comprising the sequence as shown in SEQ ID NO: 344;
(148) the nuclease-related sequence is an amino acid sequence comprising the sequence as shown in SEQ ID NO: 148 or a nucleic acid encoding the amino acid sequence; and the guide RNA-related sequence is a nucleotide sequence comprising the sequence as shown in SEQ ID NO: 345;
(149) the nuclease-related sequence is an amino acid sequence comprising the sequence as shown in SEQ ID NO: 149 or a nucleic acid encoding the amino acid sequence; and the guide RNA-related sequence is a nucleotide sequence comprising the sequence as shown in SEQ ID NO: 346;
(150) the nuclease-related sequence is an amino acid sequence comprising the sequence as shown in SEQ ID NO: 150 or a nucleic acid encoding the amino acid sequence; and the guide RNA-related sequence is a nucleotide sequence comprising the sequence as shown in SEQ ID NO: 347;
(151) the nuclease-related sequence is an amino acid sequence comprising the sequence as shown in SEQ ID NO: 151 or a nucleic acid encoding the amino acid sequence; and the guide RNA-related sequence is a nucleotide sequence comprising the sequence as shown in SEQ ID NO: 348;
(152) the nuclease-related sequence is an amino acid sequence comprising the sequence as shown in SEQ ID NO: 152 or a nucleic acid encoding the amino acid sequence; and the guide RNA-related sequence is a nucleotide sequence comprising the sequence as shown in SEQ ID NO: 349;
(153) the nuclease-related sequence is an amino acid sequence comprising the sequence as shown in SEQ ID NO: 153 or a nucleic acid encoding the amino acid sequence; and the guide RNA-related sequence is a nucleotide sequence comprising the sequence as shown in SEQ ID NO: 350;
(154) the nuclease-related sequence is an amino acid sequence comprising the sequence as shown in SEQ ID NO: 154 or a nucleic acid encoding the amino acid sequence; and the guide RNA-related sequence is a nucleotide sequence comprising the sequence as shown in SEQ ID NO: 351;
(155) the nuclease-related sequence is an amino acid sequence comprising the sequence as shown in SEQ ID NO: 155 or a nucleic acid encoding the amino acid sequence; and the guide RNA-related sequence is a nucleotide sequence comprising the sequence as shown in SEQ ID NO: 352;
(156) the nuclease-related sequence is an amino acid sequence comprising the sequence as shown in SEQ ID NO: 156 or a nucleic acid encoding the amino acid sequence; and the guide RNA-related sequence is a nucleotide sequence comprising the sequence as shown in SEQ ID NO: 353;
(157) the nuclease-related sequence is an amino acid sequence comprising the sequence as shown in SEQ ID NO: 157 or a nucleic acid encoding the amino acid sequence; and the guide RNA-related sequence is a nucleotide sequence comprising the sequence as shown in SEQ ID NO: 354;
(158) the nuclease-related sequence is an amino acid sequence comprising the sequence as shown in SEQ ID NO: 158 or a nucleic acid encoding the amino acid sequence; and the guide RNA-related sequence is a nucleotide sequence comprising the sequence as shown in SEQ ID NO: 355;
(159) the nuclease-related sequence is an amino acid sequence comprising the sequence as shown in SEQ ID NO: 159 or a nucleic acid encoding the amino acid sequence; and the guide RNA-related sequence is a nucleotide sequence comprising the sequence as shown in SEQ ID NO: 356;
(160) the nuclease-related sequence is an amino acid sequence comprising the sequence as shown in SEQ ID NO: 160 or a nucleic acid encoding the amino acid sequence; and the guide RNA-related sequence is a nucleotide sequence comprising the sequence as shown in SEQ ID NO: 357;
(161) the nuclease-related sequence is an amino acid sequence comprising the sequence as shown in SEQ ID NO: 161 or a nucleic acid encoding the amino acid sequence; and the guide RNA-related sequence is a nucleotide sequence comprising the sequence as shown in SEQ ID NO: 358;
(162) the nuclease-related sequence is an amino acid sequence comprising the sequence as shown in SEQ ID NO: 162 or a nucleic acid encoding the amino acid sequence; and the guide RNA-related sequence is a nucleotide sequence comprising the sequence as shown in SEQ ID NO: 359;
(163) the nuclease-related sequence is an amino acid sequence comprising the sequence as shown in SEQ ID NO: 163 or a nucleic acid encoding the amino acid sequence; and the guide RNA-related sequence is a nucleotide sequence comprising the sequence as shown in SEQ ID NO: 360;
(164) the nuclease-related sequence is an amino acid sequence comprising the sequence as shown in SEQ ID NO: 164 or a nucleic acid encoding the amino acid sequence; and the guide RNA-related sequence is a nucleotide sequence comprising the sequence as shown in SEQ ID NO: 361;
(165) the nuclease-related sequence is an amino acid sequence comprising the sequence as shown in SEQ ID NO: 165 or a nucleic acid encoding the amino acid sequence; and the guide RNA-related sequence is a nucleotide sequence comprising the sequence as shown in SEQ ID NO: 362;
(166) the nuclease-related sequence is an amino acid sequence comprising the sequence as shown in SEQ ID NO: 166 or a nucleic acid encoding the amino acid sequence; and the guide RNA-related sequence is a nucleotide sequence comprising the sequence as shown in SEQ ID NO: 363;
(167) the nuclease-related sequence is an amino acid sequence comprising the sequence as shown in SEQ ID NO: 167 or a nucleic acid encoding the amino acid sequence; and the guide RNA-related sequence is a nucleotide sequence comprising the sequence as shown in SEQ ID NO: 364;
(168) the nuclease-related sequence is an amino acid sequence comprising the sequence as shown in SEQ ID NO: 168 or a nucleic acid encoding the amino acid sequence; and the guide RNA-related sequence is a nucleotide sequence comprising the sequence as shown in SEQ ID NO: 365;
(169) the nuclease-related sequence is an amino acid sequence comprising the sequence as shown in SEQ ID NO: 169 or a nucleic acid encoding the amino acid sequence; and the guide RNA-related sequence is a nucleotide sequence comprising the sequence as shown in SEQ ID NO: 366;
(170) the nuclease-related sequence is an amino acid sequence comprising the sequence as shown in SEQ ID NO: 170 or a nucleic acid encoding the amino acid sequence; and the guide RNA-related sequence is a nucleotide sequence comprising the sequence as shown in SEQ ID NO: 367;
(171) the nuclease-related sequence is an amino acid sequence comprising the sequence as shown in SEQ ID NO: 171 or a nucleic acid encoding the amino acid sequence; and the guide RNA-related sequence is a nucleotide sequence comprising the sequence as shown in SEQ ID NO: 368;
(172) the nuclease-related sequence is an amino acid sequence comprising the sequence as shown in SEQ ID NO: 172 or a nucleic acid encoding the amino acid sequence; and the guide RNA-related sequence is a nucleotide sequence comprising the sequence as shown in SEQ ID NO: 369;
(173) the nuclease-related sequence is an amino acid sequence comprising the sequence as shown in SEQ ID NO: 173 or a nucleic acid encoding the amino acid sequence; and the guide RNA-related sequence is a nucleotide sequence comprising the sequence as shown in SEQ ID NO: 370;
(174) the nuclease-related sequence is an amino acid sequence comprising the sequence as shown in SEQ ID NO: 174 or a nucleic acid encoding the amino acid sequence; and the guide RNA-related sequence is a nucleotide sequence comprising the sequence as shown in SEQ ID NO: 371;
(175) the nuclease-related sequence is an amino acid sequence comprising the sequence as shown in SEQ ID NO: 175 or a nucleic acid encoding the amino acid sequence; and the guide RNA-related sequence is a nucleotide sequence comprising the sequence as shown in SEQ ID NO: 372;
(176) the nuclease-related sequence is an amino acid sequence comprising the sequence as shown in SEQ ID NO: 176 or a nucleic acid encoding the amino acid sequence; and the guide RNA-related sequence is a nucleotide sequence comprising the sequence as shown in SEQ ID NO: 373;
(177) the nuclease-related sequence is an amino acid sequence comprising the sequence as shown in SEQ ID NO: 177 or a nucleic acid encoding the amino acid sequence; and the guide RNA-related sequence is a nucleotide sequence comprising the sequence as shown in SEQ ID NO: 374;
(178) the nuclease-related sequence is an amino acid sequence comprising the sequence as shown in SEQ ID NO: 178 or a nucleic acid encoding the amino acid sequence; and the guide RNA-related sequence is a nucleotide sequence comprising the sequence as shown in SEQ ID NO: 375;
(179) the nuclease-related sequence is an amino acid sequence comprising the sequence as shown in SEQ ID NO: 179 or a nucleic acid encoding the amino acid sequence; and the guide RNA-related sequence is a nucleotide sequence comprising the sequence as shown in SEQ ID NO: 376;
(180) the nuclease-related sequence is an amino acid sequence comprising the sequence as shown in SEQ ID NO: 180 or a nucleic acid encoding the amino acid sequence; and the guide RNA-related sequence is a nucleotide sequence comprising the sequence as shown in SEQ ID NO: 377;
(181) the nuclease-related sequence is an amino acid sequence comprising the sequence as shown in SEQ ID NO: 181 or a nucleic acid encoding the amino acid sequence; and the guide RNA-related sequence is a nucleotide sequence comprising the sequence as shown in SEQ ID NO: 378;
(182) the nuclease-related sequence is an amino acid sequence comprising the sequence as shown in SEQ ID NO: 182 or a nucleic acid encoding the amino acid sequence; and the guide RNA-related sequence is a nucleotide sequence comprising the sequence as shown in SEQ ID NO: 379;
(183) the nuclease-related sequence is an amino acid sequence comprising the sequence as shown in SEQ ID NO: 183 or a nucleic acid encoding the amino acid sequence; and the guide RNA-related sequence is a nucleotide sequence comprising the sequence as shown in SEQ ID NO: 380;
(184) the nuclease-related sequence is an amino acid sequence comprising the sequence as shown in SEQ ID NO: 184 or a nucleic acid encoding the amino acid sequence; and the guide RNA-related sequence is a nucleotide sequence comprising the sequence as shown in SEQ ID NO: 381;
(185) the nuclease-related sequence is an amino acid sequence comprising the sequence as shown in SEQ ID NO: 185 or a nucleic acid encoding the amino acid sequence; and the guide RNA-related sequence is a nucleotide sequence comprising the sequence as shown in SEQ ID NO: 382;
(186) the nuclease-related sequence is an amino acid sequence comprising the sequence as shown in SEQ ID NO: 186 or a nucleic acid encoding the amino acid sequence; and the guide RNA-related sequence is a nucleotide sequence comprising the sequence as shown in SEQ ID NO: 383;
(187) the nuclease-related sequence is an amino acid sequence comprising the sequence as shown in SEQ ID NO: 187 or a nucleic acid encoding the amino acid sequence; and the guide RNA-related sequence is a nucleotide sequence comprising the sequence as shown in SEQ ID NO: 384;
(188) the nuclease-related sequence is an amino acid sequence comprising the sequence as shown in SEQ ID NO: 188 or a nucleic acid encoding the amino acid sequence; and the guide RNA-related sequence is a nucleotide sequence comprising the sequence as shown in SEQ ID NO: 385;
(189) the nuclease-related sequence is an amino acid sequence comprising the sequence as shown in SEQ ID NO: 189 or a nucleic acid encoding the amino acid sequence; and the guide RNA-related sequence is a nucleotide sequence comprising the sequence as shown in SEQ ID NO: 386;
(190) the nuclease-related sequence is an amino acid sequence comprising the sequence as shown in SEQ ID NO: 190 or a nucleic acid encoding the amino acid sequence; and the guide RNA-related sequence is a nucleotide sequence comprising the sequence as shown in SEQ ID NO: 387;
(191) the nuclease-related sequence is an amino acid sequence comprising the sequence as shown in SEQ ID NO: 191 or a nucleic acid encoding the amino acid sequence; and the guide RNA-related sequence is a nucleotide sequence comprising the sequence as shown in SEQ ID NO: 388;
(192) the nuclease-related sequence is an amino acid sequence comprising the sequence as shown in SEQ ID NO: 192 or a nucleic acid encoding the amino acid sequence; and the guide RNA-related sequence is a nucleotide sequence comprising the sequence as shown in SEQ ID NO: 389;
(193) the nuclease-related sequence is an amino acid sequence comprising the sequence as shown in SEQ ID NO: 193 or a nucleic acid encoding the amino acid sequence; and the guide RNA-related sequence is a nucleotide sequence comprising the sequence as shown in SEQ ID NO: 390;
(194) the nuclease-related sequence is an amino acid sequence comprising the sequence as shown in SEQ ID NO: 194 or a nucleic acid encoding the amino acid sequence; and the guide RNA-related sequence is a nucleotide sequence comprising the sequence as shown in SEQ ID NO: 391;
(195) the nuclease-related sequence is an amino acid sequence comprising the sequence as shown in SEQ ID NO: 195 or a nucleic acid encoding the amino acid sequence; and the guide RNA-related sequence is a nucleotide sequence comprising the sequence as shown in SEQ ID NO: 392;
(196) the nuclease-related sequence is an amino acid sequence comprising the sequence as shown in SEQ ID NO: 196 or a nucleic acid encoding the amino acid sequence; and the guide RNA-related sequence is a nucleotide sequence comprising the sequence as shown in SEQ ID NO: 393;
(197) the nuclease-related sequence is an amino acid sequence comprising the sequence as shown in SEQ ID NO: 197 or a nucleic acid encoding the amino acid sequence; and the guide RNA-related sequence is a nucleotide sequence comprising the sequence as shown in SEQ ID NO: 394;
(198) a variant of any one of the aforementioned groups (1)-(197),
wherein the nuclease-related sequence is the amino acid sequence of the variant of the nuclease in each group or a nucleic acid sequence encoding the variant, and the variant has a variant sequence of the aforementioned nuclease having a nuclease activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the nuclease in each group;
(ii) at least one of amino acid sequences having at least 70%, 80%, 90%, 95% or 99% identity to the amino acid sequence as shown in any one of SEQ ID NOs: 1-197; and
(iii) at least one of sequences obtained by further fusing the amino acid sequence as shown in any one of SEQ ID NO: 1-197 with other sequences.

33. The composition according to claim 32, wherein the guide RNA-related sequence further comprises a targeted sequence that can recognize a targeting gene adjacent to a transposon-associated motif.

34. The composition according to claim 33, wherein the targeted sequence is of at least one of 10-50, 10-40, 10-30, or 15-25 nucleotides in length.

35. The composition according to claim 33, wherein the transposon-associated motif comprises a nucleotide sequence as shown in the following formula:
(X₁₇)ₕ(X₁₈)(X₁₉)A(X₂₀)
wherein,
h is the number of nucleotides;
A is an adenine deoxyribonucleotide;
(X₁₇) is any deoxyribonucleotide, and h is 0 or 1;
(X₁₈) is a cytosine deoxyribonucleotide or thymine deoxyribonucleotide;
(X₁₉) is a cytosine deoxyribonucleotide, thymine deoxyribonucleotide, or guanine deoxyribonucleotide; and
(X₂₀) is any deoxyribonucleotide.

36. A recombinant vector, wherein, the recombinant vector comprises a nucleic acid encoding the nuclease according to claim 12.

37. The recombinant vector according to claim 36, wherein the recombinant vector includes a recombinant cloning vector, a recombinant eukaryotic expression plasmid, or a recombinant viral vector.

38.-39. (canceled)

40. A recombinant host cell, wherein, the recombinant host cell comprises the nuclease according to claim 12.

41. The recombinant host cell according to claim 40, wherein the recombinant host cell includes an animal cell, a plant cell, an algal cell, a fungal cell, a yeast cell, or a bacterial cell.

42.-44. (canceled)

45. A method for deleting, replacing or inserting a targeting gene of a host cell, wherein the method comprises: delivering the nuclease according to claim 12, or a nucleic acid encoding the nuclease according to claim 12 into a host cell.

46. (canceled)

47. The method according to claim 45, wherein the delivery method includes cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun.

48.-56. (canceled)

57. A kit, wherein, the kit comprises the nuclease according to claim 12.

58. A nucleic acid, wherein, the nucleic acid encodes the guide RNA according to claim 19.


SEQUENCE LISTING (600 sequences)

SEQ ID NO: 1 - protein, 378 aa, synthetic construct
MLLRKAFKFEIRPNGEQVRKMKQFCGCSRFVFNKALAWQNEQYEQDNHHKFSYTKLANLLPQWKKELVWLKDCHSQVLQQSLKDLESAFKNFFQKRADLPKFKKKGVKESFRFPQGCKIEQKNDRLYLPKIGWVRYRNSREIVGEVKNVTVSMKCGRFFVSIQTEFKQEIPTHKGGEIGIDMGIARFATLSNGEFFEPLNAFKTHKGKLAKLQKQLKNKVKFSKNWQKLKDKIGKLHHKIANCRKDFLHKISTQISKNHAMIYVEDLQVANMSKSAKGTAEVHGKNVKQKSGLNRVILDQSWFEFRRQLDYKTQWLGGFLLAVPPQNTSRTCPCCNYTAKENRQTQENFECVECGYTENADVVGAMNVLTRGQAIVQA

SEQ ID NO: 2 - protein, 402 aa, synthetic construct
MQLRYSFRLYPSVGQRMALARAFGCARVVYNDALRARETARSEGLPFPKTGDLSKALITEAKSTPERAWLGEVSAVVLQQSLRDLEAAYRNFFDGLKGKRPRMGAPRYKSKRDTRQSVRFTANARWSITPGGRLRLPKIGDVKVKWSRSLPSTPSTVTVVKDAAGRYFASFVVETGPEEVLPQVAPEVGIDLGLGHFAVLSDGTKIDSPRFLRRAEKRLKKAQRSLSRKEKGSKNRDKARIRVARAHARVADARREFHHQLSTQLIRDNQAVAVEDLAVKGLARTRLAKSVHDAGWSAFVSMLEYKAARYGRTFVKIGRFEPTSRVCSVCGVKDGPKPLHIREWTCQACGAVLDRDINAAVNVAKAAGLAASACGAQVRPGLVPAPRGEAGTHSTPQPQAAR

SEQ ID NO: 3 - protein, 363 aa, synthetic construct
MHRAYKFRLYPNKEQTTLINKTIGCTRFVFNHFLAKRKDAYEQEKKTLGYHDCSAMLTQLKKEIEWLKEVDSTALQSTLKDLDFSYKKFFKEKKGYPKFKSKQNPKQSYTCKMNIKVEGNRIKLPKLGWVAFAKSREVDGRILSATIRRNPSGKYFVSVLCETEVLLLPQVEQTVGIDLGIKDFAILSTGEKVENPKYYRKYEKQLAKWQRIQSRRQKGSKNRNKARMKVARLHEKIANTRNDFLHKLSTKLINENQVICLEDLQVENMVKNHKVAKSIADASWSMFRTMLEYKSEWYGRKVSIVGKQFPSSQLCSTPNCGYCNKDVKNLNLRSWICPNCGVQHDRDQNAAINIEQEGLRLLA

SEQ ID NO: 4 - protein, 399 aa, synthetic construct
MLLRKVYRFRMEPTQAQAEALLRMAGARRFVWNWGLARRKEAYAATGKGLTYNGQATELTALKKRPEMAWLREADSQLLQQALQDLDRAFKAFFERRAGFPRFKTRKKDPPRFRIPQRVRVEEGKVYLPKVGGVKIRQSQPIDCVIKGATFKRDTEGHWHVTLTAEFEMPDVPLPPVNPEHVVGIDLGLKDFAVLSDGTRIAPPKFYRKAERKLRRAQRELSRKQKGSKNREKARHRLNRVHAKVRNQRQDWLHKLTTGLVQKYDGLCIEDLNLKGMAKTKLSKSVLDAAPGEFRRQLEYKAVWYRKHLVVIDRYFPSSKLCRECGTIHTALTLSDRVWTCECGAVHDRDLNAALNIRAEGIRAIPVAVGHPETLNAWGEGVRPTQRRQSSSNQESHVL

SEQ ID NO: 5 - protein, 367 aa, synthetic construct
MVVNKAYKFRIYPTPEQATLINKTIGCARFVFNRFLALWSDTYKETGKGLSYGACSAELTKLKQEIDWLKEPDKFSLQNALRNLSDAYHRFFKKQNDAPKFKSKKNPVQSYQTNYTNGNIEIVDNQIKLPKLGLVKFAKSREVEGRILNATIRRHPSGKYFVSVLVEANIQELPKTGKEVGIDVGLKDFVILSNGVVVGNPRFFRRLEEKLARAQRKLSRMMKGSSNWNKQRIKVARIHEKITNARTDYLQKISTHIVKNHDIIAIEDLQVANMVKNHKLAKAISEVSWSMFRSMLEYKAKWYGRIVVAVGKTFASSQLCSCCGYKHKDVKDLKLREWDCPECGAHHDRDFNASLNILAEGKRLLAV

SEQ ID NO: 6 - protein, 365 aa, synthetic construct
MQKGIKFRIYPNREQKNFIHQTLGCCRFIYNRGLAMRKEGYENGEKIGYSQTSAMLTELKKQEEFAFLKAADSIALQQSLRDLDRGFVNFFQKRASYPKFKSKHNRFQSYRTVNQKDNIRIVGRYIKLPKLGSVKIRQSMEVEKINHVIIEHTPAGKYFAVLNVDFEPEPRSNAGGTIGIDVGIKAFYSDSNGNTVSNPRYLERSMRKLIREQRRLSRKQKDSHNREKQRIRVARVYEKVTNQRNDFLQKQSTMLVRENQTICIEDLNVKGMIRNHKLSKSIASVSWAKFFEMLEYKAGWYGNEIHRVPTMYPSSQTCSCCGYRNPRIKNLGIRIWECPKCHAVHDRDTNASINILKKGLQMQSA

SEQ ID NO: 7 - protein, 391 aa, synthetic construct
MERGFKYRIYPNESQRDQIARMFGCCRFVYNRALDVKKTAYSETGKSIATNDLIKMIPAWKRDPETSWLAQVDSMALQQSIRDLDRAYKNFFRRVREGGKPGFPKFKSRRHARQSYRTNGGKVLDRNHIALPKLGTVRAKVSRPLQGRFMSVTVSLDAAGRYFATFLCTDVPAKNTTATDREVGIDLGVETLATLSDGTKIENPRHLKKYERKLAREQRRLSRRKGARRGEKPSGRYMKQRKRVSRIHANIADARTDALHKVTTMLADENQVLCMEDLNAKGMVRNHHLAKAVSDASFGEFARLLEYKCAERGRSLVKIGRFYPSSKTCSACGHRLDALPLSVRSWDCPVCGAHHDRDVNAARNILTEGKRILSNTQGTAGHAGTVAAITA

SEQ ID NO: 8 - protein, 364 aa, synthetic construct
MEKAFKYRIYPNREQRILLAKTFGCTRFVYNHYLAKRKDAYEKDGITLSYSACAKDLVSLKKEYEWLKEVDSVALQSSVKNLDTAYINFFKKKAEYPRFKSKKTHRYSYTTKYTNGNIELFDNKVKLPKIGLVKIKKTRTPKGRLLSATVSQVPSGKYYVSLCYTDVEEVLFPLTYNETGIDLGIKDFIVLSNGEKVENPKYLKKSIEKLKKLQKQQSRKTKGGRNWIKNRIKIARLHEKIANQRMDFINKLSTRIIREYDIICIEDLKVENMLKNHNLAQSISDVSWSKFTTQLEYKAEWYGKVIKKVDTFYASSQTCHCCGYKNEETKDLKVREWTCPKCHSNHDRDVNASINILMQGILAN

SEQ ID NO: 9 - protein, 403 aa, synthetic construct
MHREPEERVAMERAYEIRIYPNAAQRKLIERTFGCARWVYNRCLETRKAEYERTGRSPSVYQLQKLVTAWKRTDAPWLAEVDSHALQQAAVNLGRAFDGFFRRCRAGGKPGYPRFKSKRDARQSYRTNWGISVPDARHVKLPIIGVVKARVSRPVEGRVLNATVKRVPSGKYFCVLTCTDCPKPDIPEGRIEVLGVDVGIRCLMARSDGNAIANPKSLARSEKKLARAQRRLSRKKRGSRRRAKQRARVARIHEKIASQRKDALHKATTEAVRESQAIAVEDLDVRGMEKNRRLAKSVADASMSEMARQLEYKCSWYGRRFVKVGRFYPSSKTCSACGQVNQAVVLGVEAWTCPACGCRHDRDLNAALNIAREGARLLNEADGTAGLAGTGDAKASRTLVEQA

SEQ ID NO: 10 - protein, 409 aa, synthetic construct
MLGMQRLQAFKYELMPTGEQQRLMHRFAGSCRFVFNQALALQQANREAGGKFIGYVAMARQLTAWRNSTETVWLKEAPVHPLQHALKDLEKAYRNFFEQRADFPRFKRKGQRDSFRYPDPKQIKLDQANCRIFLPKLGWLRYRNSRPVLGELRNVTVSLSAGKWHVSIQTRREVETPLPQGRAVGIDLGIARFATLSDGTVYAPLNSFKRHETALRKAQQAMSRKVKFSANWKKAKARVQRLHARIGNARRDYLHKTSTAISQNHAMVCIEDLQVRNMSKSAAGTTEQPGRNVRAKSGLNKAILDQGWFEFRRQLDYKLAWRGGWLVTVPPQNTSRTCPCCGHVSADNRQTQARFACVECGFEDNADVVGAINVLRAGHARFACEVSGEVMPPAAGTHRSDSGAAQCRA

SEQ ID NO: 11 - protein, 423 aa, synthetic construct
MPNIVVVAHRLYYNIRMQRLQAFKYELRPDGQQERQIRRFAGSCRFVFNKALALQKERYERGEKKLGYTGLCKLLTEWRNSTETAWLADAPVHPLQQTLKDLERAYSNFFARRADFPRFKKKGQSDSFRYPDPKQIKLDQANSRVFLPKLGWLRYRNSRKVLGTVRNVTVSQSCGKWFVSIQTEREVEQPVPQATTAVGIDMGVTRFATLSDGTFYAPLNSFKRHETALRKAQQAMSRKTKFSNNWKKAKARVQHIHSRIGNARRDYLHKCSTTISQNHAMVCIENLQVRNMSKSAAGTADAPGRNVRAKSGLNKSILDQGWFEFRRQLDYKLAWNGGYLIAVPPQNTSRSCPCCGHVSAENRQTQARFECVECGFEENADLVGAINVLRAGHARFACEVSGAVMPPAAGTHRSDSGAAQCRS

SEQ ID NO: 12 - protein, 397 aa, synthetic construct
MLTGRRYLLAFTPEQEEFAEKIGDACRVVWNTALEQRRAYRRRGAFIGYVEQARQMAEAKKDFPWLAEAPSHTLQQTLRDLERACTRHGTFKVRWRSKRKNAPTFRFPDAKQITVERISRRWGRVKLPKLGWTRFRWTRPLGGQLRNATVLKDGGRWYISFCVEDGLVESAPNGKPPVGVDRGVTVAVATSSGWMRDREFVTPGEAVRLKRLQQQLARQRKGSNRRAATRAKLAKLNARIRARRTDFLAWTANRLTRDHGLVVVEDLNIKNMTASAKGTVEQPGQNVRQKAGLNRAILAKGWGGLLTALEHKARYNGSRILRVPPAFTSQTCHACGHCAPDNRESQAVFRCRACGHQDNADVNAAKNILAAGLAVTGRGDLAVGRSVKRQPPEAEVA

SEQ ID NO: 13 - protein, 391 aa, synthetic construct
MIRTYRYRLYPTKAQARTLDFLLWQGRTLYNAALEQRITVYKETGKGVSYYQQWAYFRDLRNANPDTLGQLNATSVQQMLRRLDKAFVAFFRRLKAGETPGFPRFKGRDRFHSLEYRHGDGCKLRMGERVMLYIQNVGEVKVKYHRPLPDDAAIKHVVIKRRAHRWYACLMLDLPDPVILEHTGPAVGIDMGLSHLLALSDGTLVENPRWLRQSLAQLRVAQRRLARRQRGSARRRKAAAQVARLHERVANQRRDFWHKLTRQLADTYSLIALEDLTLAFMTANHHLALSAHDAGLAEFQQLLAYKAEEAGTQVVTVNPAYTSQACSGCGVVVKKDLSVRVHVCPDCGLTLDRDVNAARNILMLAVNPARTGPPERNVAGHGERALGSSPL

SEQ ID NO: 14 - protein, 378 aa, synthetic construct
MSNATSSLKETRNYKFRLYPNKEQESLLLRWLETCRWIYNTALAQRKEAWEREKRSVTRIEQQVWLKEAKKTDERLREIHSQVAQEVLFRVEKAFAAFFRRVKNGKTPGYPRFKGRGRYKSITFTQFGEGKGASFQDGKLRLSKIGLVKIRLHREIPGTIKTVTVKRDAAGKWWAIFAAEIELAQSQAHTGPAAGLDAGLEKFAALSDGSIIENPKYLRKMEKRLKHTQRNLSRKKKDSRNRKKARQKLAKLHAKVCNQRRDFLHKQSRRLVETYGLIAVEKLNIKGMVKNHHLAKSISDAGWGEFMAMLEYKAAEAGTKLVKVNPSGTSQVCSGCGMDVPKELSERTHCCPYCGLILDRDVNAARNILKKALALEAA

SEQ ID NO: 15 - protein, 402 aa, synthetic construct
MERLQAYKFQLRPKAGQESLMRRFAGCCRFLWNKALVLEKETYEKEGKRLGFSLLCEALRDWKKENDTSFLAEAHSQILQQALKDLDRAYKNFFAKRADFPRFKRKGVHDAFRYPQGFRLDEDNSRIFLPKIGYVRYRNSRRIEGTPKQVTVSLSAGRWFVSVQTEREVPEPVHTSQSAVGIDMGVARFATLSDGSCIKPLHSFRKHEKRLAKLQRKLAKRVKFSANWQKLKAKIQRLHRTIANVRNDFLHKTTTIISKNHALVVIEDLKVRNMSRSASGTSELPGKNVRAKAGLNKSILDQGWFEFRRQLEYKLKWLGGKLVVIPPQYTSQTCSHCGCVDRANRPAQAKFKCVACGFECNADYNAALNILAAGQAVSACGAGRPQAPALKQEPAYGVARYR

SEQ ID NO: 16 - protein, 435 aa, synthetic construct
MGTEPDRRPTEGQERTAAQAGVPIPAADAVSVDVLVLLFYGRQRVLGHDPPVYRGAAHPRLMATKTFVYKLRPTRSQEATMAETLETCRRLYNRALGERKDAYEQEQRSVCFAEQCASLPLAKKESPFLPRVHSQVLQDVLHRLDHAFAAFFRRVKAGEKPGYPRFRGAGWYDSFTYRQYGNGAKFDGNRLVLSKIGSIKFCKDRPLEGKPKTCTIIRKANGWFAHITCEVEPEPLPKTGETVGVDLGIESFATLSTGEQIANPRHYRTAERRLKTAQRRLSRRQKGSNGRRKARQLLAKAHLKVGRTRRDFCHKVAHDLVTRFDHIVVEDLNIRGMVKNHPLAKSISDAGWGTFVTILSEKAESAAKTVEKVNPAYTSQNCSACGERVLKALSVRWHSCPYCGAELHRDHNAALNIRKKGGGTALVDVAALAAA

SEQ ID NO: 17 - protein, 415 aa, synthetic construct
MQRLQAFKYELMPTCEQERNMRRFAGSCRFVFNRALALQKERYEQGEEKLGYAGLCKLLTEWRNGPETPWLADAPVHPLQQTLKDLERAYANFFAKRANFPRFKKKGQHDSFRYPDPKQIKLDQANSRIFLPKLGWVRYRNSREVLGTVKNVTVSQSCGKWFVSIQTEREIQAPVPKATSAVGIDMGVTRFATLSDGTFYAPLNSFKRHEDRLRKAQQAMSRKTKFSNNWKKAKARVQRIHFRIGNARRDYLHKSTTAISQNHAMVCIEDLQVRNMSKSAAGTTEQPGKHVRAKSGLNKAILDQGWFEFRRQLDYKLAWAGGHLIVVPPQNTSRTCPACGHVSADNRQTQARFLCVECGFEENADLVGAINILRAGHAHCACEVSGAAMPPAAGTHRSDSFRLDAGVGAVGIPRL

SEQ ID NO: 18 - protein, 426 aa, synthetic construct
MQRLQAYRYELMPTGEQQRKMRRFAGSCRFVYNKALALQKARYEQGEKKLGYAGLCKLLTEWRNSSDTAWLADAPVHPLQQALKDLERAYNNFFAKRASFPRFKRKGQSDSFRYPDPKQIKLDQANSRLFLPKLGWLRYRNSREVLGAVKNITVSQSCGKWFVSIQTEREVEPPIPQGGVVGIDMGIARFATLSDGTFYAPLNSFKRHESRLRKAQQALSRKVKFSNNWKKAKARVQRIHARISHARRDFLHKISTAISKNHAVVCVEDLQIRNMSKSAAGTTEQPGRNVRAKAGLNKSILDQGWFEFCRQLDYKLAWRGGWLVVVPPQNTSRTCPCCGHVSADNRQTQDRFACVECGFEANADLVGAINILSRGMQKLRDEGRDTLDAFSGTAQAVSPDGLWIEPRWRSEAGTHRSESGAAQCRA

SEQ ID NO: 19 - protein, 408 aa, synthetic construct
MGMRRLQAYKYELRPDGQQERQMRRFAGSCRFVFNQALALQKERYEQGEKKLGYAGLCKLLTEWRNSSETAWLADAPVHPLQQTLKDLERAYSNFFAKRADFPRFKKKGQSDSFRYPDPKQVKLDQANSRVFLPKLGWLRYRNSREVLGAVKNITVSQSCGKWFVSIQTEREVEQPLPQGGAVGIDMGITRFATLSDGTCYSPLNSFKRHETALRRAQQSMSRKTKFSNNWKKAKARVQRIHSRIGNARRDYLHNATTTISQNHAMVCIEDLQVRNMSKSAAGTTEKPGKKVRAKSGLNKSILDQGWFEFRRQLDYKLAWSGGYLIVVPPQNTSRTCPACGHVSAENRTSQAKFACVECGFEENADLVGAINILRAGHARFACEVSDAVRSPAAGTHRSDSEAVQCRT

SEQ ID NO: 20 - protein, 417 aa, synthetic construct
MFSSIPINHLCTVIRVQLRYSFRVYPSTGQRTALARAFGCARVVYNDALRARETARSEGLPFPKTGDLSKTLITEAKQTPGRAWLGEVSAVVLQQSLRDLDTAYRNFFDGLKGKRPRMGAPRYKSRKDTRQAVRFTANARWSITTGGKLRLPKIGDLKVKWSRALPSVPSTVSVVKDSAGRYFASFVVETGPGELLPQTTPGIGIDLGLGHFAVLSDGRKVDSPRFLRRAEKRLKKAQRALSRKEKGSSNRNKARLKVARAHAKVTDARREFHHRLSTKLIRENQAVAVEDLAVKGLARTRLAKSVHDAGWSAFVSMLEYKAARYGRAFHRIGRFEPTSQVCSQCGVKDGPKPLNVRVWTCQACGAVLDRDINAAVNVAKAAGLAVSACGAQIRPGLVPAPRSETGTHPTPQPSTAR

SEQ ID NO: 21 - protein, 365 aa, synthetic construct
MNLHRAYKFRLYPNKEQATLINKTIGCTRFVFNYFLAKRKDAYEQENKTLNYNDCSALLTQLKKEIEWLKEVDSTALQSTLKDLDSSYKKFFKEKKGYPKFKSKKNQKQSYTCKMNIKIEGNRIKLPKLGWVSYAKSREVEGRILSATIRCNPSGKYFVSVLCETECKLLPQVERTVGVDLGIKDFAILSTGEKVANPKYYRKYEKQLAKWQRIQSRRKKGSNNRNKARTKVAILHEKIANTRNDFLHKLSTKLINESQVICLEDLQVENMVKNHKLAKSIADASWSMFRTMLEYKAEWYGRKVSIVGKQFPSSQLCSTPNCGYRNKEVKSLNLRSWICPHCGVQHDRDQNAAINIEQEGLRLLA

SEQ ID NO: 22 - protein, 363 aa, synthetic construct
MSMHRAYKFRLYPNREQATLINKTIGCTRFVFNHFLAKRKDAYEQEQKTLNYNDCSTLLTQLKKEIEWLKEVDSTALQSTLKALDSSYKKFFKEKKGYPKFKSKKNPKQSYTCKMNIKVEGDRIKLPKLGWVTFAKSREVEGRILSATVRKNPSGTYFVSVLCETEIQPLLQAEKTVGVDLGIKDFAILSTGEKIANPKVLRYYEKKLATWQQKLSRRKKGGQNREKSRKQVARLHERISNTRNDFLHKLSTQLIRENQTICLEDLRVENMIKNHKLAKSIADASWSAFRTMLEYKAAWYGRTISIVGKQFPSSQLCSGCGYQNKEVKNLNLRNWTCPNCGGQHDRDKNAAINIEQEGLRLLA

SEQ ID NO: 23 - protein, 391 aa, synthetic construct
MERGFKYRIYPNESQRDQIARTFGCCRFVYNRALDVKKTAYSETGKSIATNDLIKMIPAWKRDPETSWLAQVDSMALQQSIRDLDRAYKNFFRRVREGGKPGFPKFKSRRHARQSYRTNGGKILDRNHIALPKLGNVRAKVSRPLQGRFMSVTVSLDAAGRYFATFLCTDVPSKDAPATDREVGIDLGVEALATLSDGTKIENPRHLKKYERKLAREQRRLSRRKGARRGEKPSGRYLKQRKRVARIHAKIADARTDALHKVTTMLADENQVLCMEDLNAKGMMKNHHLAKAVTDASFGEFARLLEYKCAERGRSLVKIGRFYPSSKTCSVCGHRLDALSLSVRSWDCPSCGVHHDRDVNAARNILAEGKRILSNTEGTAGHAGTVAAVVA

SEQ ID NO: 24 - protein, 391 aa, synthetic construct
MERGFKYRIYPNESQRDQIARTLGCCRFVYNRALDVKKSAYAKTGKTVSWAELSRMLPAWKRNPETSWLAQVDSMALQQSIRDLDRAYKNFFRRVREGGKPGFPKFKSRRHARQSYRTNGGKVLDRNHIALPKLGNVRAKVSRPLQGRFISVTVSLDAAGRYFATFLCTDVPAKDTTTTDREVGIDLGVETLATLSDGTKIENPRHLKKYERKLAREQRRLSRRKGARRGEKPSGRYMKQRKRISRIHAKIADARTDALHKVTTMLADENQVLCMEDLNAKGMVRNRRLAKAVSDASFGAFARLLEYKCAERGRTLVRVDRFYPSSKTCSACGHRLDALPLSVRSWDCPACGAHHDRDVNAARNILTEGKRILSNTEGTAGHAGTVAAVVA

SEQ ID NO: 25 - protein, 391 aa, synthetic construct
MERGFKYRIYPNESQRDQIARTLGCCRFVYNRALDVKKSAYAKTGKTVSWAELSRMLPAWKRDPATSWLAQADSMALQQSIRDLDRAYKNFFRRVREGGKPGFPKFKSRRHARQSYRTNGGKIVDRNHIALPKLGTVRAKVSRPLQGRFMSVTVSLDAAGCYFATFLCTDVPAKDMPATDREVGIDLGVETLATFSDGTKIGNPRHLKKYERKLAREQRRLSRRKGARRGEKQSMRYLKQRKRVARVHAKIPDARTDALHKVTTMLADENQVLCMENLNAKGMVRNHHLAKAVSDASFGEFARLLEYKCAERGRTLVKVGRFYPSSKTCSACGHRLDVLPLSVRSWDCPVCGAHHDRDVNAARNILTEGKRILSNTQGTAGHAGTVAAITA

SEQ ID NO: 26 - protein, 397 aa, synthetic construct
MLTGRRYLLAFTPEQEEFAERIGDACRVVWNTALEQRRAYRRRGAFIGYVEQARQVAEAKRDFPWLAEAPSHTLQQTLRDLDRACARHGTFKVRWRSKRKNAPTFRFPDANQITVERVSRRWGRVKLPKLGWTRFRWTRPLGGQLRNATVLKDGGRWYVSFIVEDGLVESAPNGKPPVGVDRGVALAVATSSGWMRDREFITPGEAARLKRLQQQLARQQKGSNRRMATRAKLAKLNARIRARRTDFLAWTANRLTRDHGLVVVEDLRTKNMTASAKGTVEQPGQNVRQKAGLNRAILAKGWGKLLTALEHKARYNGSRILRVPPAFTSQTCHACGHCAPDNRESQAVFRCRACGHQDNADVNAAKNILAAGLAVTGRGDLAVGRSVKRQPPETEAA

SEQ ID NO: 27 - protein, 372 aa, synthetic construct
MTHQHKAFKFRLYPHEEQKELLEKTFGCVRFVYNQMLAERKERYEKFKNDKERLKKQKWPTPAKYKEEFPFLKEVDSLALANAQINLQNAYKNFFKGRTEFPKFKSKKHKQSYTTNMVNGNIKLENGHIQLPKFKKPIKMKQHREIPAEYKIKSCTISKTKSGQYYISILTEYEKEMEPVKIQKVVGLDFAMDGLYVESERGEKANYPRYYRQALDKLAKAGRILSRRKKGSSRWEKQRLKVAQLHEKIANQRMNFLHHKSKELAITYDAVVIEDLDMKGMSQALHFGKSVHDNGWGMFTRFLEYKLKEQGKQLIKIDKWFPSTKKCSCCGKEKEMPLSERVYQCSCGLVLDRDHNSAINIKNEGLRLVAFA

SEQ ID NO: 28 - protein, 369 aa, synthetic construct
MAIHNKAYKFRLYPTEEQAYLIRKTFGCVRFVYNKMLAERKETYEQFKNDKEALKKQKFPTPAKYKGEFTWLKEVDSLALANAQLHLQTAYKNFFGGKADFPTFKSKKARKSYTTNVVNGNIMLLDGYIKLPKLKLVKIKQHRMIPDEHTIKSCTISMTPTGKYYVSILTEYEANVIPKKLNTFVGLDFAMTELYVDSEGEKANYPRFYRQEFVRLAKAQRVLARRKKGSSRWHKQRIKVAKLHEKITNQRKDFLHKKSYNLAKTYDCIVIEDLNMKGMSQALNFGKSVADNAWGMFTAFLQYKLEEQGKRLIKIDKWFPSSKTCSCCGQIKESLSLSERTFHCDCGFVADRDWNASINIKNEGLRLLA

SEQ ID NO: 29 - protein, 426 aa, synthetic construct
MQRLQAYRYELMPTGEQQRKMRRFAGSCRFVYNKALALQKARYEQGEKKLGYAGLCKLLTEWRNSSDTAWLADAPVHPLQQALKDLERAYNNFFAKRASFPRFKRKGQSDSFRYPDPKQIKLDQANSRLFLPKLGWLRYRNSREVLGVVKNITVSQSCGKWFVSIQTEREVEPPIPQGGVVGIDMGIARFATLSDGTFYAPLNSFKKHESRLRKAQQALSRKVKFSNNWKKAKARVQRIHARISHARRDFLHKISTAISKNHAVVCVEDLQIRNMSKSAAGTTEQPGRNVRAKAGLNKSILDQGWFEFCRQLDYKLAWRGGWLVVVPPQNTSRTCPCCGHVSADNRQTQDRFACVECGFEANADLVGAINILSRGMQKLRDEGRDTLDAFSGTAQAVSPDGLWIEPHWRSEAGTHRSESGAAQCRA

SEQ ID NO: 30 - protein, 400 aa, synthetic construct
MRRLQAFKFELMPNGEQQRDMRRFAGSCRFVYNKALALQKENYEAGGKFIGYVAMAKHLTEWRNGGETPWLKDAPVHPLQQALKDMERAYKNFFAKRAAFPKFKKRGQRDSFRYPDPKQFKLDQPNGRIFLPKLGWMRLRLSRPVLGELRNATVSFNAGKWCVSIQTEREVEQPVPHATSVIGIDVGIARFATMSDGTFLTPLNSFKKHEARLRRCQRAMSRKVKFSNNWKKAKARVQRIHTRIGNARRDYLHKATTTISKNHAMVCIEDLQVRNMSRSAAGSTDAPGKNVRAKSGLNKAILDQGWFEFRRQLEYKLAWNGGWLVAVPPANTSRTCPACGYVSADNRTTQKKFTCVECSYEENADVVGALNILARGHRVAACGEPVKSGRSVKQEPAEAI

SEQ ID NO: 31 - protein, 438 aa, synthetic construct
MPHTKYTQGVVTLRYRYRAYPTRGQEKSLARLLGCCRVVFNDAVAAREAAHANGHAYPTRAELSAALAAAKHTTERHYLTEVSAVPLQQVLADVESAYRNFFASLKGERRRAARHPKFRSRSDRRQAARFTKNARFSVETRGRDGVCTLPKIGPVRFVCSRELPSTPSSVTVIREADGRYYLSFVVQVAEQAAEPNGSAVGIDVGLSAYVTMLSQCGDSEQVAKVASPLYLRRRERALKRSQRSLCRKQKGSKNREKQRRRVAVSHRKVREARLDHAHKVAASIVDAHDVICVEDVNVAAMAKSRLAKSIADQGIGQFLRLLEEKAERRGRHLVRVGRFFPSTQLCSSCTAPVGPKGQQELGVRHWTCRNCGVSHDRDVNAARNILTEGLRLLAMREQQVADGQSETINARGADVRQGCSPAVGREAGRNAGDLSCVA

SEQ ID NO: 32 - protein, 417 aa, synthetic construct
MLFRQGFLFKLKPNGRQARLMSRFAGCCRFVYNKGLTWAKEQYESKTGKHPTYKNLSALLPEWKKDPELPWLSECYSQCLQQSMKDLSRAYSNFFSGRADFPVYHKKFGTSDGFRYPQGFKIDETGKQIYLPGIGFVKYRRSRYIEGKAKNVTVMRKADGWYVSIQTEYEKEIPKHNGDEVGMDMGVVWFCTLSDGTQIAPCDALKKHLKKLEQLQRALSRKEKFSSNWKKAQQKVAKLHQRIGNIRQDFINKVALDLSKKHAVIYREDLRIKNMTASAAGTMEDPGHNVRQKSGLNRAILDQAWGKFFQRLQWKCEQRGGIVIKVAPQYTSRTCPLCGHESKENRKSQASFQCVACGYQDNADVVGARNIKVRGQRMSACGELGEEGRARVNRWMESIHRAGQQQEPIEVIIGESR

SEQ ID NO: 33 - protein, 469 aa, synthetic construct
MPFDISSRRPNAHTSRIRSVFQFEYSLSFHKTKCYNTHMILRKAYQFKLKPDGATGRKLSRFCGCARFVYNKALAWNRDQRSSDPNFKISYAKLCEQLPEWKSAEETKWLSECHSQVLQQSLKDLMRAFTNFFEGRTDFPKFHKKFRNEDSIRYPQGFKVDEARRQIFLPSIGWVKYRRSRFIEGIPKNVTVSRKVDGWYVSVQTEREVPDPVHPKAGTEAGIDMGVVRLYTCSDGTFKEGVRCLRDKLEKLAKEQRRLKRMVKFSKNWIKQQQRIARLHKKVADSRRDLLQKAANRLCQNHAVLYREDLKIRNMTASAKGTVEEPGKNVKQKRGLNRAILDQGWGMFFSFLNAKMTELGGLVFAVPPQNTSRTCPVCGCVSKENRRTQAKFRCTSCGYEGNADAVAAVNILGRGQRLRACGELVPVNAAQVSRPSRKRSVVQQQEPVEATVGGSADNGTVGNHRPSGR

SEQ ID NO: 34 - protein, 372 aa, synthetic construct
MEYSYKFRLYPNRKQETQMLRTFGCCRFVFNHYLADRIETYKETGKSPTRFEQDRNLTVLKKQEETSWLREVDATALQSSLRDLDDAYQNFFRRVKHGEKPGFPRFKSKHDHRQSYKSKCVGTNIKVLDKAIQLPKLGAVKCRISKEVKGRILSATVSRNPSGKYFVSLCCTDAEVAALPRTGSVIGIDMGIRDFAVSSDGIAYENHKYLAKSEKKLAKLQRQLSRKAKGSCNRDKARLQIARLHEHISNQRKDMLHKLSSQLIRENDVICIEDLASKNMVKNHKLAKSIQDASWGEFRRQLEYKAKWYGKKVVVIDRFFPSSQLCCNCHGLWSGTKDLSVRKWTCPVCGHTHDRDRNAAINILNEGLRLLA

SEQ ID NO: 35 - protein, 370 aa, synthetic construct
MEYSYKFRIYPTKAQENLIQRTFGCCRFVFNHFLAERIEQYKATGKSPTRFQQDKSLTALKKELEWLREVDATALQASVQTLDTAYQNFFRRVKQGGKPGFPRFKSKRDHRKSYKSKRVGENIKVLDKAVQLPKLGLVKCRVSKEVKGRILSATISQNPAGRYFVSLCCTDVDVAPMLSTGAVVGLDMGLKAFAITSDGTEFPNHKYLAKSQKKLAKLQRKLSRKSKGSNRRDKARVQVARLHEHISNQRQDMLHKLSTELIRQYDVICIEDLATSNMVKNHKLAQSISDASWSEFRRQLEYKAAWYGKQVVAIDRFFPSSQLCSACGTTWPGTKDLSVREWICPECGTIHDRDLNAAKNILNEGLRLLA

SEQ ID NO: 36 - protein, 369 aa, synthetic construct
MVNKAYQFRLYPTKEQEQLLAKTFGCVRFVYNKMLEERIQIYEKFKDDKEALKKQTFPTPAKYKKEFPWLKEVDSLALANAQLNLQKAFQNFFSGRAGFPKFKNRKAKQSYTTNVVNGNIQLSDGYIKLPKRKWVKFKQHREIPAHHIIKACTITKTKTGKYYVSILTEYEHQPVPKEIQTVVGLDFSMNTLYVDSEGKRANYPRFYRQALEKLAKEQRILSRRKKGSNRWHKQRLKIAKLHEKMANQRKDFLHKESHQLAKLYDCVVIEDLNMKGMSQVLNFGKSVHDNGWGMFTTFLQYKLEEQGKKLIKIDKWFPSSKTCSCCGQVKESLSLSERTFRCECGFESDRDVNAAINIKHEGMKRLAIA

SEQ ID NO: 37 - protein, 369 aa, synthetic construct
MANKAYQFRLYPTKEQEELLAKTFGCVRFVYNKMLEERIQMFEKFKDDQESLKQQTFPTPAKYKKEFPWLKEVDSLALANAQLNLQKAFQHFFSGRAGFPKFKNRKAKQSYTTNMVNGNIKLSDGYIKLPKLKWIKFKQHREIPAHHIIKSCTITKTKTGKYYISILTEYEHQPALKEVQTVVGLDFSMSTLYVDSEGKRANYPRFYRKALETLAKEQRKLSRKKKGSNRWHKQRLKVAKLHEKIANQRKDFLHKESHKLAKRYDCVVIEDLNMKGMSQALHFGQGVHDNGWGMFTTFLQYKLAEQGKKLIKIDKWFPSSKTCSCCGRVKESLSLSERTFRCECGFESDRDVNAAINIKHEGMKRLAIV

SEQ ID NO: 38 - protein, 368 aa, synthetic construct
MANKAYKFRLYPTQEQEQFLVKTFGCVRFVYNKMLAERKETYEKFKDDKETLKMQKFPTPAKYKKEFAWLKEVDSLALANAQLNLQKAFKNFFSGRAEFPKFKKRKTRQSYTTNVVNGNIMLLDGYIKLPKLKCIKIKQHREIPSHHVIKSCTISRTKTGKYYVSILTEYEHQPVQKEVQAVVGLDFSMNGLFVDSEEGKTANYPRFYRQALGKLVKEQRILSRRKKGSNRWHKQRLKVAKLHERIANQRKDFLHKESHKLAKRYDCVVIEDLNMKGMSQALNFGKSVTDNAWDMFTTFLQYKLEEQGKKLIKIDKWFPSSKTCSCCGQVKESLSLSERTFRCDCGFVADRDWNASINIKREGLRLLV

SEQ ID NO: 39 - protein, 420 aa, synthetic construct
MLTATKIRLYPTADQERHLAVQFGCARFVWNRALAMKRAAWQERKESLSCYTIKGMLPVWKGGEFPWLKDADSQVMQEVIRHLDRAYRNFFEKRARLPRFKKKHAARQSIAYPQRVKLEGNLVYLPKVGWVKAVVHREIVGKIKTVTVSRESTGKYYASILADDGLPETEPLSRIERITGIDLGLKDALVSSAGHKSGNPKPLRRALKNLKRKQQAMSRKIEAAKKRFEAAKAADDGKAYRLRDFFGANIAKDRKRVAVAHERVRNARDDWQHKVSRQLADENQAVAAETLNVKGMMKNRRLSRAIADVGWGSLLLKIDYKLKQHGGRLVKIDRWFPSTKTCSCCGAIHEGLTLSDRSWLCTACGSLHDRDENAAENIRRQGILKLKAEGLSVSAHGGGVSRVSSAPATASEVGSLRLQA

SEQ ID NO: 40 - protein, 455 aa, synthetic construct
MEPRCRPCAHAVPRPAEDRAVEVHLRLQEREQPPTEEGVPRDQAQALEGVLLEQELLPAYDRRSPHRGHPPLHREPGAEMTVTMHKAVVYRAYPTREQAALIERTCGCARFVYNRMLADKIAHYEKTGEMLKVTPARYKREFPWLKEVDSFALCNAQLNLDAAYRNFFRRVKAGEKHGFPKYKAKHRSRCCYKTNFVNGNIRFEGDERYLRLPKLGSVRVRQKREIDPSWRIKSVTVEHTRSGRFEVSVLFEFETQEPEQVKPESFVGLDYSSHDLYVTSDGERPGYPRYFRHAERKLAREQRKLSRMQKGSANWRKQKLRVARVHEKMKNQRKDFHHKAANGLVERYDCVCVENLDMRGMSRSLKLGKSTMDNGFGMFRALLGYKLEDRGKRLVTVGRLFPSSQLCHECGYRFSGTKDLSVREWTCPLCGRTHDRDVNAARNIRDEGMRLALSA

SEQ ID NO: 41 - protein, 370 aa, synthetic construct
MGKSYKAYRFRLYPNKQQEQQFARTFGCCRFLYNHMLADKIEFYRRTGGMLKNRPADYKKEFPWLKEVDSMALCNVQMHLESAYRKFFTEPKTGFPKFHSKHHGTASYTTNVINGNIRLVSGRLKLPKMAPLKIILHRDVPTKARLKSVTVTREASGKYYASLLYEQENCENQTVLPVRADKVLGIDFAMAGLAVFSDGTKAEYPMYYRRGEQRLARAQRKLSRCKKGSRNYEKQKRRVAICHEKIRNQRADFQNKLIYRIAVSYDAVCVENLDMKAMSRGLHFGKSVMDNANGRFRRRLEEKMAEMGKTFIKEDRFFPSSKRCSCCGRVKAYLPLNERTYRCECGYVEDRDVNAAINLKMEGIRILGAV

SEQ ID NO: 42 - protein, 390 aa, synthetic construct
MTMRKVAQRIPFTPSRTQAALLERCFGDRRFVYNQQVEAFNAYDRETNPNPEYPDVSGMKNANGWLKDSPIPSNALSNAIMDFRKARSAYFRKAQYGKHRPRFASRNDSIQSFRNAMPIRRMDGNRYPLSRKLGSVRIRKRDRLRYPIENLSSWTVKRENRTYCLVLLFDVDIQPKTQAEGRIGIDLGVKDLLTLSTGEKIDYPNRLRRLEEDVRREQRKLSRRTKGSNNYRRQRAIVAKAYARLRRYRDNFQHQLSHRLIEENQFIGMETLMVRNMTRKARKRLDADGMPMRNGQSRKRAMNRSILRDGWSGLVDKLSYKAEWYGRILVQVDRFYPSSKLCHDCGHKYKGLRLSEREWVCERCGIPHDRDVNAALNIRDEALRLSREKA

SEQ ID NO: 43 - protein, 378 aa, synthetic construct
MIRTYKYRLRPTRKQRHILNDLLFQMQTVYNDALNERRVRWRRSRRAVTYFDQWERIRDERHALPDEMGLLNVTSIQQMLRRVEKSYKTFYKGQRGLPRFKGRNRFKSVEYRYGDGCKLDGDRLYIQHVGHIKIRLHRDIPDNAKIKHVVIKRSNGNWDVALMLELPAPKPAIHPGPAVGIDVGLKSLLALSDGTLIDNPRWLRKSLRDLRVAQRRLSRRKKSGSGWKNAKRQVAKLQNHIANQRRDFWHKLTITLAQTYSFIAIEDLNLAFMTRNGNLSLSAHDAALGMFRQMLTYKVEKTGTHLVAVNPSNTTRMCSRCGEIVPKSLNIRIHHCPHCGLTLDRDVNAARNILALGRRVETLTYPVGESVVSEAPPL

SEQ ID NO: 44 - protein, 411 aa, synthetic construct
MDALLFCSYSGKRVSGSDSTVYRCSKGIVMILKTYKYRLYPSKAQNRMLEQTVETCRRWYNTGLAERKTAWENEKRSLSKFEQLGKVKDYRKENPYAAQLHSHVLQVVVADLDKAFQAFFRRVQSGEAPGYPRFKGRNRFDSFGLKEYGNGFKVDGRRLKISGIGRVRVRWHRPIEGKIKTVRIRRQAGDWYACFTCEVPEHPLPPTGKAVGIDVGVHHLLATSEGEVVENPRWYRAEQQKLRVIQRRVSRRKKGGSNRRKAVLELQRQHLHIANCRKDYLDKLVHHFIMHYDIIALEDLQIKGMVRNHHLSKSILDAGWGYLKTHLSGKAVEAGREVVLVNPAYTSKTCCSCGTVFSELSLGDRWVNCPCGLSLDRDVNAALNILKRAGRALWDESTAIGLRLSHEAPLL

SEQ ID NO: 45 - protein, 395 aa, synthetic construct
MIISYRYPIFPSREVEQKLVEALDTCRWLYNRLLEEINKARESGKTLRTYDTQNLIPLLKTENPNLNKVYSKVLQMVNYTLWSNIKGLSALKKNGRKVGHLRFKGKGWYNTLNYNQSGFKLDQDHSILKLSKIGEIKIKLHRKVEGNIKAVLIKRAGDRWFALIQAEQEPKQLSETGNVVGLDVGLTSFVVDSDGNSVENPRFAEKAATKVKNIQRKLSRAKKGSNNRRKLVDKLDKAHERINNQRSDFLHKLSRMYVNRYDIICAEDLDVMGLKETGNNKGIHRNIHDASWSRFMSMLSYKAQSAGRKLIVVDPRNTSQRCSFCGSIVRKELSDRVHECPYCGFSSNRDYNAAVNILSTGMEQPVAPIESKPLHHVSVMQVLAMKWEALPFRVG

SEQ ID NO: 46 - protein, 375 aa, synthetic construct
MKPREGKVLNIAYRFRIYPTEEQKILLGKTFGCCRFLYNQMLNDKIREYEKTKKMLNNTPAMYKKEYPFLKEVDSLALANVQLHLEKAYKNFFRDSKIGFPRFKSKHHSQNSYTTNVVNGNILVESKQIRLPKLKWIAMKKHREPAEGLRLKSVTVSMEPSGKYFASLLYEGYSCENQAAEPDYSTAKILGIDYAMQGMAVFSEKVETEEAGFFRKNEKRLAREQRKLSRCVRGSHNYELQKKKVARCHEKIRNQRRDHLHKLSRKIADGYDAAAVEDIDMKAMGQCLHFGKSVQDNGYGMFREMLDYKLAWKRKKMVKVNRFFPSSKKCCKCGRIKKELKLSERVYHCVCGNKMDRDRNAAINIREEARRMLTA

SEQ ID NO: 47 - protein, 366 aa, synthetic construct
MNIAYKYRIYPDRKQTLLIEKTFGCCRKVWNLMLSDKIAYYKSTGKSLMTTPAMYKKEYPYLKEADSLALANVQINLQDAYKNFFRDNQISFPKYKSKKHCKKSYTTNMVNGNIVLDFENKTIRLPKLGKVKAVFHRLPASVWKIKSVTVSRNVINEYCISVLFEYEDTVIPMAYDRSDDILGLDYKSNGLYMDSEGHCADMPHYYRKSQKVLAKKQRRLSKKKLHSNNWYKQQFKVNKTVIKTARQRTDFLHKESRKIANSYDFVSVEDLNMKGMSQSLKLGKATMDNGYGMFINMLAYKLEAKGGRLIKTDKWFPSSQLCSKCGSIKKIPLSQRIYICECGNIIDRDFNSAINIALEGRRIVSA

SEQ ID NO: 48 - protein, 377 aa, synthetic construct
MNKAIRYRIYPTNEQCIMFAKTFGCCRKVYNLMLADKIASYKETKSFGRQTPAMYKKDYPFLKEVDSLALANAQLNLQRAMKNRFDKKRKSRCGFPKFKSAKHSRKSYTTNNQNGTVAIIDGRYIKLPKIGMVKAVIHRAPDTDWNIKSATVSQDCDGRYYVSVLFEFEKVIEKVEISDRALGLDYASDGLYVDSNGRRGSNHKYYRESHKKLAREQRRLSRKKGSKKGEAKSSNYMKQLRKVNRIYKHIANQRLDNLHKISAEIANQYDVVCVESLNMRAMSNRGFGNGKATLDNGYGMFLNMLEYKLADRGKYFVKVDKWFPSSQICSKCGRIHHEMKNLANRVMKCECGFVMDRDCNAAVNIKEEGLRMLKSAQ

SEQ ID NO: 49 - protein, 399 aa, synthetic construct
MERPSLESVLLCWNVRGYDKRGYSEVHRDSESDVTMQMKAYRYRLFPTKSQVSQMEQTLEICRWVYNETLAMRKNAWEQEQRHISYYESKRQIPLWKKERPELSTVYSQVLQNVTLRVDLAFKAFFRRLKSGEKPGYPRFKGKGRYDSFTYPQSGFKLDGDRLHLSKIGDVKIILHRPVEGTIKTLTIRRSATGKWYACFSVEYDPTPAPQKESVVGIDVGLESFATLSNGEKIENPRFFRTDEKALAKAQRKLSKAEKGTPERKKARKIVAHVHERIANRRLNFAHQTSRKVVDRFGTIVFEDLNITNMQKNHHLAKSIADVAWNMFITITESKAEDAGSRVILVNPRNTSQICSRCGMIVAKTLSDRVHSCSHCGLVMDRDQNAAINIMRLGLQSQG

SEQ ID NO: 50 - protein, 370 aa, synthetic construct
MEYSYKFRVYPTAAQAEQIQRTFGCCRFVWNHYLALRKDLYEQDGKTMNYNACSGDMTQLKKTLLWLREVDATALQSSLRDLDTAYQNFFRRVKKGEKPGYPKFKSKHHSKKSYKSKCVGTNIKVLDKAVQLPKLGLVKCRISKEVKGRILSATISQNPSGKYFVAICCTDVELEPLTSTGAVAGIDMGLKAFAITSDGVEYPNHKYLTKSQKKLAKLQRQLSRKSKGSKRREKARIQVARLHEHVANQRQDMLHKLSTDLVRNYDLIAIEDLAPSNMVKNHMLAKAISDASWGEFQRQLKYKAEWHGKKVVTVGRFFPSSQLCSNCGAQWSGTKDLSVRQWTCPVCGAIHDRDMNAARNILNEGLRLMA

SEQ ID NO: 51 - protein, 382 aa, synthetic construct
MKAMLVHKAYKFRIYPTKEQEILIAKTFGCARFVFNYFLAEWERAYRTTGKGLTYKVCSSQLTSLKKQFVWLKEVDSIALQTSLKHLSDAYTRFFQKQNNKPRFKSKKNKVQSYTTKYTNGNIAVVGNRIKLPKLGLVRVAKSREVEGRILSATIRRNASGKYFVSILAEVDVQPLPKTNRAVGIDLGVKHFAILSDGTTYANPKFLRKLEEKLAKAQRILSRRQIGSSNWHKQRIKVARLHEKMTNSRNDYLHKLSTEIVKNHDIIGVEDLQVANMMKNHNLAKAIADVSWSTFTNMLMYKATWYGKQVVVVAKNFPSSQLCSHCGYKNKDVKHLHLREWECPSCGTHHDRDVNASMNLKNEAIRLLTAGTAGLACSFSLR

SEQ ID NO: 52 - protein, 377 aa, synthetic construct
MKEMETYTLGYKFRIYPNATQARLIHRTLGCARFVYNHFLAVRRDQWNANRKSIGYTESSRLLTDLKKREETAWLSEVDSMALQEALRNLDRAFQNFFDKRARYPRFKSKHSHAQSYRTRNQSNGVRIVGKRIKLPKIGLVRIKQSREFSGRILSATVSRTASGKYFVSLCVEQDKAKLLRPNAGGQIGIDVGIREFYTDSNGNTVENPKPLKKLLRKLKREQRRLSRKLPRSQNRGKARVRLARVHERIANIRKDFLHKCTTRLARENQTAAVEHLNVKGMLKNHRLAQAISDVSWSEFFRQLAYKMELRGGELLKVETFYPSSQTCSVCGYQNTEVKNLGVREWTCPQCGAHHDRDHNAAKNILRRALENKAKAA

SEQ ID NO: 53 - protein, 406 aa, synthetic construct
MRTAYKVRAYPDTEQAALLRRTFGCVRLVWNKTLAERQQRYTTEQKSTSYKQTDAALSEWKKTEDLAFLSEVSSVPLQQTLRHQHSAFAAFFKGLAKYPRFKNRHGRQSAHFTRSAFRIKDGALWLAKTSAPLRIVWTWPGVDLAALDPTMVIVSREPDGRWFVSFAVDQPDPQPLPVAGESVGVDLGIKDFAVLSTGEKIANPRHMARHERGLRRQQRRLSRMKKGSENRARQRVKVARKHARVRDARRDFLHKTSTELVRRFDTIAVEDLAPKNMVRNRSLAKSISECGWGEFRSMLEYKAEKAGRRVAVINRWYPSSKTCSACGHLLAALSLGTRHWMCPDCGTRHDRDINAAKNILVAAGLAETQNACGGDVRPHGASHRQSPVKQEPSQATAGIPVLQGGE

SEQ ID NO: 54 - protein, 401 aa, synthetic construct
MARQVKRAFRYRFYPTGEQAAELSRTFGCARLVYNRALEERTRAWYSEQRRVSYVETSALLTEWKKTGELAFLGEVSSVPLQQALRHLQTAFTNFFAGRAAYPRLKSRKKSRQSAEYTRSAFTWRDGRLRLAKMSEPLDIRWSRPLPEGAEPSTVTVSRDAAGRWFVSLLCQDAIARLDPVETAVGIDAGVRSLVTLSTGEKVTNPRHERRDRERLARAQKDLARKTKGSKNREKARVRVARIHARITDRRRDFLHKLSTRLVHENQVVVIEDLNVAGMVGNHSLARAISDAAWRDLRAMLEYKADWYGRELVVIDRWFPSSRLCGACGTVAGAMPLEVREWECGCGARHDRDVNAAKNILAAGLAVTACGADVRPKRRKPSGQTATKQETQPATVGIPRL

SEQ ID NO: 55 - protein, 364 aa, synthetic construct
MKEIAIKQRFYPTDEQAELLARTFGCVRTVYNRILHWRTDAYRNDKIKVNYSAASSRLTELKKDPELSWLNDVSCVPLQQSLRHQQAAFKNFFEGRAKYPVEKKKRHKQSAEFTKSAFKFRDGQLFIAKSKQPLNIRWSRELPSEPTTVTISKDTAGRYFVSMLCRFEPKPMPVTAKTVGIDLGLNNLFITDSGEKVDNPRHFKRYQNKLAYLQRQLAKKKKGSNNRAKVRQKVARLHAKIADSRLDNLHKLSRKLINENQVVCVESLQVKNMLRNPKLAKHIADASWGEFVRQLKYKAEWAGRTIVEIDRFFPSSKRCSCCGFVHESMPLNIRNWTCPECNTHHDRDINAAKNIKTAGLAGVA

SEQ ID NO: 56 - protein, 393 aa, synthetic construct
MLSMHGCLYSVHMTKRAYRYRFYPTPEQEQLLARTFGCVRFVYNAVLRYRTDAFHERRERIGYVAANAELSRMKKAADTAFLNEVSSVPLQQCLRHQQTAFKNFFEGRAKYPNFKSKRHRQSAEFTRSAFKYRDGKLFIAKCKEPLPVRWSRELPSEPTTITVSKDSAGRYFVACLCEFDPESLPVTPKMVGIDLGLKDLFVTSDGHRIGNPRHTAKYATRLAKAQRRLSRKTLGSKNRAKARQKVARLHAKISDCRMDRLHKLSRQIVNENQVICVESLKVKNMLRNRALAKSISDAGWGEFVRQLEYKAAWAGRQVSAIDPWYPSSKRCSGCGHVMPEMPLQVRAWTCPECAAEHDRDINAAINIKAAGLAVLALGENVSGREPASVSDSR

SEQ ID NO: 57 - protein, 394 aa, synthetic construct
MIMPTVNRAIKYRIHPSEEQMTQLLQTFGCVRKVFNAAIELQEGLYAAKMGSMSKMDLNNHVNRVMKEDAPYLKEVDKFALTNAVYHVAAGYKNFFEGRAKHPRFKSRKDSKQSYTTNWTNGNIKVIPPEKKGTVRGKVKLPKLGLVDAVIHRVPPEDWVIKSATVSMNNSGQYFVSVLFAYEQEPTPYIPMPNKGRAIGLDYSSPDFYVDSDGNDAGVPHFYRTMEAKLAREQRRLSKMTKGSNNYAKQKIKVDKLHRKIANQRKDFCHKLSRKIANSYDAVCVEDIDLRAMSRSLNLGKSTTDNGFGMFRVFLQYKLERQGKYFVVIDKWFPSSKTCNHCGYINRELQLSDRVWVCPHCGEVILRDPNAAKNILEEGLGILYDLLVPLPMTA

SEQ ID NO: 58 - protein, 413 aa, synthetic construct
MSSKRVRRAYRFRFYPTPQQEELLRRTLGCARLVYNKALALRSESWTRDRRNVTFKDTSRMLTEWKRDENLSFLNDVSCVPLQQALRHLQQAYANFFNHKGDYPTFKRKSRGGSASFTRYAFTWNESKRELKLAKMKEPLPIRWSRTLPKGCVPSSATISLDAAQRWHISILVEDAVRELPASNRSVGVDLGLESFAITSDGEKIGNPRFRAKEAKHLRKAQKSLSRKRKGSNNQRKARLKIARIHARIADRRRDFLHKLSTRLIRENQTVVVEDLNVQGMSRRCKPVPDPNHPGRYLKNGQSRKKGMNRSITDTGWRELRTMLEYKADWYGRQLVVIDRWYPSSQTCSTCGKRTGRKALNIREWTCPYCGTHHDRDVNAAKNIQAAGLAVIACGDGRTAERRNMRPADPIRP

SEQ ID NO: 59 - protein, 403 aa, synthetic construct
MKVKVIKIIKHKAYRFRLYPNKAQQVIFAKTFGCVRFIYNKMLEDKIDYYQAHKMMLNTTPAQYKSEFEWLNEVDSLALANAQLNLQRAFKNFFQHQTEFPKFKSKHRGDKSYTTNNQNGSIRIEDGKYIKLPKIGLVRIKVHRQIPQSYNIKSCTISQSPSGKYYVSILTEYETDTQPVKVIEEDKVIGLDMDMKNLFTDSQGNRAGYPRYYRKMQDKLSKWQRKLSKCVNGSNNYYKQKRTVAKLHEKVANQRRDFLHKLSNRIANDYDAVCIEDLNMKAMGQCLNLGKSVFDNGWGMFTSFLKYKLEDKGKVLVRVDKWYPSSKRCSVCGYEKENLTLDIREWECPNCHTIHDRDINAAVNIKRQGLRMMSYEFENRGTHGDSSLILSTGVLLSEKPPLL

SEQ ID NO: 60 - protein, 374 aa, synthetic construct
MAEKAYKFRLYPNKQQEELIRKTCGCARFVYNTTLAARQAAYKAGTPLSGNDCVKMLPSLKVQYPWLKEVDSTALQSSVKNMDRAYQNFFASKGGSRKGRAVGFPKFKAKHHCRDSFTCKVGFAVRDGAVKLPKLGWIRAKTSTLVKGRVLNATVSMTRSGKFFVSLCCTDVDISQLPSTGAAVGLDVGIKDLVITSDGDKYPNHKYLAKNEKKLAKLQRQLSRKPKGSRNREKARLKVARLHEKIFNQRNDYLHNLTTQLIRDYDIICVESLNVQGMMRNHSLAKAIADASWGEFCRQLKYKAAWQHKQLVEVGIFFPSSQLCGRCGYKNPDVKDLSVREWTCPVCGAHHDRDCNASANILAEGLRLLSTAVA

SEQ ID NO: 61 - protein, 379 aa, synthetic construct
MQGGDFMEHSYRFRIYPTKEQTIQIQRTFGCCRFVYNYYLAKRIDAYKSDKTTLGYKACSADMTALKKQLPWLGEVDSIALQSSLRDLDTAYQNFFRRVKQGEKPGFPCFKSKRKHRRSYKSKCVGTNIKVLDGAMQLPKLGNVKCRVSRKVEGRVLSATVSQEASGKYYVSLCCTDVDIKPPPKTGAAVGIDLGIKDLAITSDGVKYDNLHSYRRNLKKLARLQRQLSRKSKGSRNREKARVKIVRLHEHIANQRNDAIHKMTTQLVRSNDIICMEDLSSKNMMKNHKLALHVADAAWGEIRRQLAYKDDWYGKELVVIDRFFPSSQTCGCCGCRNKEVKDLSIRFWTCPQCGAFHDRDINAAQNILEEGLRKRASAA

SEQ ID NO: 62 - protein, 370 aa, synthetic construct
MAKQNKAYKFRLYPTEEQALVIRKTFGCVRFVYNKMLAERKATYERLKEDKEALKKVKHPTPAKYKSEFEWLKEVDSLSLANAQLNLDKAYKAFFKGNAKFPKFKNKRHKQSYTTNVVNGNIQLLGGHIKLPKLKKVKIKQHRAIPAEYVIKSCTLSMTASEKYYISILTEYEKQIESKKIENVVGLDFAMDGLYVDSEGKKANYPKFYRQMLDKLAQEQRKLSRKKKGSSNWNKQRVRVAKIQEKVANQRKNFLHHKSKEMVKTYDAVIIEDLDMKGMSQALKFGKSVADNGWGMFTSFLKYKLNEQGKKLVKIDKWFPSTKTCSGCGNTQSMPMNLRTYTCSCGLHLDRDVNAAINIKKEGIHLLAIA

SEQ ID NO: 63 - protein, 370 aa, synthetic construct
MAKLNKAYKFRIYPTDEQSLMIRKTFGCVRFVYNKMLAERKEAYENLKDDKEALKSVKHPTPAKYKKEYEWLKEVDSLALANAQLNLDKAYKAFFKGNAKFPKFKSKRHRQSYTTNVVNGNIELLDGHIKLPKLKKVKIKQHREIPFVHKIKSCTLSITASGKYYLSILTEYEKEIETKEIKEVIGLDFAMDGLFVDSEGEKANYPKFYRQMLDKLAKEQRKLSRKKKGSSNWNKQRIRVAKIQEKVANQRKNFLHHTSKKLVTHFDAVVMEDLDMKRMSQALKFGKSVTDNGWGMFTSFLQYKLNEQGKQLIKIDKWFPSTKTCSKCGSVKEIKLSERTYQCTCGLNIDRDYNSALNIKKEGIRLLASA

SEQ ID NO: 64 - protein, 370 aa, synthetic construct
MAKQNKAYKFRLYPTEEQELVIRKTFGCVRFVYNKMLDERKATYELLKEDKEVLKKVKHPTPAKYKREFEWLKEVDSLSLANAQLNLDKAYKAFFKGNGKFPKFKSKRHKQSYTTNVVNGNIQLLDSHIKLPKLKMVKIKQHRTIPSECIIKSCTISLSSSGKYYISILTEYEKEIESKKIESVVGLDFAMNGLFVDSEGKKANYPKFYRQMLDKLAQEQRKLSRKKKGSSNWNKQRIRVARIQEKVANQRKNFLHHKSKELVSNYDAVIIEDLDMKGMSQALKFGKSVADNGWGMFTSFLKYKLNEQGKQLVKIDKWFPSTKTCSCCGNTQPMPMNLRSYICSCGLHLDRDVNAAINIKKEGIRLLAIA

SEQ ID NO: 65 - protein, 371 aa, synthetic construct
MAKQNKAYKFRLYPTDEQALMIRKTFGCVRFVYNKMLAERKETYENLKDDKEALKKVKHPTPAKYKKEFAWLKEVDSLALANAQLNLNKAYKAFFNRNAKFPNFKSKRHNQSYTTNVVNGNIQLLDGHIKLPKLKMVKIKQHRNIPSDHTIKSCTVSMTSSGKYYISILTEYEKEIKSKDIENVVGLDFAMNGLFVDSETGKKANYPRFYRQMIDKLAIEQRKLSRKKKGSSNWNKQRIRVAKIQEKVANQRKNYLHHQSKALVTSYDAVIIEDLDMKGMSQALKFGKSVADNGWGMFTSFLHYKLKEQGKQLVKIDKWFPSTKTCSSCGSIREIRLSERTYQCDCGLNLDRDYNSALNIKKEGIRLLATA

SEQ ID NO: 66 - protein, 370 aa, synthetic construct
MAKQNKAYKFRIYPTDEQALIMRKTFGCVRFVYNKMLAERIETYESLKDDKEALKKVKHPTPAKYKKEFEWLKEVDSLALANAQLNLDKAYKAFFKGNAKFPKFKSKRHKQSYTTNVVNGNIQLLDGHIKLPKLKMVKIKQHRKIPSEYKIKSCTLSMTASGKYYISILTEYEKKIDCKEIEKIVGLDFAMDGLFVDSEGEKANYPKFYRQMLEKLTKEQRKLSRKKKGSSNWDKQRIRVAKIQEKVANQRKNFLHHKSKELVTTYDAVIIEDLDMKGMSQALRFGKSVADNGWGMFTSFLQYKLKEQGKQLVKIDKWFPSTQSCSNCGNIQPMPMNIRTYACSCGLNLDRDYNSALNIKKEGIRLLASA

SEQ ID NO: 67 - protein, 369 aa, synthetic construct
MANKAYKFRLYPTKEQEQLLAKTFGCVRFVYNQMLAERIEIHEKFKDNKEALKKQKFPTPAKYKQEYPWLKEVDSLALANAQLNLQKAYQNFFSGRAEFPKFKTRKAKQSYTTNMVNGNIKLLNGYIQLPKLKRVKIKQHREIPSHHMIKSCTVSRTKTGKYYISILTEYEHQPVPKEVQSVVGLDFSMNTLYVDSEGKRANYPRFYRQALEKLAKAQRVLSRRKKGSNRWHKQRLKVAKLHEKIANQRKDFLHQTSRQLANRYDAVVIEDLNMKGMSQALHFGQSVHDNGWGMFTSFLQYKLAEQGKKLIKIDKWFASSKTCSHCGRVKASLSLSERLFRCACGFVADRDINAATNIHKEGLKQLGIA

SEQ ID NO: 68 - protein, 372 aa, synthetic construct
MTKQNKAFKFRLYPNKEQEELLAKTFGCVRFVYNKMLAERKEIYEKFKDDKEKLKEQKFPTPAKYKDEFPFLKEVDSLALANAQIHLQNAYKNFFEGRAAFPKFKSKKHKQSYTTNMVNGNIKMEDGHIKLPKFKKPMKMKQHREIPADYKIKSCTISKTKTGKYYISILTEYDHQPVPKETENVVGLDFSMNTLYVDSEGKRANYPRFYRQALEKLAREQRVLSRRRKGSNRWHKQRLKVARLHEKIANQRKDFLYKTSRQLANRYDAVVIEDLNMKGMSQALNFGKSVHDNGWGMFTTFLQYKLEEQGKKLIKIDKWFPSSKTCSCCGRVKVSLSLSERLFRCECGFVADRDTNAAINIKKEGLKQLGIA

SEQ ID NO: 69 - protein, 370 aa, synthetic construct
MANKAYKFRIYPTKDQEDFLVKTFGCVRFVYNKMLAERKETYEKLKHDMVLLKKQKLPTPAKYKKEFEWLKEVDSLALANAQLNLQTAFSKFFSGKTKYPKFKNRKSKQSYTTNMVNGNIKILDAHIKLPKLTPLRMKQHREIPSHHVIKSCTISRSATGKYHISILTEYEHNPVKKEINHVVGLDFAMNGLFVESEKGKKANYPRFYRQTEEKLAKEQRKLSKMMNDSSRWKKQRTKVAKLHERVANQRKNYLHQESRNLVNKYDCVVIEDLDMKGMSKAFRFGKSVADNGWGMFTTFLRYKLDEQGKRLIKIDKWFPSSKTCSACGYVNADLRLSDRTFDCDCGFVIDRDWNAAINIKIEGKLMLDLS

SEQ ID NO: 70 - protein, 368 aa, synthetic construct
MANKAYKFRLYPTKKQEQLLAKTFGCVRFVYNKMLAERKEMYEQFKDDKEMLKKQKFPTPAKYKKEFAWLKEVDSLALANAQLHLQKAYKSFFSGRAEFPKFKSRKARQSYTTNVVNGNIMLLDGYIKLPKLKLVKIKQHREIPPHHIIKSCTVSRTKTGQYYISVLTEYEHQPIQKEVQAVVGLDFSMNGLFVDSEEGKKTNYPRFYRQALEKLAKEQRILSRRNKGSNRWHKQRLKVAKLHEKIANQRKDFLHKESHKLPKRYDCVVIEDLNMKGMSQALNFGKSVADNAWGMFTTFLQYKLEGQGKKLIKINKWFPSSKTCSSCGQVKEKLSLSERVFQCECGLVIDRDWNASINIKNEGLRLLA

SEQ ID NO: 71 - protein, 370 aa, synthetic construct
MAKQNKAYKFRMYPTDEQAIFIRRTFGCVRFVYNKMLAERKETYELLKEDKEALKQVKHPTPAKYKKEYEWLKEVDSLALANAQLNLDKTYKAFFKGNAKFPKYKSKRHKQSYTTNVVNGNIQLLDGHIKLPKLKMVKIRQHREIPAKYKIKSCTVSTTSSGKYYISILTEYEKEIKSKEIEKIVGLDFAMDGLFVDSEGEKANYPKFYRRLLEKLASEQRKLSRKKKGSSNWNKQRIRVAKIQERVANQRKNFLHHKSKELVTNYDAVVIEDLDMKGMSQALKFGKSVADNGWGMFTSFLQYKLKEQGKQLIKIDKWFPSTKTCSSCGNTQPMPMAMRTYTCSCGVNLDRDYNSALNIKKEGIRLLASA

SEQ ID NO: 72 - protein, 368 aa, synthetic construct
MANKAYKFRLYPTQEQEQLLTKTFGCVRFVYNKMLADRKETYEKFKDDKEAFKKQKFPTPAKYKKEFVWLKEVDSLALANAQLNLQKAYQNFFSGRAEFPKFKSRKARQSYTTNVVNGNIMLLDGSIKLPKLKLVKMKQHREIPSHHVLKSCTVSRTKTGTYHVSILTEYEHQPTQKDVQAVVGLDFSMNGLFVDSEEGETANYPRFYRQALAKLAKEQRILSRRKKGSSRWHKQRLKVAKLHEKIANQRKDFLHKESHQLAKRYDCVVIEDLNMKGMAQALHFGKSVADHAWGMFTTFLQYKLEDQGKQLIKIDKWFPSSKTCSCCGQVKESLALSERIFHCDCGFVADRDWNAAINIKREGLRRFV

SEQ ID NO: 73 - protein, 370 aa, synthetic construct
MTKHNKAYKFRLYPTEEQAYLIRKTFGCVRFVYNKMLIERKEVYEKYKENKEELKKQKFPTPAKYKAEFEWLKEVDSLALANAQLNLQTAYKNFFSGQNDFPTFKSKKDRKSYTTNVVNGNIMLLNGHIKLPKLKIVRIKQHREIPQDHMIKSCTISMTPTGKYYVSILTEYEKEIVQKEVETFVGLDFAMDGLYVSSEDERANYPKFYRNMLDRLAKAQRVLSRKTKGSIRWNKQRIRVAKLHEKVANQRKNFLHHKSKELAAHFDVVAIEDLNMKGMSQALHFGKSVTDNGWGMFTSFLAYKLNEQGKQLVKIDKWFPSTKACSNCGNVKNMSLSERVYSCVCGVNLDRDYNAAINIKNEAIRLLALA

SEQ ID NO: 74 - protein, 367 aa, synthetic construct
MANKAYKFRLYPTDAQKHLIAKTFGCVRFVYNQILSTCKERYELHKEDKESLKKLKFPTPAQYKTSFPWLKEVDSLSLANAQLHVQKAYKNFFAKRASFPRFKSKRAKQSYTTNVVNGNIMLFHSYIKLPKLKMVKIKQHREIPSHHKIKSCTISQSKSGKYYISILVEFEQHVTKKEVQHIVGLDFSMHDLYVASDGEKANYPRFYRHALEKLVKEQRSFSRKKVQSNRWYKQKRKIAILHERIANQRKDFLHKKSRKLATMYDAVVIEDLHMKGMSQALNFGKSVHDNGWGMFTTFLHYKLEEQGKQLVRIDKWFPSSKQCSSCGQIKESLSLSERIYHCECGLSIDRDYNASLNIKKEGISYIS

SEQ ID NO: 75 - protein, 368 aa, synthetic construct
MANKAYKFRLYPTKEQEQLLAKTFGCVRFVYNQMLAERKETYELYKDNKAALKKQKLPTPAKYKNEFPWLKEVDSLALANAQLNLQKAYQNFFAGRTEFPKFKSRKARQSYTTNVVNGNIKLLDGYIQLPKLKLVKIKQHRVIPSHHIIKSCTVSRTKTGKYDISILTEYEHQPAPKEVQTVVGLDFSMKGLFVDSEEGKTANYPRFYHQTLEKVAKEQRILSRRKKGSSRWQKQRLKVAKLHERIANQRKDFLHKKSHQLAKRYDCVVIEDLNMKGMSQTLHFGKSVADNAWGMFTAFLRYKLDEQGKKLIQIDRWFPSSKTCSRCGQVKESLSLSERTFCCPCGFVADRDWNASINIKNEGLRMLA

SEQ ID NO: 76 - protein, 369 aa, synthetic construct
MANKAYKFRLYPTNEQEQLLLKTFGCVRFVYNKMLAERKEIYEQFKDDKELLKKQKFPTPAKYKTDFEWLKEVDSLALANAQLNLQKAYTNFFAGRAKFPKFKSRKTKQSYTTNRVNGNILLLASQIKLPKLGLVRLKLHREIPSCHLIKSCTISRSATGKYHISILTEFECTPIKKEIKKIIGLDFAMNGLYVESEQGEIANYPRFYRQAQQQLAKEQRILSRRTKGSSRYEKQRIKVARLHEHVANQRKDFLHKTARRLVTHYDCVVIEDLQMQGMAQTLRFGKSVGDNGWGMFTAFLHYKLGEQGKKLVKINKWFPSTKTCSSCGHRRDMPLSERTFSCACGFVSDRDWNAAINIKNEGRLMLELS

SEQ ID NO: 77 - protein, 370 aa, synthetic construct
MANKAYKFRLYPTQEQEQLLAKTFGCVRFVYNKMLAERKEIYEKFKEDQEALKKQKFPTPAKYKKEFAWLKEVDSLALANAQLNLQKAYQNFFSGRAEFPKFKSRKARQSYTTNMVNGNIMLLDGYIKLPKLKLVKMKQHREIPSHHIIKSCTVSRTKTGKYYVSILTEYEHQPTQKEVQAVVGLDFSMNGLFVDSEEGKTANYPRFYHQALEKLAKEQRILSRRKKDSNRWHKQRLKVAKLHEKIANQRKDFLHKESHKLAKRYDCVVIEDLNMKGMSQALNFGKSVADNAWGMFTTFLQYKLEEQGKKLIKIDKWFPSSKTCSCCGQAKASLSLSERTFRCECGFASDRDVNAAINIKKEGMKQLGIA

SEQ ID NO: 78 - protein, 369 aa, synthetic construct
MPNKAYKFRLYPTKEQEQLLAKTFGCVRFVYNKMLEDRLRIYDTFKGDKEALKKHKFPTPAKYKKDFPWLKEVDSLALANAQLNLQKAFTNFFSGRAGFPKFKSRKAKQSYTTNVVNGNMKLSDGSIKLPKLKWVKFKQHREIPVHHIIKACTITKTKTGKYFISILTEYEHQPVPKEIENVVGLDFSMNTLYVDSEGKRANYPRFYRQALEKLARAQRVLSRRRKGSNRWHKQRLKVAKFHEKIANQRKDFLHKESHQLAKRYDCVVIEDLNMKGMSQALHFGKSVHDNGWGMFTTFLQYKLEEQVKKLIKIDKWFPSSKTCSCCGQVKESLSLSERLFRCACGFVVDRDINAAINIHKEGLKQLGIA

SEQ ID NO: 79 - protein, 369 aa, synthetic construct
MANKAYKFRVYPTQEQAQLLAKTFGCVRFVYNKMLEERIQIYEKFKDDKEAVKKHKFPTPAKYKRDFPWLKEVDSLALANAQLNLQKAFTNFFSGRAGFPKFKSRKAKQSYTTNMVNGNIKLADGYIKLPKLKWVKLRQHRQIPVHHIIKACTITKTKTGKYFISILTEYEHQPVPKEIENVVGLDFSMNTLYVDSEGKRANYPRFYRQALEKLAQAQRVLSRRRKGSNRWHKQRLKVAQLHEKIANQRQDFLHKTSRQLANRYDAVVIEDLNMKGMSQALHFGQSVHDNGWGLFTTFLQYKLAEQGKKLIKIDKWFPSSKTCSHCGRMKASLSLTERQFRCECGFVADRDTNAAINIKKEGLKQLGTA

SEQ ID NO: 80 - protein, 369 aa, synthetic construct
MANKAYKFRLYPTQEQAQLLAKTFGCVRFVYNKMLEERKQIYEKFKDDKEALKQYKFPTPAKYKKDFPWLKEVDSLALANAQLNLQKAFTNFFSGRAGFPKFKSRKAKQSYTTNMVNGNMKIADGYIKLPKLKWIKIKQHRDIPPHHMIKSCTITKTKTGKYFISILTEYEHQPVPKEIENVVGLDFSMNTLYVDSEGKRANYPRFYRQALEKLARAQRVLSRRRKGSNRWHKQRRKVAQLHEKIANQRQDFLHQASRQLANRYDAVVIEDLNMKGMAQALHFGQSVHDNGWGLFTTFLQYKLAEQGKKLIKIDKWFPSSKTCSCCGRVKVSLSLSERLFRCECGFVADRDTNAAINIKKEGLKQLGIV

SEQ ID NO: 81 - protein, 369 aa, synthetic construct
MANKAYKFRLYPTQEQEQLLAKTFGCVRFVYNKMLEERLHIYDTFKDDKEALKQHKFPTPAKYKRDFPWLKEVDSLALANAQLNLQKAFTNFFSSRAGFPKFKSRKAKQSYTTNVVNGNIKLADSYIKLPKLKWVKLKQHREIPVHHMIKSCTITKTKTGKYFISILTEYEHQPVPKEIENVVGLDFSMNRLYVDSEGKRANYPRFYRQALEKLARAQRVLSRRRKGSNRWHKQRRKVAQLHEKIANQRQDFLHKTSRQLANRYDAVMIEDLNMKGMSQALHFGQSVHDNGWGLFTTFLQYKLAEQGKKLIKIDKWFPSSKTCSCCGRVKVSLSLSERLFRCACGFVADRDTNAAINIKKEGLKKLGTA

SEQ ID NO: 82 - protein, 371 aa, synthetic construct
MTKQYKAFQFRLLPNKVQEVLLTKTFGCVRFVYNKMLEERIETYEKCKDDKEMLKKQKFPTPAKYKSDFPFLKEVDSLALANAQINLQNAYKNFFEDRAEFPKFKSRKNKQSYTTNMVNGNIKLKAGHIKLPKLKLVKIKQHREIPADYKIKSCTITKTKSGKYYISILTEYEKEITPAIIQHVVGLDFSMDGLYVESEQGKKANYPRFYRQSLDKLAKEQRILSRRKKGSLRWEKQRMKVAKLQEKTANQRKNFLHHKSKKLSTNYDAVIMEDLDMKGMSQALHFGKSVHDNGWGMFTGFLRYKLEEQGKQLIKIDKWFPSTKRCSHCGAEKEMPLSERVYQCSCGFVLDRDHNSAINIKREGLRMLAKS

SEQ ID NO: 83 - protein, 370 aa, synthetic construct
MAKQNKAYKFRLYPNDEQALLIRKTFGSVRFVYNKMLAERNETYQNLKEDKEALKKVKHPTPAKYKKEFKWLKEVDSLALANAQLNLDKAYKAFFKENARFPKFKSKRHKQSYTTNAVNGNIQLLDGHIKLPKLKMVKIKQHRVIPSEYKIKSCTISITASGKYYISVLTEYEKEIKRKELEKVVGLDFAMDGLFVDSEGRKANYPKFYRQSLDKLAKEQRKLSRKKKGSSNWHKQRIRVAEIQEKVANQRKNFLHHKSKELVSSFDAVIIEDLDMKGMSQALKFGKSVADNGWGMFTSFLQYKLKEKGKQLVKINKWFPSTKTCSSCGSIQPMPMHIRTYTCSCGLNLDRDYNSALNIKKEGIRLLTTA

SEQ ID NO: 84 - protein, 370 aa, synthetic construct
MAKRNKAYTFRLYPTEKQALLIRKTFGCVRFVYNKMLAERKEMYALLKEDKDALKKVKLPTPAKYKKEYEWLKEVDSLALANAQLNLDKAYKAFFKGKAKFPKFKSKRHKQSYTTNVVNGNIQLLGGHIKLPKLKRVKIRQHREIPSEHKIKSCTISMTSSGKYYISILTEYEKEIKNKEIQNVVGLDFAMDGLFVDSEGKKANYPKFYRQMLDRLAKEQRKLSRKKKGSSNWNKQRIRVANIHEKVANQRKNFLHHQSKELAANFDAVVIEDLDMKGMSQALKFGRSVADNGWGMFTSFLQYKLNEQGKQLIKIDKWFPSTKTCSQCGSIQEMALSARTYQCTCGLTLDRDYNSALNIKKEGIRLLTTA

SEQ ID NO: 85 - protein, 372 aa, synthetic construct
MTKQNKAFKFRLYPNKEQEELLAKTFGCVRFIYNKMLAERKEIYEKFKDDKEALKEQKFPTPAKYKNEFPFPKEVDSLALANAQIHLQNAYENFFEGRAEFPKFKTKKKKQSYTTNMVNDNIKLENGYIKLPKIKKPIRVKQHREIPADYKIKSCTISKTKTGKYYISILTEYEKNISTVKIQKVVGLDFVMDGLYVDSERGEKANYPRYYRQALDKLAKAQRILSRRKKGSTRWEKQRLKVAKLYEKTANQRRNFLHHKSKELAANYDAVIVEDLDMKSMSQALNFGKSVHDNGWGMFTTFLEYKLKEQGKQLVKISKWFPSTKKCSRCGNEKEMPLSERVYKCICGLVLDRDYNSAINIKKEGLCLLALA

SEQ ID NO: 86 - protein, 391 aa, synthetic construct
MIRTYRYRLYPAKAQARTLDFLLWQGRTLYNAALEQRITVYKETGKGVSYYQQWAYFRDLRNANPDTLGQLNATSVQQMLRRLDKAFVAFFRRLKAGETPGFPRFKGRDRFHSLEYRHGDGCKLRMGERVMLYIQNVGEVKVKYHRPLPDDAAIKHVVIKRRAHRWYACLMLDLPDPVILEHTGPAVGIDMGLSHLLALSDGTLVENPRWLRQSLAQLRVAQRRLARRQRGSARRRKAVAQVARLHERVANQRRDFWHKLTRLLADTYSLIALEDLTLAFMTANHHLALSAHDAGLAEFQQLLAYKAEEAGTQVVTVNPAYTSQACSGCGVVVKKDLSVRVHVCPDCGLTLDRDVNAARNILMLAVNPARTGPPERNVAGHGERALGSSPL

SEQ ID NO: 87 - protein, 391 aa, synthetic construct
MIRTYRYRLYPTNAQARTLDFLLWQGRTLYNAALEQRITVYRDTGQGVSYYQQWAHFRDQRNASPDTLGQLNATSVQQMLRRLDKAFVAFFRRVKAGETPGFPRFKGRDRFHSLEYRHGDGCKLRMGERVMLYVQNVGEVKVKYHRPLPDDAAIKHVVIKRRAHRWYACLMLDLPDPVILEHTGPAVGIDMGLSHLLALSDGTLVENPRWLRQSLAQLRVAQRRLARRQKGSARRRKAAAQVARLHERVANQRRDFWHKLTRQLADTYSLIALEDLTLAFMTANHHLALSAHDAGLAEFQQLLAYKAEEAGTQVVTVNPAYTSQACSGCGVVVKKDLSVRVHVCPDCGLTLDRDVNAARNILMLAVNPARTAPPERNVAGHGERALGSSPL

SEQ ID NO: 88 - protein, 391 aa, synthetic construct
MIRTYRYRLYPTKAQARTLDFLLWQGRTLYNAALEQRITVYKERGKGVSYYQQWAYFRDLRNANPDTLGQLNATSVQQMLRRLDKAFVAFFRRVKAGETPGFPRFKGRDRLRSLEYRHGDGCKLRMGERVMLYIQNVGEVKVKYHRPLPDDAAIKHVVIKRRAHRWYACLMLDLPDPVVPEHRGPVVGVDMGLSHLLALSDGTLVENPRWLRRSLAQLRVAQRRLSRRQKGSARRRKAAAQVARLHERLANQRRDFWHKLTRQLADTYSLIALEDLTLAFMTANHHLALSAHDAGLAEFQHLLAYKAEEAGTQVVTVKPAYTSQACSACGVVVKKDLSVRVHVCPDCGLTLDRDVNAARNILMLAVNSARTGPPERNVAGYGERALGSSPL

SEQ ID NO: 89 - protein, 391 aa, synthetic construct
MIRTYRYRLYPTKAQARTLDFLLWQGRTLYNAALEQRITAYKETGKGVSYPQQWAHFRDLRNANPATLGQLNATSVQQMLRRLDKAFVAFFRRLKAGEAPGFPRFKGRDRFHSLEYRHGDGCKLRLGERVMLYVQNVGEVKVKCHRPLPEDAAIKHVILKRRANRWYACLMLDLPGPVIPEHTGPAVGIDMGLSHLLAFSDGTLVENPRWLRQSLAQLRVAHRRLARRQKGSARRRKAAAQVARLHERVANQRRDFWHKLTRQLADTYSLIALEDLTLAFMTANHRLALSAHDAGLAEFQQLLAYKAESAGTQVVTVNPATTSQACSGCGVIVPKNLSVRVHVCPDCGYTADRDVNAARNILMRAVNPARTGPPARNVAGCGERALGSSPL

SEQ ID NO: 90 - protein, 391 aa, synthetic construct
MIRTYRYRLYPAKAQARTLDFLLWQGRTLYNAALEQRIAVYKETGKGVSYYQQWAYFRDLRNANPDTLGQLNATSVQQMLRRLDKAFAAFFRRLKSGETPGFPRFKGRDRFNSLEYRHSDGCKMRFGDRVMLYVQNVGEVKVKFHRPLPDDAAIKHVVIKRRAHRWYACLMLDLPNPVVPEHRGPVVGVDMGLSHLLALSNGTLVENPRWLRRSLAQLRVAQRRLSRRQKGSARRRKAAAQVARLHERIANQRRDFWHKLTRQLTNTYSLIALEDLTLAFMTANHHLALSAHDAGLAEFQQLLAYKAEEAGTQVVTVNPAYTSQACSACGVVVKKDLSVRVHVCPDCGYTADRDVNAARNILMLAVNSARTGPPERNVAGYGERALGSSPL

SEQ ID NO: 91 - protein, 391 aa, synthetic construct
MIRTYRYRLYPTNAQARALDFLLWQGRTLYNAALEQRITVYRETGKGVSYPQQWAHFRDLRHANPDTLGQLNATSVQQMLRRLDKTFAAFFRRLKAGEKPGFPRFKGRDRFHSLEYRHGDGCKLRLGARTMFYVQNAGEVKVKFHRPLPDDATIKHVVIKRRASKWHVCLMLDLPDPVIPERTGPAVGIDMGLAHLLALSDGTLVENPRWLRQSLAQLRVAQRRLSRRQKGSAHRRKAAAQVARLHERVANQRRDFWHKLTRQLADTYSLIALENLTLAFMTANRHLALSAHDAGLAEFQQLLAYKAEEAGTLVVTVNPAYTSQACSACGVVVQKELSVRVHVCPDCGLTLDRDVNAARNILMCAVNPARTGPPGRNVAGCGARALESSLL

SEQ ID NO: 92 - protein, 391 aa, synthetic construct
MIRTYRYRLYPTSAQTRALDFLLWQGRTLYNAALEQRITVYKETGKGVSYYQQWAHFRDLRNASPDTLGYLNATSVQQMLRRLDKAFIAFFRRLKAGEKPGFPRFKGSNRFNSLEYRHGDGCKLRPGGRVMLYVQNVGEIKVKYHRRLPDGATINHVVIKRCAHRWYACLMIDLPVPVVPEHTGPEVGVDMGISHLLALSDGTLISNPRWLRGRLSQLRVAQRRLSRRKKGSNRHRKAAAQVARLHERIANQRRDFWHKITRQLVDTYRLIALEDLTLNFMTANHHLALSTYDAGLSEFQQLLAYKAESAGTRIVTVNPAYTSQTCSDCGAIVPKDLRIRVHVCPDCGLTLDRDVNAARNILKQAVNGARTGPPGRNVAGSGERALGSSLL

SEQ ID NO: 93 - protein, 392 aa, synthetic construct
MIRTWKYRLYPTNAQEKALDFLLWQGRLLYNAALEQRITVYEETGEGVTYPQQWAHFRDLRNANPDTLGQLNATSVQQMLRRLDKAFRAFFRRLKAGERPGFPRFKGHHRFNSLEYRHGDGCKLRLGKRVMLYVQNVGEIKVKYHRTLPDDAAIKHVVIKRSNRKWYVCLMLDLPDPAIEPHAGPAVGIDMGLHSLLALSDGQTVDNPRWLRQSLAKLRVAQRRLARRQKGSARRRKAAAQVARLHEQVANQRRDFWHKLTRELANTYGLIALEDLTLKFMTANHHLALSAHDAGLGEFQQLLQYKAESAGAEVVTVNPAYTSQLCSGCGVMVEKDLSVRVHQCPHCGLTLDRDVNAARNILFLAVESARTGRSGDNVPEPTRLRSLRSSLL

SEQ ID NO: 94 - protein, 399 aa, synthetic construct
MIRTYKYRLYPRNAQKRTLDFLLWQGRKLYNAALEQRINAYQETGKAVSYVEQWAHFRDERHANPDTLGQLNATCVQQMLRRLDKAFKAFFRRVKAGETPGFPRFKGRYRFHSLEFRHGDGCKLRFDDQGRALFYVQNAGEIKVKYHRPLPDDATIKHVVVKRKDNKWYVCLQVDLNRSDELPNPESVAVGGPAVGIDMGLESLLALSDGTLIENPRWLRRSQRKLRVAQRRLSRRQKGSKRRRKAAGQVARLHERISNQRRDFWHKLTRNLVDTYGLIGLEDLTLDFMTRNHHLALSAHDAGLGEFQQLLRYKAEEAGIEVVTVNPRNTSQTCSGCGVIVKKGLSVRVHCCPHCGLELNRDVNAARNVLTLALNAARTERSEPNVDQRVERALRSRRL

SEQ ID NO: 95 - protein, 396 aa, synthetic construct
MLRTYQYRLYLSNAQERAATVVLAQARGVYNAALEQRITTYRETGRSVTYQQQWAHFRDLRRANPATLGLLNASSLQHVLRRLDKAYQGTFRRIKVGEKAGFPRFKGANRFHSLEFTYGDGCKLRFEKNGRALLYVQHVGEIKVKFHRLLPKGAVIKHAVLVRKTRKWYVNLQLDLPDPNPRPAEGPAVGIDVGLKNLLALSDGTLVDNPRWLRGSLRKLRVAQRHLARCQKGSRGRQQAAFRVARLHEHVANQRRDFWHKTTAHLVKTYAVIALEDLSLGFMTHNPRLALSAHDAGLAQFRQMLAYKAESAGTQVLTVNPANTSQMCSGCGEMVEKPLSERVHTCPHCGLVLDRDLNAARNILYRALPELKPARTERPGRNVGQWVRRALGSRRL

SEQ ID NO: 96 - protein, 388 aa, synthetic construct
MIRTYKYRLRPNKQQNEVLDLLFWQARNLYNAALEQRITVYQETGKGINYPAQWTHFRDIRNNNPDSFGMLNATSLQQMLRRLDKSFIAFFRRLKAGETPGFPRFKGRNRFKSVEYRYGDGCKLRPKENGQVRFYIQNVGEIKVIYHRPIPDGALIKHVVIKRVNNKWYVCLMMHVPEPEFKPVPTGSAIGIDMGLKSLLATSEGYLFDNPHWLRESLAKLRIAQRRVSRRKKGSKRWYKAVKQLVRLHEKIANQRNDYWHKITRALAEAHSLIAIEDLNLKFMNRNRHLSLASHDAGLGIFTNLLAYKVEETGCQLVIVNPAYTSQMCSGCGELVEKDLSVRVHNCHHCGLELDRDVNAARNILDKAFNPLGLSGQDGTWAVTPSVS

SEQ ID NO: 97 - protein, 390 aa, synthetic construct
MIRTFKYRLYPHARQSKKLNYLLDLSRNVYNAALEQRRDIYEETGAGLAYTQQSKHFGEMRRDDPDGLGLLNFSCMQHTLRKLDKAFRAFFRRVKVGETPGYPRFKGRNRFHSFEFTYGDGCKLRFDDQERALLYVQNVGEIKIKYHRPIPSDAKMKHVVIKRSVGKWYVCFQIELSDIGLIERETSEMGIDVGLHSLLAFSDGELVDNPRWLRSSLSELRRKQRRLARRKKGSHRRRKAALQVAKQHEHIANQRRDFWHKVTRELAETHTLMAIEDLTLSFMTHNHHIALSAHDAGLGLFRQLLESKAEEAGSQVVAVNPAHTSQRCSGCGKIVRKDLSIRTHHCPHCDLVLDRDVNAAVNILHLARTGPSGRNVGGSNAERVPRSSPL

SEQ ID NO: 98 - protein, 379 aa, synthetic construct
MIRTYNYRLFTSAAQDAALDRLLWRSRNVYNAALAQRRDAWESEKRSVTYTQQWAHFRDERRANSETLGILNATCLQQLLRRLDKAFSAFYRRYKKGEMPGYPRFKGRNRFKSIEYRHGDGCKLRFDSAGRTMLYVQNVGEVKVKYHRPVPDGAVVKHVVIKRVLDKWYVSLMLELPDAEPVDVPTDCVGIDVGLTSLLALSTGELVENPRWLQRSLAKLRRAQRCMSRRRKGSGGWKKAGYQVAKLHQRVANQRRDFWHKKTTELVSRYGLIAIEDLTLAFMTQHPHLAKSAHDAALGMFRSLLEQKAGSAAVRVAAVNPWGTSQECSGCGAVVKKDLSVRVHRCSCGLVLNRDVNAARNILQRELGHGLCLGAITWQ

SEQ ID NO: 99 - protein, 386 aa, synthetic construct
MIRTYKYRLRPNGTQNKLLDQLFWQSRGLYNNALEQRIVTYKESGKGYRYTDQWAYFRDERKSNPDYNLLNATSVQQMLRRLDKAFSAFFRRIKKGENPGFPRFKGYRRFKSVEYRYGDGCKMRHKENGQTRFYIQNVGEIKVVYHRPIPETANIKHVVVKRFNEKWYVCLMMEIPDAPKATHQGGCVGIDMGLKSLLATSDGVLVENPQWLRESQSQLRIAQRKAARRKIGSNRRRKAVQQVAILHEKIANQRKDYWHKVARELANHYSLVALEDLNLKFMNRNKYLALSSYDAGLGMFTKLIAYKVEETGGQLVIVNPAYTSQACSQCGAIVEKSLAVRVHRCPECGLELDRDVNAARNILQKAFVALGLSVQDLTWANAPCVS

SEQ ID NO: 100 - protein, 405 aa, synthetic construct
MIRTYNYRLFTNAAQDSALDRLLWQSRNVYNAALAQRRDAWEGEKRSVTYAQQWAHFRDERRANPETLGILNATSLQQLLRRLDKAFSAFYRRCKKGETPAQGHPPRPSGRTVGYPRFKGRNRFKSIEYRHGDGCKLRFDAAGRTMLYVQNVGEVKVKYHRPVPEGAVIKHVVIKRVLDKWFVSLMLELPEAESVDVPTDCVGVDVGLTSLLALSNGELVENPRWLQRSLAKLRRAQRRMSRRRKGSSGWKKAGYQVAKLHQHVANQRRDFWHKKTTELVSRYGLIAIEDLTLAFMTQHPHLAKSAHDAALGMFRSLLEQKAGSAAVRVAAVNPWGTSQECSGCGAVVKKDLSVRVHRCSCSLVLNRDVNAARNILQRELGHGPVPWGDNVAVVDASVAPEAALL

SEQ ID NO: 101 - protein, 394 aa, synthetic construct
MLRTYKYLLRPNKEQAEQLDFLLWQSRRIYNAALEQRIKAYQETGKGMNYGAQWAHFRDQRHENSDTLGQLNASSVQHMLRRLDKSFAAFFRRLKTGERAGFPRFKSRHRFHSLEYTYGDGCKLRQNKQGRQSFYVQNVGETRMCYHRAIPAEAKIKHAVIKRVGERWYVCLMLQLPDPENSGIQTGKQIGIDVGLKSLAALSTGELIENPRWLRENLAKLRRLQRRSARQIRGSHRQRETYRQIARLHEHVANQRADYLHKVSHRLVTGHDFIAIEQLSLGFMNQNRHLSLSSHDAGFGLFRQMLEYKAESAGIPVIAVNPSNTTQACSGCGRMVPKDLSVRVHACPDCGLILDRDVNAARNILSLALHRSVGSQPLGRSGQPGTWVATPCVG

SEQ ID NO: 102 - protein, 386 aa, synthetic construct
MILSYKYRLYPNRAQREMLDRTLDMHRWVYNAALEQRITAYKESGESVRYVQQWAYFRDLRHENPNTLGALNASSMQHTMRKLDKAFRAFFKRAKNGEKAGFPRFKSWKRFKSFEYTYGDGVKAFFDKRGRLMLRLQNIGNIKVKFHRDFPEGAVIKHVIIKKSLDKWYVIFQIETPDVEFEITTNNPVGIDVGLHHLLAFSNNTVIENPRWLRHSLRKLRVAQRSLARKKKGSNRRRKAAYRVAKIHEKIANQRRDFWHKVTRSIVNEFDFIAIEDLSLSFMTRNHHLALSAHDAGLGMFRQFLAYKAAEAGSWVTAENPAYSSQECSGCGEIVEKPLSQRWHSCTCGTELDRDVNAATVILKRGLETVFHPPGLGGQDVTYTVG

SEQ ID NO: 103 - protein, 389 aa, synthetic construct
MLRTYKYLLRPDTEQLKHLDFLLWQSRLAYNAALEQRIATYQESGKGINYGAQCTHFRDLRRENPDTLGRVNASSLQQTLRRLDKAFASFFRRVKAGDTPGFPRFKGRSRFKSIEYTYGDGCKLRQNNNGRMSFYIQNVGEMRMCYHRAIPVEAKIKHVIVKQVNQRWFACLMIELPDPVKTRVPTEQMIGIDVGLKSLAALSTGVLIDNPRPLQANLAKLRRLQRHAARQVRGSSRLRKTYAQIAALHERISSQRSDFLHKTANSIVSQFDLIAIENLSLAFMNRNRHLSRASYDAGFGLFRQYLEYKAEEAGVQVIAVNPSNTTQACSRCGSIVPKDLSVRSHNCPDCGLILDRDVNAARNILSLALPFPLGRSGQAVTWVGTPCVV

SEQ ID NO: 104 - protein, 389 aa, synthetic construct
MLRTYKYLLRPDTEQLKHLDFLLWQSRLVYNAALEQRIATYQESGKGIGYGAQWTHFRDLRRENPDTLGRVNASSLQQTLRRLDKAFASFFRRVKAGDTPGFPRFKGRSRFKSIEYTYGDGCKLRQNNNGRMSFYIQNVGEMRMCYHRAIPVQAKIKHVIVKQVNQRWFACLMIELPDPVKMRVPIKQMIGIDVGLKSLAALSTGVLIDNPRPLRANLAKLRRLQRHAARQVRGSSRLRKTYVQIAALHELISNQRSDFLHKTANSIVRQFDLIAIEDLSLAFMNRNRHLSRASYDTGFGLFRQYLEYKAEEAGVQVIAVNPSNTTQACSRCGSIVPKDLSVRTHNCPDCGLILDRDVNAARNILSLALPFPLGRSGQAVTWVGTPCVA

SEQ ID NO: 105 - protein, 393 aa, synthetic construct
MIRTYRYRLYPTNAQTRALDFLLWQGRTLYNAALEQRITVYKETGKGVSYPQQWAHFRDLRNANPDTLGYLNATSVQQMLRRLDKAFTAFFRRLKAGEKPGFPRFKGRDRFHSLEYRHGDGCKLRLDERKRAALYVQNVGEIKVKFHRPLPDDATIKHVVIKRRASKWHVCLMLDLPDPVIPERTGPAVGIDMGLAHLLALSDGTLVENPRWLRQSLAQLRVAQRRLSRRQKGSAHRRKAAAQVARLHERVANQRRDFWHKLTRQLADTYSLIALENLTLAFMTANRHLALSAHDAGLAEFQQLLAYKAEEAGTLVVTVNPAYTSQACSACGVVVQKELSVRVHVCPDCGLTLDRDVNAARNILMCAVNPARTGPPGRNVAGCGERALGSSPL

SEQ ID NO: 106 - protein, 391 aa, synthetic construct
MIRTYKYRLYPTNAQARTLDFLLWQGRMLYNAALEQRIATYRDTGKGVSYYQQWVHFRDLRHEQPDTLGYLNATSVQQLLRRLDKAFVAFFRRIKAGETPGFPRFKGRDRFHSLEYRHGDGCKLRFGSRVMLYLQNVGEIKVKYHRPLPEDAVIKHVVIKRSNNKWTVCLMLDLPDPAPPAHSGPAVGIDMGLYHLLALSDGTLVENPRWLRQSLAQLRVAQRRLARRQKGSARRRKAAAQVARLHERVANQRRDFWHKVTRQLADTYALIALEDLTLAFMTANHHLALSAHDAGLAEFQQLLAYKAEEAGTRVVTVNPAFTSQACSACGVVVKKDLSVRVHVCPDCGLTLDRDVNAARNILMLAVNPARTGPPEQNVAGYGERALGSSPL

SEQ ID NO: 107 - protein, 382 aa, synthetic construct
MIRTYKYRLYPTNAQARTLDFLLWQGRMLYNAALEQRIATYRDTGKGVSYPQQWAHFRDLRNANPDTLGYLNATSVQQMLRRLDKAFVAFFRRVKAGETPGFPRFKGRDRFHSLEYRHGDGCKLRMGERVMLYIQNVGEVKVKYHRPLPDDAAIKHVVIKRRAHRWYACLMLDLPDPVILEHTGPAVGIDMGLSHLLALSDGTLVENPRWLRQSLAQLRVAQRRLARRQRGSARRRKAVAQVARLHERVANQRRDFWHKLTRQLADTYSLIALEDLTLAFMTANHHLALSAHDAGLAEFQQLLAYKAEEAGVTVIAVPPHNTTQACSYCGTLPDVPKTLADRVHSCSQCGYVADRDLNAAQNILRLGSSLQDRTCADGRSVS

SEQ ID NO: 108 - protein, 393 aa, synthetic construct
MIRTYRYRLYPTEAQARALDFLLWQGRTLYNAALEQRITVYRETGQGVSYPQQWAYFRDLRHANPETLGQLNATSVQQLLRRLDKAFRAFFSRLRAGETPGFPRFKGRDRFHSLEYRHGDGCKLRFDPRNRTLFYIQNVGEIKVKYHRPVPDEAEIRHVVIKRRAHRWYVCLMLALPDPVVPLHTGPVVGIDMGLVHLLALSDGSTVENPRWLRHSLAQLRVAQRRLARRQKGSARRRKAAAQVARLHERVTNQRRDFWHKLTRQLAETYSLIALEDLTLAFLTTNHHLALSAHDAGLAEFQQLLAYKAEEAGTQVVTVNPAYTSQACSECGMVVRKDLSVRVHVCPNCGLTLDRDVNAARNILKLGVYPARTGPPERNVAGCGARALGSSPF

SEQ ID NO: 109 - protein, 393 aa, synthetic construct
MIRTYQYRLYPTNAQARTLDFLLWQGRNVYNAALEQRITVYQETGKGVTYPQQWTHFRDQRRENPDTLGQINATSMQQLLRRLDKTFGAFFRRVQAGEKPGYPRFKGRHRFNSLEYRYGDGCKLRFDDRDRALFYVQNVGEVKVKYHRPLPDDAGIKHVILKRRNRKWYVCLQLELPDPVVAQHPGPAVGVDMGLHSLLALSDGATVDNPRWLRWSLAQLRVAQRRLSRRKKGSHRRRKAAEQVARLHERIANQRRDFWHKLTRELTATYSVIALEDLTLRFMTVNHHLALSAHDAGLGEFQQLLEYKAESAGTEVVTVNPAYTSQVCSGCGALVEKDLRVRVHICPHCGLTLDRDVNAARNILSLAVESARTGRSGANVARWGARSLRSSLL

SEQ ID NO: 110 - protein, 389 aa, synthetic construct
MIRTYKYLLRPTSEQEQSLDYLLWQSRRLYNVALEQRINVYQETGKGINYQAQWAHFRDLRHSEPETFGKLNATSMQQLLRRLDKAFSAFFRRLKAGETPGFPRFKGRNRFKSMEYTYSDGCKLRCNEQGRFAFYVQNVGEMRMCYHRAIPENASIKHVVVKCVNNRWYACLMIEVPDKPIAPRTEPRAIGVDVGLKSLLALSDGSLIENPRWLRESLAELRVVQRKASRRMKGSNRKRKAYEKVAKLYEHITNQRGDYLHKITNTLVSKYSLIGIEDLTLAFMNRNRSLALSSHDAGLGMFRQLLQYKAEEAGTLVVAVNPAYTSQVCSGCGEIVQKDLSVRIHECPSCGLVLDRDMNAARNILSLALQKQPGWGCQDVTWAAAPCVS

SEQ ID NO: 111 - protein, 395 aa, synthetic construct
MLRTYKYRLRPNTGQTEQLLILFQQSRRLYNNALEQRINTYKETGKGIKYAQQWTYFRDDRNNKPDIYGMLNASSVQQMLRRLDKAYSAFFRRIKAGETPGHPRFKNAQRFKSVEYRYGDGCKLRSQENGQVRLYIQNVGEIKIIYHRIVPETAVLKHLVLKHINTKWYVCLMLELPDPEPVLPIGKTVGIDMGVKSLLATSDGVLYENPHWLRESLAQLRVAQRTVARRKKGSNRRHKAVRRVSALYEKITSQRADYWHKITRNLAEQYSFIALENLNLNFMNRNKNLSLSSYDAGLGIFTQLLAYKVEETGSRLVLVNPANTSQACSACGELVEKDLSVRVHKCPHCGLIINRDINAARNILSKAVNLHPLGLSGQDLTWNNSSCVSCEAVIL

SEQ ID NO: 112 - protein, 389 aa, synthetic construct
MLRTYKYLLRPNNQQARQLDFLLWQSRQVYNAALEQRITIYLETGKGLNYGAQWVHFRDQRRSFPETLGQLNASSMQHLLRRLDKSFAAFFRRLKAGEKPGFPRFKSRHRFKSMEFTYRDGCKLRQKEHGPMSFYVQNVGEMRMCYHRAVPGEAKIKHAVIKRVNNRWFVCLMLELPQPEPKYTANRRQVGIDIGLKTLAALSTGELIDNPRWLRESLAKLRRLQRHAARQAKGSQRQRKTFRKIAHLHEQIANQRADFLHKVSSQIIAEYDLVAIENLSLAFMNRNRHLSRSSHDAGFGEFRHMLEYKAESAGIPVIAVNPSNTTQMCCECGRMVPKDLSVRVHKCPGCGLILNRDVNAARNILNLALHNPHGRCGQPVTWAVAPGVG

SEQ ID NO: 113 - protein, 379 aa, synthetic construct
MADNEMETYTLGYKFRIYPNRTQKNLINRTLGCARFVFNHFLAVRRDEWKANHNSLTYVKTSKLLTDLKKREETAWLSEVDSMALQESLRNLDRAFQNFFAKRARYPRFKSKHSHNQSYRTRNQANGIRIIDKRIKLPKIGLVKIKQSRDFDGRILSATVSRAASGKYYVSLCVEAEKGSLSCPNDGKQIGIDVGLKVFYSDSNGDTVENPHPLKKLQSKLRREQRRLSRKMPKSVNRGRARVRVARVHERIANIRRDFLHKISTRLARENQTVAVEHLNIKGMLRNHKLAKSISDVSWSEFFRQLSYKTERCGGELLQVDTFYPSSQTCSVCGYQNPLTKDLSVREWNCPACGTHHDRDVNAAKNILRKALEDKSKVA

SEQ ID NO: 114 - protein, 379 aa, synthetic construct
MADNEMETYTLGYKFRIYPNRTQENLINRTLGCARFVFNHFLAVRRDEWKANHNSLTYVKTSKLLTDLKKREETAWLSEADSMALQESLRNLDRAFQNFFAKRARYPRFKSKHSHNQSYRTRNQTNGIRIIDKRIKLPKIGLVRIKQSRDFDGRILSATVSRAASGKYYVSLCVEAEKESLLCANNGKQIGIDVGLKVFYSDSNGDTVENPHPLKKIQRKLRREQRRLSRKMPKSVNRERARVRVARVHERIANIRRDFLHKISTRLARENQTVAVEHLNIKGMLRNHKLAKSISDVSWSEFFRQLSYKTERHGGELLKVDTFYPSSQTCSVCGYQNPLTKDLSVREWDCPACGSHHDRDVNAAKNILRKALEDKSKVA

SEQ ID NO: 115 - protein, 379 aa, synthetic construct
MADNEMETYTLGYKFRIYPNRTQENLINRTLGCARFVFNHFLAVRRDEWKANHNSLTYVKTSKLLTDLKKREETAWLSEVDSMALQESLRNLDRAFQNFFAKRARYPRFKSKHSHSQSYRTRNQTNGIRIIDKRIKLPKIGLVRIKQSRDFDGRILSATISRAASGKYYVSLCVEAEKGVLSCPNDGKQIGIDVGLKVFYSDSNGDTIENPHPLKKLQRKLRREQRRLSRKMPKSVNRERARVRVARVHERIANIRRDFLHKISTRLARENQTVAVEHLNIKGMLRNHKLAKSISDVSWSEFFRQLSYKTERHGGELLKVDTFYPSSQTCSVCGYQNPLTKDLSVREWDCPACGTHHDRDVNAAKNILRKALEDKYQVA

SEQ ID NO: 116 - protein, 379 aa, synthetic construct
MADNEMETYTLGYKFRIYPNRTQENLINRTLGCARFVFNHFLAVRRDEWKANHNSLTYVKTSKLLTDLKKREETRWLSEVDSMALQESLRNLDRAFQNFFAKRARYPRFKSKHSHSQSYRTRNQTNGIRIIGKRIKLPKLGLVRIKQSRDFGGRILSTTVSRAASGKYYLSLCVEVEKGSLSCPNDGKQIGIDVGVKVFYSDSNGDIVENPHPLKKLQRKLRREQRRLSRKMPKSVNRERARVRVARVHERIANIRRDFLHKISTRLARENQTVAVEHLNIKGMLRNHRLAKSISDASWSEFFRQLTYKTELHGGELLKVDTFYPSSQTCSVCGYQNPLTKDLSVREWDCPACGTHHDRDVNAAKNILRKALEDKSKVA

SEQ ID NO: 117 - protein, 379 aa, synthetic construct
MADNEMETYTLGYKFRIYPNRTQENLINRTLGCARFVFNHFLAVRRDEWKVNHNSLTYVKTSKLLTDLKKREETAWLSEADSMALQESLRNLDRAFQNFFAKRARYPRFKSKHSHSQSYRTRNQTNSIRIIGKRIKLPKIGLVRIKQSRDFDGRILSATVSRAASGKYYVSLCVEAEKGSLSCPNDGKQIAIDVGLKVFYSDSNGDTVENPHPLKKLQRKLRREQRRLSRKMPKSVNRGRARVRVARVHERIANIRRDFLHKISTRLARENQTVAVEHLNIKGMLRNHKLAKSISDVSWSEFFRQLTYKTELHGGELLKVDTFYPSSQTCSACGYHTPLTKDLSVREWDCPACGTHHDRDVNAAKNILRKALEDKSKVA

SEQ ID NO: 118 - protein, 379 aa, synthetic construct
MADNEMETYTLGYKFRIYPNKTQENLINRTLGCARFVFNHFLAVRRDEWKVNHNSLTYVKTSKLLTDLKKREETAWLSEADSMALQESLRNLDRAFQNFFAKRARYPRFKSKHSHSQSYRTRNQTNSIRIIGKRIKLPKIGLVRIKQSRDFDGRILSATVSRAASGKYYVSLCVEAEKGSLSCPNDGKQIGIDVGLKVFYSDSNGDIVENPHPLKKLQRKLRREQRRLSRKMPKSVNRERERVRVARVHERIANIRRDFLHKISTRLARENQTVAVEHLNITGMLRNHRLAKSISDASWSEFFRQLTYKTELHGGELFKVDTFYPSSQTCSVCGYQNQLTKDLSVREWDCPACGTHHDRDVNAAKNILRKALEDKSKVA

SEQ ID NO: 119 - protein, 379 aa, synthetic construct
MADNEMETYTLGYKFRIYPNRTQENLINRTLGCVRFVFNHFLAVRRDEWKANHNSLTYVKTSKLLTDLKKREETAWLSKADSMALQESLRNLDRAFQNFFAKRARYPRFKSKHSHSQSYRTRNQTNGIRIIDKRIKLPKIGLVRIKQSRDFDGRILSATVSRAASGKYYVSLCVEAEKGSLSCPNDGKQIGIDVGLKVFYSDSNGDTVENPHPLKKLQRKLRREQRRLSRKMSRSVNRERARVRVARVHERIANIRRDFLHKISTRLARENQTVAVEHLNIKGMLRHHKLAKSISDVSWSEFFRQLSYKTERHGGELLQVDTFYPSSQTCSVCGCQNPLTKDLSVREWDCPACGTHHDRDVNAAKNILRKALEDKSKVA

SEQ ID NO: 120 - protein, 374 aa, synthetic construct
METYTLGYKFRIYPNKTQARLINRTLGCVRFVFNHFLAVRRDEWKANHHSLTYVKTSQLLTNLKKREDTSWLSEADSMALQESLRNLDRAYENFFQKRAGYPRFKSKHSRSQSYRTRNQGNGIRIEGNRIKLPKIGLVKIKQSRTFEGRILNATVSRADSGTYFVSLCIEMDKTALLHPNGGRQIGIDVGLKAFYSDSDGNTVASPRPLKSLQRQLRREQRRLSRKMPKSRNREKARICVARLHERIANIRKDFLHKLSTRLARENQTVAVEHLNIKGMLRNHRLAKSISDASWSEFFRQLAYKTELYGGELLKVDTFYPSSQTCSVCGYQNPLTKNLSVREWDCPACGTHHDRDVNAAKNILRKALEDRSTAA

SEQ ID NO: 121 - protein, 374 aa, synthetic construct
METYALGYKFRIYPNRTQENLINRTLGCARFVFNHFLAVRRDEWKENHHSLTYAKTSKLLTDLKKREETRWLSEVDSMALQESLRNLDRAFQNFFAKGSGYPRFKSKHHHSQSYRTRNQANGIRIIGKRIKLPKVGLVRIKQSRAFEGKIQNATVSRTASGKYFVSLCVETDKASLLQTNRGKQIGIDVGLKAFCSDSNGNTVENPHPLKKLQRKLRREQRRLSRKMPKSVNREKARIRVARVHERIANIRRDFLHKISTRLARENQTVAVEHLNIKGMLRNHKLAKPISDASWGELFRQLSYKTELHGGDLLKVDTFYPSSQTCSICGYQNPLTRNLSIREWDCPACGTHHDRDVNAAKNILRKALEDKSKVA

SEQ ID NO: 122 - protein, 374 aa, synthetic construct
METYTLGYRFRIYPNKTQTRLINRTLGCVRFVFNHFLAVRRDEWKANHHSLTYVKTSQLLTDLKKREDTSWLSEADSMALQESLRNLDCAYENFFKKRAAYPRFKSKHSRSQSYRTRNQGNGIRIEGNRIKLPKVGLVKIKQSRTFEGRILNATVSRAASGTYFVSLCVETDKTALLHPNGGRQIGIDVGLKAFYSDSDGNTVASPRPLKSLQRKLRREQRKLSRKMPQSRNREKARICVARLHERIANIRKDFLHKLSTRLARENQTVAVEHLNIKGMLRNHRLAKSISDASWSEFFRQLAYKTELHGGELLKVDTFYPSSQTCSLCGCQNPLMKDLSVREWDCPACGTHHDRDVNAAKNILRKALEDRSTAA

SEQ ID NO: 123 - protein, 374 aa, synthetic construct
METYTLGYKFRIYPNKTQARLINRTLGCARFVFNHFLAVRRDEWKANHHSLTYVKTSQLLTNLKKREDTSWLSEADSMALQESLRNLDRAYENFFKKRAGYPRFKSKHSRSQSYRTRNQGNGIRIEGNRIKLPKIGLVKIKQSRMFEGRVLNATVSRAASGTYFVSICVETDKTALLHPNGGKQIGIDVGLKAFYSDSDGNTVASPRPLKSLQRKLRREQRRLSRKMPRSRNQENARIRVARAHERIANIRRDFLHKLSTRLARENQTVAVEHLNIKGMLRNHRLAKSISDASWSEFFRQLAYKTELYGGELLKVDTFYPSSQTCSVCGYQNPLTKNLSVREWDCPACGTHHDRDVNAAKNILRKALEDRSTAA

SEQ ID NO: 124 - protein, 379 aa, synthetic construct
MAVRRMETHTLGYKFRIYPSKTQENLINRTLGCVRFVFNHFLAVRRDEWKVNRNPLTYVKTSRLLTDLKKREETAWLSEVDSMALQEALRDLDRAYENFFAKRARYPRFKSKRSHSQSYRTRNQSGGIRIEGNRIKLPKVGFVKIKQSREFGGRILNATVRRAVSGKYFISLCVETEKESLLRPNGGKQIGIDVGLRAFCSDSDGNVTQSPRPLKKLQRKLCREQRRLSRKLPKSMNRDKARVRVARVHERIANIRRDFLHKLSTRLTRENQTVAVEHLNVKGMLRNHRLAKSISDASWSEFFRQLTYKAELHGGELLKVDTFYPSSQTCSACGYQNPLTKDLSVREWDCPKCGTHHDRDVNAAKNILCKALEDKSTVA

SEQ ID NO: 125 - protein, 379 aa, synthetic construct
MAMRRMETYTLGYKFRIYPSKTQEILIHRTLGCVRFVFNHFLAVRRDEWKVNRNPLTYVKTSRLLTDLKKREETAWLSEVDSMALQEALRDLDHAYENFFAKRARYPRFKSKRSHSQSYRTRNQSGGIRIEGNRIKLPKVGFVKIKQSREFVGRILNATVRRAASGKYFISLCVETEKESLLRPNGGKQIGIDVGLRAFCSDSDGNVTQSPRPLKKLQRKLCREQRRFSRKLPKSMNRDKARVRVARVHERIANIRRDFLHKLSTQLARENQTVAVEHLNVKGMLRNHRLAKSVSDASWSEFFRQLTYKTELHGGELLKVDTFYPSSQTCSACGYQNPLTKDLSVREWDCPKCGTHHDRDVNAAKNILCKALEDKSTVA

SEQ ID NO: 126 - protein, 374 aa, synthetic construct
MEKYTLGYRFRIYPDKLQENLINRTLGCVRFVFNHFLAVRRDEWKANHHSLTYVKTSHLLTNLKKREDTSWLSEADSMALQESLRNLDRAYENFFKKRAGYPRFKSKHSRSQSYRTRNQGNGIRIEGNRIKLPKVGLVKIKQSRTFEGKILNATVSRVASGTYFVSLCVEMDKTVLLHPNGGRQIGIDVGLKAFYSGSDGNTVASPHPLKSLQRKLRREQRRLSRKMPKSRNRENARIRVARVHERIANIRRDFLHKLSTRLARENQTVAVEHLNVKGMLRNHRLAKSISDVSWSEFFRQLAYKTELHGGELLKVDTFYPSSQTCSLCGYQNPLTKDLKVREWDCPACGTHHDRDVNAAKNILRKALEDRSTAA

SEQ ID NO: 127 - protein, 372 aa, synthetic construct
METYTMGYKYRIYPNKEQENLINRTLGSCRFVFNHFLAVRRDQWKANHQSVTYVQTASMLTDLKRREETCWLKEVDSMALQESLRNLDTAYQNFFAKRARYPRFKSKHCHSQSYRTRNQSNGIRLVDGKLKLPKIGLVKIKQHRTFEGRILNATVSRTASGKYFVSLCVEQDIENLLHSNGGGQVGIDVGLKEFYSDSNGNTVANPRVLRKLTRKLIREQRRLSRKMPRANNRNKQRVRVARVHEKIANIRKDFLHKVSTQLVRENQTIAVENLQVKNMLKNHKLAKAISDVSWSEFFRQLEYKVKFYGSEILKVPTFYPSSQTCHVCGYQNAGTKDLSVREWTCPQCGTVHDRDINAARNILAKALADKAA

SEQ ID NO: 128 - protein, 373 aa, synthetic construct
MEKYTMGFKFRIYPNKTQQELINRTLGCCRFVYNHFLAIRRDEWTANHNPVTYNQTSAMLTNLKRHEEYNWLKQVDSMALQESLKNLDTAYQNFFKHQSKYPCFKSKHNHSQSYRTRNQGNGVRIIGRRIKLPKIGTVKIKQSREFEGRILNATVSRNASGKYFVSLCVELDKAMLFKANAGGEVGIDVGLKEFYSDSNGNAVANPKILRRLTRKLVRAQRNLSRKMPKSVNRGKARIRVARVHERIANIRKDFLHKTSTMLVRRNKTIAIEHLQIKNMMKNHKLARAISDVSWSEFFRQLGYKAELYGSEVLKVDTFYPSSQTCSECGYQNTETKNLGIREWDCPQCGAHHDRDTNAAINILHRALEEKQSA

SEQ ID NO: 129 - protein, 373 aa, synthetic construct
MDKYTMGFKFRIYPNHAQQERINHTLGCCRFVFNHFLAIRRDEWTANHKSVTYNQTSAMLTDLKKYKEYSWLKEVDSMALQESLKNLDTAYQNFFKHQAKYPRFKSKHNHRQSYRTRNQSKGVRIVGNRIKLPKIGTVKIKQSREFDGRILNATVGRNASGKYFVSLCVELDRGILLKTNDGGEIGIDVGLKEFYSDSNGNTVANPRILRRLTRKLAREQRRLSRKMPRSVNRDKARSRVARVHERIANIRKDFLHKTSAALVRQNKTIAIEHLQIKNMMRNHKLARAISDVSWSEFFRQLEYKAELHGSEVLKVDTFYPSSQTCSKCGHQNVETKNLGVRKWICPECGSHHDRDTNAAVNILRKALEQKQVA

SEQ ID NO: 130 - protein, 373 aa, synthetic construct
MDKYTMGFKFRIYPNHAQQELINRTLGCCRFVFNHFLAIRRDEWTANHKSVTYNQTSAMLTDLKKYEEYSWLKEVDSMALQESLKNLDTAYQNFFKHRSRYPRFKSKHNHSQSYRTRNQGNGIRIIRNRIKLPKIGAVKIKQSREFDGRILNATVSRAASGKYFVSLCVELDRGSLLKTNDGGEIGIDVGLKEFYSDSNGNTVANPRILRCLTRKLVREQRKLSRKMPRSANRGKARIRVARVHERISNIRRDFLHKTSTVLVRQNKTIAIEHLQIKNMLKNHKLARAISDVSWSEFFRQLEYKAELHGSEVLKVDTFYPSSQTCSECGYQNTETKNLGIRDWTCPACSSHHDRDTNAAINILRKAIEQKQIA

SEQ ID NO: 131 - protein, 373 aa, synthetic construct
MDKYTMGFKFRIYPNHAQQELINRTLGCCRFVFNHFLAIRRDEWTANHKSVTYNQTSAMLTDLKRYEEYSWLKEVDSMALQESLKNLDTAYQNFFKHQSRYPRFKSKHNHSQSYRTRNQGNGVRIIGNRIKLPKIGAVKIKQSRKFEGRILNATVSRNASGKYFVSLCVELDKETLLKTNDGGEIGIDVGVKEFYSDSNGNTVANPRILRRLTRKLVREQRRLSRKMPRSMNRDKARIRVARVHERISNIRRDFLHKTSTALVRQNKTIAIEHLKIKNMMRNHKLARAISDVSWSEFFRQLEYKAELHGSEVLKVDTFYPSSQTCSECGYQNTETKNLGIRKWTCPKCGSHHDRDTNAAINILRKAIEQKQVS

SEQ ID NO: 132 - protein, 372 aa, synthetic construct
MEYTKGYKFRLYPNKEQAILLNKTLGCCRFIFNHFLSVRRDSWENEKKSVSYSETSSLMTQLKSDENHLWLKEADSVALQQSLRDLDIAYQNFFKHGRGYPKFKSKRNRQQSYRTQNDGKKIRVEGKRIKLPKVGLVRFEQSRRFNGRILNATVTRTATGKYFVSLCVQEDIADNLRQNDGGQIGLDVGLKEFCTDNHGNTFANPKPLRRLSKKLSKEQRKLSRRKLDSHNRKRQRIILARVHEKITNIRRDFLHKLTTRLCRENQTIAVESLNVQGMMKNHKLARAIADVSWGEFFRQLQYKAILYGCDVIKVPTFYPSSQTCCTCGYKNLLTKDLSVRRWTCPVCGTVHDRDQNAAINILRKALELKNSA

SEQ ID NO: 133 - protein, 374 aa, synthetic construct
METYTLGYKFRIYPNATQARLIHRTLGCARFVYNHFLAVRRDQWNANRKSIDYTESSRLLTDLKKREETAWLSEVDSMALQEALRNLDRAFQNFFDKRARYPRFKSKHSHAQSYRTRNQSNGVRIVGKRIKLPKIGLVRIKQSREFSGRILSATVSRTASGKYFVSLCVEQDKAKLLRPNAGGQIGIDVGIREFYTDSNGNTVENPKPLKKLLRKLKREQRRLSRKLPRSQNRGKARVRLARVHERIANIRKDFLHKCTTRLARENQTAAVEHLNVKGMLKNHRLAQAISDVSWSEFFRQLAYKMELRGGELLKVETFYPSSQTCSVCGYQNTEVKNLGVREWTCPQCGAHHDRDHNAAKNILRRALENKAKAA

SEQ ID NO: 134 - protein, 374 aa, synthetic construct
MEPYTLGYKFRIYPNKTQVRLINRTLGCARFVFNHFLAVRRDERKANHHSLTYVKMSQLLTDLKKREDTSWLSEADSMALQEALRNLDRAYENFFKKRASYPRFKSKHSRSQSYRTRNQGNGIRIEGSRIKLPKVGLVKIKQSRTFEGRILNATVSRAASGTYFVSLCVETDKTALLHPNGGRQIGIDVGLKAFYSDSDGNTVANPRPLKGLQRKLCREQRRLSRKMPQSRNREKARICVARLHERIANIRKDFLHKLSTRLARENQTVAVEHLNIKGMLRNHRLAKSISDVSWSEFFRQLAYKTELHGGELLKVDTFYPSSQTCSLCGYQNPLTKDLKVREWDCPACGMHHDRDVNAAKNILRKALEDKSTAA

SEQ ID NO: 135 - protein, 374 aa, synthetic construct
MEPYVLGYKFRIYPNKTQQRLINRTLGCCRFVYNHFLAVHRDQWQANHRSVSYFETCKLLTDLKRQEETSWLKEADSMALQESLRNLDNAFQNFWKLHTGYPRFKSKHSHSQSYRTRNQKNGIRIVDKRIKLPKIGFVKIKQSRDCDGRILSATISRTAFSKYFVSFCMEQDKENLVRPNAGGMIGIDVGIRAFYTDSLGHIVENPRHLNRFLAKLKREQRRLSRKMPKSQNRDKARIRVARIHEKIANIRKDFLHKHSTQLARENQTIAVEQLNIKGMLRNHKFAQAISDVSWSEFFRQLTYKTELHGGELLKVDTFYPSSQTCSVCGYQNALAKDLSVREWDCPECGMHHDRDVNAAKNILRKALEDKSKVA

SEQ ID NO: 136 - protein, 374 aa, synthetic construct
METYVLGYKFRIYPNKTQQRLINRTLGCCRFVYNHFLAVRRDQWQANHHSVSCFETSRLLTDLKRQKETSWLKEADSMALQESLRNLDNAFQNFWKSHTDYPRFKSKHGHSQSYRTRNQKNGIRIVDKRIKLPKIGFVKIKQSRDCDGRILSATISRVASGKYFVSLCMEQDKKNLVRPNAGGMIGIDVGIRAFYTDSLGHIVGNPRHLNRFRTRLKREQRRLSRKMPKSRNRDKARIRVARIHEKIANIRKDFLHKHSTQLARENQTIAVEHLNIKGMLRNHKLAQALSDVSWSEFFRQLTYKTELHGGELLKVDTFYPSSQTCSVCGYQSPLTKNLSIREWDCPECGTHHDRDANAAKNILRKALEDKSKVA

SEQ ID NO: 137 - protein, 373 aa, synthetic construct
MEKYTMGFKFRIYPNKTQQELINRTLGCCRFVYNHFLAIRRDEWTANHKSVTYNQTSAMLTDLKRYEEYNWLRQVDSMALQESLKNLDTAYQNFFKRQSRYPRFKSKHNHSQSYRTRNQGKGIRIVDKRIKLPKIGTVKIKQSREIEGRILNATVSRTASGKYFVSLCVELDKAILLCANSVGEIGIDVGLKEFYSDSNGNTVANPRILRRLTKKLVREQRKLSRKMPRSVNRSKARIRVARVHERIANIRKDFLHKTSTALVSQNKTIAIEHLQIKNMLKNHKLARAISDVSWSEFFRQLEYKAELHSAEVLRVDTFFPSSQTCSECGYQNTETKNLGIREWTCPECGSHHDRDTNAAINILRRALEEKQSA

SEQ ID NO: 138 - protein, 373 aa, synthetic construct
MEKYTMGFKFRIYPNKTQQELINRTLGCCRFVYNHFLAIRRDEWTANHKSVTYNQTSAMLTDLKRYEEYNWLRQVDSMALQESLKNLDTAYQNFFKHQSKYPRFKSKHNHSQSYRTRNQGNGIRIADKRIKLPKIGTVKIKQSREIEGRILNATVSRTASGKYFVSLCVELDKENLLCANDGGEIGIDVGLKEFYSDSNGNTVANPRILRRLTKKLVREQRKLSRKMPKSVNRSKVRIRVARVHERITNIRKDFLHKTSTMLVRQNKTIAIEHLQIKNMLKNHKLARAISDVSWSEFFRQLEYKAELHGAEVLRIDTFYPSSQTCSECGYQNTETKNLGVREWTCPECGSHHDRDTNAAINILRRALEEKQSA

SEQ ID NO: 139 - protein, 372 aa, synthetic construct
MKYAKGFKFRIYPNAEQQKIINQTLGCARFVYNHFLAVRRDSWQEKRESVTYVKSSHMLTELKCHPDFIWLKSVDSMALQEALRNLDRAFQNFFKKQSGYPKFKSKHSHNQSYRTRNQSGGIRIIDGNIKLPRIGIVKTKLSREFEGRILNATVSRTASGKYFVSLCVEMDIELLLGRNNGRQIGIDVGIKDFYSNDQGNTVANPRILKGMEKKLKRLQRSLSRKVKGSNNRNKARIKVARLQERIANIRKDFLHKESTRLVRENQLIAVENLKVKNMLRNHKLAKAVSDVSWGEFFRMLEYKAKLYGCDVVKVDTFYPSSQTCSCCGYQNKATKNLGIRKWTCPQCHTYHDRDVNAARNILRKALEMQKSA

SEQ ID NO: 140 - protein, 372 aa, synthetic construct
MEYAKGFKFRIYPNAQQQKIINQTLGCARFVYNHFLAIRRDNWQEKHESVTYVKSSRLLTELKCNPDFVWLKSVDSMALQEALRNLDRAFQNFFKKQSGYPKFKSKHSHNQSYRTRNQSGGIRIIDGNIKLPRIGVVKTKLSREFEGRILNATVSRTPAGKYFVSLCVEMDIELLLRRNNGRQIGIDVGIKEFYSNDQGNTVANPHILKGMEKKLKRLQRSLSRKVKGSNNRNKARIKVARLQERIANIRRDFLHKESTKLVRENQLIAVENLKIKNMLRNHKLSKAISDVSWAEFFRMLEYKAKLYGCDLIKIDTFYPSSQTCSCCGYQNKATKNLGIRKWICPQCNTQHDRDVNAARNILSKALEMQKSA

SEQ ID NO: 141 - protein, 372 aa, synthetic construct
MEYAKGFKFRIYPNAEQQKIINQTLGCARFVYNHFLAVRRDNWQEKHESVTYVKSSRMLTELKCHPDFVWLKSVDSMALQEALRNLDRAFQNFFKKQSGYPKFKSKHSYNQSYRTRNQSGVIRIIDGNIKLPRIGIVKTKLSREFEGRILNATVSRTASGKYFVSLCVEMDIELLLGRNNGQQIGIDVGIKDFCSDDQGNTVANPRVLKRMEKKLKHLQRKLSRKAKGSNNRNKARIKVARLQERIANIRKDFLHKESTKLVRENQLIAVENLKIKNMLRNHKLAKAISDVSWAEFFRMLEYKAKLYGCDVVKVDTFYPSSQTCSCCGYQNKATKNLGIRKWTCPQCNTQHDRDVNAARNILSKALEMQKSA

SEQ ID NO: 142 - protein, 372 aa, synthetic construct
MKYAKGFKFRIYPNAEQQKIINQTLGCARFVYNHFLAVRRYNWQEKHEAVTYVKSSRLLTELKCNPDFVWLKSVDSMALQEALRNLDKAFQNFFKKQSGYPKFKSKHSHNQSYRTRNQSGGIRIIDGNIKLPRIGIVKTKLSREFEGRILNATVSRTPSGKYFVSLCVEMDIESLLGRNNGQQIGIDVGIKDFCSDDQGNTVANPQALKRMEKKLKRLQKKLSRKAKGSNNRNKARIKVARLHERIANIRKDFLHKESTNLVRENQLIAVENLKIKNMLRNHKLAKAISDVSWAEFFRMLEYKAKLYGCDLVKVDTFYPSSQTCSCCGYQNRATKNLGIRKWTCSQCNTQHDRDVNAARNILRKALEMQKSA

SEQ ID NO: 143 - protein, 372 aa, synthetic construct
MEYAKGFKFRIYPNAEQQKIINQTLGCARFVYNHFLAVRRDNWQEKHESVTYVKSSRMLTELKCHPDFVWLKSVDSMALQEALRNLDRAFQNFFKNQSGYPKFKSKHSHNQSYRTRNQSGGIRIIDGNIKLPRIGIVKTKLSREFEGRILNATVSRTASGKYFVSLCVEMDIELLLGRNNSRQIGIDVGIKDFYSNDQGNTVANPHILKGMEKKLKRLQRSLSRKAKGSNNRNKARIKVARVQERIANIRRDFLHKESTKLVRENQLIAVENLKIKNMLRNHKLAKAISDVSWAEFFRMLEYKAKLYGCDVVKVDTFYPSSQTCSCCGYQNKATKNLGIRKWTCPQCNTQHDRDVNAARNILSKALEMQKSA

SEQ ID NO: 144 - protein, 371 aa, synthetic construct
MVWMSNTRGYKYRIYPNASQRSLLERIFGCCRYVRNHFLALRRDEWKANQRSITYKDTSRLLTEMKQREETSWLKEADSMALQEALRNLDAAFQNFFQKRARYPRFHSKHAHQQSYRTRNQKGGIRFVGNRLKLPKVGLVRIRKSRNFDGRILHATVSRTASGEYFVSLCVEEEAAFAPCGKGSVGIDVGLKAFCTMSDGAEARNPKYLPKYQKKLARAQRRLSRTMPRSKNHEKQRIRVARIHEYIRNMRRDTLQKLTTQLVNENQVIAVEHLKIRNMLRNHKLARAISDASWSEFFRELEYKAAWRGATILKVDTFYPSSQTCSTCGCQNPLVKNLAVREWTCPKCGTHHDRDVNAAKNILAKALEEAS

SEQ ID NO: 145 - protein, 363 aa, synthetic construct
MEYTKGYKFRLYPNKTQKNLLNKILGCCRFVYNRFLTVRKESWENEKKSVSYTQTSSMLTQLKKEMAWLNDADSIALQQSLRNLDTAYQNFFKNDRGYPRYKSKKNHRQTYRTMNIRIEGKRIRLPKVGSVKFEQSRNFHGKILNATITRTATEKYFVSLCVEEDIADNLKLNADKQIGIDVGLKEFYTDNFGNTVSNPRILKSLTLRLRREQRRLSKKKIGSHNRDKQRIKVARVHEKISNARLDFLHKESTRLCRENQTIAVESLQVKNLMKNHKLAKAISDVSWGEFFRQLSYKAILYGCDVIKIPTFYPSSQTCSCCGFKNPMTKNLSVRNWRCPKCGTFHDRDLNAAKNILYKALEIK

SEQ ID NO: 146 - protein, 372 aa, synthetic construct
MKYAKGFKFRIYPNAEQQKIINQTLGCARFVYNHFLAVRRDNWQEKHEAVTYVKSSRLLTELKCNPDFVWLKSVDSMALQEALRNLDRAFQNFFKKQSGYPKFKSKHSHNQSYRTRNQSGGIRIIDGNIKLPRIGIVKTKLSREFEGRILNATVSRTPAGKYFVSLCVEMDIELLLGRNNSRQIGIDVGIKDFYSNDQGNTVANPRILKGMEKKLKRLQRSLSRKVKGSNNRNKARIKVARLQERIANIRKDFLHKESTKLVRENQLIAVENLKIKNMLRNHKLAKAISDVSWAEFFRMLEYKAKLYGCDVVKVDTFYPSSQTCSCCGYQNKATKNLGIRKWTCPQCNTQHDRDVNAARNILRKALEMQKSA

SEQ ID NO: 147 - protein, 372 aa, synthetic construct
MKYAKGFKFRIYPNAEQQKIINQTLGCARFVYNHFLAIRRDNWQEKHESVTYVKSSRMLTELKCHPDFVWLKSVDSMALQEALRNLDRAFQNFFEKQSGYPKFKSKRSHNQSYRTRNQSGGIRIIDGNIKLPRIGIVKTKLSREFEGRILNATVSRTASGKYFVSLCVEMDIELLLGRNNGRQIGIDVGIKEFYSNDQGNTVANPHILKGMEKKLKRLQRSLSRKVKGSNNRNKARIKVARLQERIANIRKDFLHKESTKLVRENQLIAVENLKIKNMLRNYKLAKAISDVSWAEFFRMLEYKAKLYGCDVVKVDTFYPSSQTCSCCGYQNKATKNLGIRKWTCPQCNTQHERDVNAARNILRKALEMQKSA

SEQ ID NO: 148 - protein, 368 aa, synthetic construct
MANKAYKFRLYPTQEQEQLLAKTFGCVRFVYNKMLAEWKETYEQFKDDKEALKKQKFPTPAKYKEEFAWLKEVDSLALANAQLNLQKAYQNFFFGRAEFPKFKSRKARQSYTTNVVNGNIMLLDGYIKLPKLKLVKIKQHRDIPSHHVIKSCTVSRTKTGKYYVSILTEYEHQPVQKEIKAVVGLDFSMNGLFVDSEEGKTANYPRFYCQALEKLAKEQHVLSRRKKGSNRWNRQRLKVAKLHEKIANQRKDFLHKESHKLAKRYDCVVIEDLNMKGMSQALNFGKSVADNAWGMFTTFLQYKLEDQGKKLIKIDKWFPSSKTCSCCGQVKESLSLSERTFRCECGFVANRDWNAATNIKNEGLRLLA

SEQ ID NO: 149 - protein, 369 aa, synthetic construct
MANKAYKFRLYPTKEQEQLLTKTFGCVRFVYNKMLAERKETYEKYKGDKEALKKQKFPTPAKYKKEFAWLKEVDSLALANAQLNLQKAFQNFFSGRAGFPKFKSRKAKQSYTTNMVNGNIKLSDGYIKLPKLKWIKFKQHREIPANHIIKSCTITKTKTGKYYISILTEYEHQPALKEVQTVVGLDFSMSTLYVDSEGKRANYPRFYRQALEKLAKEQRILSRRKKGSNRWHKQRLKVAKLHEKIANQRKDFLHKESHKLAKRYDCVVIEDLNMKGMSQALHFGQGVHDNGWGMFTTFLQYKLAEQGKKLIKIDKWFPSSKTCSCCGRVKESLSLSERTFRCECGFESDRDVNAAINIKHEGMKRLAIV

SEQ ID NO: 150 - protein, 369 aa, synthetic construct
MANKAYQFRLYPTKEQEQLLAKTFGCVRFVYNKMLEERIQMFEKFKDDQESLKQQTCPTPAKYKKEFPWLKEVDSLALANAQLNLQKAFQHFFSGRAGFPKFKNRKAKQSYTTNVVNGNIKLSDGYIKLPKLKWIKLKQHREIPAHHIIKSCTITKTKTGKYYISILTEYEHQLAPKEVQTVVGLDFSMSTLYVDSEGKRANYPRFYRKALETLAKEQRKLSRKKKGSNRWHKQRLKVAKLHEKIANQRKDFLHKESHKLAKRYDCVVIEDLNMKGMSQALHFGQRVHDNGWGMFTSFLRYKLEEQGKKLIKIDKWFPSSKTCSCCGRVKESLSLSERTFRCECGFESDRDVNAAINIKHEGMKRFAIV

SEQ ID NO: 151 - protein, 369 aa, synthetic construct
MANKAYKFRLYPTKEQEQLLLKTFGCVRFVYNKMLAERKEIYEQFKDDKGLLKQQKLPTPAKYKQEFAWLKEVDSLALANAQLNLQKAYTNFFAGRAKFPKFKSRKAKQSYTTNRVNGNIQLLESHIKLPKLGLVRLKQHREVPSHHMIKSCTISRSATGKYYISILTEYEHEPKLKEINEIIGLDFAMDGLYVESEQGEKANHPRFYRQTQQKLAKEQNILSRKTKGSARYEKQRIKVARLHEHVANQRKDFLHKTARRLVTRYDCVVIEDLNMKSMSQALQFGKSVADNGWGMFTAFLQYKLFEQGKKLIKISKWFPSTKTCSSCGHRRDMPLSERTFSCACGFVSDRDWNAAINIKNEGRLLLGLS

SEQ ID NO: 152 - protein, 368 aa, synthetic construct
MANKAYKFRLYPTPQQEQLLAKTFGCVRFVYNKMLAERKETYEQFKDDQAVLKKRKLPTPAKYKSEHPWLKEVDSLALANAQLHLQKAYQNFFSRRAEFPKFKSRKVKQSYTTNVVNGNIMLLDGYIRLPKLKLVKMKQHREIPSHHIIKSCTVSRTKTGKYYISILTEYEHQPVPKEVQAVVGLDFSMNGLFVESEEGKTANSPHFYRQALEKLAKEQRILSRRKKGSNRWHKQRLNIAKLHEKIANQRKDFLHKESHRLAKRYDCVIIEDLNMKVMSQALHFGKSVADNAWGMFTTFLQYKLEEQGKRLIKINKWFPSSKTCSCCGQVKESLSLSERTFHCDCGFVADRDWNASINIKREGLRLLA

SEQ ID NO: 153 - protein, 369 aa, synthetic construct; identical to SEQ ID NO: 76

SEQ ID NO: 154 - protein, 368 aa, synthetic construct
MANKAYKFRLYPTPQQEQLLAKTFGCVRFVYNKMLAERKETYEQFKDDQDALKKQKLPTPAKYKSEHPWLKEVDSLALANAQLHLQKAYQNFFSRRAEFPKFKSRKAKQSYTTNVVSGNIMLLDGYIKLPKLKLVKMKQHREIPSHQVIKSCTVSRTKTGKHYISILTEYEHQPVPKEVQAVIGLDFSMNGLFVESEEGKTANSPHFYRRALEKLAKEQRILSRRKKGSNRWHKQRLNIAKLHERIANQRKDFLHKESHRLAKRYDCVVIEDLNMKGMSQALHFGKSVADNAWGMFTTFLQYKLEEQGKRLIKINKWFPSSKTCSSCGQVKESLTLSERTFHCNCGFVADRDWNASINIKREGLRLLA

SEQ ID NO: 155 - protein, 368 aa, synthetic construct
MANKAYKFRLYPTLQQEQRLAKTFGCVRFVYNKMLAERKETYEQFKDDQAALKKQKLPTPAKYKSEHPWLKEVDSLALANAQLHLQKAYQNFFSRRAEFPKFKSRKARQSYTTNVVNGNIMLLDGYIKLPKLKLVKMKRHREIPSHQIIKSCTVSRTKTGKYYISILTEYEHQPVPKEVQAVVGLDFSMNGLFVDSEEGKTANSPHFYRRALEKLAKEQRILSRRKKGSNRWHKQRLNIAKLHERIANQRKDFLHKESHRLAKRYDCVVIEDLNMKGMSQALNFGKSVADNAWGMFTTFLQYKLEEQGKRLIKIDKWFPSSKTCSSCGQVKESLPLSERTFHCDCGFVADRDWNASINIKREGLRLLA

SEQ ID NO: 156 - protein, 370 aa, synthetic construct
MANKAYKFRLYPTEEQEQLLAKTFGCVRFVYNKMLTEQQETYEKYKDDKETLKKQKFPTPAKYKKEFAWLKEVDSLALANAQINLQKAYTNFFSGRAEFPKFKNRKARQSYTTNVVNGNIMLLDGYIKLPKLKLVKIKQHREIPSHHIIKSCTVSRTKTGKYYVSILTEYEHPPVQKEVQAVVGLDFSMNGLFVDSEKGKKANYPRFYRQALEKLAKEQRILSRRKKGSSRWHKQRLKVAKLHEKIANQRKDFLHKESHKLVKRYDCVVIEDLNMKGMSQALHFGKSVADNAWGMFTTFLGYKLEEQGKKLIKIDKWFPSSKTCSCCGQVKKSLSLSERKFRCDCGFVADRDWNASINIKREGLRLLALI

SEQ ID NO: 157 - protein, 370 aa, synthetic construct
MANKAYKFRLYPTGEQEQLLAKTFGCVRFVYNKMLTEQQETYEKYKDDKETLKKQKFPTPAKYKEEFAWLKEVDSLALANAQINLQKAYKNFFSGRAEFPKFKNRKARQSYTTNVVNGNIMLLDGYIKLPKLKLVKMKQHREIPSHHIIKSCTVSRTKTRKYYVSILTEYEHPPVQKEVQAVVGLDFSMSGLFVDSEKGKKANYPRFYRQALEKLAKEQRILPRRKKGSTRWHKQRLKVAKLHERIANQRKDFLHKESHKLAKRYDCVVIEDLNMKGMSQALHFGKSVADNAWGMFTTFLGYKLEEQGKQLIKIDKWFPSSKTCSCCGQVKESLSLSERTFRCDCGFVADRDWNASINIKNEGLRLLALI

SEQ ID NO: 158 - protein, 370 aa, synthetic construct
MANKAYKFRLYPTGEQEQLLAKTFGCVRFVYNKMLTERRETYEQFKDDKVALKKQKFPTPAKYKEEFAWLKEVDSLALANAQLNLQKAYKNFFSGRAEFPKFKNRKARQSYTTNVVNGNIMLLDGYIKLPKLKLVKMKQHREIPSHHIIKSCTVSRTKTGKYYVSILTEYERQPVQKQVQAVVGLDFSMNGLFVDSEKGKKANYPRFYRQALEKLAKEQRILSRRKKGSTRWHKQRLKVAKLHEKIANQRKDFLHKESHKLAKRYDCVVIEDLNMKGMSQALHFGKSVADNAWGMFTTFLGYKLEEQGKKLIKIDKWFPSSKTCSCCGQIKESLSLSERTFRCDCGFVVDRDWNASINIKREGLRLLALI

SEQ ID NO: 159 - protein, 369 aa, synthetic construct
MANKAYQFRLYPTKEQEQLLAKTFGCVRFVYNKMLEERIQMFEKFKDDQESLKQQTCPTPAKYKKEFPWLKEVDSLALANAQLNLQKAFQHFFSGRAGFPKFKNRKAKQSYTTNVVNGNIKLSDGYIKLPKLKWIKLKQHREIPAHHIIKSCTITKTKTGKYYISILTEYEHQPAPKEVQTVVGLDFSMSTLYVDSEGKRANYPRFYRKALETLAKEQRKLSRKKKGSNRWHKQRLKVAKLHEKIANQRKDFLHKESHKLAKRYDCVVIEDLNMKGMSQALHFGQRVHDNGWGMFTSFLRYKLEEQGKKLIKIDKWFPSSKTCSCCGRVKESLSLSERIFRCECGFESDRDVNAAINIKHEGMKRFAIV

SEQ ID NO: 160 - protein, 376 aa, synthetic construct
MWAGDADMPNKAYKFRLYPTQEQEQLLAKTFGCVRFVYNKMLEERQHIYDTFKDDKEALKQHKFPTPAKYKKDFPWLKEVDSLALANAQLNLQKAFTNFFSGRAGFPKFKNRKAKQSYTTNVVNGNIKLSDGYIKLPKLKWIKLKQHREIPAHHIIKSCTITKTKTGKYYISILTEYEHQPVPKEIENVVGLDFSMNTLYVDSEGKRANYPRFYRQALEKLARAQRVLSRRRKGSKRWHKQRLKVAQLHEKIANQRQDFLHQASRQLANRYDAVVIEDLNMKGMAQALHFGQSVHDNGWGLFTTFLRYKLAEQGKKLIKIDKWFPSSKTCSCCGRVKVSLSLSERLFRCACGFVADRDINAAINIKKEGLKQLGTA

SEQ ID NO: 161 - protein, 369 aa, synthetic construct
MANKAYQFRLYPTKEQEQRLAKTFGCVRFVYNKMLEERIQMFEKSKDDQESLKQQTFPTPAKYKKEFPWLKEVDSLALANAQLNLQKAFQHFFSGRAGFPKFKNRKAKQSYTTNVVNGNIKLSDGYIKLPKLKWIKLKQHREIPAHHIIKSCTITKTKTGKYYISILTEYEHQPALKEVQTVVGLDFSMSTLYVDSEGKRANYPRFYRKALEKLAKEQRKLSRKKKGSNRWHKQRLKVAKLHEKIANQRKDFLHKESHKLAKRYDCVVIEDLNMKGMSQALHFGQRVHDNGWGMFITFLRYKLEEQGKKLIKIDKWFPSSKTCSCCGRVKESLSLSERTFRCECGFESDRDVNAAINIKHEGMKRLAIV

SEQ ID NO: 162 - protein, 370 aa, synthetic construct
MANKAYKFRLYPTKEQEQLLAKTFGCVRFVYNKMLAERKETYEQFKDDKEALKKQKFPTPAKYKKKFAWLKEVDSLALANAQLNLQKAYQNFFSGRAEFPKFKSRKARQSYTTNVVNGNIMLLDGYIKLPKLKLVKMKQHREIPSDHIIKSCTVSRTKTGKYYVSILTEYEHQPIQKEVQAVVGLDFSMNGLFVDSEEGKTANYPRFYHQALEKLAKEQRILSRKKKDSNRWHKQRLKVAKLHERIANQRKDFLHKESYKLAKLYDCVVIEDLNMKGMSQALNFGKSVADNAWGMFTTFLQYKLEEQGKRIIKIDKWFPSSKTCSCCGQVKESLSLAERIFHCDCGFVADRDWNASINIKLEGLRLLALA

SEQ ID NO: 163 - protein, 370 aa, synthetic construct
MANKAYKFRLYPTQEQEQLLQKTFGCVRFVYNKMLAERKETYEQFKGDKEALKKQKFPTPAKYKEEFAWLKEVDSLALANAQLNLQKAYENFFSGRAEFPNFKNRKARQSYTTNVVNGNIMLLDGYIKLPKLKLVKMKQHREIPSFHIIKSCTVSRTKTGKYYVSILTEYEHHPVQKEVQAVVGLDFSMNGLFVDSEEGERANYPRFYRQALEKLAKEQRILSRRKKGSSRWHKQRLKIAKLHEKIANQRKDFLHKESQKLVKLYDCVVIEDLNMKGMSQALHFGKSVADNAWGKFTTFLQYKLEEQGKKLIKIDKWFPSSKTCSCCGQVKESLSLSERKFHCDCGFVADRDWNASINIKREGLRLLAFV

SEQ ID NO: 164 - protein, 370 aa, synthetic construct
MANKAYKFRLYPTQEQEQLLAKTFGCVRFVYNKMLAERKETYEKFKDDKEALKKQKYPTPAKYKKEFAWLKEVDSLALANAQLNLQKAYQNFFSGRAEFPKFKSRKARQSYTTNVVNGNIMLLDGYIKLPKLKLVKMKQHREIPSHHIIKSCTVSRTKTGKYYVSILTEYEHQPVQKEVQAVVGLDFSMNGLFVDSEEGKTANYPCFYRQALEKLAKEQRILSRRKKGSNRWHKQRLKVAKLHEKIANQRKDFLHKESHKLAKRYDCVVIEDLNMKGMSQALNFGKSVADNAWGMFTTFLQYKLEEQGKKLIKIDKWFPSSKTCSCCGQVKESLSLSERTFHCDCGFVADRDWNASINIKHEGLRLLASS

SEQ ID NO: 165 - protein, 368 aa, synthetic construct
MANKAYKFRLYPTQEQEQLLAKTFGCVRFVYNKMLDERKKTYEKFKDDKEGLKKQKFPTPAKYKKEFTWLKEVDSLALANAQLNLQKAYKNFFSGRAEFPKFKTRKARQSYITNVVNGNIMLLDGYVKLPKLKLVKMKQHREIPSHHIIKSCTVSRTKTGKYYVSILTEYEHQPIQKEVQAVVGLDFSMNGLFVDSEEGKTANYPRFYRQALEKLAKKQRILSRRKKGSNRWYKQRLEVAKLHERIANQRKDFLHKESHKLAKRYDGVIIEDLNMKGMSQALNFGKGVADHAWGMFTTFLQYKLEEQGKKLIKINKWFPSSKTCSCCGRKKESLSLSERTFRCECGFVVNRDWNAAVNIKREGLRLLA

SEQ ID NO: 166 - protein, 369 aa, synthetic construct
MANKAYQFRLYPTKEQEQLLTKTFGCVRFVYNKMLEERIQMYEKFKDNKESLKQQTFPTPAKYKKEFPWLKEVDSLALANAQLSLRKAFQNFFSGRAGFPKFKNRKAKQSYTTNVVNGNIKLSDGYIKLPKLKWIKLKQHREIPAHHIIKACTITKTKTGKYYISILTEYEHQPAPKEVQTVVGLDFSMSTLYVDSEGKRANYPRFYRKALETLAKEQRKLSRKKKGSNRWHKQRLKIAKLHEKIANQRKDFLHKESHKLAKRYDCVVIEDLNMNGMSQALHFGQRVHDNGWGMFTTFLRYKLEEQGKKLIKIDKWFPSSKTCSCCGRVKESLSLSERTFRCECGFETDRDVNAAINIKHEGIKQLSIV

SEQ ID NO: 167 - protein, 368 aa, synthetic construct
MANKAYKFRLYPTQEQEQLLAKTFGCVRFVYNKMLSEQKETYEQFKDDKETLKKQKFPTPAKYKEEFAWLKEVDSLALANAQLNLQKAYKNFFSGRAEFPKFKNRKARQSYTTNVVNGNIMLLDGYIKLPKLKLVKMKQHREIPSHHIIKSCTVSRTKTGKYYVSILTEYEHQPVPKEVQAVVGIDFSMNGLFVDSEEGKTANYPRFYRQALEKLAKEQRILSRRKKGSNRWHKQRLKVAKLHERIANQRKDFLHKESHKLSKQYDCVVIEDLNMKGMSQALNFGKSVADNAWGMFTTFLQYKLEEQGKKIIKIDKWFPSSKTCSCCGQVKESLSLSERIFHCECGFVADRDWNASINVKHEGLRLLA

SEQ ID NO: 168 - protein, 370 aa, synthetic construct
MANKAYKFRLYPTGEQEQLLAKTFGCVRFVYNKMLTERRETYEQFKGDKEALKKQKFPTPAKYKEEFAWLKEVDSLALANAQLNLQKAYKNFFSGRAEFPKFKNRKARQSYTTNVVNGNIMLLDGYIKLPKLKLVKIKQHREIPSHHIIKSCTVSRTKTGKYYVSILTKYEHQPVQKEVQAVVGLDFSMNGLFVDSEKGKKANYPCFYRQALEKLAKEQRILSRRKKGSTRWHKQRLKVAKLHEKIANQRKDFLHKESHKLVKRYDCVVIEDLNMKGMSQALNFGKSVADHAWGMFTTFLGYKLEEQGKKLIKIDKWFPSSKICSCCGQIKESLSLSERTFHCDCGFVADRDWNASINIKREGLRLLALT

SEQ ID NO: 169 - protein, 368 aa, synthetic construct
MANRAYKFRLYPTPQQEQLLAKTFGCVRFVYNKMLAERKETYEQFKDDKDALKQQKLPTPAKYKSEHPWLKEVDSLALANAGLHLQKAYQNFFSGRAAFPKFKSRKARQSYTTNVVNGNIMLLDGYIKLPKLKLVKMKQHRDIPSHQVIKSCTVSRTKTGKHYISILTEYEHQPVQKEVQDVIGLDFSMNGLFVDSEEGKTANYPHFYRQALEKLAKEQRILSRRKKGSNRWHKQRLNIAKLHERIANQRKDFLHKESHKLAKRYDCVVIEDLNMKGMSQALNFGKSVADNAWGMFTTFLQYKLEEQGKRLIKINKWFPSSKTCSCCGRVKESLSLSERTFHCECGFVADRDWNASINIKREGLRWLA

SEQ ID NO: 170 - protein, 368 aa, synthetic construct
MANKAYQFRLYPTPRQEQLLAKTFGCVRFVYNQMLAERKDTYEQFKADQETLKKQKFPTPAKYKKEFVWLKEVDSLALANAQLHLQKAYQSFFSGRAKFPKFKSRKARQSYTTNVVNGNIMLLDGYLKLPKLKLVKIKQHREIPSHHIIKSCTVSRTKTGKYYVSILTEYEHRPVQKEIQAVVGLDFSMSALYVDSEEGKTANSSRFYRQALEKLAKEQRILSRRKQGSQRWHKQRLKVAKLHEKIANQRRDFLHKESHKLAKRYDCVVIEDLNMKGMSQALHFGKSVSDQAWGMFTRFLQYKLEDQGKKLIEIDKWFPSSKTCSSCGHIKESLSLSDRIFHCDCGFVADRDWNASINIKNEGLRMLA

SEQ ID NO: 171 - protein, 370 aa, synthetic construct
MANKAYKFRLYPTQKQEQLLAKTFGCVRFVYNKMLAERKEIYEKFKNDKEALKKQKFPTPAKYKKEFAWLKEVDSLALANAQQNLQKAYQNFFSGRAEFPKFKSRKARQSYTTNVVNGNIMILDGYIKLPKLKLVKIKQHREIPSCHTIKSCTVSRTKTGKYYVSILTEYEHQPIQKKVQTVVGLDFSMNGLFVDSEEGKTANYPRFYRQALEKLAKEQRILSRRKKGSNRWHKQRLKLAKLHERIANQRKDFLHKESHKLAKRYDCVVIEDLNMKGMSQALNFGKSVADNAWGMFTTLLQYKLEEQGKKLIKIDKWFPSSKTCSCCGQVKESLSLSERTFRCDCGYVADRDWNASINIKNEGIRQLGIA

SEQ ID NO: 172 - protein, 382 aa, synthetic construct
MLQLKAYEYRIYPDKKQETLIARTIGSSRFVYNHFLELWNREYEETGKGLTYFACSKLLTKMKRDPETVWLCEVDKFSLQNSLRNLSDAFSRFFKGQNEHPQFKSKKSPRQSYTTQYTNNNIAVSGNFLKLPKLGLVKFADSRDMKGRILNATVRRKSSGKFFVSIICEEEICELPKTDSSVGIDLGIIDFAVMSDGSRHDNNHFTRRMEERLRREQRKLAKRALIAEKRGIRLSEARNYQKQRRKVARLYEKVANQRNEFLNKLSTEIVKNHDIICIEDLNVKGMMRNHKLAKSISDVSWTSLVSKLQYKASWYGKEVISIGRWFPSSQICSECGHKDGKKPLYVREWTCPVCHTHHDRDVNAARNILAEGLRIRALTPGS

SEQ ID NO: 173 - protein, 382 aa, synthetic construct
MLQHKAYEYRIYPNKKQETQIAKTIGSSRFVYNHFLEMWNKEYEETGKGLTYVACSKLLTDLKRAPETKWLSEVDKFPLQNSLQNLSDAFSRFFKGQNEHPRFKSKKSSRQSYTTQFTNNNIAVSGNFLKLPKLGLVKFIKSQELKGRILNATVRRNPSGKYFVSILCEEKISEMTKTDSAIGIDLGISDFAVLSDGEKKDNNHFTLQMEKRLKREQRKLSRMLLGAKKKGISLSDAKNYQKQKRKVARLHEKVANQRNEFLNRLSTEIIKNHDIICIEDLNTKGMMRNHKLAKSISDVSWSGFVSKLQYKAAWYGREVIKVGRWFPSSQICSECGRNDGKKPLNIREWTCPVCHANHDRDVNAARNILAEGLRLRALKLES

SEQ ID NO: 174 - protein, 422 aa, synthetic construct
MQRLQAFKYELKPTGEQQCLMRRFAGSCRVVFNEGLALQKARYERGEKKLGYASLCKELTSWRNGTPMPSGRVAPWLAEAPVHPLQQALKDLERAYSNFFSKREGFPRFRKKGRHDSFRYPDPKQIKLDQVNSRIFLPKLGWLRYRKSREVLGTVKNVTVRHSCGKWFVSIQTEREFEQPIPSGGAVGIDLGVARFATLSDGTFYAPLNSFKRHEAALRKAQQALSRKVKCSCNWRKAKTRVQRIHARIANVRRDFLHKCSSAISKNHAIVCIEDLQVRNMSKSAVGTADAPGKNVRAKSGLNKSILDQGWFEFRRQLEYKLTWNGGQLIVVPPQNTSRTCPCCGHVSAENRRTQAQFRCVQCGFEDNADVVGAKNILARGMAIVASEMKGGARPDGLWIEPQWRSEAGTHRSDSGAPQCCA

SEQ ID NO: 175 - protein, 415 aa, synthetic construct
MQRLQAYKYALMPDGQQKRRMSRFAGSCRFVYNKALALQKARYESGEKKHGYAALCKLLTQWRGSADTPWLADAPTHPLQQSLKDLERAYANFFAKRADFPRFKRKGVRDSFRYPDAKQIKLDETNSRIFLPKLGWLRYRNSRKIQGEVRNATVSLRAGKWFVSIQTEREVDKPLPEAASAIGIDMGIARFATFSDGCYLAPSNSFKRHEAALRKAQQSLSRKEKFSRNWSKAKSKIQRIQARIGNTRHDYLHKATTAISQNHAMVCIEDLQVKNMSKSAAGTTEVPGHNVKAKSGLNKAILDQGWFEFRRQLQYKLEWSGGILIAVPPQNTSRTCPTCGHISKDNRQTQAHFVCVECGYTNNADVVGAINILRAGHARFACEVNPDVGDQQQEPAEVIQYGNNATLDAVGIPLL

SEQ ID NO: 176 - protein, 406 aa, synthetic construct
MQRLQAFRFELRPNGQQQRQMRRFAGACRFVFNKALALQKERYANGEKKLGYAGLCKELTAWRNGAETPWLADAPIHPLQQSLKDLDRAYANFFAKRADFPRFKKKGQGDSLRYPDPKQIKLDAGNARIFLPKLGWLRYRNSRDVLGAVKNVTVSASCGKWFVSICTSREVEQPIASGDAVGIDMGIARFATLSDGTFVAPLSSFKRHENRLRKAQQSLSRKVKFSRNWSKAKARVQRIHTRIGNARRDFLHKTSTTISQNHAMVCIEDLAVRNMSRSAAGTVEKPGRNVRAKSGLNKSILDQGWFEFRRQLDYKVAWRGGWLVAVPPQYTSRTCPCCGHVSAENRKTQAQFLCVACGYANNADVVGAINILRAGHARCACPVSGAAMPPAAGTHRSDSGVAPCHA

SEQ ID NO: 177 - protein, 430 aa, synthetic construct
MQRLQAFKFELRPNGQQERQMRFYAGACRFVYNKALALQKERYEQGEKKLSYVQLSRLLTEWRNSEELAWLANAPVHPLQQKLKDLEKAYTNFFAKRADFPRFKKKGRCQDSFRYPDPKAFKLDQDNNRIYLPKLGWIRYRNSRKVLGELRNVTVSFKSGKWFVSIQTRREVEKPIPKGGIVGIDLGVAQFVTLSDGTFYAPINSFKRHQVALRKAQQSLSRKVKFSNNWKKAKARVQRIHTRIANIRQDFLHKTSTAISKSHAIVCIEDLQVQNMSKSAAGTKESPGKNVRAKSGLNKSILDQGWYEFRRQLEYKLAWNGGKLILVFPQNTSRTCPCCGYVSEDNRLTQDKFECVECGFKNNADLVGAINILSRGIQILRDEGQDTTDASVGMLAGSLLASARMACGSNHASDRKQEPTETVAYGGNYA

SEQ ID NO: 178 - protein, 406 aa, synthetic construct
MQRLQAFKFELMPNGEQVRKMRQFAGMARFVFNRGLALQKERHEAGEKKLSYAALCKVLTGWRNSVETPWLKDGPIHPLQQSLKDLERAYANFFAKRADFPRFKKKGQRDSFRYPDPKQIRLDQGNRRLYLPKLGWLRFRKSREVLGTVKNVTVSQSAGRWFVSIQTEREVAVPAPTGAAVGIDLGVARFATLSDGSYLAPLNSTKKHHVALRKAQQAMSRKTQFSNNWKKAKARVQKVQARIANARRDFLHKASATICQNHALVVLEDLQVGNMSRSARGSVEQPGRHIRQKSGLNRSILDQGWVEFRRQLDYKLAWRGAHLVVVPPQNTSRTCPECGHIAAENRRSQARFACVECGFAANADHVGAINVLRAGHARLACEVSGAVRPPAAGTHRSDSGAAQCRA

SEQ ID NO: 179 - protein, 413 aa, synthetic construct
MQRFQAFKFELMPTGEQARDMRRFAGACRFVFNQALALQKARYERGEKKLGYAGLCQQLTGWRNSTETPWLKAAPTHPLQQSLKDLERAYSNFFAKRANFPRFKKKGRRESFRYPDPKQIKLDQANRRIFLPKLGWLRYRKSRDVLGTLKNVTVSQASDKWFVSIQTARAVEPPVPLATSAIGIDVGIARFATMSDGAFLVPLNSFKKHEMRLRRYQRAMSRKVKFSKNWRKAKAHVQRIHARIGNARRDYLHKATTTISKSHAMVSIEDLQVRNLSRSAAGSVEAPGKNVKAKSGLNQRILDQGWFEFRRQLEYKLAWNGGWLIAVPPQNTSRTCPACRCVSADNRRTQAQFACVACGYQAHADVVGAMNVLTRGLQLLEGQDDARIARGEMAQSGHSVKREPADATTWNIT

SEQ ID NO: 180 - protein, 406 aa, synthetic construct
MQRLQAYKFELRPNGQQQSQMRRFAGSCRVVFNEALALHKARYAHGEKKLGFAALCKELTTWRGGVPLPSGRTAPWLSDAPIHPLQQSLKDLERAYTNFFAGRAAFPKRKERGEHDSFRYPDPKQFKLDQAGSRIFLPRLGWMRLRLSRPVPGALRNVTVSLSAGRWFVSIQTEREVETPRPCATSAVGIDVGVARFATFSDGSFLAPLNSFKRHEARLRRYQRAMSRKVKGSSNWKKARARVQRIHARIANVRRDFLHKATTDIARTRALVCIEDLAVRNMSRSAAGTVERPGRNVRAKSGLNKAILDQGWFEFRRQLEYKLAWSGGHLIAVPPANTSRTCPACGHVAAENRPTQARFHCVACGHAAHADVVGAINILARGHRVAACGEPVQSGRSTKQEPAEAI

SEQ ID NO: 181 - protein, 370 aa, synthetic construct
MEYSYKFRIYPNAAQENLIQRTFGCSRFVFNHFLAERMEQYEKTGKAPTRFQQDKELTALKKELLWLKEVDATALQASLQMLDTAYQNFFRRVKQGEKPGYPRFKSKHNHRRSYKSKCVGTNIKVLEKAVQLPKLGLVKCRISREVKGRILSATVSQAPSGRYFVAICCTDVEINPWPAAGTVVGLDMGLKSFAIASDGKKYPNHRCLFKGQKKLARLQRQLSRKTKGSNRREKARIKVARLHEQIANQRLDMLHQLSADIVRNYDRIAIEDLAPANMKKNRKLARSISDASWSEFRRQLKYKAAWYGKQVVIIDRFFPSSQICSSCGVQWPGTKDLSIRTWICPACGTVHDRDINAAKNILKEGLRMLA

SEQ ID NO: 182 - protein, 371 aa, synthetic construct
MEYSYKFRLYPNKEQEKLIRRTCGCCRFVYNYFLAQRIEQYKQAGKAPTRFQQDKALTGLKTKLEWLKEVDATALQSALQNLDAAYQNFFRRVKQGSGKPGFPQFKTKHERRRSYKSKRIGNNINVLTNAVRLPKLGLVKCRVSKQVKGRILSATVSQNPSGKYFVALCCTDVERKPLPSTGAAVGVDMGLKAFAITSEGVEYPNHRYLARNGKKLARLQRQLSRKTKGSKRREKARLRVARLHEHVANQRRDMLHKLSTEMIRQNDVVCIEDLAPSNMVKNRKLARAISDVSWYEFRRQLEYKAVWYGKQVVTVERFFPSSQLCSVCGAQWSGTKDLSVREWICPHCGAVHDRDVNAAKNILKEGLRLLA

SEQ ID NO: 183 - protein, 370 aa, synthetic construct
MEYSYKFRIYPSREQENLIRRIFGCCRFVYNHFLAQRIEQYKEDKKVPTRFQQDKSLTTLKRERIWLREADSTALQAALQNLDAAYQNFFRRVKQGGEPGFPRFKCKHDHRQSYKSKCVGNNIKVLAKAVRLPKLGPVKCRVSKKVEGRILSATVSRNPSGKYFVALCCTDVEIEPLPSTGAAIGMDMGLKAFAISSDGTEYPNHRYLVKSRKKLARLQRRLSRKTKGSHRREKARLQVARLYEHVANQRRDMLHKLSTQLVRENDVICIEDLAPQNMMKNHSLARSIADAGWGEFRRQLEYKAEWHGRRVVAIDRFFPSSQICSVCGAQWPGTKDLSVRAWTCPFCGTVHDRDLNAAKNIWKEGLRLLV

SEQ ID NO: 184 - protein, 419 aa, synthetic construct
MEYSYKFRLYPTDAQRNQIARNFGCCRYVFNHFLAQRQEHYKETGKAPTRFQQDKALTSMKQELQWLKEADSTSLQAALQDLDAAFQNFFRRVKRGEKPGYPRFKSRKDRRQSYKSKCVGTNIKVLEKAVRLPKLGLVKCRVSNEVRGRILSATVSCSPSGKYFVALCCTDVDIETLPSTGAVVGLDMGLKSFAVTSNGVEYPNHKFLTKSIRKLARLQRQLSRKSKGSKRREKVRLKVARLQEHISNQRQDTLHKLSTQLVQGYDLISIEDLAPKNMVKNHKLARSISDASWGEFRRQLEYKAAWYGKQLVTVDRFFPSSQLCSVCGAQWPGTKDLSVREWTCPSCGAAHNRDTNAAKNILNEGLRLLAQQKHTVGRDTPKPIRSGRPYKSSHVAGCGQRTENPPALAVGSVKFATKS

SEQ ID NO: 185 - protein, 371 aa, synthetic construct
MEKSYKFRIYPNKEQEILIQKTFGSTRYVFNHYLAQRIEQYKEVGKAPTRFEQDKQLTELKQELIWLREVDATALQSSLQNLDTAYQNFFRRIKQGGKPGFPKFKSKRDKHKSYKSKKVGENITVLDKHVKLPKLGLVKCCVSKRVEGRILSATVSQNPSGKYFVSICCTDVDISPFESTEAAVGIDLGLKDFAITSDGEIISNPKYLAKSQKKLAKLQRQLSRKSIGSNNRNKARIRVARQHEKVFDQRQDFLQKLSTNLIRNYDLICLEDLQVKNMVKNHKLAKSISDVSWSEFVGQLQYKADWYGRYILKVDKFFPSSQLCSNCGTKWSGTKDLGVREWVCPECGTNHDRDINAASNILKEALRLLSA

SEQ ID NO: 186 - protein, 372 aa, synthetic construct
MEYAYRFRLYPTAVQREQMSRNFGCCRYVYNHFLAQRQEQYRETGKSPTRREQEKSLTALKQELIWLKEADSTSLQATLRDLDDAYENFFRRVKEGGNPGYPRFKSKHEHRQSYKSKRVGTNINIPKTGGAIQLPKLGLVKCRISKEVRGKILSATISRNPSGKYFVSICCTDVDIEPLAPTGKEVGLDLGIRSFAVSSDGVEYPNHKYLARSEKKLAKLQRELSRKSKGSKRWEKARLKVARLNEHIANQRSDMLHRLSVHIIRENDVICIEDLAPKNMVRNHKMAKSIADVSWSEFRRQLTYKAEWYGRQLVTVDRFYPSSQLCSVCGAQWAGTKDLTVREWVCPVCGAVHDRDINAAKNILTEGLRLLA

SEQ ID NO: 187 - protein, 370 aa, synthetic construct
MEYSYKFRIYPNREQQTLIQKTFGCARYVYNHFLAQRIAEYKTTGKAPTRFQQDKELTSMKKELPWLCEVDATALQSSVQTLDVAYQNFFRRVKGGEKPGYPKFKSKKNRRRSYKSKCVGTNIQVLDKAVRLPKLGLVKCSVSKEVRGRILSATVSQNPSGKYFVALCCTDVDIEPVPSTGAAIGLDMGLKAFAITSDGVEYQNHKFLSKSTRKLARLQRQLSRKSKGGKRREKARVKVAKLQEHVANQRQNTLHKLSTQFVRDYDFIAIEDLAPKNMVRNHKLARSISDASWGEFRRQLEYKAAWYGKVVVTVDRFYPSSQLCSACGAQWPGTKDLSVREWTCPVCGTVHDRDVNAAKNILNEGLRLLA

SEQ ID NO: 188 - protein, 370 aa, synthetic construct
MEYSYQFRLYPTVEQREQISRNFGCCRYVFNYFLAQRQEQYQATGKAATRFQQDKQLTALKQEFPWLKEADSTSLQAAVQDLETAYHNFFRRVKHGEKPGYPRFKSKHNHRQSYKSKCVGTNIKVFENAVQLPKLGKVKCRISKEVKGRILSATVSRNPSGKYFVSLCCTDVEIEPLPSTGAVTGLDMGIKAFSVDAEGKEYPNHRYLAKSEKKLTRLQRQLSRKTKGSQRREKARIKVARLHERITNQRKDLLHKLSTELLRKNDVVCIEDLTPKNMLKNHRLAKSISDASWGEFRRQLEYKVAWYGKKIVVIDRFYPSSQLCSDCGIQWSGTKDLSVRQWICPTCGTVHDRDCNAAKNILNEGLRLLV

SEQ ID NO: 189 - protein, 373 aa, synthetic construct
MEFSYKFRLYPTKAQEEQITKNFGCCRYVFNHFLTQRQAQYKETGKAPTRFQQEKSLTELKRELPWLKEADSTSLQASIQDLDMAYQNFFRRVKHGEKPGYPRFKSKRQHRRSYKSKCVGTNIKVLDGAVQLPKLGEVKCRVSRKVEGRVLSATVSQEPSGKYFVSICCTDVDIKPLPKTGAAVGIDLGIKDLAITSDGVKYDNLHSYRRNLKKLARLQRQLSRKSKGSRNREKARIQVARLQEHIANQRKDTIHKMTTQLVHNNDVICIEDLSSKNMMRNHKLALHIADASWGEIRRQLAYKTDWYGKELVVIDRFFPSSQTCGCCGYRNKEVKNLGVRFWTCPQCGAFHDRDINATQNILEEGLRKRASTA

SEQ ID NO: 190 - protein, 402 aa, synthetic construct
MRRAYKVRAYPDADQQAVFRRTFGCVRLVWNKTLANRQRRYTTEQRSTSYRETDAALTEWKKTEDLAFLSDVSSVPLQQTLRHQHSAFAAFFAKRGRYPRFKSRHGKQSAHYTRSAFRMKADGLWIAKTASPLRVAWSWPDIDMTAIQPTMVVVSRDPDGRWFVTFAVDEDDPAPLPDTGETVGIDLGLKDFAVLSTGERIANPMHMERHERGLKKQQRRLARCQKGSNNRAKQKIKVARKHSKVRDARRDFLHKTSTDIVQRFDVIAVEDLAVKNMVRNRSLAKSISRVGWAEFRSLLEYKAEQAGRRFAVIDRWYPSSKTCSHCGHLLATLSLGTRHWSCPDCGTRHDRDVNAAKNIIVAAGLVETQNACGADARPKRATVRRSAVKQEPQPAKVGIPCL

SEQ ID NO: 191 - protein, 402 aa, synthetic construct
MRTAYKCRAYPAPGQVVVLNRTFGCVRVVWNRTLAARHARWHAEHKPTSYAETDRALTEAKKLLELAFLNEVSSVPLQQTLRHQHTAMTAFFQKRARYPRYKSRHGRQSASYTRSAFRMRDGQLSLGKTPGVLAFVWSWPDIDVTQLDPTMVTVSREPDGRWFVTFAVDVEAPAAPESTGDVVGVDLGLADFAVLSTGERIAHPKHMERRERRLKRYQRMMARKEKGSANRAKAKRKVARAHSRVRDARRDFLHKASTDLVRRFDAVAVEDLAVANMVRNRSLAKAISRTGWAEFRSMLTYKAHRDGRTVAVVDRWYPSSKTCSVCGNLLAKLSLGTRHWACPGCGTRHDRDVNAARNIMLAAGLVESRNACGADVRRDGQPFALSALNQEIRPARVGNPGT

SEQ ID NO: 192 - protein, 400 aa, synthetic construct
MRTAYKCRAYPDPEQAAIFARTFGCVRLVWNKTLAERRRAWQTDRTPTTYKQTDAALTAWKKTQDLAFLNEVSSVPLQQALRHQHTAFANFFAGRARHPRFKSRTGRQSAHYTRSAFRMKDGHLWMARTTAPLAFIWSFQDVDPAGLNPTMVVISREPDGRWYVTFAIDADVPEPLPATGHAIGIDLGVKDFAVTSDGDRVANPRHLDRKAANLARYQRRMARKQPGSNNRRKAKTKVARACRKVRNARHDFLHRTSTNLVRQADIIVIEDLAVANMVKNRSLARAISDCGWGEFRRQLEYKAGRAGRVLIVIDRWYPSSKTCSACGHLLTKLSLSTRHWTCPGCRTRHDRDHNAAKNILAAGRAVAGAGPGDVCGADVRRQGPSLPQSAAKQKPPRREP

SEQ ID NO: 193 - protein, 401 aa, synthetic construct
MRTAYKCRAYPTPEQAAMLNRTFGCVRLVWNKTLSERNHRYSTNGTRTSYRQTDSALTAWKRTAELAFLSEVPSVPLQQTLRHQHTAFQNFFAGRARRPRFKARTGRQSAHYTRSAFRMRDGTLMLAKTSAPLRFVWSFGIDVTEVDPTMVVVSREADCRWYVTFAVDTDAPAQTAPIGKVVGVDLGLTDFLVTSDGERIANPRNLARKAHNLARYQRRLARCQRGSSNRSKAKIKVARAHRKVRNARRDFLHRTTTTLVRSADVIAIEDLSVKNMVRNRYLARAISDAGWGEFRRQLEYKCRRAGRTLVVIDRWYPSSKTCSACGYALTELKLATRHWTCPDCRTRHDRDLNAAKNILAAGRAVARGDSGDACGADVRRQGPSLPRSAVKQESRAARRSP

SEQ ID NO: 194 - protein, 411 aa, synthetic construct
MRTAYRCRAYPDQGQRAALNRTFGCVRVVWNHSLAERHRRWHTDHTGMTYAQSDRALTTMKQDPGLAFLSEVSSVPLQQTLRHQHQAFSAFFAKRARYPRFKSRRSRQSAHYTRSASSLRSGVLRLAKMSTPLPFVWSWPDVDVTGLDPTMVIVSREEDGRWYVTFTIDTDDPEPLPAAGRAVGIDLGVTDFAATSHGDKIANPRHLDRKSRNLARYQRRLARCQNGSANRAKAAAKLARAHRKVRNARRDFLHRVSTRLVRQNDVVAIEDLNVAGMVRNRHLARAISGCGWGEFRRQLEYKCQRYGRRLVVINRWYPSSKTCSACGHLLADLRLSTRQWTCPSCGTRHDRDINAAKNILAAGLAVAPGSPGDACGAGIRHPGTSWVRPAMKQEPQPVTAGIPVLQLQGEE

SEQ ID NO: 195 - protein, 400 aa, synthetic construct
MRTAYRVRAYPTAEQAAALNRTFGCVRLVWNTVLAWRQARYRAEGVKTSYAQTDRYLTELKRDGGHDFLYEVSSVPLQQALRHQHKAFANFFAGRAKHPRFKSRHGRQSATYTRSAFRWGDGRLHLAKMDDPIAFVWSWPEVDPTTINPTTVTVSRDSDGRWYASIAIETETEPDPLPATGGQAGIDLGVSDFAVLSTGEKIPNPRHLERKAKNLARYQRRMARKQRGSNNRAKARAKVARAHTKVRDARADFLHRTSARLVRDHDLIVLEDLNVRGMVRNRSLARVISDCGWGEFRRQLEYKTQRYGRRLVVIDRWYPSSKTCSACGHLLPALPLSTRHWMCPGCGTRNDRDVNAAKNILAAGRAVSACGADVSHSGSSRMRSAVKQEAQPARAGVPRL

SEQ ID NO: 196 - protein, 417 aa, synthetic construct
MRTAYKVRVYPDSEQAAQLGRTFGCVRLVWNKTLAERHTAYHQRGEKTSYKQTDHALTEWKRGEDLAFLSEVSSVPLQQALRHQHTAFGNFFAGRAKYPRFKSRTARQAAHYTRSAFRIRNGQVFLAKQTRPLEVAWSWPQVDLTALNPTMVVLSREPDGRYYATFAVQTHDPQPAPECNSQVGVDLGVKDFATLSTGEKIANPRHVERKAKNLARYQRRMARKQRGSNNRRKAKRKVAAAHRKVRHARQDFLHKTTTRLVREHDLVAVEDLNVSGMTRSCRGAVDAPGKQVAQKSGLNRSVLDAVLGEFRRQLEYKAHRAKRTIAVVDRWFPSSKTCSDCGHLLAKLSLGTRHWTCPACRTRHDRDVNAAKNILAAGRVAAREHSGDACGAGVSRQGSSLPQSATKQETGPVRAGP

SEQ ID NO: 197 - protein, 402 aa, synthetic construct
MRTAYRCRAYPTAEQAAVLNRTFGCVRLVWNKTLSERSRRYRIDGARTSYRETDAALTTWKRSDELAFLSEVSSVPLQQTLRHQHSAFLHFFSGRSRYPRFKSRTGYQSAHYTRSAFRMRDGVVTLAKTDVPLRFTWSFARVEVATLDPTMVVVSREPDGRWYVTFAVDAETPAPAEPVSHGVGVDLGLTNFLVTSDNERIAAPQHLARKARNLARYQRRLARCRPGSSNRLKAKVRVARAHRKIRHARQDFLHRTSTALVRCADVIAIEDLNIAGMVRNRRLSRAISDAGWGEFRRQLQYKCERAGRVLVVIDRWFPSSKTCSTCGHLLTELKLSARHWTCPGCRTRHDRDLNAAKNILAAGRAVARDNSGDACGADVRRQGPSLPRSAVKQEPRVVRHGA

SEQ ID NO: 198 - DNA, 203 bp, synthetic construct
  source 1..203: mol_type = other DNA
gccgatgtagtgggagcaatgaatgttcttacacgtgggcaagcaatagtccaagcataataaatatcagggtaggacatacccgaagcgtctatgaagtgaatctcaatagagatcagcagtagaaacccaccgagagtagcccacagcttgctgtgggagctagtaggaatcccctgcctttaggcaggggaggatgtcaa

SEQ ID NO: 199 - DNA, 202 bp, synthetic construct
  source 1..202: mol_type = other DNA
gcggggcggtcctggaccgggacatcaacgcggcggtcaacgtcgccaaggccgccggactggcggcatcagcctgtggagcgcaggtaagaccgggactcgtcccggcaccgcgcggcgaagcaggaacccactcgacaccgcagccacaggcggcgcggtaggcgggaatcgccgtccttcagggcggcgaggatgtcaa

SEQ ID NO: 200 - DNA, 214 bp, synthetic construct
  source 1..214: mol_type = other DNA
gtaaaaaatctaaatttacgtagttggatatgcccgaactgcggcgtacaacatgatcgtgaccaaaacgctgccatcaatattgagcaagaaggacttcgattgttggcgtagccaactaaactgtgggactcacagggatagcttggtatatttcactctgttaggagtgactacccaagaatcccctgcctttaggcatggggagtgtcaa

SEQ ID NO: 201 - DNA, 204 bp, synthetic construct
  source 1..204: mol_type = other DNA
gtttggacctgtgagtgcggggcggtgcacgaccgagacctgaacgcggccctgaacatccgggccgaagggatacgagctatccccgtcgccgtggggcacccggagacgctaaacgcttggggagagggtgtaagacctacacaacgtaggcagtcctcgtcgaaccaagaatcccacgtgctttagccgtgggagtgtcaa

SEQ ID NO: 202 - DNA, 201 bp, synthetic construct
  source 1..201: mol_type = other DNA
gcgtgaatgggactgcccagaatgtggagcgcaccacgaccgagactttaacgcaagcctgaacattcttgccgaaggcaaaagacttcttgctgtgtaatccttacacgaactgtcggaacgacagggttagcttggtaaatttctcttcgttgaagggactacccaagaatctcgtgactttagtcatgagaggttcaa

SEQ ID NO: 203 - DNA, 204 bp, synthetic construct
  source 1..204: mol_type = other DNA
gatacgaatgcaagtatcaatattctgaaaaaaggactgcagatgcagtctgcataaagataaaaactgtaccgtagggcatacgggaacagtataaatatagcttgtggacactgtgtaagacattgcagtaccgtaaggtgtttgccaatgcagtagtgggagaagcaagaatccccctgctttagctgtggggagtgtcaa

SEQ ID NO: 204 - DNA, 205 bp, synthetic construct
  source 1..205: mol_type = other DNA
gtcaacgcggcgagaaacatactgacggaagggaaacgcatcctctccaacacgcaaggtaccgcggggcacgcgggaaccgtggcggctataaccgcctaaacgctcggggagaggacgtaagaccctagaggactatatcggtccccgacggcagacctcgcagaactgagaatcccctggtttcaactacggggagtgtcaa

SEQ ID NO: 205 - DNA, 202 bp, synthetic construct
  source 1..202: mol_type = other DNA
gtaagagaatggacatgtccaaaatgccattcaaatcatgacagagatgtaaatgcatccatcaatatattgatgcaaggcatattagcaaattaaataaaaacaaaaacaaccgcaggaactgcggggatagcttggtaagaaaggtttcaatagaaacctgttcccaagaatctcgtcacttcagtgatgagaggttcaa

SEQ ID NO: 206 - DNA, 207 bp, synthetic construct
  source 1..207: mol_type = other DNA
gatctcaatgcggccttgaacatcgcgcgggaaggagcgcggcttctaaatgaagcagacggtaccgcagggcttgcgggaaccggggacgcgaaagcgtcccgaacgctcgtggagcaggcgtaagaccagcttccgccgcaaggcggaggaaggctatctgcgtcgaagcgagaatcccccggcttcagccgtggggagtgtcaa

SEQ ID NO: 207 - DNA, 202 bp, synthetic construct
  source 1..202: mol_type = other DNA
gtggaatgtggcttcgaggacaacgccgatgtggtcggcgcgatcaatgtgctaagggcgggacacgcccggttcgcctgtgaagtgagcggtgaggtaatgccgccagcagcaggaacccaccgaagcgactcaggggcggctcaatgccgcgcctgagcgccgtaggaatctccggccttcaggccggggaggatgtcaa

SEQ ID NO: 208 - DNA, 202 bp, synthetic construct
  source 1..202: mol_type = other DNA
gtggaatgcggtttcgaggaaaacgccgatctcgtcggcgcgatcaatgttctaagggcgggacacgcccggttcgcctgtgaagtgagcggtgcagtcatgccgccagcagcaggaacccaccgaagcgattcaggtgcggctcaatgccgcagctgagcgccgtaggaatctccggccttcaggccggggaggatgtcaa

SEQ ID NO: 209 - DNA, 203 bp, synthetic construct
  source 1..203: mol_type = other DNA
gttccggtgccgcgcctgcggccaccaggacaacgccgacgtgaacgccgcgaagaacatcctcgccgccgggctggcggtgacagggcgtggagacctcgccgttgggcggtctgtgaagcgccaaccacccgaggccgaggtcgcgtgaacgcgcctcagccacgggaatcccccgccttcaggcgtggggaggaagtcaa

SEQ ID NO: 210 - DNA, 202 bp, synthetic construct
  source 1..202: mol_type = other DNA
gtggtcgtcaagaaggatttgagcgttcgcgtacatgtgtgtcctgactgcggtctcacgcttgaccgtgacgtgaatgccgctcggaatatcctgatgcttgcggtcaacccggcccggacggggccgccagagcgtaacgtggccggtcacggcgagcgtgctctgggaagctccccgctttagcggggagagtcgtcac

SEQ ID NO: 211 - DNA, 201 bp, synthetic construct
  source 1..201: mol_type = other DNA
ggcctaatactcgaccgagatgtgaacgccgcccggaatatcctgaaaaaggcgttggctctggaagcggcatgatgcttccctaccgtgggaaccacgggacgttacgcctgtggagctgacccgttggggttacgatgaagcaggaaaccctaccgaagcccctccctaacccggaggggttagggaggggtagttcac

SEQ ID NO: 212 - DNA, 206 bp, synthetic construct
  source 1..206: mol_type = other DNA
gaaattcaaatgtgtggcctgtggatttgaatgcaacgccgaccataatgcggcgttaaacattctggcggccgggcaggccgtgtcagcctgtggagcgggaaggcctcaggcgcccgctttgaagcaggaacccgcctatggtgtggctcgttagagccgctaccgataggaatcccccgtctttaggcgggggaggatgtcaa

SEQ ID NO: 213 - DNA, 204 bp, synthetic construct
  source 1..204: mol_type = other DNA
gttcagcgtgcggcgagagagtgctgaaggccctctctgtccggtggcactcctgcccctactgcggggcagaactccaccgcgaccacaacgccgccctgaacatccgaaagaagggcgggggcaccgccttggtggatgtggcagcactggctgccgcgtgagaaaccagaaccccacaggctttagccgtgggagtgtcag

SEQ ID NO: 214 - DNA, 202 bp, synthetic construct
  source 1..202: mol_type = other DNA
gcgtcgaatgcggtttcgaggaaaacgccgatctggtcggcgcgatcaacattctcagggcgggacacgcccattgcgcctgtgaagtgagcggtgcagcaatgccgccagcagcaggaacccaccgaagcgactcattccggctcgatgccggggtgggcgcagtaggaatcccccgactttaggcgggggaggatgtcaa

SEQ ID NO: 215 - DNA, 201 bp, synthetic construct
  source 1..201: mol_type = other DNA
ggatgcaaaaactgcgagacgaagggcgggacacgctggacgctttcagcgggacggcgcaagccgtcagcccggatggcctgtggatcgaaccgcactggcggtcggaagcaggaacccaccgaagcgaatcaggggcggctcaatgccgtgcctgagcgccgtaggaatccctgtcctttagggcagggaggatgtcaa

SEQ ID NO: 216 - DNA, 202 bp, synthetic construct
  source 1..202: mol_type = other DNA
gtggaatgcggcttcgaggaaaacgccgatctggtcggcgcgatcaatatcttaagggcgggacacgcccggttcgcctgtgaagtgagcgatgcggtaaggtcgccagcagcaggaacccaccgaagcgactcagaggcggttcaatgccgcacctgagcgccgtaggaatctccggccttcaggccggggaggatgtcaa

SEQ ID NO: 217 - DNA, 202 bp, synthetic construct
  source 1..202: mol_type = other DNA
gcggggcggtcctggaccgggacatcaacgcggcggtcaacgtcgccaaggccgccggactggcggtatcagcctgtggagcgcaggtaagaccgggactcgtcccggcaccgcgcagcgaagcaggaacccacccgacaccgcagccttcaacggcgcggtaggcgggaatcgccgtccttcagggcggcgaggatgtcaa

SEQ ID NO: 218 - DNA, 207 bp, synthetic construct
  source 1..207: mol_type = other DNA
gtctcaacttgcgcagttggatatgcccgcactgtggcgtacaacatgatcgtgaccaaaacgctgcgatcaatattgagcaagaaggacttcgattgttggcgtagtcaactaaactgtgggacacacagggatagcttggtaaaattcactccgttaggagtgactacccaagaatcccccgcctttaggcatggggagtgtcaa

SEQ ID NO: 219 - DNA, 209 bp, synthetic construct
  source 1..209: mol_type = other DNA
gaatctcaatttgcgtaattggacatgcccgaactgcggcgtacaacatgatcgtgacaaaaacgctgccatcaatattgagcaagaaggacttcgattgttggcataaccaactgaactgtgggacacacagggatagcttggtaaatttcactccgttaggagtgactacccaagaatcccctgcctttaggcatggggagtgtcaa

SEQ ID NO: 220 - DNA, 205 bp, synthetic construct
  source 1..205: mol_type = other DNA
gtcaatgcggcgagaaacatactggcggagggaaaacgcatcctctccaacacggaaggtaccgcggggcacgcgggaaccgtggcggccgtagtcgcctaaacgctcggggagaggacgtaagaccctagaggaccatatcggtccccgacggcagacctcgcagaactgagaatcccctggtttcaactacggggagtgtcaa

SEQ ID NO: 221 - DNA, 205 bp, synthetic construct
  source 1..205: mol_type = other DNA
gtcaacgcggcgagaaacatactgacggaggggaaacgcatcctctccaacacggaaggtaccgcggggcacgcgggaaccgtggcggccgtagtcgcctaaacgctcggggagaggacgtaagaccctagaggactatatcggtcctcgatggcagacctcgcagaactgagaatcccctggtttcaactacggggagtgtcaa

SEQ ID NO: 222 - DNA, 205 bp, synthetic construct
  source 1..205: mol_type = other DNA
gtcaacgcggcgagaaacatactgacggaagggaaacgcatcctctccaacacgcaaggtaccgcggggcacgcgggaaccgtggcggctataaccgcctaaacgctcggggagaggacgtaagaccctagaggactatatcggtccccgacggcagacctcgcagaaccgagaatcccctggtttcaactatggggagtgtcaa

SEQ ID NO: 223 - DNA, 204 bp, synthetic construct
  source 1..204: mol_type = other DNA
gttccggtgccgagcctgcggccaccaggacaacgccgacgtgaacgccgcaaagaacatcctcgccgccgggctggcggtgacagggcgtggagacctcgccgttgggcggtccgtgaagcgccaaccacccgagaccgaggctgcatgaacggcacctcagccacgggaatcccccaccttcaggtgtggggaggaagtcaa

SEQ ID NO: 224 - DNA, 206 bp, synthetic construct
  source 1..206: mol_type = other DNA
gttcttgtggtttagtattggatcgcgatcacaattctgcgatcaatatcaaaaatgaaggattacgcttagtagcgttcgcataaaaaacaacaactcttggaacaagagggttcgctcggtccatttcctatcgttagataggactacccgagaagcccccacttcaatcaaaccgcaaggttgataagtggtgggtagttcac

SEQ ID NO: 225 - DNA, 205 bp, synthetic construct
  source 1..205: mol_type = other DNA
gtacattccattgcgattgcggttttgtggcagatagagactggaatgcttcaatcaatatcaagaatgaaggattacgactattagcgtaatcaaataaatagagcatggttcaggctcgatagctaggtcaatttcgtaccactaggtgcgattaccctagaagcccccacttcaagcgatagctaagtggtgggtagttcac

SEQ ID NO: 226 - DNA, 201 bp, synthetic construct; identical to SEQ ID NO: 215
  source 1..201: mol_type = other DNA

SEQ ID NO: 227 - DNA, 201 bp, synthetic construct
  source 1..201: mol_type = other DNA
gtcgaatgcagttacgaggagaacgccgatgtcgtcggcgctctgaacattctggcgcggggacaccgcgttgcagcctgtggagagccggtgaagtcgggccgctccgtgaagcaggaacctgccgaggcgatctgagataagaattatcgtttcagacgcggtaggaatcccccgtcttcaggcgggggaggatgtcaa

SEQ ID NO: 228 - DNA, 201 bp, synthetic construct
  source 1..201: mol_type = other DNA
gttgcgcctgctcgcaatgcgcgagcagcaggtcgccgatgggcagtcggagaccataaacgcccgtggagctgacgttagacagggatgttcccctgcggtcggccgcgaagcgggaaggaacgcgggagacctgtcgtgcgtcgcatgacgggccgagcgttccccgaatccccgcccttcagggcggggaggtgtcaa

SEQ ID NO: 229 - DNA, 202 bp, synthetic construct
  source 1..202: mol_type = other DNA
gtgggagcaagaaatatcaaggtgcgcgggcagcgcatgtcagcctgtggggagctcggcgaagaaggtcgagctcgagtgaatcggcggatggagtcaatccatcgcgccggtcagcagcaggaacccatcgaagtgatcatcggtgagagccgatgacacagcagggaatccccgtcgttcacgacggggaggatgtcaa

SEQ ID NO: 230 - DNA, 201 bp, synthetic construct
  source 1..201: mol_type = other DNA
gctgcggtgaacattctcgggcgcgggcagcgcctcagagcctgtggggagctcgttcctgtgaacgcagctcaagtgagtcgaccttcccgtaagagaagcgtcgtccagcaacaggaacccgtcgaagcgaccgttggcggctctgccgacaacggcacagtagggaatcaccgtccttcagggcggtgaggatgtcaa

SEQ ID NO: 231 - DNA, 202 bp, synthetic construct
  source 1..202: mol_type = other DNA
gtgtggacatacacacgaccgggaccgtaatgcagcaatcaatatcttaaacgagggattgcgcctactggcgtgatctatatacatacggtagggcgggacacgcccgaacctatacgcttggggagaccgtgtaagtcctgcttgcaggccgtgatctacgaaccaagaatccccctgctttagctgtggggagtgtcaa

SEQ ID NO: 232 - DNA, 202 bp, synthetic construct
  source 1..202: mol_type = other DNA
gcgggaccatccacgatagagatctcaatgccgcgaagaacatcttgaacgaaggcttgcgtctgttggcgtagccgatccacacggtagggcgggacacgcccgaacctatacgctcggggagaccatgtaagacctctcatggtgaaggctacggtcagcgaaccgagaatcccccggcttcagccgtggggagtgtcaa

SEQ ID NO: 233 - DNA, 202 bp, synthetic construct
  source 1..202: mol_type = other DNA
gtgaatgtggattcgagagcgacagggacgtcaatgcggcaatcaatatcaaacatgagggcatgaaacgattagcgatagcctaacttgtcctcgaaccgtgggacacacggggatcgctcagtcaacttcccgtcatgagatgggattacctgagaagcccccacctctaagcgaagcgtaggtggtgggagcatgtcac

SEQ ID NO: 234 - DNA, 202 bp, synthetic construct
  source 1..202: mol_type = other DNA
gtgaatgtggattcgagagcgacagggacgtcaatgcggccatcaatatcaaacatgagggcatgaaacgattggcgatcgtctaatttgtcctcgaaccgtggggcacacggggatcgctcggtcaacttcccatcatgagatgggattacccgagaagcccccacctctaagcgaagcgtaggtggtgggagtatgtcac

SEQ ID NO: 235 - DNA, 204 bp, synthetic construct
  source 1..204: mol_type = other DNA
gtacatttcgctgtgattgtggttttgtagcagaccgtgattggaatgcttctatcaatatcaagcgtgaaggattgcgactgttagtataatcataaatagagcatggaacaggctcgatagctaagtgaatttcgtaccgttaggtgcgactaccttagaagcccccacctctaagcgaagcgtaggtggtgggtagttcac

SEQ ID NO: 236 - DNA, 205 bp, synthetic construct
  source 1..205: mol_type = other DNA
gttcctggctctgtaccgcctgcgggtcgttgcatgaccgtgacgaaaatgccgccgagaacatccggcggcaagggattctgaaactcaaggccgaaggactgtcggtctcagcccatggaggcggtgtcagtcgggtgtctagcgcacccgcaaccgccagtgaagtgggaagcctccgccttcaggcgtgaggagcagtcac

SEQ ID NO: 237 - DNA, 201 bp, synthetic construct
  source 1..201: mol_type = other DNA
gtgcggtcgcacccatgaccgcgacgttaacgctgctcggaacatcagggacgaaggtatgaggctcgcgctttcggcctagaggaagcgaaaccgtggggcacacggggatagctcgtcgatactggcgacatcggttgccttgagcgagaagcccccacctctagcagcgaagttgcaaggtggatgggagcatgtcac

SEQ ID NO: 238 - DNA, 206 bp, synthetic construct
  source 1..206: mol_type = other DNA
gaaaatggaagggatccgcattctgggagccgtataagaaaaaggaagtatagggggaaagatattcctctattacgaatacgcccgtaaccgggcaaaagaaccgggggacgcccggggatagcttgcgaatgctcagtccataaggactgtcgaacaagaagcccccacttctacgcgaagcgaaagtggtgggagtacgtcac

SEQ ID NO: 239 - DNA, 203 bp, synthetic construct
  source 1..203: mol_type = other DNA
gacagggacgtgaacgccgcgttgaacattcgcgacgaggcattgcggctcagccgggaaaaagcatgacgaaagaagaggaaccggccgacaaccggggatagcccgctaaaaatgggaagcctctggattcagacgtatcgtctgaatccaagcaagcccagttcgcaggaatcccgtggtttcaaccacgggagtgtcaa

SEQ ID NO: 240 - DNA, 202 bp, synthetic construct
  source 1..202: mol_type = other DNA
gcagcaggtgtggagagattgttcccaaaagtttgaatattcgtattcatcactgccctcattgtgggctcacacttgaccgagatgtaaacgctgcccgtaatatccttgccctcggacggagggttgaaacgctaacgtatcccgttggggaaagcgtagtttcagaagctcccccgctttagcggggagagtgcgtcac

SEQ ID NO: 241 - DNA, 202 bp, synthetic construct
  source 1..202: mol_type = other DNA
gttcgtgtggtacggtgttctccgaactctccctggcagatcgctgggtcaactgcccctgcgggctttcgctggatcgtgatgtcaacgctgcactgaatatcctaaaaagggcgggacgcgccctgtgggacgaaagcactgccattgggctaaggctgtcccatgaagccccgctgctttagcagcaggggagcgtcac

SEQ ID NO: 242 - DNA, 201 bp, synthetic construct
  source 1..201: mol_type = other DNA
ggaactgtcagatagagtccacgaatgcccatattgtggtttctcatccaaccgcgactacaatgcagcagtaaacattctctccacagggatggaacagcccgtagcgcccatagagtcaaaacctctacatcacgtttctgtgatgcaagtcttggcgatgaagtgggaagccctaccctttagggtagggtagttcac

SEQ ID NO: 243 - DNA, 209 bp, synthetic construct
  source 1..209: mol_type = other DNA
gcaatcaacatacgtgaagaggcaagaaggatgttgacagcataaactgtccgtatccggacaagctgataataagaaaatatgcccgtatccgggcaaaagaatcgcggggcacgcgaggatagcttgtagatacttggctcagtagagccattgagcaagaagcccccacttcaaaatctatgatttaagtggtgggagcatgtcac

SEQ ID NO: 244 - DNA, 202 bp, synthetic construct
  source 1..202: mol_type = other DNA
gatctatatctgtgaatgcggaaacatcatcgatcgcgacttcaacagcgcgatcaacatagctttagaaggccgaagaatagtatcggcatagggctgggacagcccgaatataagcctgcggagcctatgtaagacttatgaataataagcagaaggctatgaaacaggaagctcggtcatttatggccgagcagttcac

SEQ ID NO: 245 - DNA, 201 bp, synthetic construct
  source 1..201: mol_type = other DNA
gcaatgcggcagtgaatataaaaaaagaaggcctgcgaatgctgaaatcagcgcagtaacctttcaccataaaggtaggctaggaactagccgaacctatacgcctgtggacattgtgtaagacctggcggtgcgcttctgcgctttcaggcagtagtggatgaagcaggaagctcggtaacttgttgccgagtagttcac

SEQ ID NO: 246 - DNA, 206 bp, synthetic construct
  source 1..206: mol_type = other DNA
gtcaaccccagaaacacatctcagatatgctccagatgtgggatgatcgtcgcaaagactctctctgatcgcgtccactcctgctcgcattgtgggttggtgatggatcgcgaccagaacgcggcgatcaatattatgagattggggctgcaatctcagggataacatcccaagatgcccccgacttcagtcgggggagcagtcac

SEQ ID NO: 247 - DNA, 201 bp, synthetic construct
  source 1..201: mol_type = other DNA
ggcgctatccacgacagggacgtgaacgccgcaagaaacattctaaacgaagggctgcggctaatggcgtagccaaaaacacttggtagggcgggacacgcccgaaccgatacgctcggggagaccatgtaagacctcacacggtgcaggcaacggtcagcgaaccgagaatcccccggctttagccgtggggagtgtcaa

SEQ ID NO: 248 - DNA, 202 bp, synthetic construct
  source 1..202: mol_type = other DNA
gacgtgaaacatcttcaccttcgtgaatgggagtgcccttcatgtggcacacatcacgatcgagatgtgaacgcaagtatgaatttaaaaaacgaagccattcggcttctaaccgcaggaactgcggggctagcctgctcattctcccttcgttagaagggacagcgcaggaatctcgtggcttcagccatgagaggttcaa

SEQ ID NO: 249 - DNA, 206 bp, synthetic construct
  source 1..206: mol_type = other DNA
gcatcacgaccgcgaccataacgcggcaaagaatatcctgcggcgagccttggagaacaaagccaaggcagcctagcatagcaaaagtaccgtggggcacacggaaactcacgcttggggagatcgtgtaagacgccaagtcctaggatgcggcgcagtggtcgtcgaaccaagaatcccccggatttatccgtggggagtgtcaa

SEQ ID NO: 250 - DNA, 201 bp, synthetic construct
  source 1..201: mol_type = other DNA
gccccgactgcggcacccgacatgatcgggacatcaacgccgcgaagaacatccttgtcgccgccgggctggcggagacacaaaacgcctgcggaggtgacgtcagaccgcatggggcatcccatcggcagtcgcctgtgaaacaggaaccctcgcaggcgactgcgagaatccccgtccttcagggcggggagtagtcaa

SEQ ID NO: 251 - DNA, 204 bp, synthetic construct
  source 1..204: mol_type = other DNA
gaatgggagtgcgggtgcggcgcccgccatgaccgcgacgtcaacgccgcgaagaacattctggccgccgggctggcggtcacggcctgtggagccgacgtaagacccaaacggcgcaagccgagcgggcagacggcgacgaagcaggaaacccaaccggcgacggttggaatcccccgcctttaggcgtggggaggaagtcaa

SEQ ID NO: 252 - DNA, 203 bp, synthetic construct
  source 1..203: mol_type = other DNA
gcttaatatccgaaactggacttgtccagaatgcaatacgcaccatgatcgtgatattaacgcagcgaagaatataaaaaccgctggactggcggggttagcttagggagcgactggatcggggatagcctcaaaagaggctatctagtgaaagcgtgttgaactaagaatcccctagcgaattcgctaggggagtatgtcaa

SEQ ID NO: 253 - DNA, 202 bp, synthetic construct
  source 1..202: mol_type = other DNA
gagatgccgttgcaggttcgcgcctggacgtgccccgagtgcgcagccgaacacgaccgcgacatcaacgccgcgatcaacattaaagccgccgggctggcggtgttagcccttggagagaatgtaagcggcagggagccagcctccgtgtccgattctcggtgaattgggaatctccgtccttcagggcgggggcagtcaa

SEQ ID NO: 254 - DNA, 202 bp, synthetic construct
  source 1..202: mol_type = other DNA
gccgacagcatagcccccatcaaacaatagaaagctatccacaaaaccgggtaggaacaccggggttagcctgtactactacggcatggacggtcggcatcgtcctataagaccggttaaacaggaaaatagagggagccggtcgggacattcgtccctcccacctcccgaagcccacgactttagtcgtgtggtagttcac

SEQ ID NO: 255 - DNA, 206 bp, synthetic construct
  source 1..206: mol_type = other DNA
gcccatactgcggcacgcatcatgaccgggacgtgaacgccgccaaaaacatccaagccgccggactggcggtcatagcctgcggagacggcaggacggcggaacgcaggaacatgcgtcccgccgatccgatccgtccgtgaaacaggaaaaccctatcgcgagatagggaatcccccgcctctccaggcgggggaggaagtcaa

SEQ ID NO: 256 - DNA, 207 bp, synthetic construct
  source 1..207: mol_type = other DNA
gatatcagggaatgggaatgtccgaactgtcataccatccatgacagagacataaacgccgcgataaatataaagcgtcaaggattacgcatgatgagtcatgaatttgagaaccgtgggacacacggggatagctcgttgatacttagcacaggggtgctattgagcgagaagcccccacttctataagtggtgggagcatgtcac

SEQ ID NO: 257 - DNA, 205 bp, synthetic construct
  source 1..205: mol_type = other DNA
gccaatattttggccgaggggctgcgactgctggcagtggttgccacctaataaaatatggcccatagatagcctatgagccatgctttggtagggtagggcatacccgaacctatacgcttggggacattgcgtaagacctcgctggggcaggctgcgatgaatgagccaagaatcccctgccttcaggcatggggagtgtcaa

SEQ ID NO: 258 - DNA, 201 bp, synthetic construct
  source 1..201: mol_type = other DNA
gaccgcgatatcaatgcggctcagaacatccttgaagaagggcttcgcaagcgggcgtccgcagcctgatatactttctgctcatggtagggcgggacacgcccgaaccaatacgcctggggagaccatgtaagacctcgctcgtgcaggctgtggtcgttgaaccaggaatcccccggctttagccgtggggagtgtcaa

SEQ ID NO: 259 - DNA, 118 bp, synthetic construct
  source 1..118: mol_type = other DNA
gaactcttggaacaagagggttagcttggtccatttcgtcagctggtaaaagtggcgagtacccaagaagcccccacttcaagcaacctgtaagggtggtaagtggtgggtagttcac

SEQ ID NO: 260 - DNA, 120 bp, synthetic construct
  source 1..120: mol_type = other DNA
gaacttttggaacaagagggttagcttggtcaatttcgtcagctgataaaagtggcgattacccaagaagcccccacttcaagcaactcgtaagggtggtaagtggtgggagtagttcac

SEQ ID NO: 261 - DNA, 118 bp, synthetic construct
  source 1..118: mol_type = other DNA
gaactcttggaacaagagggttagcttggttgatttcgtcagctatcaaaagtggcgattacccaagaagcccccacttcaagcaacccgtaagggtggtaagtggcgagtagttcac

SEQ ID NO: 262 - DNA, 118 bp, synthetic construct
  source 1..118: mol_type = other DNA
gaactcttggaacaagagggttagcttggtcaaattcgtcagctaccaaaagtggcgattacccaagaagcccccacttcaaacaacccgtaagggtggtaagtggtgggtagttcac

SEQ ID NO: 263 - DNA, 120 bp, synthetic construct
  source 1..120: mol_type = other DNA
gaactcttggaacaagagggttagcttggtccatttcgtcagctaccaaaagtggcgattacccaagaagcccccacttcaagcaacccgtaagggtggtaagtggtgggagtagttcac

SEQ ID NO: 264 - DNA, 109 bp, synthetic construct
  source 1..109: mol_type = other DNA
gaaccgtgggacacacggggatcgctcggtcaacttctcttcgctggaagggattacccgagaagccccccacctctaagcgtcagcgtaggtggtgggagtatgtcac

SEQ ID NO: 265 - DNA, 108 bp, synthetic construct
  source 1..108: mol_type = other DNA
gaaccgtggggcacacggggatcgctcggtcaacttcccatcatgagatgggattacccgagaagcccccacctctaagcgttagcgtaggtggtgggagtatgtcac

SEQ ID NO: 266 - DNA, 113 bp, synthetic construct
  source 1..113: mol_type = other DNA
gaactcttggaacaagagggttagctcggtctgttttcgccggctacttggagcggcgataagtcgagaagcccccacttcaaacgcgaagcgttaagtggtgggtagttcac

SEQ ID NO: 267 - DNA, 106 bp, synthetic construct
  source 1..106: mol_type = other DNA
gaagagcatggaataggctcgatagctaagtaaatttcgtaccgttaggtacgactaccttagaagcccccacctctaagcgaagcgtaggtggcgggtagttcac

SEQ ID NO: 268 - DNA, 118 bp, synthetic construct
  source 1..118: mol_type = other DNA
gaactcttggaacaagagggttagcttggtccatttcgtcagctaccaaaagtggcgattacccaagaagcccccacttcacgcaacctgtaagggtggtaagtggtgggtagttcac

SEQ ID NO: 269 - DNA, 105 bp, synthetic construct
  source 1..105: mol_type = other DNA
gaagagcatggaacaggctcgatagctaagtcaatttcgtaccattaggtgcgattaccttagaagcctccacctctaagcaagcgtaggtggcaggtagttcac

SEQ ID NO: 270 - DNA, 128 bp, synthetic construct
  source 1..128: mol_type = other DNA
gaacctttggaacaagggagttagctcggctgtcttaggactaattcgttagctatcaaaagtaacgactaaccgagaagcccccacttcaaacagtccgttaggacattaagtggcgggtagttcac

SEQ ID NO: 271 - DNA, 106 bp, synthetic construct
  source 1..106: mol_type = other DNA
gaaccgtagggactacggggatagctaagtccatttcgttccgttaggtacgactaccttagaagcccccacctctaagcataagtgtaggtggtgggtagttcac

SEQ ID NO: 272 - DNA, 105 bp, synthetic construct
  source 1..105: mol_type = other DNA
gaagagcctggaacaggctcaatagctaagtcaattcgtaccagcaggtacgactaccttagaagcccccacctccaagcgtagcgtaggtggagggtagttcac

SEQ ID NO: 273 - DNA, 114 bp, synthetic construct
  source 1..114: mol_type = other DNA
gaactcgtggaacacgaggggtagctcggtccgttttcgtcagctagcagaagtggcgataagtcgagaagcccccacttcaaaatctgaaagatttaagtgggggtagttcac

SEQ ID NO: 274 - DNA, 108 bp, synthetic construct
  source 1..108: mol_type = other DNA
gaaccgtggggcacacggggatcgctcggtcaacttcccttcacaagaagggattacccgagaagcccccacctctaagcgttagcgtaggtggtgggagtatgtcac

SEQ ID NO: 275 - DNA, 108 bp, synthetic construct
  source 1..108: mol_type = other DNA
gaaccgtggggcacacggggatcgctcggtcaactttccatcatgagatgggattacccgagaagcccccacctctaagcgtcagcgtaggtggtgggagtatgtcac

SEQ ID NO: 276 - DNA, 108 bp, synthetic construct
  source 1..108: mol_type = other DNA
gaaccgtggggcacacggggatcgctcggtcaattttccttcgttagaaggaattacccgagaagcccccacctctaagcgttagcgtaggtggtgggagtatgtcac

SEQ ID NO: 277 - DNA, 108 bp, synthetic construct; identical to SEQ ID NO: 265
  source 1..108: mol_type = other DNA

SEQ ID NO: 278 - DNA, 108 bp, synthetic construct
  source 1..108: mol_type = other DNA
gaaccgtggggcacacggggatcgctcggtcaacttcccatcatgagatgggattacccgagaagcccccacctctaagcgttagcgcaggtggtgggagtatgtcac

SEQ ID NO: 279 - DNA, 120 bp, synthetic construct
  source 1..120: mol_type = other DNA
gaactcttggaacaagagggttagctcggtcaacttagtcagctagtaaaagtggctattacccgagaagcccacaacttcaagcagaccgaaaggttagctaagtggtgggtagttcac

SEQ ID NO: 280 - DNA, 118 bp, synthetic construct
  source 1..118: mol_type = other DNA
gaacttttggaacaagagggttagcttggtttatttcgtcagctaccaagagtgacgattacccaagaagcccccactccaagcaccccgtaaaggtggtaagtggtgggtagttcac

SEQ ID NO: 281 - DNA, 118 bp, synthetic construct
  source 1..118: mol_type = other DNA
gaactcttggaacaagagggttagcttggtccatttcgtcagctaccaaaagggcgattacccaagaagctcccacttcaagcgacctcgcaagggtgataagtggtgggtagttcac

SEQ ID NO: 282 - DNA, 113 bp, synthetic construct
  source 1..113: mol_type = other DNA
gaactcctggaacaggagggttagctcggtcaattttctgtcatgagacaggattacccgagaagcccccacttcaagcaactcgaaagggtggtaagtggagggtagttcac

SEQ ID NO: 283 - DNA, 93 bp, synthetic construct
  source 1..93: mol_type = other DNA
gaacggcccggacggggccgccagagcgtaacgtggccggtcacggcgagcgtgctctgggaagctccccgctttagcggggagagtcgtcac

SEQ ID NO: 284 - DNA, 93 bp, synthetic construct
  source 1..93: mol_type = other DNA
gaacggcccggacggcgccgccagagcgtaacgtggccggtcacggcgagcgtgctctgggaagctccccgctttagcggggagagtcgtcac

SEQ ID NO: 285 - DNA, 93 bp, synthetic construct
  source 1..93: mol_type = other DNA
gaacggcccggacggggccgccagagcgtaacgtggccggttacggcgagcgtgctctgggaagctccccgctttagcggggagagtcgtcac

SEQ ID NO: 286 - DNA, 93 bp, synthetic construct
  source 1..93: mol_type = other DNA
gaacggcccggacggggccgccagcgcgtaacgtggccggttgcggcgagcgtgctctgggaagctccccgctttagcggggagagtcgtcac

SEQ ID NO: 287 - DNA, 93 bp, synthetic construct; identical to SEQ ID NO: 285
  source 1..93: mol_type = other DNA

SEQ ID NO: 288 - DNA, 93 bp, synthetic construct
  source 1..93: mol_type = other DNA
gaacggcccggacggggccgccagggcgtaacgtggccggttgcggcgcgcgtgctctggaaagctccctgctttagcggggagagtcgtcac

SEQ ID NO: 289 - DNA, 93 bp, synthetic construct
  source 1..93: mol_type = other DNA
gaagggcccggacggggccaccagggcgtaacgtggccggtagcggtgagcgtgccctgggaagctccctgctttagctgggagagtcgtcac

SEQ ID NO: 290 - DNA, 96 bp, synthetic construct
  source 1..96: mol_type = other DNA
gaaccgcccggacggggcggtcaggcgataacgtgccggaaccaactcggttgcgtagcctgagaagctccctgctttagcggggagagtcgtcac

SEQ ID NO: 291 - DNA, 93 bp, synthetic construct
  source 1..93: mol_type = other DNA
gaaccgctcggacggagcggtcagagcctaacgtggaccaacgggtcgagcgtgctctgagaagccgtcgcctttaggcgacggagtcgtcac

SEQ ID NO: 292 - DNA, 93 bp, synthetic construct
  source 1..93: mol_type = other DNA
gaaccgctcggacggagcggccagggcgtaacgtcggccagtgggtcaggcgtgccctgggaagccgccgcctttaggcggcggagtcgtcac

SEQ ID NO: 293 - DNA, 93 bp, synthetic construct
  source 1..93: mol_type = other DNA
gaaccgctcggactgagcggtcaagacggaacgtgggcggttacgccgagcgtgtcttgagaagctgtcgtctttaggcgacagagtggtcac

SEQ ID NO: 294 - DNA, 96 bp, synthetic construct
  source 1..96: mol_type = other DNA
gaatggcccggacggggccttcgggacgcaacgtgggcggaagtaacgccgagcgtgtcccgagaagctccccgctttagcggggagagtcgtcac

SEQ ID NO: 295 - DNA, 95 bp, synthetic construct
  source 1..95: mol_type = other DNA
gaaggcacggactgtgccttggggcgataacgtggcagtagttgacgcgagcgtagcctcagaagccgccttgctttagctggcggagttgtcac

SEQ ID NO: 296 - DNA, 93 bp, synthetic construct
  source 1..93: mol_type = other DNA
gaagcgctcggactgagcgttcaggacttaacgtgggcgaacgcgccgtgcgtgtcctgagaagctgtcgctcttaagcaacagagtagtcac

SEQ ID NO: 297 - DNA, 96 bp, synthetic construct
  source 1..96: mol_type = other DNA
gaaggcacggacctgtgccttggggcgataacgtggcggtagttgacgcgagcgtagccccagaagccgccttgctttagctggcggagttgtcac

SEQ ID NO: 298 - DNA, 94 bp, synthetic construct
  source 1..94: mol_type = other DNA
gaaccgctcggacggagcggtcagccgggaacgtgggtggctacaccgtgcgttggctgagaagccgccgcctttaggcgacggagaacgtcac

SEQ ID NO: 299 - DNA, 93 bp, synthetic construct
  source 1..93: mol_type = other DNA
gaaccgcctgggctgggcggtcaggacgtaacgtacaccgttgggtgatgcgtgtcctgagaagccgcctgctttagcaggcggagtagtcac

SEQ ID NO: 300 - DNA, 94 bp, synthetic construct
  source 1..94: mol_type = other DNA
gaaccgctcggacggagcggtcaggcggtaacgtgggtgggtacaccgtgcgtcgtctgagaagctgtcggctttagccgatagagaacgtcac

SEQ ID NO: 301 - DNA, 94 bp, synthetic construct
  source 1..94: mol_type = other DNA
gaaccgctcggacggagcggtcaggcggtaacgtgggtgggtacaccgtgcgtcgcctgagaagctgtcggctttagccgatagagaacgtcac

SEQ ID NO: 302 - DNA, 93 bp, synthetic construct
  source 1..93: mol_type = other DNA
gaacggcccggacggggccgccagggcgtaacgtggccggttgcggcgagcgtgctctgggaagctccccgctttagcggggagagtcgtcac

SEQ ID NO: 303 - DNA, 93 bp, synthetic construct
  source 1..93: mol_type = other DNA
gaacggcccggacggggccgccagagcagaacgtggccggttacggcgagcgtgctctgggaagctccccgctttagcggggagagtcgtcac

SEQ ID NO: 304 - DNA, 93 bp, synthetic construct
  source 1..93: mol_type = other DNA
gaacggctcggatcgagccttcaggacagaacgtgcgccgatgggcggagcgtgtcctgagaagctgtctgcttgagctgacagagtgatcac

SEQ ID NO: 305 - DNA, 93 bp, synthetic construct
  source 1..93: mol_type = other DNA
gaacggcccggacggggccgccagagcgtaacgtggccggctgcggcgcgcgtgctctgggaagctccccgctttagcggggagagtcgtcat

SEQ ID NO: 306 - DNA, 93 bp, synthetic construct
  source 1..93: mol_type = other DNA
gaaccgcccggacggggcggtcaggcgctaacgtggcccgttggggcgcgcgtagcctgagaagctccctgctttagcggggagagtcgtcac

SEQ ID NO: 307 - DNA, 90 bp, synthetic construct
  source 1..90: mol_type = other DNA
gaacagcctggatggggctgtcaggacgtaacgtgggcggctgcgccgtgcgtgtcctgagaagccgccgcatacggcggagaacatcac

SEQ ID NO: 308 - DNA, 94 bp, synthetic construct
  source 1..94: mol_type = other DNA
gaaccgcttgggctgagcggacaggacttaacgtggaacaatagttcgtgcgtgtcctgtgaagctgtcattctttaggagacagagtagtcac

SEQ ID NO: 309 - DNA, 94 bp, synthetic construct
  source 1..94: mol_type = other DNA
gaaccgcatggacgatgcggtcagccggtaacgtgggcggttgcgccgggcgttggctgagaagccgcctcgtttacggggcggagaacgtcac

SEQ ID NO: 310 - DNA, 168 bp, synthetic construct
  source 1..168: mol_type = other DNA
gcaaggctttggaagacaaatccaaagttgcatgaacggaaatagaacataccgtggggcacacgggaattcacgcttggggagatcgtgtaagacgccaattcctcggaggcggcgcagtggtcgtcgaaccaagaatcccctgcctttaggcatggggagtgtcaa

SEQ ID NO: 311 - DNA, 168 bp, synthetic construct
  source 1..168: mol_type = other DNA
gcaaagctttggaagacaaatccaaagttgcatgaacgaaaataaaacataccgtggggcacacgggaattcacgcttggggagatcgtgtaagacgccaattcctcggaggcggcgcagtggtcgtcgaaccaagaatcccctgcctttaggcatggggagtgtcaa

SEQ ID NO: 312 - DNA, 168 bp, synthetic construct
  source 1..168: mol_type = other DNA
gcaaggctttggaagacaaataccaagttgcgtgaacggaaatagaacataccgtggggcacacgggaatttacgcttggggagatcgtgtaagacgccaattcctcggaggcggcgcagtggtcgtcgaaccaagtatcccctgcctttaggcatggggagtgtcaa

SEQ ID NO: 313 - DNA, 168 bp, synthetic construct
  source 1..168: mol_type = other DNA
gcaaggctttggaagacaaatccaaagttgcatgaacggaaataaaacataccgtggggcacacgggaattcacgcttggggagatcgtgtaagacgccaattcctcggaggcggcgcagtggtcgtcgaaccaagaatcccctgcctttaggcatggggagtgtcaa

SEQ ID NO: 314 - DNA, 168 bp, synthetic construct
  source 1..168: mol_type = other DNA
gcaaggctttggaagacaaatccaaagttgcatgaacggaaataaaacataccgtggggcacacgggaattcacgcttggggagatcgtgtaagacgccaattcctcggaggcggcgcagtggtcgtcgaaccaagaatcctctgcctttaggcatggggagtgtcaa

SEQ ID NO: 315 - DNA, 168 bp, synthetic construct; identical to SEQ ID NO: 313
  source 1..168: mol_type = other DNA

SEQ ID NO: 316 - DNA, 168 bp, synthetic construct; identical to SEQ ID NO: 310
  source 1..168: mol_type = other DNA

SEQ ID NO: 317 - DNA, 169 bp, synthetic construct
  source 1..169: mol_type = other DNA
gcaaggctttggaggacagatccacagcggcataagctgaaatgaaaatgtaccgtggggcacacgggaattcacgcttggggaaatcgtgtaagacgccaattcttcggaggcggcgcagtggttgtcgaaccaagaatcccctgccttcaggcatggggagtgtcaa

SEQ ID NO: 318 - DNA, 168 bp, synthetic construct
  source 1..168: mol_type = other DNA
gcaaggctttggaagacaaatccaaagtggcgtgagctagaataaaacgtaccgtgggacacacgggaactcacgcttggggagatcgtgtaagacgccaattccttggaggcggcgcagtggtcatcgaaccaagaatcccctggatttatccatggggagtgtcaa

SEQ ID NO: 319 - DNA, 170 bp, synthetic construct
  source 1..170: mol_type = other DNA
gcaaggctttggaggacagatccacagcggcataagctgaaataaatatgtaccgtggggcacacgggaattcacgcttggggaaatcgtgtaagacgccaattcttcggaggcggtgcagtgattgttgaaccaagaatcccctgcctttcaggcatggggagtgtcaa

SEQ ID NO: 320 - DNA, 169 bp, synthetic construct
  source 1..169: mol_type = other DNA
gcaaggctttggaggacagatccacagcggcataagctaaaatgaaaatgtaccgtggggcacacgggaattcacgcttggggaaatcgtgtaagacgccaattcttcggaggcggcgcagtggttgtcgaaccaagaatcccctgccttcaggcatggggagtgtcaa

SEQ ID NO: 321 - DNA, 168 bp, synthetic construct
  source 1..168: mol_type = other DNA
gcaaggctttggaagataaatccacagtggcataaactggaataaagtgtaccgtggggcacacgggaattcacgcttggggaaatcgtgtaagacgccaattcttcggaggcggcgcagtggttgtcgaaccaagaatcccctgccttcaggcatggggagtgtcaa

SEQ ID NO: 322 - DNA, 168 bp, synthetic construct
  source 1..168: mol_type = other DNA
gcaaggctttggaagataaatccacagtggcataaactggaataaagtgtaccgtggggcacacgggaattcacgcttggggaaatcgtgtaagacgccaattcttcggaggcggcgcagtggttgtcgaaccaggaatcccctgccttcaggcatggggagtgtcaa

SEQ ID NO: 323 - DNA, 169 bp, synthetic construct
  source 1..169: mol_type = other DNA
gcaaggctttggaggacagatccacagcggcataagctgaaataaatatgtaccgtggggcacacgggaattcacgcttggggaaatcgtgtaagacgccaattctttggaggcggcgcagtggttgtcgaaccaagaatcccccggctttagccgtggggagtgtcaa

SEQ ID NO: 324 - DNA, 120 bp, synthetic construct
  source 1..120: mol_type = other DNA
gaaccgctggacgagcggaaatctacgcttggggaaatcgtgtaagtcgttccggctacggctggaatgcagtggttgttgaaccaagaagcccctgcctttaggcatggggtgcgtcac

SEQ ID NO: 325 - DNA, 120 bp, synthetic construct
  source 1..120: mol_type = other DNA
gaaccgtggggcacacgggaattcacgcttggggagaacgtgtaagtcggaataactacggttaggccgcagtattcagcgaaccaagaatcccctggcttcagccatggggagtgtcaa

SEQ ID NO: 326 - DNA, 120 bp, synthetic construct
  source 1..120: mol_type = other DNA
gaaccgtggggcacacgggaattcacgcttggggagaacgtgtaagtcagtctggctacggtcaaaccgcagtgttcatcgaaccaagaatcccctgcctttaggcatggggagtgtcaa

SEQ ID NO: 327 - DNA, 120 bp, synthetic construct
  source 1..120: mol_type = other DNA
gaaccgtgggacacacggaaattcacgcttggggagaacgtgtaagtcggaataactacggttaggccgcagtgttcagtgaaccaagaatcccctgcgtttacgcatggggagtgtcaa

SEQ ID NO: 328 - DNA, 120 bp, synthetic construct
  source 1..120: mol_type = other DNA
gaaccgtgggacacacgggaattcacgcttggggagaacgtgtaagtcggaataactacggttagtccgcagtgttcagcgaaccaagaatcccctgcctttaggcatggggagtgtcaa

SEQ ID NO: 329 - DNA, 108 bp, synthetic construct
  source 1..108: mol_type = other DNA
gaaccgtggggcacacgggaagttacgcttgtggagagagtgtaagacgcattgcgcagttctctgtgaagcaagaatccctccggctttagccgtggggagtgtcaa

SEQ ID NO: 330 - DNA, 165 bp, synthetic construct
  source 1..165: mol_type = other DNA
ggcgagccttggagaacaaagccaaggcagcctagcatagcaaaagtaccgtggggcacacggaaactcacgcttggggagatcgtgtaagacgccaagtcctaggatgcggcgcagtggtcgtcgaaccaagaatcccccggatttatccgtggggagtgtcaa

SEQ ID NO: 331 - DNA, 169 bp, synthetic construct
  source 1..169: mol_type = other DNA
gcaaggctttggaggataaatccacagcggcataagctgaaataaatatgtaccgtgggacacacgggaattcacgcttggggagatcgtgtaagacgccaattcttcggaggcgacgcattggtcgtcgaaccaagaatcccctgcctttaggcatggggagtgtcaa

SEQ ID NO: 332 - DNA, 168 bp, synthetic construct
  source 1..168: mol_type = other DNA
gcaaggctttggaagacaaatccaaagtagcgtgaactgaaataaaacgtaccgtggggcacacgggaactcacgcttggggagatcgtgtaagacgccaattccttggaggcggtgcagtggtcgtcgaaccaagaatctctcggctttagccgtagggagtgtcaa

SEQ ID NO: 333 - DNA, 167 bp, synthetic construct
  source 1..167: mol_type = other DNA
gcaaggctttggaagacaaatccaaagtagcgtgaactgagataaaacgtaccgtggggcacacgggaactcacgcttggggagatcgtgtaagacgctaattcctcggaggcggcgcagtggtcgtcgaaccaagaatccccggctttagccgtggggagtgtcaa

SEQ ID NO: 334 - DNA, 120 bp, synthetic construct
  source 1..120: mol_type = other DNA
gaaccgtggggcacacgggaaatcacgcttggggagaacatgtaagtcgctttggctacggtcagaacgctgtgttcgtcgaaccaagaatcccctggatttatccatggggagtgccaa

SEQ ID NO: 335 - DNA, 120 bp, synthetic construct
  source 1..120: mol_type = other DNA
gaaccgtggggcacacgggaaatcacgcttggggagaacgtgtaagtcgctttggctacggtcagaacgctgtgttcgtcgaaccaagaatcctctggatttatccatggggagtgtcaa

SEQ ID NO: 336 - DNA, 120 bp, synthetic construct
  source 1..120: mol_type = other DNA
gaaccgtgggacacacggaaatctacgcttggggagagcgtgtaagacatataggccacggcctatatgcagtgctcagggaaccaagaatcccctgcctttaggcatggggagtgtcaa

SEQ ID NO: 337 - DNA, 120 bp, synthetic construct
  source 1..120: mol_type = other DNA
gaaccgtgggacacacggaaatctacgcttggggagagcgtgtaagacatataggccacggtctatatgcaatgctcagggaaccaagaatcccctgcctttaggcatggggagtgtcaa

SEQ ID NO: 338 - DNA, 120 bp, synthetic construct
  source 1..120: mol_type = other DNA
gaaccgtgggacacacggaaatctacgcttggggagaacgtgtaagacatataggccacggcctatatgcagtgctcatggaaccaagaatctcctgcctttaggcatggggagtgtcaa

SEQ ID NO: 339 - DNA, 120 bp, synthetic construct; identical to SEQ ID NO: 336
  source 1..120: mol_type = other DNA

SEQ ID NO: 340 - DNA, 120 bp, synthetic construct
  source 1..120: mol_type = other DNA
gaaccgtgggacacacggaaatctacgcttggggagagcgtgtaagacatataggccacggcctatatgcagtgctcagggaaccaagaatcccctgcctttaggcattgggagtgtcaa

SEQ ID NO: 341 - DNA, 121 bp, synthetic construct
  source 1..121: mol_type = other DNA
gaaccgaaggacggtcgggaattcacgcctgtggagattctgtaagacgttgcggcatctgccgtggcgcaggggtcagcgaatcaggaatcccctgcctttaggcatggggagccgtcaa

SEQ ID NO: 342 - DNA, 112 bp, synthetic construct
  source 1..112: mol_type = other DNA
gaaccgtgggacacacgggaagttacgcttgcggagattatgtaagacgcaatcacggcgcaacagtctttgaaacaagaatccctccgcatttatgcgtggggagtgtcaa

SEQ ID NO: 343 - DNA, 120 bp, synthetic construct
  source 1..120: mol_type = other DNA
gaaccgtgggacacacggaaatctacgcttggggagagcgtgtaagacatataggccacggcctatatgcagtgctcagggaaccaagaatccccagcctttaggcatggggagtgtcaa

SEQ ID NO: 344 - DNA, 120 bp, synthetic construct
  source 1..120: mol_type = other DNA
gaaccgtgggacacacggaaatctacgcttggggagagcgtgtaagacatataggccatggcctatatgcagtgctcagggaaccaagaatcccctgcctttaggcatggggagtgtcaa

SEQ ID NO: 345 - DNA, 108 bp, synthetic construct
  source 1..108: mol_type = other DNA
gaaatagagcatggaacaggcttgatagctaagtaaatttcgtaccgttaggtacgattaccttagaagccccacctctaaatgaagcgtaggtggcgggtagttcac

SEQ ID NO: 346 - DNA, 107 bp, synthetic construct
  source 1..107: mol_type = other DNA
gaaccgtggggcacacggggatcgctcggtcaacttcccatcatgagatgggattacccgagaagcccccacctctaagcgaagcgtaggtggtgggagtatgtcac

SEQ ID NO: 347 - DNA, 107 bp, synthetic construct; identical to SEQ ID NO: 346
  source 1..107: mol_type = other DNA

SEQ ID NO: 348 - DNA, 115 bp, synthetic construct
  source 1..115: mol_type = other DNA
gaactcgtggaacacgagggatagctcggtccgttctcgtcagctggtaaaagtggcgagaagtcgagaagcccccacttcaaaatctgaaagatttaagtggtgggtagttcac

SEQ ID NO: 349 - DNA, 109 bp, synthetic construct
  source 1..109: mol_type = other DNA
gaaatagagcatggaacaggctcgatagctaagtaaatttcgtaccattaggtacgattaccttagaagcccccacctctaagcaaagcgtaggtggcgggtagttcac

SEQ ID NO: 350 - DNA, 114 bp, synthetic construct
  source 1..114: mol_type = other DNA
gaactcgtggaacacgaggggtagctcggtccgttatcgtcagctagcagaagtggcgataagtcgagaagcccccacttcaaaatctgaaagatttaagtgggggtagttcac

SEQ ID NO: 351 - DNA, 110 bp, synthetic construct
  source 1..110: mol_type = other DNA
gaaatagagcatggaacaggctcgatagctaagtaaatttcgtaccgttaggtacgattaccttagaagccccccacctctaagcgaagcgtaggtggcgggtagttcac

SEQ ID NO: 352 - DNA, 109 bp, synthetic construct
  source 1..109: mol_type = other DNA
gaaatagagcatggaacaggctcgatagctaagtaaatttcgtaccgttaggtacgattaccttagaagcccccacctctaagcaaagcgtaggtggcgggtagttcac

SEQ ID NO: 353 - DNA, 116 bp, synthetic construct
  source 1..116: mol_type = other DNA
gaactcttggaacgagagggatcgctcggtccgttatcgttggctactaaaagcaacgataagtcgagaagcccccacttcaataatcgtcaggttataagtggtgggtagttcac

SEQ ID NO: 354 - DNA, 116 bp, synthetic construct
  source 1..116: mol_type = other DNA
gaactcttggaacgagagggatcgctcggtccgttatcgttggctactaaaagcaacgataagtcgagaagcccccacttcaataatcgttaggttataagtggtgggtagttcac

SEQ ID NO: 355 - DNA, 116 bp, synthetic construct
  source 1..116: mol_type = other DNA
gaactcttggaacgagagggatcgctcggtcagttatcgttggctactaaaagcaacgataagtcgagaagcccccacctcaataatcgtcaggttataagtggtgggtagttcac

SEQ ID NO: 356 - DNA, 107 bp, synthetic construct; identical to SEQ ID NO: 346
  source 1..107: mol_type = other DNA

SEQ ID NO: 357 - DNA, 108 bp, synthetic construct
  source 1..108: mol_type = other DNA
gaaccgtggggcacacggggatcgctcggtcaatttcccttcgttagaagggattacccgagaagcccccacctctaagcgttagcgtaggtggtgggagtatgtcac

SEQ ID NO: 358 - DNA, 106 bp, synthetic construct
  source 1..106: mol_type = other DNA
gaaccgtggggcacacggggatcgctcggtcaacttcccatcatgagatgggattacccgagaagccccacctctaagcgaagcgtaggtggtgggagtatgtcac

SEQ ID NO: 359 - DNA, 116 bp, synthetic construct
  source 1..116: mol_type = other DNA
gaactcttggaacaagagggatagctcggtcagttatcgttggctactaaaagcaacgataagtcgagaagcccctacttcaataaccgcaaggttataagtggtgggtagttcac

SEQ ID NO: 360 - DNA, 116 bp, synthetic construct
  source 1..116: mol_type = other DNA
gaactcttggaacgagagggatagctcggtcagttatcgttggctactaaaagcaacgataagtcgagaagcccccacttcaataatcgttaggttataagtggaggggagttcac

SEQ ID NO: 361 - DNA, 108 bp, synthetic construct
  source 1..108: mol_type = other DNA
gaatagagcatggaacaggctcgatagctaagtaaatttcgtaccgttaggtgcgattaccttagaagcccccacctctaagcgaaacgtaggtggcgggtagttcac

SEQ ID NO: 362 - DNA, 109 bp, synthetic construct
  source 1..109: mol_type = other DNA
gaatagagcatggaacaggctcaatagctaagtaaatttcgtaccgttaggtatgactaccttagaagcccccacctctaagcgttagcgaaggtggtgggtagttcac

SEQ ID NO: 363 - DNA, 107 bp, synthetic construct
  source 1..107: mol_type = other DNA
gaaccgtgggacacacggggatagctcggtcaatttctcatcatgagatgagattacccgagaagcccccacctctaagcgaagcgtaggtggtgggagcatgtcac

SEQ ID NO: 364 - DNA, 108 bp, synthetic construct
  source 1..108: mol_type = other DNA
gaatagagcatggaataggctcgatagctaagtaaatttcgtaccgtgaggtacgattaccttagaagcccccacctctaagcgaagcgtaggtggcaggtagttcac

SEQ ID NO: 365 - DNA, 116 bp, synthetic construct
  source 1..116: mol_type = other DNA
gaactcttggaacaggagggatagctcggtcagttatcgttggctactaaaagcaacgataagtcgagaagcccccacttcaataatcgtcaggttataagtggtgggtagttcac

SEQ ID NO: 366 - DNA, 108 bp, synthetic construct
  source 1..108: mol_type = other DNA
gaatagagcatggaacaggctcgatagctaagtcaatttcgcaccgttaggttcgattaccttagaagcccccacctctaagcgaagcgtaggtggtgggtagttcac

SEQ ID NO: 367 - DNA, 110 bp, synthetic construct
  source 1..110: mol_type = other DNA
gaatagagcatggaacaggctcgggaagctaagtcaatttcgtaccgataggtgcgattaccttagaagcccccacctcaaagagtttgcgtaggtggagggtagttcac

SEQ ID NO: 368 - DNA, 108 bp, synthetic construct
  source 1..108: mol_type = other DNA
gaaccgtggggcacacggggatcgctcggtcaacttcccttcgctagaagggattacccgagaagcccccacctctaagcgttagcataggtggtgggagtatgtcac

SEQ ID NO: 369 - DNA, 204 bp, synthetic construct
  source 1..204: mol_type = other DNA
gtaaatgcagcaagaaatatactggctgaaggactcaggataagagctctgactccagggtcttagtagtacagaagcaaacaggaaccgcaggaattgcgggggtagcttggtaaacaagagaaacctctgccggaaaagaaataagccggtaagtacgctctgttcccaagaatctcgtgactttagtcatgagagtttcaa

SEQ ID NO: 370 - DNA, 204 bp, synthetic construct
  source 1..204: mol_type = other DNA
gtcaacgcagccagaaacatactggcagaaggactcagactaagagctctaaagctggagtcttagagaaataaatgcatctgcgaaccgcaggaaatgcggggatagcttggtaaacaagagaaacctctgccggcaaagaaataagccggtaagtatgctctcttcccaagaatctcgtgacttcagtcatgagaatttcaa

SEQ ID NO: 371 - DNA, 202 bp, synthetic construct
  source 1..202: mol_type = other DNA
gtggtcggcgcaaagaacatccttgctcgcgggatggccattgttgcaagcgagatgaagggcggggcacgcccggatggcctgtggatcgaacctcaatggaggtcggaagcaggaacccaccgaagcgactcgggagcgcctcaatgctgcgcctgagcgccgtaggaatctccggccttcaggccggggaggatgtcaa

SEQ ID NO: 372 - DNA, 202 bp, synthetic construct
  source 1..202: mol_type = other DNA
gcgtggaatgcggttatacgaataatgccgatgtggtcggcgcaatcaatattctaagggcaggacatgcccggttcgcctgtgaagtgaatcctgatgtaggggatcagcagcaggaacccgccgaggtgattcagtatggcaacaatgccacgttggacgcagtaggaatccccttgctttagcaaggggaggatgtcaa

SEQ ID NO: 373 - DNA, 203 bp, synthetic construct
  source 1..203: mol_type = other DNA
gccgatgtcgtcggcgcgatcaatattctcagggcgggacacgcccgttgcgcctgtccagtgagcggtgcagcaatgccgccagcagcaggaacccaccgaagcgactcaggggtggcaccatgtcacgcctgagcgccgtaggaatccccgcccttcccgcgcaagcggtggccgaaggccaaggacggggaggatgtcaa

SEQ ID NO: 374 - DNA, 209 bp, synthetic construct
  source 1..209: mol_type = other DNA
gattcaaatattgcgagacgaagggcaggacacgactgacgcttcggtcgggatgctggcaggtagtctgcttgcatcagcccggatggcctgtggatcgaatcacgctagtgatcggaagcaggaacccaccgaaacggttgcgtatggaggtaactatgcgtaatcgtggtaggaatcctcggcctttagaccggggaggatgtcaa

SEQ ID NO: 375 - DNA, 206 bp, synthetic construct
  source 1..206: mol_type = other DNA
gaccacgtcggcgccatcaatgtgttaagggcggggcacgcccggttagcctgtgaagtgagtggtgcggtaaggccgccagcagcaggaacccaccgaagcgactcaggagcggctcaatgccgcgcctgagcgccgtaggaatccccttcctttccgccgcaggcggcagcttcctaagaagctgagggaggggaggacgtcaa

SEQ ID NO: 376 - DNA, 204 bp, synthetic construct
  source 1..204: mol_type = other DNA
gaatgtcctcacacgaggattgcaactgttggaagggcaggacgatgcccggatagcccgtggagagatggcgcagtcaggccactcggtgaagcgggaacccgccgacgcgactacgtggaacatcacgtagcgcagtaggaattctcccccttcccggccttgggccggcgagcgtcagcgagagggggagaaggatgtcaa

SEQ ID NO: 377 - DNA, 202 bp, synthetic construct
  source 1..202: mol_type = other DNA
gccgacgtggtgggcgcgatcaacattctggcgcggggacaccgcgttgcagcctgtggagagccggtgcagtcaggccgctcgacgaagcaggaacccgccgaggcgatctgaaatggcaatcatcgtgccagacgcagtaggaatcctcgcccttcccgcgcaagcggtggccgaaggccaagggcggggaggatgtcaa

SEQ ID NO: 378 - DNA, 201 bp, synthetic construct
  source 1..201: mol_type = other DNA
gcggaaccgtccatgaccgggatatcaatgcagcgaagaatattttaaaagaaggactgcgcatgctggcgtagtatgcctctatggtagggcgggacacgcccgaaccgaacgctcggggaccttgtataagaccttgcagatgcagggctgtgagggaagaaccgagaatcccctgcctttaggcatggggagtgtcaa

SEQ ID NO: 379 - DNA, 201 bp, synthetic construct
  source 1..201: mol_type = other DNA
gcggagcagtccacgaccgggatgtgaacgccgcgaagaatattttgaaggaagggcttcgcctgctggcgtagtcagacgatacggtagggcgggacacgcccgaacctgaacgctcggggacatcgtgtaagacctcgcatgggcaggcagtgatggatgagccgagaatcccccggctttagccgtggggagtgtcaa

SEQ ID NO: 380 - DNA, 201 bp, synthetic construct
  source 1..201: mol_type = other DNA
gttcgggcgtggacatgtcctttctgcggaacggttcacgatcgggacttaaacgccgcgaaaaacatttggaaggaagggctacgcctgttggtgtagctaaggcatacagtagggcgggacacgcccgaacctacgctcagggacatcgtcgtatggatgaactgagaatcccccggctttagccgtggggagtgtcaa

SEQ ID NO: 381 - DNA, 202 bp, synthetic construct
  source 1..202: mol_type = other DNA
gtggcgccgcccataatcgggacaccaacgctgcaaagaacatcctgaacgaagggctgcgcctcttggcgcagcagaaacatacggtagggcgggacacgcccaaacctatacgctcggggagaccctataagtcctcgcacgttgcaggctgcggtcaacgaaccgagaatcccccggctttagccgtggggagtgtcaa

SEQ ID NO: 382 - DNA, 201 bp, synthetic construct
  source 1..201: mol_type = other DNA
gtgcggtactaaccacgaccgcgatattaacgccgcaagtaatattttaaaagaggcactaagattgctgagtgcctaaagataatgagtaccgtgggacgcacgggaattaacgcttggggagatcatgtaagacctcgcaagtgcaggcggtgatcggtgaaccaagaatcccccagatttatccgtggggagtgtcaa

SEQ ID NO: 383 - DNA, 201 bp, synthetic construct
  source 1..201: mol_type = other DNA
gcggagccgtccacgaccgggatatcaacgctgcgaaaaacatcctgactgaaggccttcgtctgctggcttgaatcgcaggcaaggtagggcgggtcacgtccgaaccaatacgctcggggacatcgtgtaagacctcgcatcagcaggctgtggtggttgaaccgagaatcccccggatttatccgtggggagtgtcaa

SEQ ID NO: 384 - DNA, 205 bp, synthetic construct
  source 1..205: mol_type = other DNA
gccccgtttgcggcactgtccacgaccgggacgtaaatgccgccaaaaacatcttgaacgaaggtctgcgtctcttggcgtaggctatacatggtagggcgggacacgcccgaacctaacgctcggggagagtgcataagacgcaaccacggcgctatactcagagaaccgagaatcccccggattcatccgtggggagtgtcaa

SEQ ID NO: 385 - DNA, 201 bp, synthetic construct
  source 1..201: mol_type = other DNA
gcggcacagtccacgacagggattgcaacgccgcaaagaacattctcaacgaaggacttcgcctgctggtgtagtcagaacatacggtagggtgggacacacccgaacccatacgctcggggagaccatgtgagacctcgcaagtgcaggctgcggtcagcgaaccgagaatcccctgcctttaggcatggggagtgtcaa

SEQ ID NO: 386 - DNA, 202 bp, synthetic construct
  source 1..202: mol_type = other DNA
gaccgcgatatcaatgcgacgcagaacatcctggaagaagggcttcgcaagcgggcatccacagcctgatatacttttctgctcgtggtagggcgggacacgcccaaacctatacgcttggggagaccatgtaagacctcgctcgtgcaggctacggtcgttgagccaagaatcccctgtctttagacatggggagtgtcaa

SEQ ID NO: 387 - DNA, 203 bp, synthetic construct
  source 1..203: mol_type = other DNA
gtcccgattgcggcacccggcacgaccgggatgtgaacgccgctaagaacatcatcgtcgcggccggactggtcgagacacaaaacgcctgcggagcagatgcaagaccgaaacgggcaaccgttcggcgatctgcggtgaaacaggaaccccagcctgcgaaggttggaatcccatgcctttagacatggggaggaagtcaa

SEQ ID NO: 388 - DNA, 202 bp, synthetic construct
  source 1..202: mol_type = other DNA
gtccgggctgcggcacccgacatgatcgggacgtcaacgccgctaggaacatcatgctcgccgccggactggtggagagtcgaaacgcctgcggagccgacgtcagacgcgacgggcaaccgttcgcgctgtctgcgttgaaccaggaaatccgacccgcaagggtaggaaatcccggcacttaaggccgggaggatgtcaa

SEQ ID NO: 389 - DNA, 201 bp, synthetic construct
  source 1..201: mol_type = other DNA
gcacccggcacgaccgggaccacaacgcggccaagaacatccttgcggctggtcgagccgtagccggggcaggccccggcgatgtctgtggagccgatgtcagacggcaagggccttcccttccgcagtcggctgcgaagcagaaacccccacggcgcgagccgtaggaatcccttccatccggggaggggaggaagtcaa

SEQ ID NO: 390 - DNA, 201 bp, synthetic construct
  source 1..201: mol_type = other DNA
gccaagaacatccttgcggctggtcgagccgtagcccgaggtgactcgggtgatgcctgcggagctgatgtcagacggcaagggccctcccttccgcgatcggcagtgaaacaggaatcccgcgccgcgaggcgtagcccgtgagggttacccgccgcaaggcgggtgggaatctccgtccttcagacggagagaagtcaa

SEQ ID NO: 391 - DNA, 204 bp, synthetic construct
  source 1..204: mol_type = other DNA
gcacccggcacgaccgggacatcaacgccgccaagaacatcctcgcggcaggtcttgccgtagccccgggctccccgggcgatgcctgcggagctggcataagacaccccggaacctcctgggtgcggccggcgatgaaacaggaaccccagccggtgacggctggaatccccgtccttcagcttcagggcgaggagtagtcaa

SEQ ID NO: 392 - DNA, 203 bp, synthetic construct
  source 1..203: mol_type = other DNA
gcactggatgtgcccgggatgcggcacccggaacgaccgggacgtcaacgccgccaagaacatcctggccgctggtcgagcggtgtccgcctgtggagctgacgtaagtcactccgggtcctcccggatgcggtcggccgtgaaacaggaagcccagcccgcgagggcgggagtcccccgcctgtaggcgggggggaagtcaa

SEQ ID NO: 393 - DNA, 205 bp, synthetic construct
  source 1..205: mol_type = other DNA
gccaagaacatccttgcggctggtcgagtcgcagcccgagaacactcgggtgatgcctgtggagctggtgtcagtcggcaagggtcctcccttccgcagtcggcaacgaagcaggaaaccgggcctgtgagggccgggccttagggcccatacgcgcgcaagcgcgtatcggaatcccctcccttcggggagaggaggaagtcaa

SEQ ID NO: 394 - DNA, 202 bp, synthetic construct
  source 1..202: mol_type = other DNA
gcgaagaacatccttgcggctggtcgagccgtagcccgagacaactcgggtgatgcctgcggagctgatgtcagacggcaagggccctcccttccgcgatcggctgtgaaacaggaaccccgtgtcgtgaggcacggcgcgtgagcgctacctgccgcgaggcaggtgggaattcctgtccgtcaggacgggaagaagtcaa

SEQ ID NO: 395 - protein, 438 aa, synthetic construct
MSVPRKKNKEPDNIIEITHRYRAYLTALQEYKAESWLYTLCNLYNSAIKERRNAYRTEKKTITYSQQQNGLLLLKDSDPTLKKVHSQLLQNCLQRVDRAYQKFFEDLKRKKNGKHVKVGYPRLKKLSKYRSFTYPQVWMKQKGELKQVIKFRQDNNKFGTTVLPGFGEVRIRVHRPLDWKQAKTVILKREKSGKWYLCITLKKAVELQLQDNGKSTGIDLGLKKIVNTSDTNYQEHPKFIYRSEARIKRAQKRLSRKQKGSANYEKQRIRLARLHEKVANQRRDFLHKLALWLVVNYSFIAFERLNIRGMVKNHHLAKAILDAGWATLITLTTYKSVMLRGNEVVRVDPRYTSQECSACHALVPKALAERIHKCPYCGIELDRDTNAARVIEQKAFLRETTPFRSPRAGAVRSHACGDRASTHPYGNGQARSLKQESH

SEQ ID NO: 396 - protein, 364 aa, synthetic construct
MLRTYKFRIEPTKEQRERINETLMYCRWLYNALLEQRITAYKKCGVSLTFYSQKKELPALKKVCEEYKDVHSQVLQNVVERLDKAYQAFFRRLKRGEKAGVPRFKGMNRYHSFTYPQSGFSLEGKYITLSKVGTVRIKLHRQVQGIIKTCTVIAKNGRYYVCLSCEMETKTQSTGKKIGVDLGLQHLAITSDGDFYHVPKYLRKSERQLKKLQRIVSRRKKGSNRRKKAVALLAKKHEYIANQRRNNAHQISRQLVNRYDLIAFEDLKITNMVKNHHLAKSIADAGWNQLVQFTTYKAESAGKRVVLVDPRNTSQQCSSCDEIVKKTLAVRTHHCPSCGFECDRDVNAAKNILKRAVQQLQNVS

SEQ ID NO: 397 - protein, 407 aa, synthetic construct
MQRAFKVTLIPNHNQEVLINKSIGCARFVYNHFLALKQELYQSEQKTLNYNACSQRLTLLKKEIEWLKEVDKFALQNSLKNLETAYKNFFADLKKVKGKKGVGFPKFKKKHGCKQSYKTNLTNGNIQVIENRLKLPKLGWVKFHKSQEVTGKLINVTITRTSSGKYVASILCDTEIEKHPQVSQNIGIDLGIKSYLVTSNGEVVDNPKYYRTQTRKLRKAHKKLSRSVKGSTNRVKAKIKLARSYERITNLRDDFLHKLSTRLIKENSIICIEDLRVANMVKNHKLSLSISDASWSKFVAMLEYKAFWHDRIVQKVGTFYPSSQTCNHCGFINPLVKDLKLREWACPGCSGYNLRDNNAALNILGEGLRLIAAVGIPEALNACGELVSPGAIQAEIVEAGITRLQVV

SEQ ID NO: 398 - protein, 413 aa, synthetic construct
MQRLQAYKYELMPNGEQERDMRRFAGSCRFVYNKALAMQQENHEAGNKFIGYVAMAKQLTGWRNGPDTPWLKEAPCHPLQHALKDLEKAYKNFFEKRADFPKFKRKGSGDTFRYPDSKQIKLDQGNGRIFLPKLGWLRYRNSRDVLGEVRNVTVSQSGGKWFASIQTQREIKQPLPTATTAIGIDVGISRFATMSDGAFIAPLNSFKKHQQRLARYQRRMSRKVKFSSNWKKAKARVQKIHTRIANARKDFLHKTTTTISQNHALVCIEDLQVRNMSKSSKGNSEQPGKMVKQKSGLNRSILDQGWGEFRRQLAYKVAWHGGMLLAVPPHHTSQTCPACGHVSKDNRQTQAKFLCVDCGYENHADMVGAINILERGYRLLACGESEQSGRSKKQEPTEATRKSSRSAVGIPVL

SEQ ID NO: 399 - protein, 389 aa, synthetic construct
MRKTYKYRVYPSRDQESRLLETSETCRVLYNSCLVDRKNHYQSTGKGLSRIRQQEILAADKKRVLLLKEIHSQVLQDVLFRVERAYQGFFRRLQERSGKAGYPRFKSEGRYDSITFPQEPGFRIEDGKLKLSKIGQLKIKMHRQIVGTVKTCTVRKDGTHWYACFSAEYEPTPRPIPLKAVGIDVGLKSFATLSDGSEIANPKHLRKAESSLKRVQRSLSRKKKGGKNRKKAKVVVANLHRKVRNQRSDFHHKTARAIVDANGLIVAEDLKIKNMVRNRHLSKSISDAGWGNFLLILASKAEEAGCRFEKVKPHHTSINCSCCGTAVLKTLATRTHRCHVCGLVLDRDHNAAINILKRATAGTAESYAWGESVLSDLSMIQEAPSLAAG

SEQ ID NO: 400 - DNA, 203 bp, synthetic construct
  source 1..203: mol_type = other DNA
gaacagaaagcatttttgagggagacaaccccttttagaagccctagggccggggcggtccgaagtcacgcctgtggagatcgagcctctactcatccctacgggaatgggcaagctcggtcgctgaagcaggaatcccactagcgggttttccgggtgggcaacctaccggaagccccttccgtaagggaggggtagttcac

SEQ ID NO: 401 - DNA, 203 bp, synthetic construct
  source 1..203: mol_type = other DNA
gtacggacacatcattgcccttcttgtggatttgaatgtgaccgtgatgtaaatgccgccaaaaacattctcaaacgggcagtacaacaactacaaaacgtgtcgtaagacacgctaggctaggaactagccttcgtggagagtgcgacaatagtcggctcgatgaagcgagaagctctccaatttattggagagaggttcac

SEQ ID NO: 402 - DNA, 201 bp, synthetic construct
  source 1..201: mol_type = other DNA
gggcttgtcctggttgtagtggttacaatttgagagacaataacgcagcgttgaacatattaggtgagggattgagattaatagccgccgtgggtatcccggaggctctaaacgcctgtggagaactcgtcagtcctggagcaattcaggcagagatcgttgaagcaggaatcacgcgacttcaagtcgtgtgaggttcaa

SEQ ID NO: 403 - DNA, 209 bp, synthetic construct
  source 1..209: mol_type = other DNA
gcaaaattcctgtgcgtcgattgcggttacgaaaatcacgccgatatggtcggtgcgatcaatattttagagcggggataccgcttgttagcctgtggagagtcggagcagtcaggccgctcgaagaagcaggaacccaccgaagcgactcgcaaatcatcgcgcagcgccgtaggaatccccgtcctttagggcggggaggatgtcaa

SEQ ID NO: 404 - DNA, 205 bp, synthetic construct
  source 1..205: mol_type = other DNA
gcactgcagtactaaagacgctggcaaccagaacacatcggtgccatgtttgcggtctggttctggatcgagaccacaacgctgcaataaacattctgaaaagagctaccgcaggaactgcggaaagttacgcctggggagagtcggtgctgtcagacctctcgatgatccaggaagccccgtcgcttgcggcggggtagttcac

SEQ ID NO: 405 - DNA, 588 bp, synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 406 - DNA, 27 bp, synthetic construct
  source 1..27: mol_type = other DNA
ggatctccaaaaaagaaacggaaggtg

SEQ ID NO: 407 - DNA, 48 bp, synthetic construct
  source 1..48: mol_type = other DNA
aaaaggccggcggccacgaaaaaggccggccaggcaaaaaagaaaaag

SEQ ID NO: 408 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 409 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 410 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 411 - DNA, 620 bp, synthetic construct
  source 1..620: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

SEQ ID NO: 412 - DNA, 588 bp, synthetic construct
  source 1..588: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagat

SEQ ID NO: 413 - DNA, 675 bp, synthetic construct
  source 1..675: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttccagtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcc

SEQ ID NO: 414 - DNA, 718 bp, synthetic construct
  source 1..718: mol_type = other DNA
agtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 415 - DNA, 20 bp, synthetic construct
  source 1..20: mol_type = other DNA
attcacagattgtaacttat

SEQ ID NO: 416 - DNA, 20 bp, synthetic construct
  source 1..20: mol_type = other DNA
ttgtagagcctgacctttaa

SEQ ID NO: 417 - DNA, 20 bp, synthetic construct
  source 1..20: mol_type = other DNA
gtacaccccaaatgcccaat

SEQ ID NO: 418 - DNA, 20 bp, synthetic construct
  source 1..20: mol_type = other DNA
gatttccctgacagcagcaa

SEQ ID NO: 419 - DNA, 20 bp, synthetic construct
  source 1..20: mol_type = other DNA
gagcatgcagaggtgagtat

SEQ ID NO: 420 - DNA, 20 bp, synthetic construct
  source 1..20: mol_type = other DNA
tctctacccccaacccttac

SEQ ID NO: 421 - DNA, 20 bp, synthetic construct
  source 1..20: mol_type = other DNA
cctctcccagaacctgagct

SEQ ID NO: 422 - DNA, 20 bp, synthetic construct
  source 1..20: mol_type = other DNA
ccagcaggaacaagcccttc

SEQ ID NO: 423 - DNA, 20 bp, synthetic construct
  source 1..20: mol_type = other DNA
ctctctagtctgtgctagct

SEQ ID NO: 424 - DNA, 20 bp, synthetic construct
  source 1..20: mol_type = other DNA
gcctgtaatcccagcactta

SEQ ID NO: 425 - DNA, 20 bp, synthetic construct
  source 1..20: mol_type = other DNA
agaattgcataggggacagg

SEQ ID NO: 426 - DNA, 20 bp, synthetic construct
  source 1..20: mol_type = other DNA
tggaaggcacttggcatctc

SEQ ID NO: 427 - DNA, 20 bp, synthetic construct
  source 1..20: mol_type = other DNA
agccaaagccttcaagtaca

SEQ ID NO: 428 - DNA, 20 bp, synthetic construct
  source 1..20: mol_type = other DNA
gagcaacagtgctgtggcct

SEQ ID NO: 429 - DNA, 20 bp, synthetic construct
  source 1..20: mol_type = other DNA
ttcgcgcacctactccaagt

SEQ ID NO: 430 - DNA, 20 bp, synthetic construct
  source 1..20: mol_type = other DNA
tgggagagggagagtgcttc

SEQ ID NO: 431 - DNA, 20 bp, synthetic construct
  source 1..20: mol_type = other DNA
agagggtggatattcatgct

SEQ ID NO: 432 - DNA, 20 bp, synthetic construct
  source 1..20: mol_type = other DNA
gaagacaaacatttctattt

SEQ ID NO: 433 - DNA, 20 bp, synthetic construct
  source 1..20: mol_type = other DNA
agagtcaccttccaaattac

SEQ ID NO: 434 - DNA, 20 bp, synthetic construct
  source 1..20: mol_type = other DNA
ggtttgcagtgtcttttagg

SEQ ID NO: 435 - DNA, 20 bp, synthetic construct
  source 1..20: mol_type = other DNA
cttacagccacttgaactct

SEQ ID NO: 436 - DNA, 20 bp, synthetic construct
  source 1..20: mol_type = other DNA
gtaggtgatgaaaagtaaac

SEQ ID NO: 437 - DNA, 20 bp, synthetic construct
  source 1..20: mol_type = other DNA
cacaggctccaggaagggtt

SEQ ID NO: 438 - DNA, 20 bp, synthetic construct
  source 1..20: mol_type = other DNA
ggttctgtcacaggatgtct

SEQ ID NO: 439 - DNA, 20 bp, synthetic construct
  source 1..20: mol_type = other DNA
ggcattggccacgaagaaaa

SEQ ID NO: 440 - DNA, 20 bp, synthetic construct
  source 1..20: mol_type = other DNA
ctcacaggtaaaactgacgc

SEQ ID NO: 441 - DNA, 20 bp, synthetic construct
  source 1..20: mol_type = other DNA
cttagacaccttttattgaa

SEQ ID NO: 442 - DNA, 20 bp, synthetic construct
  source 1..20: mol_type = other DNA
gtaagataacctggaaagtt

SEQ ID NO: 443 - DNA, 20 bp, synthetic construct
  source 1..20: mol_type = other DNA
cttagacaccttttattgaa

SEQ ID NO: 444 - DNA, 20 bp, synthetic construct
  source 1..20: mol_type = other DNA
cctgctaagaataccaggat

SEQ ID NO: 445 - DNA, 20 bp, synthetic construct
  source 1..20: mol_type = other DNA
gtaagataacctggaaagtt

SEQ ID NO: 446 - DNA, 20 bp, synthetic construct
  source 1..20: mol_type = other DNA
aagtgacatgtgatgggaac

SEQ ID NO: 447 - DNA, 20 bp, synthetic construct
  source 1..20: mol_type = other DNA
gtaatacaatgttcttgtca

SEQ ID NO: 448 - DNA, 20 bp, synthetic construct
  source 1..20: mol_type = other DNA
cctgctaagaataccaggat

SEQ ID NO: 449 - DNA, 20 bp, synthetic construct
  source 1..20: mol_type = other DNA
atcccttagttaatatagca

SEQ ID NO: 450 - DNA, 20 bp, synthetic construct
  source 1..20: mol_type = other DNA
aagtgacatgtgatgggaac

SEQ ID NO: 451 - DNA, 20 bp, synthetic construct
  source 1..20: mol_type = other DNA
cttagacaccttttattgaa

SEQ ID NO: 452 - DNA, 20 bp, synthetic construct
  source 1..20: mol_type = other DNA
gtaagataacctggaaagtt

SEQ ID NO: 453 - DNA, 20 bp, synthetic construct
  source 1..20: mol_type = other DNA
gagcctttctctggaggctc

SEQ ID NO: 454 - DNA, 20 bp, synthetic construct
  source 1..20: mol_type = other DNA
cctgctaagaataccaggat

SEQ ID NO: 455 - DNA, 20 bp, synthetic construct
  source 1..20: mol_type = other DNA
atcccttagttaatatagca

SEQ ID NO: 456 - DNA, 20 bp, synthetic construct
  source 1..20: mol_type = other DNA
ctttcatactgctcaagtgt

SEQ ID NO: 457 - DNA, 20 bp, synthetic construct
  source 1..20: mol_type = other DNA
cagggaagcgtatttcctca

SEQ ID NO: 458 - DNA, 20 bp, synthetic construct
  source 1..20: mol_type = other DNA
gtaatacaatgttcttgtca

SEQ ID NO: 459 - DNA, 20 bp, synthetic construct
  source 1..20: mol_type = other DNA
ttcctccacttcaacacctg

SEQ ID NO: 460 - DNA, 20 bp, synthetic construct
  source 1..20: mol_type = other DNA
gattcacgctaaatgaagta

SEQ ID NO: 461 - DNA, 20 bp, synthetic construct
  source 1..20: mol_type = other DNA
cacgagacccccacggggct

SEQ ID NO: 462 - DNA, 20 bp, synthetic construct
  source 1..20: mol_type = other DNA
cttagacaccttttattgaa

SEQ ID NO: 463 - DNA, 20 bp, synthetic construct
  source 1..20: mol_type = other DNA
tgtgtctaaggttagcagtg

SEQ ID NO: 464 - DNA, 20 bp, synthetic construct
  source 1..20: mol_type = other DNA
gagaaggaaaaagggtggac

SEQ ID NO: 465 - DNA, 20 bp, synthetic construct
  source 1..20: mol_type = other DNA
gtaagataacctggaaagtt

SEQ ID NO: 466 - DNA, 20 bp, synthetic construct
  source 1..20: mol_type = other DNA
ttcctccacttcaacacctg

SEQ ID NO: 467 - DNA, 20 bp, synthetic construct
  source 1..20: mol_type = other DNA
gagcctttctctggaggctc

SEQ ID NO: 468 - DNA, 20 bp, synthetic construct
  source 1..20: mol_type = other DNA
cctgctaagaataccaggat

SEQ ID NO: 469 - DNA, 20 bp, synthetic construct
  source 1..20: mol_type = other DNA
cttagacaccttttattgaa

SEQ ID NO: 470 - DNA, 20 bp, synthetic construct
  source 1..20: mol_type = other DNA
gttatccataaaagaattcc

SEQ ID NO: 471 - DNA, 20 bp, synthetic construct
  source 1..20: mol_type = other DNA
gagaaggaaaaagggtggac

SEQ ID NO: 472 - DNA, 20 bp, synthetic construct
  source 1..20: mol_type = other DNA
atcccttagttaatatagca

SEQ ID NO: 473 - DNA, 20 bp, synthetic construct
  source 1..20: mol_type = other DNA
ttcctccacttcaacacctg

SEQ ID NO: 474 - DNA, 20 bp, synthetic construct
  source 1..20: mol_type = other DNA
aagtgacatgtgatgggaac

SEQ ID NO: 475 - DNA, 20 bp, synthetic construct
  source 1..20: mol_type = other DNA
gtaatacaatgttcttgtca

SEQ ID NO: 476 - DNA, 20 bp, synthetic construct
  source 1..20: mol_type = other DNA
atcccttagttaatatagca

SEQ ID NO: 477 - DNA, 20 bp, synthetic construct
  source 1..20: mol_type = other DNA
cttagacaccttttattgaa

SEQ ID NO: 478 - DNA, 20 bp, synthetic construct
  source 1..20: mol_type = other DNA
gtaagataacctggaaagtt

SEQ ID NO: 479 - DNA, 20 bp, synthetic construct
  source 1..20: mol_type = other DNA
aacttggggagtcataatag

SEQ ID NO: 480 - DNA, 20 bp, synthetic construct
  source 1..20: mol_type = other DNA
ttcctccacttcaacacctg

SEQ ID NO: 481 - DNA, 20 bp, synthetic construct
  source 1..20: mol_type = other DNA
cctgctaagaataccaggat

SEQ ID NO: 482 - DNA, 20 bp, synthetic construct
  source 1..20: mol_type = other DNA
gattcacgctaaatgaagta

SEQ ID NO: 483 - DNA, 20 bp, synthetic construct
  source 1..20: mol_type = other DNA
tataagtggaggcgtcgcgc

SEQ ID NO: 484 - DNA, 20 bp, synthetic construct
  source 1..20: mol_type = other DNA
atcccttagttaatatagca

SEQ ID NO: 485 - DNA, 20 bp, synthetic construct
  source 1..20: mol_type = other DNA
cttagacaccttttattgaa

SEQ ID NO: 486 - DNA, 20 bp, synthetic construct
  source 1..20: mol_type = other DNA
gtaagataacctggaaagtt

SEQ ID NO: 487 - DNA, 20 bp, synthetic construct
  source 1..20: mol_type = other DNA
ttcctccacttcaacacctg

SEQ ID NO: 488 - DNA, 20 bp, synthetic construct
  source 1..20: mol_type = other DNA
ttgaaccccaagaaccacat

SEQ ID NO: 489 - DNA, 20 bp, synthetic construct
  source 1..20: mol_type = other DNA
gagcctttctctggaggctc

SEQ ID NO: 490 - DNA, 20 bp, synthetic construct
  source 1..20: mol_type = other DNA
gtaatacaatgttcttgtca

SEQ ID NO: 491 - DNA, 20 bp, synthetic construct
  source 1..20: mol_type = other DNA
cctgctaagaataccaggat

SEQ ID NO: 492 - DNA, 20 bp, synthetic construct
  source 1..20: mol_type = other DNA
atcccttagttaatatagca

SEQ ID NO: 493 - DNA, 20 bp, synthetic construct
  source 1..20: mol_type = other DNA
cttagacaccttttattgaa

SEQ ID NO: 494 - DNA, 20 bp, synthetic construct
  source 1..20: mol_type = other DNA
aagtgacatgtgatgggaac

SEQ ID NO: 495 - DNA, 20 bp, synthetic construct
  source 1..20: mol_type = other DNA
gtaatacaatgttcttgtca

SEQ ID NO: 496 - DNA, 20 bp, synthetic construct
  source 1..20: mol_type = other DNA
gtaagataacctggaaagtt

SEQ ID NO: 497 - DNA, 20 bp, synthetic construct
  source 1..20: mol_type = other DNA
ttcctccacttcaacacctg

SEQ ID NO: 498 - DNA, 20 bp, synthetic construct
  source 1..20: mol_type = other DNA
gattcacgctaaatgaagta

SEQ ID NO: 499 - DNA, 20 bp, synthetic construct
  source 1..20: mol_type = other DNA
cctgctaagaataccaggat

SEQ ID NO: 500 - DNA, 20 bp, synthetic construct
  source 1..20: mol_type = other DNA
gagaaggaaaaagggtggac

SEQ ID NO: 501 - DNA, 20 bp, synthetic construct
  source 1..20: mol_type = other DNA
gatttccctgacagcagcaa

SEQ ID NO: 502 - DNA, 20 bp, synthetic construct
  source 1..20: mol_type = other DNA
attcacagattgtaacttat

SEQ ID NO: 503 - DNA, 20 bp, synthetic construct
  source 1..20: mol_type = other DNA
gtacaccccaaatgcccaat

SEQ ID NO: 504 - DNA, 20 bp, synthetic construct
  source 1..20: mol_type = other DNA
cgtaagcaaaccttagaggt

SEQ ID NO: 505 - DNA, 20 bp, synthetic construct
  source 1..20: mol_type = other DNA
gatgctgcttacatgtctcg

SEQ ID NO: 506 - DNA, 20 bp, synthetic construct
  source 1..20: mol_type = other DNA
ttgtagagcctgacctttaa

SEQ ID NO: 507 - DNA, 20 bp, synthetic construct
  source 1..20: mol_type = other DNA
gagcatgcagaggtgagtat

SEQ ID NO: 508 - DNA, 20 bp, synthetic construct
  source 1..20: mol_type = other DNA
tgcggtgcagaaaaccacat

SEQ ID NO: 509 - DNA, 20 bp, synthetic construct
  source 1..20: mol_type = other DNA
agacctcatgtctagcacag

SEQ ID NO: 510 - DNA, 20 bp, synthetic construct
  source 1..20: mol_type = other DNA
gtacaccccaaatgcccaat

SEQ ID NO: 511 - DNA, 20 bp, synthetic construct
  source 1..20: mol_type = other DNA
gatttccctgacagcagcaa

SEQ ID NO: 512 - DNA, 20 bp, synthetic construct
  source 1..20: mol_type = other DNA
attcacagattgtaacttat

SEQ ID NO: 513 - DNA, 20 bp, synthetic construct
  source 1..20: mol_type = other DNA
cgtaagcaaaccttagaggt

SEQ ID NO: 514 - DNA, 20 bp, synthetic construct
  source 1..20: mol_type = other DNA
gagcatgcagaggtgagtat

SEQ ID NO: 515 - DNA, 20 bp, synthetic construct
  source 1..20: mol_type = other DNA
ttgtagagcctgacctttaa

SEQ ID NO: 516 - DNA, 20 bp, synthetic construct
  source 1..20: mol_type = other DNA
tctctgctggatgacgtgag

SEQ ID NO: 517 - DNA, 20 bp, synthetic construct
  source 1..20: mol_type = other DNA
gacaagaacattgtattact

SEQ ID NO: 518 - DNA, 20 bp, synthetic construct
  source 1..20: mol_type = other DNA
gatttccctgacagcagcaa

SEQ ID NO: 519 - DNA, 20 bp, synthetic construct
  source 1..20: mol_type = other DNA
ttgtagagcctgacctttaa

SEQ ID NO: 520 - DNA, 20 bp, synthetic construct
  source 1..20: mol_type = other DNA
gacaagaacattgtattact

SEQ ID NO: 521 - DNA, 20 bp, synthetic construct
  source 1..20: mol_type = other DNA
gtacaccccaaatgcccaat

SEQ ID NO: 522 - DNA, 20 bp, synthetic construct
  source 1..20: mol_type = other DNA
gatgctgcttacatgtctcg

SEQ ID NO: 523 - DNA, 20 bp, synthetic construct
  source 1..20: mol_type = other DNA
gagcatgcagaggtgagtat

SEQ ID NO: 524 - DNA, 20 bp, synthetic construct
  source 1..20: mol_type = other DNA
cgtaagcaaaccttagaggt

SEQ ID NO: 525 - DNA, 20 bp, synthetic construct
  source 1..20: mol_type = other DNA
ggtccaggaagagcccttaa

SEQ ID NO: 526 - DNA, 20 bp, synthetic construct
  source 1..20: mol_type = other DNA
gatttccctgacagcagcaa

SEQ ID NO: 527 - DNA, 20 bp, synthetic construct
  source 1..20: mol_type = other DNA
gacaagaacattgtattact

SEQ ID NO: 528 - DNA, 20 bp, synthetic construct
  source 1..20: mol_type = other DNA
gtacaccccaaatgcccaat

SEQ ID NO: 529 - DNA, 20 bp, synthetic construct
  source 1..20: mol_type = other DNA
ggtccaggaagagcccttaa

SEQ ID NO: 530 - DNA, 20 bp, synthetic construct
  source 1..20: mol_type = other DNA
agacctcatgtctagcacag

SEQ ID NO: 531 - DNA, 20 bp, synthetic construct
  source 1..20: mol_type = other DNA
gatgctgcttacatgtctcg

SEQ ID NO: 532 - DNA, 20 bp, synthetic construct
  source 1..20: mol_type = other DNA
gggtagacaaccaggagcct

SEQ ID NO: 533 - DNA, 20 bp, synthetic construct
  source 1..20: mol_type = other DNA
gagcatgcagaggtgagtat

SEQ ID NO: 534 - DNA, 20 bp, synthetic construct
  source 1..20: mol_type = other DNA
cgtaagcaaaccttagaggt

SEQ ID NO: 535 - DNA, 20 bp, synthetic construct
  source 1..20: mol_type = other DNA
tgtctttcctagcggaatgg

SEQ ID NO: 536 - DNA, 20 bp, synthetic construct
  source 1..20: mol_type = other DNA
attcacagattgtaacttat

SEQ ID NO: 537 - DNA, 20 bp, synthetic construct
  source 1..20: mol_type = other DNA
ttgtagagcctgacctttaa

SEQ ID NO: 538 - DNA, 20 bp, synthetic construct
  source 1..20: mol_type = other DNA
gacaagaacattgtattact

SEQ ID NO: 539 - DNA, 20 bp, synthetic construct
  source 1..20: mol_type = other DNA
gtacaccccaaatgcccaat

SEQ ID NO: 540 - DNA, 20 bp, synthetic construct
  source 1..20: mol_type = other DNA
gatgctgcttacatgtctcg

SEQ ID NO: 541 - DNA, 20 bp, synthetic construct
  source 1..20: mol_type = other DNA
gatttccctgacagcagcaa

SEQ ID NO: 542 - DNA, 20 bp, synthetic construct
  source 1..20: mol_type = other DNA
cgtaagcaaaccttagaggt

SEQ ID NO: 543 - DNA, 20 bp, synthetic construct
  source 1..20: mol_type = other DNA
agacctcatgtctagcacag

SEQ ID NO: 544 - DNA, 20 bp, synthetic construct
  source 1..20: mol_type = other DNA
gagcatgcagaggtgagtat

SEQ ID NO: 545 - DNA, 20 bp, synthetic construct
  source 1..20: mol_type = other DNA
tgtctttcctagcggaatgg

SEQ ID NO: 546 - DNA, 20 bp, synthetic construct
  source 1..20: mol_type = other DNA
attcacagattgtaacttat

SEQ ID NO: 547 - DNA, 20 bp, synthetic construct
  source 1..20: mol_type = other DNA
gatttccctgacagcagcaa

SEQ ID NO: 548 - DNA, 20 bp, synthetic construct
  source 1..20: mol_type = other DNA
gacaagaacattgtattact

SEQ ID NO: 549 - DNA, 20 bp, synthetic construct
  source 1..20: mol_type = other DNA
gagcatgcagaggtgagtat

SEQ ID NO: 550 - DNA, 20 bp, synthetic construct
  source 1..20: mol_type = other DNA
agacctcatgtctagcacag

SEQ ID NO: 551 - DNA, 20 bp, synthetic construct
  source 1..20: mol_type = other DNA
cgtaagcaaaccttagaggt

SEQ ID NO: 552 - DNA, 20 bp, synthetic construct
  source 1..20: mol_type = other DNA
gatgctgcttacatgtctcg

SEQ ID NO: 553 - DNA, 20 bp, synthetic construct
  source 1..20: mol_type = other DNA
gtacaccccaaatgcccaat

SEQ ID NO: 554 - DNA, 20 bp, synthetic construct
  source 1..20: mol_type = other DNA
ttgtagagcctgacctttaa

SEQ ID NO: 555 - DNA, 20 bp, synthetic construct
  source 1..20: mol_type = other DNA
gatttccctgacagcagcaa

SEQ ID NO: 556 - DNA, 20 bp, synthetic construct
  source 1..20: mol_type = other DNA
attcacagattgtaacttat

SEQ ID NO: 557 - DNA, 20 bp, synthetic construct
  source 1..20: mol_type = other DNA
gtacaccccaaatgcccaat

SEQ ID NO: 558 - DNA, 20 bp, synthetic construct
  source 1..20: mol_type = other DNA
gatgctgcttacatgtctcg

SEQ ID NO: 559 - DNA, 20 bp, synthetic construct
  source 1..20: mol_type = other DNA
cgtaagcaaaccttagaggt

SEQ ID NO: 560 - DNA, 20 bp, synthetic construct
  source 1..20: mol_type = other DNA
gggtagacaaccaggagcct

SEQ ID NO: 561 - DNA, 20 bp, synthetic construct
  source 1..20: mol_type = other DNA
gacaagaacattgtattact

SEQ ID NO: 562 - DNA, 20 bp, synthetic construct
  source 1..20: mol_type = other DNA
gagcatgcagaggtgagtat

SEQ ID NO: 563 - DNA, 20 bp, synthetic construct
  source 1..20: mol_type = other DNA
agacctcatgtctagcacag

SEQ ID NO: 564 - DNA, 20 bp, synthetic construct
  source 1..20: mol_type = other DNA
tgtctttcctagcggaatgg

SEQ ID NO: 565 - DNA, 20 bp, synthetic construct
  source 1..20: mol_type = other DNA
ctcacaggtaaaactgacgc

SEQ ID NO: 566 - DNA, 20 bp, synthetic construct
  source 1..20: mol_type = other DNA
gatatgactactcatacaca

SEQ ID NO: 567 - DNA, 20 bp, synthetic construct
  source 1..20: mol_type = other DNA
cacaggctccaggaagggtt

SEQ ID NO: 568 - DNA, 20 bp, synthetic construct
  source 1..20: mol_type = other DNA
cccacttctcctcctacaga

SEQ ID NO: 569 - DNA, 20 bp, synthetic construct
  source 1..20: mol_type = other DNA
ttgatgaccacattgcttca

SEQ ID NO: 570 - DNA, 20 bp, synthetic construct
  source 1..20: mol_type = other DNA
agagtcaccttccaaattac

SEQ ID NO: 571 - DNA, 20 bp, synthetic construct
  source 1..20: mol_type = other DNA
gtaggtgatgaaaagtaaac

SEQ ID NO: 572 - DNA, 20 bp, synthetic construct
  source 1..20: mol_type = other DNA
cccacttctcctcctacaga

SEQ ID NO: 573 - DNA, 20 bp, synthetic construct
  source 1..20: mol_type = other DNA
attgttcctccgtgcgtcag

SEQ ID NO: 574 - DNA, 20 bp, synthetic construct
  source 1..20: mol_type = other DNA
agagtcaccttccaaattac

SEQ ID NO: 575 - DNA, 20 bp, synthetic construct
  source 1..20: mol_type = other DNA
cacaggctccaggaagggtt

SEQ ID NO: 576 - DNA, 20 bp, synthetic construct
  source 1..20: mol_type = other DNA
ttgatgaccacattgcttca

SEQ ID NO: 577 - DNA, 20 bp, synthetic construct
  source 1..20: mol_type = other DNA
aggaaaaccagtgagtctgg

SEQ ID NO: 578 - DNA, 20 bp, synthetic construct
  source 1..20: mol_type = other DNA
gatatgactactcatacaca

SEQ ID NO: 579 - DNA, 20 bp, synthetic construct
  source 1..20: mol_type = other DNA
agagtcaccttccaaattac

SEQ ID NO: 580 - DNA, 20 bp, synthetic construct
  source 1..20: mol_type = other DNA
cacaggctccaggaagggtt

SEQ ID NO: 581 - DNA, 20 bp, synthetic construct
  source 1..20: mol_type = other DNA
ctgtcccctccaccccacag

SEQ ID NO: 582 - DNA, 20 bp, synthetic construct
  source 1..20: mol_type = other DNA
ttgatgaccacattgcttca

SEQ ID NO: 583 - DNA, 20 bp, synthetic construct
  source 1..20: mol_type = other DNA
cccacttctcctcctacaga

SEQ ID NO: 584 - DNA, 20 bp, synthetic construct
  source 1..20: mol_type = other DNA
gtaggtgatgaaaagtaaac

SEQ ID NO: 585 - DNA, 20 bp, synthetic construct
  source 1..20: mol_type = other DNA
gaaaaaaatcaggtcttcat

SEQ ID NO: 586 - DNA, 20 bp, synthetic construct
  source 1..20: mol_type = other DNA
ttgatgaccacattgcttca

SEQ ID NO: 587 - DNA, 20 bp, synthetic construct
  source 1..20: mol_type = other DNA
cacaggctccaggaagggtt

SEQ ID NO: 588 - DNA, 20 bp, synthetic construct
  source 1..20: mol_type = other DNA
agagtcaccttccaaattac

SEQ ID NO: 589 - DNA, 20 bp, synthetic construct
  source 1..20: mol_type = other DNA
cccacttctcctcctacaga

SEQ ID NO: 590 - DNA, 20 bp, synthetic construct
  source 1..20: mol_type = other DNA
gatatgactactcatacaca

SEQ ID NO: 591 - DNA, 20 bp, synthetic construct
  source 1..20: mol_type = other DNA
gtaggtgatgaaaagtaaac

SEQ ID NO: 592 - DNA, 20 bp, synthetic construct
  source 1..20: mol_type = other DNA
ctgtcccctccaccccacag

SEQ ID NO: 593 - DNA, 1985 bp, synthetic construct
  source 1..1985: mol_type = other DNA
tgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 594 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 595 - DNA, 242 bp, synthetic construct
  source 1..242: mol_type = other DNA
ggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttg

SEQ ID NO: 596 - DNA, 20 bp, synthetic construct
  source 1..20: mol_type = other DNA
gctcggagatcatcattgcg

SEQ ID NO: 597 - DNA, 20 bp, synthetic construct
  source 1..20: mol_type = other DNA
tttttttgttttttatgtct

SEQ ID NO: 598 - DNA, 967 bp, synthetic construct
  source 1..967: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagcagcacgacttcttcaagtccgccatgcccgaaggctacgtccaggagcgcatgccccgggtaccggcgcgccgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcaatgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 599 - DNA, 346 bp, synthetic construct
  source 1..346: mol_type = other DNA
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaca

SEQ ID NO: 600 - DNA, 247 bp, synthetic construct
  source 1..247: mol_type = other DNA
cgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag